(19) United States
(12) Patent Application Publication
(10) Pub. No.: US 2014/0328930 A1
(43) Pub. Date: Nov. 6, 2014

(54) Pharmaceutical Compositions

(71) Applicant: Alpharma Pharmaceuticals, LLC, Bridgewater, NJ (US)

(72) Inventors: Alfred Liang, Edison, NJ (US); Joseph Stauffer, Skillman, NJ (US); James Jones, Somerset, NJ (US)

(73) Assignee: Alpharma Pharmaceuticals, LLC, Bridgewater, NJ (US)

(21) Appl. No.: 14/151,185

(22) Filed: Jan. 9, 2014

Related U.S. Application Data
(63) Continuation of application No. 12/204,280, filed on Sep. 4, 2008, now abandoned.
(60) Provisional application No. 60/967,365, filed on Sep. 4, 2007.
(60) Provisional application No. 61/007,941, filed on Dec. 17, 2007.

(51) Int. Cl. A61K 9/16; A61K 31/485

(52) U.S. Cl. CPC A61K 9/167; A61K 31/485
USPC 424/490; 514/282

(57) ABSTRACT

Provided herein is a pharmaceutical composition comprising an antagonist, an agonist, a seal coat, and a sequestering polymer, wherein the antagonist, agonist, seal coat and at least one sequestering polymer are all components of a single unit, and wherein the seal coat forms a layer physically separating the antagonist from the agonist from one another. Methods for manufacturing such a pharmaceutical composition are also provided.

### RELATED APPLICATIONS

This application is a continuation of U.S. application Ser. No. 12/204,280, filed Sep. 4, 2008; which claims priority under 37 U.S.C. §119(e) to U.S. Provisional Patent Application Nos. 61/007,941, filed Dec. 17, 2007 and 60/967,365 filed Sep. 4, 2007, the contents of each which are hereby incorporated by reference.

### FIELD OF THE INVENTION

This invention pertains to a sequestering subunit comprising an antagonist and a blocking agent, and related compositions and methods of use, such as in the prevention of abuse of a therapeutic agent.

### BACKGROUND OF THE INVENTION

Opioids, also called opioid agonists, are a class of drugs that exhibit opium-like or morphine-like properties. The opioids are employed primarily as moderate to strong analgesics, but have many other pharmacological effects as well, including drowsiness, respiratory depression, changes in mood, and mental clouding without a resulting loss of consciousness. Because of these other pharmacological effects, opioids have become the subject of dependence and abuse. Therefore, a major concern associated with the use of opioids is the diversion of these drugs from the illicit user, e.g., an addict.

Physical dependence may develop upon repeated administrations or extended use of opioids. Physical dependence is gradually manifested after stopping opioid use or is precipitously manifested (e.g., within a few minutes) after administration of a narcotic antagonist (referred to “precipitated withdrawal”). Depending upon the drug upon which dependence has been established and the duration of use and dose, symptoms of withdrawal vary in number and kind, duration and severity. The most common symptoms of the withdrawal syndrome include anorexia, weight loss, pupillary dilation, chills alternating with excessive sweating, abdominal cramps, nausea, vomiting, muscle spasms, hyperirritability, lacrimation, rinorrhea, goose flesh and increased heart rate. Natural abstinence syndromes typically begin to occur 24-48 hours after the last dose, reach maximum intensity about the third day and may not begin to decrease until the third week. Precipitated abstinence syndromes produced by administration of an opioid antagonist vary in intensity and duration with the dose and the specific antagonist, but generally vary from a few minutes to several hours in length.

Psychological dependence or addiction to opioids is characterized by drug-seeking behavior directed toward achieving euphoria and escape from, e.g., psychosocioeconomic pressures. An addict will continue to administer opioids for non-medicinal purposes and in the face of self-harm.

Although opioids, such as morphine, hydromorphone, hydrocodone and oxycodone, are effective in the management of pain, there has been an increase in their abuse by individuals who are psychologically dependent on opioids or who misuse opioids for non-therapeutic reasons. Previous experience with other opioids has demonstrated a decreased abuse potential when opioids are administered in combination with a narcotic antagonist, especially in patients who are ex-addicts (Weinhold et al., Drug and Alcohol Dependence 30:263-274 (1992); and Mendelson et al., Clin. Pharm. Ther. 60:105-114 (1996)). These combinations, however, do not contain the opioid antagonist that is in a sequestered form. Rather, the opioid antagonist is released in the gastrointestinal system when orally administered and is made available for absorption, relying on the physiology of the host to metabolize differentially the agonist and antagonist and negate the agonist effects.

Previous attempts to control the abuse potential associated with opioid analgesics include, for example, the combination of pentazocine and naloxone in tablets, commercially available in the United States as Talwin®Nx from Sanofi-Winthrop, Canterbury, Australia. Talwin®Nx contains pentazocine hydrochloride equivalent to 50 mg base and naloxone hydrochloride equivalent to 0.5 mg base. Talwin®Nx is indicated for the relief of moderate to severe pain. The amount of naloxone present in this combination has low activity when taken orally, and minimally interferes with the pharmacologic action of pentazocine. However, this amount of naloxone given parenterally has profound antagonistic action to narcotic analgesics. Thus, the inclusion of naloxone is intended to curb a form of misuse of oral pentazocine, which occurs when the dosage form is solubilized and injected. Therefore, this dosage has lower potential for parenteral misuse than previous oral pentazocine formulations. However, it is still subject to patient misuse and abuse by the oral route, for example, by the patient taking multiple doses at once. A fixed combination therapy comprising tilidine (50 mg) and naloxone (4 mg) has been available in Germany for the management of severe pain since 1978 (Valoron®N, Goedecke). The rationale for the combination of these drugs is effective pain relief and the prevention of tilidine addiction through naloxone-induced antagonisms at the tilidine receptors. A fixed combination of buprenorphine and naloxone was introduced in 1991 in New Zealand (Terngesic®Nx, Reckitt & Colman) for the treatment of pain.

International Patent Application No. PCT/US01/04346 (WO 01/58451) to Euroceltique, S. A., describes the use of a pharmaceutical composition that contains a substantially non-releasing opioid antagonist and a releasing opioid agonist as separate subunits that are combined into a pharmaceutical dosage form, e.g., tablet or capsule. However, because the agonist and antagonist are in separate subunits, they can be readily separated. Further, providing the agonist and antagonist as separate subunits, tablets are more difficult to form due to the mechanical sensitivity of some subunits comprising a sequestering agent.

The benefits of the abuse-resistant dosage form are especially great in connection with oral dosage forms of strong opioid agonists (e.g., morphine, hydromorphone, oxycodone or hydrocodone), which provide valuable analgesics but are prone to being abused. This is particularly true for sustained-release opioid agonist products, which have a large dose of a desirable opioid agonist intended to be released over a period of time in each dosage unit. Drug abusers take such sustained release product and crush, grind, extract or otherwise damage the product so that the full contents of the dosage form become available for immediate absorption.

Such abuse-resistant, sustained-release dosage forms have been described in the art (see, for example, U.S. Application Nos. 2003/0124185 and 2003/0044458). However, it is believed that substantial amounts of the opioid antagonist or other antagonist found in these sequestered forms are released over time (usually less than 24 hours) due to the osmotic pressure that builds up in the core of the sequestered form, as water permeates through the sequestered form into the core. The high osmotic pressure inside the core of the sequestered form causes the opioid antagonist or antagonist to be pushed out of the sequestered form, thereby causing the opioid antagonist or antagonist to be released from the sequestered form.

In view of the foregoing drawbacks of the sequestered forms of the prior art, there exists a need in the art for a sequestered form of an opioid antagonist or other antagonist that is not substantially released from the sequestered form due to osmotic pressure. The invention provides such a sequestering form of an opioid antagonist or antagonist. This and other objects and advantages of the invention, as well as additional inventive features, will be apparent from the description of the invention provided herein.

### BRIEF SUMMARY OF THE INVENTION

Provided herein is a pharmaceutical composition comprising an antagonist, an agonist, a seal coat, and a sequestering polymer, wherein the antagonist, agonist, seal coat and at least one sequestering polymer are all components of a single unit, and wherein the seal coat forms a layer physically separating the antagonist from the agonist from one another. In one embodiment, a multi-layer pharmaceutical composition comprising an agonist and an antagonist thereof, wherein the agonist and antagonist are not in contact with one another in the intact form of the composition, wherein the agonist is substantially released and the antagonist is substantially sequestered upon administration to a human being is provided.

In one embodiment, a multi-layer pharmaceutical composition comprising an antagonist in a first layer and an agonist in a second layer upon said first layer such that the antagonist is substantially sequestered when administered to a human being in an intact form, such that physical disruption of the dosage form decreases the euphoric effect of the agonist when administered to a person as compared to an immediate release agonist composition. In certain embodiments, the euphoric effect is measured by Eₘₐₓ from a standard measurement or test is one or more of VAS-Drug Liking, VAS-Overall Drug Liking, Cole/ARCI-Stimulation Euphoria, Subjective Drug Value, Cole/ARCI Abuse Potential, ARCI-MBG, VAS-Good Effects, VAS-Feeling High, and pupillometry. In some embodiments, the Eₘₐₓ is reduced by a percentage selected from the group consisting of approximately any of, for example, 10%, 20%, 30%, 40%, 50%, 60%, 70%, 80%, 90%, or 100%. In certain embodiments, the difference(s) in the euphoric effects of the different dosage forms are statistically significant.

In another embodiment, a multi-layer pharmaceutical composition comprising an antagonist in a first layer and an agonist in a second layer upon said first layer such that the antagonist is substantially sequestered when administered to a human being in an intact form, such that physical disruption of the dosage form alters one or more pharmacokinetic parameters as compared to the intact dosage form. In certain embodiments, the pharmacokinetic parameter is one or more of Cₘₐₓ, Tₘₐₓ, λ_{z}, T_{1/2}, AUC₀₋₈ₕ, AUCₗₐₛₜ, AUC_{inf}, elimination rate, clearance, and/or volume of distribution (L). In some embodiments, the difference is calculated based on the mean or median of the pharmacokinetic measurement. In certain embodiments, the difference(s) are statistically significant. In some embodiments, the median Cₘₐₓ of the intact dosage form is less than one-half the median Cₘₐₓ of the intact dosage form; the median Tₘₐₓ of the substantially disrupted dosage form is approximately one-seventh that of the intact dosage form; the median AUC₍₀₋₈ₕ₎ of the intact dosage form is approximately one-third that of the intact dosage form; and/or, the median T_{1/2} of the intact dosage form is greater than that of the intact dosage form. In some embodiments, the difference between the pharmacokinetic measurements is the mean or median of a measurement selected from the group consisting of Cₘₐₓ, Tmax, AUC₍₀₋₈ₕ₎, and T_{1/2}. In some embodiments, the Tₘₐₓ of the antagonist released from the disrupted composition following administration to a subject is approximately equivalent to the Tₘₐₓ of an equivalent amount of antagonist orally administered to the subject or the Tₘₐₓ of the antagonist released from the disrupted composition following administration to a subject is within approximately any of 30%, 20% or 10% of the Tₘₐₓ of an equivalent amount of antagonist orally administered to the subject. In some embodiments, the Cₘₐₓ of the antagonist released from the disrupted composition following administration to a subject is approximately equivalent to the Cₘₐₓ of an equivalent amount of antagonist orally administered to the subject or the Cₘₐₓ of the antagonist released from the disrupted composition following administration to a subject is within approximately any of 30%, 20% or 10% of the Cₘₐₓ of an equivalent amount of antagonist orally administered to the subject. In certain embodiments, the agonist may be morphine. In certain embodiments, the antagonist may be naltrexone.

Methods for manufacturing such a pharmaceutical composition are also provided. In another embodiment, a method for measuring the amount of antagonist or derivative thereof in a biological sample, the antagonist or derivative having been released from a pharmaceutical composition in vivo, the method comprising the USP paddle method (e.g. at 37° C., 100 rpm) which may include incubation in a buffer containing a surfactant.

### BRIEF DESCRIPTION OF THE DRAWINGS

FIG. 1. Cole/ARCI Stimulation Euphoria (Graphical Illustration).

FIG. 2. PTₘᵢₙ (hours) median was the lowest in the MSIR (3.13) and ALO-01 crushed (6.10) groups and highest in the ALO-01 whole group (12.07)

FIG. 3. Drug Liking mean (SD) raw scores plotted over time for the per protocol population

FIG. 4. Overall Drug Liking mean (SD) of raw scores for the per protocol population

FIG. 5. Subjective Drug Value (SDV) mean (SD) raw scores plotted at 12 and 24 hours post-dose (per protocol population)

FIG. 6. ARCI-MBG mean (SD) raw scores plotted over time for the per protocol population

FIG. 7. Cole/ARCI-Abuse Potential mean (SD) raw scores plotted over time for the per protocol population

FIG. 8. Cole/ARCI-Stimulation Euphoria mean (SD) raw scores plotted over time for the per protocol population

FIG. 9. VAS-High mean (SD) raw scores plotted over time for the per protocol population

FIG. 10. VAS-Good Effects mean (SD) raw scores plotted over time for the per protocol population

FIG. 11. VAS-Bad Effects mean (SD) raw scores plotted over time for the per protocol population

FIG. 12. VAS-Feel Sick mean (SD) raw scores plotted over time for the per protocol population

FIG. 13. VAS-Nausea mean (SD) raw scores plotted over time for the per protocol population

FIG. 14. ARCI-LSD mean (SD) raw scores plotted over time for the per protocol population

FIG. 15. Cole/ARCI-Unpleasantness-Physical mean (SD) raw scores plotted over time for the per protocol population

FIG. 16. Cole/ARCI-Unpleasantness-Dysphoria mean (SD) raw scores plotted over time for the per protocol population

FIG. 17. VAS-Any Effects mean (SD) raw scores plotted over time for the per protocol population

FIG. 18. VAS-Dizziness mean (SD) raw scores plotted over time for the per protocol population

FIG. 19. ARCI-A mean (SD) raw scores plotted over time for the per protocol population

FIG. 20. ARCI-BG mean (SD) raw scores plotted over time for the per protocol population

FIG. 21. Cole/ARCI-Stimulation-Motor mean (SD) raw scores plotted over time for the per protocol population

FIG. 22. VAS-Sleepy mean (SD) raw scores plotted over time for the per protocol population

FIG. 23. ARCI-PCAG mean (SD) raw scores plotted over time for the per protocol population

FIG. 24. Cole/ARCI-Sedation-Mental mean (SD) raw scores plotted over time for the per protocol population

FIG. 25: Cole/ARCI Sedation-Motor mean (SD) (raw scores) plotted over time for the per protocol population

FIG. 26: Morphine plasma concentration for the per protocol population

FIG. 27: Naltrexone Mean Plasma Concentration for the per protocol population

### DETAILED DESCRIPTION OF THE INVENTION

Provided herein are compositions and methods for administering a multiple active agents to a mammal in a form and manner that minimizes the effects of either active agent upon the other in vivo. In certain embodiments, at least two active agents are formulated as part of a pharmaceutical composition. A first active agent may provide a therapeutic effect in vivo. The second active agent may be an antagonist of the first active agent, and may be useful in preventing misuse of the composition. For instance, where the first active agent is a narcotic, the second active agent may be an antagonist of the narcotic. The composition remains intact during normal usage by patients and the antagonist is not released. However, upon tampering with the composition, the antagonist may be released thereby preventing the narcotic from having its intended effect. In certain embodiments, the active agents are both contained within a single unit, such as a bead, in the form of layers. The active agents may be formulated with a substantially impermeable barrier as, for example, a controlled-release composition, such that release of the antagonist from the composition is minimized. In certain embodiments, the antagonist is released in in vitro assays but is substantially not released in vivo. In vitro and in vivo release of the active agent from the composition may be measured by any of several well-known techniques. For instance, in vivo release may be determined by measuring the plasma levels of the active agent or metabolites thereof (i.e., AUC, Cmax).

In one embodiment, the invention provides a sequestering subunit comprising an opioid antagonist and a blocking agent, wherein the blocking agent substantially prevents release of the opioid antagonist from the sequestering subunit in the gastrointestinal tract for a time period that is greater than 24 hours. This sequestering subunit is incorporated into a single pharmaceutical unit that also includes an opioid agonist. The pharmaceutical unit thus includes a core portion to which the opioid antagonist is applied. A seal coat is then optionally applied upon the antagonist. Upon the seal coat is then applied a composition comprising the pharmaceutically active agent. An additional layer containing the same or a different blocking agent may then be applied such that the opioid agonist is released in the digestive tract over time (i.e., controlled release). Thus, the opioid antagonist and the opioid agonist are both contained within a single pharmaceutical unit, which is typically in the form of a bead.

The term “sequestering subunit” as used herein refers to any means for containing an antagonist and preventing or substantially preventing the release thereof in the gastrointestinal tract when intact, i.e., when not tampered with. The term “blocking agent” as used herein refers to the means by which the sequestering subunit is able to prevent substantially the antagonist from being released. The blocking agent may be a sequestering polymer, for instance, as described in greater detail below.

The terms “substantially prevents,” “prevents,” or any words stemming therefrom, as used herein, means that the antagonist is substantially not released from the sequestering subunit in the gastrointestinal tract. By “substantially not released” is meant that the antagonist may be released in a small amount, but the amount released does not affect or does not significantly affect the analgesic efficacy when the dosage form is orally administered to a host, e.g., a mammal (e.g., a human), as intended. The terms “substantially prevents,” “prevents,” or any words stemming therefrom, as used herein, does not necessarily imply a complete or 100% prevention. Rather, there are varying degrees of prevention of which one of ordinary skill in the art recognizes as having a potential benefit. In this regard, the blocking agent substantially prevents or prevents the release of the antagonist to the extent that at least about 80% of the antagonist is prevented from being released from the sequestering subunit in the gastrointestinal tract for a time period that is greater than 24 hours. Preferably, the blocking agent prevents release of at least about 90% of the antagonist from the sequestering subunit in the gastrointestinal tract for a time period that is greater than 24 hours. More preferably, the blocking agent prevents release of at least about 95% of the antagonist from the sequestering subunit. Most preferably, the blocking agent prevents release of at least about 99% of the antagonist from the sequestering subunit in the gastrointestinal tract for a time period that is greater than 24 hours.

For purposes of this invention, the amount of the antagonist released after oral administration can be measured in-vitro by dissolution testing as described in the United States Pharmacopeia (USP26) in chapter <711> Dissolution. For example, using 900 mL of 0.1 N HCl, Apparatus 2 (Paddle), 75 rpm, at 37° C. to measure release at various times from the dosage unit. Other methods of measuring the release of an antagonist from a sequestering subunit over a given period of time are known in the art (see, e.g., USP26).

Without being bound to any particular theory, it is believed that the sequestering subunit of the invention overcomes the limitations of the sequestered forms of an antagonist known in the art in that the sequestering subunit of the invention reduces osmotically-driven release of the antagonist from the sequestering subunit. Furthermore, it is believed that the present inventive sequestering subunit reduces the release of the antagonist for a longer period of time (e.g., greater than 24 hours) in comparison to the sequestered forms of antagonists known in the art. The fact that the sequestered subunit of the invention provides a longer prevention of release of the antagonist is particularly relevant, since precipitated withdrawal could occur after the time for which the therapeutic agent is released and acts. It is well known that the gastrointestinal tract transit time for individuals varies greatly within the population. Hence, the residue of the dosage form may be retained in the tract for longer than 24 hours, and in some cases for longer than 48 hours. It is further well known that opioid analgesics cause decreased bowel motility, further prolonging gastrointestinal tract transit time. Currently, sustained-release forms having an effect over a 24 hour time period have been approved by the Food and Drug Administration. In this regard, the present inventive sequestering subunit provides prevention of release of the antagonist for a time period that is greater than 24 hours when the sequestering subunit has not been tampered.

The sequestering subunit of the invention is designed to prevent substantially the release of the antagonist when intact. By “intact” is meant that a dosage form has not undergone tampering. As such, the antagonist and agonist are separated from one another within the intact dosage form. The term “tampering” is meant to include any manipulation by mechanical, thermal and/or chemical means, which changes the physical properties of the dosage form. The tampering can be, for example, crushing (e.g., by mortal and pestle), shearing, grinding, chewing, dissolution in a solvent, heating (for example, greater than about 45° C.), or any combination thereof. When the sequestering subunit of the invention has been tampered with, the antagonist is immediately released from the sequestering subunit. A dosage form that has been tampered with such that the antagonist has been released therefrom is considered “substantially disrupted” where, upon administration of the dosage form to a subject (e.g., a human being), the antagonist inhibits or otherwise interferes with the activity of the agonist in the subject. Whether or not the antagonist is inhibiting or otherwise interfering with the activity of the agonist may be determined using any of a pharmacodynamic (PD) or pharmacokinetic (PK) measurements available to one of skill in the art, including but not limited to those described herein. If the antagonist is interefering with the action of the agonist, a statistically significant difference in the measurements of one or more PD or PK measurements is typically observed between dosage forms.

By “subunit” is meant to include a composition, mixture, particle; etc., that can provide a dosage form (e.g., an oral dosage form) when combined with another subunit. The subunit can be in the form of a bead, pellet, granule, spheroid, or the like, and can be combined with additional same or different subunits, in the form of a capsule, tablet or the like, to provide a dosage form, e.g., an oral dosage form. The subunit may also be part of a larger, single unit, forming part of that unit, such as a layer. For instance, the subunit may be a core coated with an antagonist and a seal coat; this subunit may then be coated with additional compositions including a pharmaceutically active agent such as an opioid agonist.

By “antagonist of a therapeutic agent” is meant any drug or molecule, naturally-occurring or synthetic that binds to the same target molecule (e.g., a receptor) of the therapeutic agent, yet does not produce a therapeutic, intracellular, or in vivo response. In this regard, the antagonist of a therapeutic agent binds to the receptor of the therapeutic agent, thereby preventing the therapeutic agent from acting on the receptor. In the case of opioids, an antagonist may prevent the achievement of a “high” in the host.

Standard pharmacodynamic (PD) and pharmacokinetic (PK) measurements may be used to compare the effects of different dosage forms (e.g., intact vs. “tampered with” or “substantially disrupted”) on a subject or to determine if a dosage form has been tampered with or rendered substantially disrupted. Standard measurements include, for example, known PD standards or scales including but not limited to one or more of VAS-Drug Liking (Balster & Bigelow, 2003; Griffiths et al. 2003), VAS-Overall Drug Liking, ARCI short form (Martin et al., 1971), Cole/ARCI (Cole et al., 1982), Cole/ARCI-Stimulation Euphoria, Subjective Drug Value (Girffiths, et al, 1993; Griffiths, et al. 1996), Cole/ARCI Abuse Potential, ARCI-Morphine Benzedrine Group (MBG), VAS-Good Effects, VAS-Feeling High, VAS-Bad Effects, VAS-Feel Sick, VAS-Nausea, ARCI-LSD, Cole/ARCI-Unpleasantness-Physical, Cole/ARCI-Unpleasantness-Dysphoria, VAS-Any Effects, VAS-Dizziness, ARCI-Amphetamine, Cole/ARCI-Stimulation-Motor, VAS-Sleepy, ARCI-PCAG, Cole/ARCI-Sedation-Mental, Sedation-Motor, and/or pupillometry (Knaggs, et al. 2004), among others. Measurements may include mean and/or median Area Under the Effect Curve 0-2 h Post-dose (AUE₍₀₋₂ₕ₎), Area Under the Effect Curve 0-8 h Post-dose (AUE₍₀₋₈ₕ₎), Area Under the Effect Curve 0-24 h Post-dose (AUE₍₀₋₂₄ₕ₎), Apparent Post-dose Pupil Diameter (e.g., PCₘᵢₙ, PAOC₍₀₋₂ₕ₎, PAOC₍₀₋₈ₕ₎, PAOC₍₀₋₂₄ₕ₎), Raw Score at 1.5 hours Post-dose (HR1.5), maximum effect (Eₘₐₓ), Time to Reach the Maximum Effect (TEₘₐₓ). Particularly informative are Emax measurements for VAS-Drug Liking, VAS-Overall Drug Liking, Cole/ARCI-Stimulation Euphoria, Subjective Drug Value, Cole/ARCI Abuse Potential, ARCI-MBG, VAS-Good Effects, VAS-Feeling High, and pupillometry.

For the compositions described herein, PK measurements relating to the release of morphine and naltrexone are useful. Measurements of morphine, naltrexone and/or 6-β-naltrexol levels in the blood (e.g., plasma) or patients to whom various dosage forms have been administered are useful. Specific PK parameters that may be measured include, for example, mean and/or median peak concentration in Maximum Plasma Concentration (Cₘₐₓ), time to peak concentration (Tₘₐₓ), elimination rate constant (λ_{z}), terminal half-life (T_{1/2}), area under the concentration-time curve 0 hours post-dose to 8 hours post-dose (AUC₀₋₈ₕ) (pg*h/ml), area under the concentration-time curve from time-zero to the time of the last quantifiable concentration (AUCₗₐₛₜ) (pg*h/ml), and area under the plasma concentration time curve from time-zero extrapolated to infinity (AUC_{inf}) (pg*h/ml), elimination rate (ke) (1/h), clearance (L/h), and/or volume of distribution (L).

Samples (e.g., blood) may be withdrawn from those to whom the dosage form has been administered at various time points (e.g., approximately any of 0.5, 1, 1.5, 2, 3, 4, 6, 8, 10, 12 hours after administration). Where the sample is blood, plasma may be prepared from such samples using standard techniques and the measurements may be made therefrom. Mean and/or median plasma measurements may then be calculated and compared for the various dosage forms.

In certain embodiments, one or more of such standard measurements observed following administration of a dosage form may be considered different, reduced or increased from that observed following administration of a different dosage form where the difference between the effects of the dosage forms differs by about any of the following ranges: 5-10%, 10-15%, 15-20%, 10-20%, 20-25%, 25-30%, 20-30%, 30-35%, 35-40%, 30-40%, 40-45%, 45-50%, 40-50%, 50-55%, 55-60%, 50-60%, 60-65%, 65-70%, 60-70%, 70-75%, 75-80%, 70-80%, 80-85%, 85-90%, 80-90%, 90-95%, 95-100%, and 90-100%. In some embodiments, measurements may be considered “similar” to one another where there is less than about any of 0%, 5%, 10%, 15%, 20% or 25% difference. The difference may also be expressed as a fraction or ratio. For instance, the measurement observed for an intact dosage or substantially disrupted dosage form may be expressed as, for instance, approximately any of ½ (one-half), ⅓ (one-third), ¼ (one-fourth), ⅕ (one-fifth), ⅙ (one sixth), 1/7 (one-seventh), ⅛ (one-eighth), 1/9 (one-ninth), 1/10 (one-tenth), 1/20 (one-twentieth), 1/30 (one-thirtieth), 1/40 (one-fourtieth), 1/50 (one-fiftieth), 1/100 (one-one hundredth), 1/250 (one-two hundred fiftieth), 1/500 (one-five hundredth), or 1/1000 one-one thousandth) of that of the substantially disrupted or intact dosage form, respectively. The difference may also be expressed as a ratio (e.g., approximately any of 0.001:1, 0.005:1, 0.01:1, 0.1, 0.2:1, 0.3:1, 0.4:1, 0.5:1, 0.6:1, 0.7:1, 0.8:1, 0.9:1, 1:1, 1:2, 1:3, 1:4, 1:5, 1:6, 1:7, 1:8, 1:9, or 1:10).

To be regarded as “significant”, “statistically different”, “significantly reduced” or “significantly higher”, for example, the numerical values or measurements relating to the observed difference(s) may be subjected to statistical analysis. Baseline measures may be collected and significant baseline effect may be found. The treatment effect may be evaluated after the baseline covariate adjustment was made in the analysis of covariance (ANCOVA) model. The model may include treatment, period, and sequence as the fixed effects and subjects are nested within sequence as a random effect. For pharmacodynamic measures that have pre-dose values, the model may include the pre-dose baseline value as a covariate. The linear mixed effect model may be based on the per protocol population. A 5% Type I error rate with a p-value less than 0.05 may be considered “statistically significant” for all individual hypothesis tests. All statistical tests may be performed using two-tailed significance criteria. For each of the main effects, the null hypothesis may be “there was no main effect,” and the alternative hypothesis may be “there was a main effect.” For each of the contrasts, the null hypothesis may be “there was no effect difference between the tested pair,” and the alternative hypothesis may be “there was effect difference between the tested pair.” The Benjamin and Hochberg procedure may be used to control for Type I error arising from multiple treatment comparisons for all primary endpoints.

Statistical significance may also be measured using Analysis of variance (ANOVA) and the Schuimann's two one-sided t-test procedures at the 5% significance level. For instance, the log-transformed PK exposure parameters Cₘₐₓ, AUCₗₐₛₜ and AUC_{inf} may be compared to determine statistically significant differences between dosage forms. The 90% confidence interval for the ratio of the geometric means (Test/Reference) may be calculated. In certain embodiments, dosage forms may be said to be “bioequivalent” or “bioequivalence” may be declared if the lower and upper confidence intervals of the log-transformed parameters are within about any of 70-125%, 80%-125%, or 90-125% of one another. A bioequivalent or bioequivalence is preferably declared where the lower and upper confidence intervals of the log-transformed parameters are about 80%-125%.

The release of morphine, naltrexone and 6-β-naltrexol from the different compositions in vitro may be determined using standard dissolution testing techniques such as those described in the United States Pharmacopeia (USP26) in chapter <711> Dissolution (e.g., 900 mL of 0.1 N HCl, Apparatus 2 (Paddle), 75 rpm, at 37° C.; 37° C. and 100 rpm) or 72 hours in a suitable buffer such as 500 mL of 0.05M pH 7.5 phosphate buffer) to measure release at various times from the dosage unit. Other methods of measuring the release of an antagonist from a sequestering subunit over a given period of time are known in the art (see, e.g., USP26) and may also be utilized. Such assays may also be used in modified form by, for example, using a buffer system containing a surfactant (e.g., 72 hrs in 0.2% Triton X-100/0.2% sodium acetate/0.002N HCl, pH 5.5). Blood levels (including, for example, plasma levels) of morphine, naltrexone and 6-β-naltrexol may be measured using standard techniques.

The antagonist can be any agent that negates the effect of the therapeutic agent or produces an unpleasant or punishing stimulus or effect, which will deter or cause avoidance of tampering with the sequestering subunit or compositions comprising the same. Desirably, the antagonist does not harm a host by its administration or consumption but has properties that deter its administration or consumption, e.g., by chewing and swallowing or by crushing and snorting, for example. The antagonist can have a strong or foul taste or smell, provide a burning or tingling sensation, cause a lachrymation response, nausea, vomiting, or any other unpleasant or repugnant sensation, or color tissue, for example. Preferably, the antagonist is selected from the group consisting of an antagonist of a therapeutic agent, a bittering agent, a dye, a gelling agent, and an irritant. Exemplary antagonists include capsaicin, dye, bittering agents and emetics. The antagonist can comprise a single type of antagonist (e.g., a capsaicin), multiple forms of a single type of antagonist (e.g., a capasin and an analogue thereof), or a combination of different types of antagonists (e.g., one or more bittering agents and one or more gelling agents). Desirably, the amount of antagonist in the sequestering subunit of the invention is not toxic to the host.

In the instance when the therapeutic agent is an opioid agonist, the antagonist preferably is an opioid antagonist, such as naltrexone, naloxone, nalmefene, cyclazacine, levallorphan, derivatives or complexes thereof, pharmaceutically acceptable salts thereof, and combinations thereof. More preferably, the opioid antagonist is naloxone or naltrexone. By “opioid antagonist” is meant to include one or more opioid antagonists, either alone or in combination, and is further meant to include partial antagonists, pharmaceutically acceptable salts thereof, stereoisomers thereof, ethers thereof, esters thereof, and combinations thereof. The pharmaceutically acceptable salts include metal salts, such as sodium salt, potassium salt, cesium salt, and the like; alkaline earth metals, such as calcium salt, magnesium salt, and the like; organic amine salts, such as triethylamine salt, pyridine salt, picoline salt, ethanolamine salt, triethanolamine salt, dicyclohexylamine salt, N,N-dibenzylethylenediamine salt, and the like; inorganic acid salts, such as hydrochloride, hydrobromide, sulfate, phosphate, and the like; organic acid salts, such as formate, acetate, trifluoroacetate, maleate, tartrate, and the like; sulfonates, such as methanesulfonate, benzenesulfonate, p-toluenesulfonate, and the like; amino acid salts, such as arginate, asparginate, glutamate, and the like. In certain embodiments, the amount of the opioid antagonist can be about 10 ng to about 275 mg. In a preferred embodiment, when the antagonist is naltrexone, it is preferable that the intact dosage form releases less than 0.125 mg or less within 24 hours, with 0.25 mg or greater of naltrexone released after 1 hour when the dosage form is crushed or chewed.

In a preferred embodiment, the opioid antagonist comprises naloxone. Naloxone is an opioid antagonist, which is almost void of agonist effects. Subcutaneous doses of up to 12 mg of naloxone produce no discernable subjective effects, and 24 mg naloxone causes only slight drowsiness. Small doses (0.4-0.8 mg) of naloxone given intramuscularly or intravenously in man prevent or promptly reverse the effects of morphine-like opioid agonist. One mg of naloxone intravenously has been reported to block completely the effect of 25 mg of heroin. The effects of naloxone are seen almost immediately after intravenous administration. The drug is absorbed after oral administration, but has been reported to be metabolized into an inactive form rapidly in its first passage through the liver, such that it has been reported to have significantly lower potency than when parenterally administered. Oral dosages of more than 1 g have been reported to be almost completely metabolized in less than 24 hours. It has been reported that 25% of naloxone administered sublingually is absorbed (Weinberg et al., Clin. Pharmacol. Ther. 44:335-340 (1988)).

In another preferred embodiment, the opioid antagonist comprises naltrexone. In the treatment of patients previously addicted to opioids, naltrexone has been used in large oral doses (over 100 mg) to prevent euphorigenic effects of opioid agonists. Naltrexone has been reported to exert strong preferential blocking action against mu over delta sites. Naltrexone is known as a synthetic congener of oxymorphone with no opioid agonist properties, and differs in structure from oxymorphone by the replacement of the methyl group located on the nitrogen atom of oxymorphone with a cyclopropylmethyl group. The hydrochloride salt of naltrexone is soluble in water up to about 100 mg/cc. The pharmacological and pharmacokinetic properties of naltrexone have been evaluated in multiple animal and clinical studies. See, e.g., Gonzalez et al. Drugs 35:192-213 (1988). Following oral administration, naltrexone is rapidly absorbed (within 1 hour) and has an oral bioavailability ranging from 5-40%. Naltrexone's protein binding is approximately 21% and the volume of distribution following single-dose administration is 16.1 L/kg.

Naltrexone is commercially available in tablet form (Revia®, DuPont (Wilmington, Del.)) for the treatment of alcohol dependence and for the blockade of exogenously administered opioids. See, e.g., Revia (naltrexone hydrochloride tablets), Physician's Desk Reference, 51^{st} ed., Montvale, N.J.; and Medical Economics 51:957-959 (1997). A dosage of 50 mg Revia® blocks the pharmacological effects of 25 mg IV administered heroin for up to 24 hours. It is known that, when coadministered with morphine, heroin or other opioids on a chronic basis, naltrexone blocks the development of physical dependence to opioids. It is believed that the method by which naltrexone blocks the effects of heroin is by competitively binding at the opioid receptors. Naltrexone has been used to treat narcotic addiction by complete blockade of the effects of opioids. It has been found that the most successful use of naltrexone for a narcotic addiction is with narcotic addicts having good prognosis, as part of a comprehensive occupational or rehabilitative program involving behavioral control or other compliance-enhancing methods. For treatment of narcotic dependence with naltrexone, it is desirable that the patient be opioid-free for at least 7-10 days. The initial dosage of naltrexone for such purposes has typically been about 25 mg, and if no withdrawal signs occur, the dosage may be increased to 50 mg per day. A daily dosage of 50 mg is considered to produce adequate clinical blockade of the actions of parenterally administered opioids. Naltrexone also has been used for the treatment of alcoholism as an adjunct with social and psychotherapeutic methods. Other preferred opioid antagonists include, for example, cyclazocine and naltrexone, both of which have cyclopropylmethyl substitutions on the nitrogen, retain much of their efficacy by the oral route, and last longer, with durations approaching 24 hours after oral administration.

The antagonist may also be a bittering agent. The term “bittering agent” as used herein refers to any agent that provides an unpleasant taste to the host upon inhalation and/or swallowing of a tampered dosage form comprising the sequestering subunit. With the inclusion of a bittering agent, the intake of the tampered dosage form produces a bitter taste upon inhalation or oral administration, which, in certain embodiments, spoils or hinders the pleasure of obtaining a high from the tampered dosage form, and preferably prevents the abuse of the dosage form.

Various bittering agents can be employed including, for example, and without limitation, natural, artificial and synthetic flavor oils and flavoring aromatics and/or oils, oleoresins and extracts derived from plants, leaves, flowers, fruits, and so forth, and combinations thereof. Non-limiting representative flavor oils include spearmint oil, peppermint oil, eucalyptus oil, oil of nutmeg, allspice, mace, oil of bitter almonds, menthol and the like. Also useful bittering agents are artificial, natural and synthetic fruit flavors such as citrus oils, including lemon, orange, lime, and grapefruit, fruit essences, and so forth. Additional bittering agents include sucrose derivatives (e.g., sucrose octaacetate), chlorosucrose derivatives, quinine sulphate, and the like. A preferred bittering agent for use in the invention is Denatonium Benzoate NF-Anhydrous, sold under the name Bitrex™ (Macfarlan Smith Limited, Edinburgh, UK). A bittering agent can be added to the formulation in an amount of less than about 50% by weight, preferably less than about 10% by weight, more preferably less than about 5% by weight of the dosage form, and most preferably in an amount ranging from about 0.1 to 1.0 percent by weight of the dosage form, depending on the particular bittering agent(s) used.

Alternatively, the antagonist may be a dye. The term “dye” as used herein refers to any agent that causes discoloration of the tissue in contact. In this regard, if the sequestering subunit is tampered with and the contents are snorted, the dye will discolor the nasal tissues and surrounding tissues thereof. Preferred dyes are those that can bind strongly with subcutaneous tissue proteins and are well-known in the art. Dyes useful in applications ranging from, for example, food coloring to tattooing, are exemplary dyes suitable for the invention. Food coloring dyes include, but are not limited to FD&C Green #3 and FD&C Blue #1, as well as any other FD&C or D&C color. Such food dyes are commercially available through companies, such as Voigt Global Distribution (Kansas City, Mo.).

The antagonist may alternatively be an irritant. The term “irritant” as used herein includes a compound used to impart an irritating, e.g., burning or uncomfortable, sensation to an abuser administering a tampered dosage form of the invention. Use of an irritant will discourage an abuser from tampering with the dosage form and thereafter inhaling, injecting, or swallowing the tampered dosage form. Preferably, the irritant is released when the dosage form is tampered with and provides a burning or irritating effect to the abuser upon inhalation, injection, and/or swallowing the tampered dosage form. Various irritants can be employed including, for example, and without limitation, capsaicin, a capsaicin analog with similar type properties as capsaicin, and the like. Some capsaicin analogues or derivatives include, for example, and without limitation, resiniferatoxin, tinyatoxin, heptanoylisobutylamide, heptanoyl guaiacylamide, other isobutylamides or guaiacylamides, dihydrocapsaicin, homovanillyl octylester, nonanoyl vanillylamide, or other compounds of the class known as vanilloids. Resiniferatoxin is described, for example, in U.S. Pat. No. 5,290,816. U.S. Pat. No. 4,812,446 describes capsaicin analogs and methods for their preparation. Furthermore, U.S. Pat. No. 4,424,205 cites Newman, “Natural and Synthetic Pepper-Flavored Substances,” published in 1954 as listing pungency of capsaicin-like analogs. Ton et al., British Journal of Pharmacology 10:175-182 (1955), discusses pharmacological actions of capsaicin and its analogs. With the inclusion of an irritant (e.g., capsaicin) in the dosage form, the irritant imparts a burning or discomforting quality to the abuser to discourage the inhalation, injection, or oral administration of the tampered dosage form, and preferably to prevent the abuse of the dosage form. Suitable capsaicin compositions include capsaicin (trans 8-methyl-N-vanillyl-6-noneamide) or analogues thereof in a concentration between about 0.00125% and 50% by weight, preferably between about 1% and about 7.5% by weight, and most preferably, between about 1% and about 5% by weight.

The antagonist may also be a gelling agent. The term “gelling agent” as used herein refers to any agent that provides a gel-like quality to the tampered dosage form, which slows the absorption of the therapeutic agent, which is formulated with the sequestering subunit, such that a host is less likely to obtain a rapid “high.” In certain preferred embodiments, when the dosage form is tampered with and exposed to a small amount (e.g., less than about 10 ml) of an aqueous liquid (e.g., water), the dosage form will be unsuitable for injection and/or inhalation. Upon the addition of the aqueous liquid, the tampered dosage form preferably becomes thick and viscous, rendering it unsuitable for injection. The term “unsuitable for injection” is defined for purposes of the invention to mean that one would have substantial difficulty injecting the dosage form (e.g., due to pain upon administration or difficulty pushing the dosage form through a syringe) due to the viscosity imparted on the dosage form, thereby reducing the potential for abuse of the therapeutic agent in the dosage form. In certain embodiments, the gelling agent is present in such an amount in the dosage form that attempts at evaporation (by the application of heat) to an aqueous mixture of the dosage form in an effort to produce a higher concentration of the therapeutic agent, produces a highly viscous substance unsuitable for injection. When nasally inhaling the tampered dosage form, the gelling agent can become gel-like upon administration to the nasal passages, due to the moisture of the mucous membranes. This also makes such formulations aversive to nasal administration, as the gel will stick to the nasal passage and minimize absorption of the abusable substance. Various gelling agents may can be employed including, for example, and without limitation, sugars or sugar-derived alcohols, such as mannitol, sorbitol, and the like, starch and starch derivatives, cellulose derivatives, such as microcrystalline cellulose, sodium caboxymethyl cellulose, methylcellulose, ethyl cellulose, hydroxyethyl cellulose, hydroxypropyl cellulose, and hydroxypropyl methylcellulose, attapulgites, bentonites, dextrins, alginates, carrageenan, gum tragacant, gum acacia, guar gum, xanthan gum, pectin, gelatin, kaolin, lecithin, magnesium aluminum silicate, the carbomers and carbopols, polyvinylpyrrolidone, polyethylene glycol, polyethylene oxide, polyvinyl alcohol, silicon dioxide, surfactants, mixed surfactant/wetting agent systems, emulsifiers, other polymeric materials, and mixtures thereof; etc. In certain preferred embodiments, the gelling agent is xanthan gum. In other preferred embodiments, the gelling agent of the invention is pectin. The pectin or pectic substances useful for this invention include not only purified or isolated pectates but also crude natural pectin sources, such as apple, citrus or sugar beet residues, which have been subjected, when necessary, to esterification or de-esterification, e.g., by alkali or enzymes. Preferably, the pectins used in this invention are derived from citrus fruits, such as lime, lemon, grapefruit, and orange. With the inclusion of a gelling agent in the dosage form, the gelling agent preferably imparts a gel-like quality to the dosage form upon tampering that spoils or hinders the pleasure of obtaining a rapid high from due to the gel-like consistency of the tampered dosage form in contact with the mucous membrane, and in certain embodiments, prevents the abuse of the dosage form by minimizing absorption, e.g., in the nasal passages. A gelling agent can be added to the formulation in a ratio of gelling agent to opioid agonist of from about 1:40 to about 40:1 by weight, preferably from about 1:1 to about 30:1 by weight, and more preferably from about 2:1 to about 10:1 by weight of the opioid agonist. In certain other embodiments, the dosage form forms a viscous gel having a viscosity of at least about 10 cP after the dosage form is tampered with by dissolution in an aqueous liquid (from about 0.5 to about 10 ml and preferably from 1 to about 5 ml). Most preferably, the resulting mixture will have a viscosity of at least about 60 cP.

The “blocking agent” prevents or substantially prevents the release of the antagonist in the gastrointestinal tract for a time period that is greater than 24 hours, e.g., between 24 and 25 hours, 30 hours, 35 hours, 40 hours, 45 hours, 48 hours, 50 hours, 55 hours, 60 hours, 65 hours, 70 hours, 72 hours, 75 hours, 80 hours, 85 hours, 90 hours, 95 hours, or 100 hours; etc. Preferably, the time period for which the release of the antagonist is prevented or substantially prevented in the gastrointestinal tract is at least about 48 hours. More preferably, the blocking agent prevents or substantially prevents the release for a time period of at least about 72 hours.

The blocking agent of the present inventive sequestering subunit can be a system comprising a first antagonist-impermeable material and a core. By “antagonist-impermeable material” is meant any material that is substantially impermeable to the antagonist, such that the antagonist is substantially not released from the sequestering subunit. The term “substantially impermeable” as used herein does not necessarily imply complete or 100% impermeability. Rather, there are varying degrees of impermeability of which one of ordinary skill in the art recognizes as having a potential benefit. In this regard, the antagonist-impermeable material substantially prevents or prevents the release of the antagonist to an extent that at least about 80% of the antagonist is prevented from being released from the sequestering subunit in the gastrointestinal tract for a time period that is greater than 24 hours. Preferably, the antagonist-impermeable material prevents release of at least about 90% of the antagonist from the sequestering subunit in the gastrointestinal tract for a time period that is greater than 24 hours. More preferably, the antagonist-impermeable material prevents release of at least about 95% of the antagonist from the sequestering subunit. Most preferably, the antagonist-impermeable material prevents release of at least about 99% of the antagonist from the sequestering subunit in the gastrointestinal tract for a time period that is greater than 24 hours. The antagonist-impermeable material prevents or substantially prevents the release of the antagonist in the gastrointestinal tract for a time period that is greater than 24 hours, and desirably, at least about 48 hours. More desirably, the antagonist-impermeable material prevents or substantially prevents the release of the adversive agent from the sequestering subunit for a time period of at least about 72 hours.

Preferably, the first antagonist-impermeable material comprises a hydrophobic material, such that the antagonist is not released or substantially not released during its transit through the gastrointestinal tract when administered orally as intended, without having been tampered with. Suitable hydrophobic materials for use in the invention are described herein and set forth below. The hydrophobic material is preferably a pharmaceutically acceptable hydrophobic material.

It is also preferred that the first antagonist-impermeable material comprises a polymer insoluble in the gastrointestinal tract. One of ordinary skill in the art appreciates that a polymer that is insoluble in the gastrointestinal tract will prevent the release of the antagonist upon ingestion of the sequestering subunit. The polymer may be a cellulose or an acrylic polymer. Desirably, the cellulose is selected from the group consisting of ethylcellulose, cellulose acetate, cellulose propionate, cellulose acetate propionate, cellulose acetate butyrate, cellulose acetate phthalate, cellulose triacetate, and combinations thereof. Ethylcellulose includes, for example, one that has an ethoxy content of about 44 to about 55%. Ethylcellulose can be used in the form of an aqueous dispersion, an alcoholic solution, or a solution in other suitable solvents. The cellulose can have a degree of substitution (D.S.) on the anhydroglucose unit, from greater than zero and up to 3 inclusive. By “degree of substitution” is meant the average number of hydroxyl groups on the anhydroglucose unit of the cellulose polymer that are replaced by a substituting group. Representative materials include a polymer selected from the group consisting of cellulose acylate, cellulose diacylate, cellulose triacylate, cellulose acetate, cellulose diacetate, cellulose triacetate, monocellulose alkanylate, dicellulose alkanylate, tricellulose alkanylate, monocellulose alkenylates, dicellulose alkenylates, tricellulose alkenylates, monocellulose aroylates, dicellulose aroylates, and tricellulose aroylates.

More specific celluloses include cellulose propionate having a D.S. of 1.8 and a propyl content of 39.2 to 45 and a hydroxy content of 2.8 to 5.4%; cellulose acetate butyrate having a D.S. of 1.8, an acetyl content of 13 to 15% and a butyryl content of 34 to 39%; cellulose acetate butyrate having an acetyl content of 2 to 29%, a butyryl content of 17 to 53% and a hydroxy content of 0.5 to 4.7%; cellulose triacylate having a D.S. of 2.9 to 3, such as cellulose triacetate, cellulose trivalerate, cellulose trilaurate, cellulose tripatmitate, cellulose trisuccinate, and cellulose trioctanoate; cellulose diacylates having a D.S. of 2.2 to 2.6, such as cellulose disuccinate, cellulose dipalmitate, cellulose dioctanoate, cellulose dipentanoate, and coesters of cellulose, such as cellulose acetate butyrate, cellulose acetate octanoate butyrate, and cellulose acetate propionate. Additional cellulose polymers that may be used to prepare the sequestering subunit include acetaldehyde dimethyl cellulose acetate, cellulose acetate ethylcarbamate, cellulose acetate methycarbamate, and cellulose acetate dimethylaminocellulose acetate.

The acrylic polymer preferably is selected from the group consisting of methacrylic polymers, acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), glycidyl methacrylate copolymers, and combinations thereof. An acrylic polymer useful for preparation of a sequestering subunit of the invention includes acrylic resins comprising copolymers synthesized from acrylic and methacrylic acid esters (e.g., the copolymer of acrylic acid lower alkyl ester and methacrylic acid lower alkyl ester) containing about 0.02 to about 0.03 mole of a tri (lower alkyl) ammonium group per mole of the acrylic and methacrylic monomer used. An example of a suitable acrylic resin is ammonio methacrylate copolymer NF21, a polymer manufactured by Rohm Pharma GmbH, Darmstadt, Germany, and sold under the Eudragit® trademark. Eudragit® is a water-insoluble copolymer of ethyl acrylate (EA), methyl methacrylate (MM) and trimethylammoniumethyl methacrylate chloride (TAM) in which the molar ratio of TAM to the remaining components (EA and MM) is 1:40. Acrylic resins, such as Eudragit®, can be used in the form of an aqueous dispersion or as a solution in suitable solvents. Preferred acrylic polymers include copolymers of acrylic and methacrylic acid esters with a low content in quaternary ammonium groups such as Eudragit® RL PO (Type A) and Eudragit® RS PO (Type B; as used herein, “Eudragit® RS”) (as described the monographs Ammonio Methacrylate Copolymer Type A Ph. Eur., Ammonio Methacrylate Copolymer Type B Ph. Eur., Ammonio Methacrylate Copolymer, Type A and B USP/NF, and Aminoalkylmethacrylate Copolymer RS JPE).

In another preferred embodiment, the antagonist-impermeable material is selected from the group consisting of polylactic acid, polyglycolic acid, a co-polymer of polylactic acid and polyglycolic acid, and combinations thereof. In certain other embodiments, the hydrophobic material includes a biodegradable polymer comprising a poly(lactic/glycolic acid) (“PLGA”), a polylactide, a polyglycolide, a polyanhydride, a polyorthoester, polycaprolactones, polyphosphazenes, polysaccharides, proteinaceous polymers, polyesters, polydioxanone, polygluconate, polylactic-acid-polyethylene oxide copolymers, poly(hydroxybutyrate), polyphosphoester or combinations thereof. Preferably, the biodegradable polymer comprises a poly(lactic/glycolic acid), a copolymer of lactic and glycolic acid, having a molecular weight of about 2,000 to about 500,000 daltons. The ratio of lactic acid to glycolic acid is preferably from about 100:1 to about 25:75, with the ratio of lactic acid to glycolic acid of about 65:35 being more preferred.

Poly(lactic/glycolic acid) can be prepared by the procedures set forth in U.S. Pat. No. 4,293,539 (Ludwig et al.), which is incorporated herein by reference. In brief, Ludwig prepares the copolymer by condensation of lactic acid and glycolic acid in the presence of a readily removable polymerization catalyst (e.g., a strong ion-exchange resin such as Dowex HCR-W2-H). The amount of catalyst is not critical to the polymerization, but typically is from about 0.01 to about 20 parts by weight relative to the total weight of combined lactic acid and glycolic acid. The polymerization reaction can be conducted without solvents at a temperature from about 100° C. to about 250° C. for about 48 to about 96 hours, preferably under a reduced pressure to facilitate removal of water and by-products. Poly(lactic/glycolic acid) is then recovered by filtering the molten reaction mixture in an organic solvent, such as dichloromethane or acetone, and then filtering to remove the catalyst.

Suitable plasticizers for use in the sequestering subunit include, for example, acetyl triethyl citrate, acetyl tributyl citrate, triethyl citrate, diethyl phthalate, dibutyl phthalate (DBP), acetyltri-N-butyl citrate (ATBC), or dibutyl sebacate, which can be admixed with the polymer. Other additives such as coloring agents may also be used in making the present inventive sequestering subunit.

In certain embodiments, additives may be included in the compositions that improve the sequestering characteristics of the sequestering subunit. As described below, the ratio of additives or components with respect to other additives or components may be modified to enhance or delay improve sequestration of the agent contained within the subunit. Various amounts of a functional additive (i.e., a charge-neutralizing additive) may be included to vary the release of an antagonist, particularly where a water-soluble core (i.e., a sugar sphere) is utilized. For instance, it has been determined that the inclusion of a low amount of charge-neutralizing additive relative to sequestering polymer on a weight-by-weight basis may cause decreased release of the antagonist.

In certain embodiments, a surfactant may serve as a charge-neutralizing additive. Such neutralization may in certain embodiments reduce the swelling of the sequestering polymer by hydration of positively charged groups contained therein. Surfactants (ionic or non-ionic) may also be used in preparing the sequestering subunit. It is preferred that the surfactant be ionic. Suitable exemplary agents include, for example, alkylaryl sulphonates, alcohol sulphates, sulphosuccinates, sulphosuccinamates, sarcosinates or taurates and others. Additional examples include but are not limited to ethoxylated castor oil, benzalkonium chloride, polyglycolyzed glycerides, acetylated monoglycerides, sorbitan fatty acid esters, poloxamers, polyoxyethylene fatty acid esters, polyoxyethylene derivatives, monoglycerides or ethoxylated derivatives thereof, diglycerides or polyoxyethylene derivatives thereof, sodium docusate, sodium lauryl sulfate, dioctyl sodium sulphosuccinate, sodium lauryl sarcosinate and sodium methyl cocoyl taurate, magnesium lauryl sulfate, triethanolamine, cetrimide, sucrose laurate and other sucrose esters, glucose (dextrose) esters, simethicone, ocoxynol, dioctyl sodiumsulfosuceinate, polyglycolyzed glycerides, sodiumdodecylbenzene sulfonate, dialkyl sodiumsulfosuccinate, fatty alcohols such as lauryl, cetyl, and steryl, glycerylesters, cholic acid or derivatives thereof, lecithins, and phospholipids. These agents are typically characterized as ionic (i.e., anionic or cationic) or nonionic. In certain embodiments described herein, an anionic surfactant such as sodium lauryl sulfate (SLS) is preferably used (U.S. Pat. No. 5,725,883; U.S. Pat. No. 7,201,920; EP 502642A1; Shokri, et al. Pharm. Sci. 2003. The effect of sodium lauryl sulphate on the release of diazepam from solid dispersions prepared by cogrinding technique. Wells, et al. Effect of Anionic Surfactants on the Release of Chlorpheniramine Maleate From an Inert, Heterogeneous Matrix. Drug Development and Industrial Pharmacy 18(2) (1992): 175-186. Rao, et al. “Effect of Sodium Lauryl Sulfate on the Release of Rifampicin from Guar Gum Matrix.” Indian Journal of Pharmaceutical Science (2000): 404-406; Knop, et al. Influence of surfactants of different charge and concentration on drug release from pellets coated with an aqueous dispersion of quaternary acrylic polymers. STP Pharma Sciences, Vol. 7, No. 6, (1997) 507-512). Other suitable agents are known in the art.

As shown herein, SLS is particularly useful in combination with Eudragit RS when the sequestering subunit is built upon a sugar sphere substrate. The inclusion of SLS at less than approximately 6.3% on a weight-to-weight basis relative to the sequestering polymer (i.e., Eudragit RS) may provide a charge neutralizing function (theoretically 20% and 41% neutralization, respectfully), and thereby significantly slow the release of the active agent encapsulated thereby (i.e., the antagonist naltrexone). Inclusion of more than approximately 6.3% SLS relative to the sequestering polymer appears to increase release of the antagonist from the sequestering subunit. With respect to SLS used in conjunction with Eudragit® RS, it is preferred that the SLS is present at approximately 1%, 2%, 3%, 4% or 5%, and typically less than 6% on a w/w basis relative to the sequestering polymer (i.e., Eudragit® RS). In preferred embodiments, SLS may be present at approximately 1.6% or approximately 3.3% relative to the sequestering polymer. As discussed above, many agents (i.e., surfactants) may substitute for SLS in the compositions disclosed herein.

Additionally useful agents include those that may physically block migration of the antagonist from the subunit and/or enhance the hydrophobicity of the barrier. One exemplary agent is talc, which is commonly used in pharmaceutical compositions (Pawar et al. Agglomeration of Ibuprofen With Talc by Novel Crystallo-Co-Agglomeration Technique. AAPS PharmSciTech. 2004; 5(4): article 55). As shown in the Examples, talc is especially useful where the sequestering subunit is built upon a sugar sphere core. Any form of talc may be used, so long as it does not detrimentally affect the function of the composition. Most talc results from the alteration of dolomite (CaMg(CO₃)₂ or magnesite (MgO) in the presence of excess dissolved silica (SiO₂) or by altering serpentine or quartzite. Talc may be include minerals such as tremolite (CaMg₃(SiO₃)₄), serpentine (3MgO.2SiO₂.2H₂O), anthophyllite (Mg₇.(OH)₂.(Si₄O₁₁)O₂), magnesite, mica, chlorite, dolomite, the calcite form of calcium carbonate (CaCO₃), iron oxide, carbon, quartz, and/or manganese oxide. The presence of such impurities may be acceptable in the compositions described herein provided the function of the talc is maintained. It is preferred that that talc be USP grade. As mentioned above, the function of talc as described herein is to enhance the hydrophobicity and therefore the functionality of the sequestering polymer. Many substitutes for talc may be utilized in the compositions described herein as may be determined by one of skill in the art.

It has been determined that the ratio of talc to sequestering polymer may make a dramatic difference in the functionality of the compositions described herein. For instance, the Examples described below demonstrate that the talc to sequestering polymer ratio (w/w) is important with respect to compositions designed to prevent the release of naltrexone therefrom. It is shown therein that inclusion of an approximately equivalent amount (on a weight-by-weight basis) of talc and Eudragit® RS results in a very low naltrexone release profile. In contrast, significantly lower or higher both a lower (69% w/w) and a higher (151% w/w) talc:Eudragit® RS ratios result in increased release of naltrexone release. Thus, where talc and Eudragit® RS are utilized, it is preferred that talc is present at approximately 75%, 80%, 85%, 90%, 95%, 100%, 105%, 110%, 115%, 120% or 125% w/w relative to Eudragit® RS. As described above, the most beneficial ratio for other additives or components will vary and may be determined using standard experimental procedures.

In certain embodiments, such as where a water-soluble core is utilized, it is useful to include agents that may affect the osmotic pressure of the composition (i.e., an osmotic pressure regulating agent) (see, in general, WO 2005/046561 A2 and WO 2005/046649 A2 relating to Eudramode®). This agent is preferably applied to the Eudragit® RS/talc layer described above. In a pharmaceutical unit comprising a sequestering subunit overlayed by an active agent (i.e., a controlled-release agonist preparation), the osmotic pressure regulating agent is preferably positioned immediately beneath the active agent layer. Suitable osmotic pressure regulating agents may include, for instance, hydroxypropylmethyl cellulose (HPMC) or chloride ions (i.e., from NaCl), or a combination of HPMC and chloride ions (i.e., from NaCl). Other ions that may be useful include bromide or iodide. The combination of sodium chloride and HPMC may be prepared in water or in a mixture of ethanol and water, for instance. HPMC is commonly utilized in pharmaceutical compositions (see, for example, U.S. Pat. Nos. 7,226,620 and 7,229,982). In certain embodiments, HPMC may have a molecular weight ranging from about 10,000 to about 1,500,000, and typically from about 5000 to about 10,000 (low molecular weight HPMC). The specific gravity of HPMC is typically from about 1.19 to about 1.31, with an average specific gravity of about 1.26 and a viscosity of about 3600 to 5600. HPMC may be a water-soluble synthetic polymer. Examples of suitable, commercially available hydroxypropyl methylcellulose polymers include Methocel K100 LV and Methocel K4M (Dow). Other HPMC additives are known in the art and may be suitable in preparing the compositions described herein. As shown in the Examples, the inclusion of NaCl (e.g., in some embodiments, with HPMC or HPC) was found to have positively affect sequestration of naltrexone by Eudragit® RS. In certain embodiments, it is preferred that the charge-neutralizing additive (i.e., NaCl) is included at less than approximately 1, 2, 3, 4, 5, 6, 7, 8, 9, or 10% on a weight-by-weight basis. In other preferred embodiments, the charge-neutralizing additive is present at approximately 4% on a weight-by-weight basis.

Thus, in one embodiment, a sequestering subunit built upon a sugar sphere substrate is provided comprising a sequestering polymer (i.e., Eudragit® RS) in combination with several optimizing agents, including sodium lauryl sulfate (SLS) as a charge-neutralizing agent to reduce swelling of the film by hydration of the positively charged groups on the polymer; talc to create a solid impermeable obstacle to naltrexone transport through the film and as a hydrophobicity-enhacing agent; and a chloride ion (i.e., as NaCl) as an osmotic pressure reducing agent. The ratio of each of the additional ingredients relative to the sequestering polymer was surprisingly found to be important to the function of the sequestering subunit. For instance, the Examples provide a sequestering subunit including a sequestering polymer and the optimizing agents SLS at less than 6%, preferably 1-4%, and even more preferably 1.6% or 3.3% on a w/w basis relative to Eudragit RS; talc in an amount approximately equal to Eudragit® RS (on a w/w basis); and, NaCl present at approximately 4% on a w/w basis.

Methods of making any of the sequestering subunits of the invention are known in the art. See, for example, Remington: The Science and Practice of Pharmacy, Alfonso R. Genaro (ed), 20^{th} edition, and Example 2 set forth below. The sequestering subunits can be prepared by any suitable method to provide, for example, beads, pellets, granules, spheroids, and the like. Spheroids or beads, coated with an active ingredient can be prepared, for example, by dissolving the active ingredient in water and then spraying the solution onto a substrate, for example, nu pariel 18/20 beads, using a Wurster insert. Optionally, additional ingredients are also added prior to coating the beads in order to assist the active ingredient in binding to the substrates, and/or to color the solution; etc. The resulting substrate-active material optionally can be overcoated with a barrier material to separate the therapeutically active agent from the next coat of material, e.g., release-retarding or sequestering material. Preferably, the barrier material is a material comprising hydroxypropyl methylcellulose. However, any film-former known in the art can be used. Preferably, the barrier material does not affect the dissolution rate of the final product.

Pellets comprising an active ingredient can be prepared, for example, by a melt pelletization technique. Typical of such techniques is when the active ingredient in finely divided form is combined with a binder (also in particulate form) and other optional inert ingredients, and thereafter the mixture is pelletized, e.g., by mechanically working the mixture in a high shear mixer to form the pellets (e.g., pellets, granules, spheres, beads; etc., collectively referred to herein as “pellets”). Thereafter, the pellets can be sieved in order to obtain pellets of the requisite size. The binder material is preferably in particulate form and has a melting point above about 40° C. Suitable binder substances include, for example, hydrogenated castor oil, hydrogenated vegetable oil, other hydrogenated fats, fatty alcohols, fatty acid esters, fatty acid glycerides, and the like.

The diameter of the extruder aperture or exit port also can be adjusted to vary the thickness of the extruded strands. Furthermore, the exit part of the extruder need not be round; it can be oblong, rectangular; etc. The exiting strands can be reduced to particles using a hot wire cutter, guillotine; etc.

The melt-extruded multiparticulate system can be, for example, in the form of granules, spheroids, pellets, or the like, depending upon the extruder exit orifice. The terms “melt-extruded multiparticulate(s)” and “melt-extruded multiparticulate system(s)” and “melt-extruded particles” are used interchangeably herein and include a plurality of subunits, preferably within a range of similar size and/or shape. The melt-extruded multiparticulates are preferably in a range of from about 0.1 to about 12 mm in length and have a diameter of from about 0.1 to about 5 mm. In addition, the melt-extruded multiparticulates can be any geometrical shape within this size range. Alternatively, the extrudate can simply be cut into desired lengths and divided into unit doses of the therapeutically active agent without the need of a spheronization step.

The substrate also can be prepared via a granulation technique. Generally, melt-granulation techniques involve melting a normally solid hydrophobic material, e.g., a wax, and incorporating an active ingredient therein. To obtain a sustained-release dosage form, it can be necessary to incorporate an additional hydrophobic material.

A coating composition can be applied onto a substrate by spraying it onto the substrate using any suitable spray equipment. For example, a Wurster fluidized-bed system can be used in which an air flow from underneath, fluidizes the coated material and effects drying, while the insoluble polymer coating is sprayed on. The thickness of the coating will depend on the characteristics of the particular coating composition, and can be determined by using routine experimentation.

Any manner of preparing a subunit can be employed. By way of example, a subunit in the form of a pellet or the like can be prepared by co-extruding a material comprising the opioid agonist and a material comprising the opioid antagonist and/or antagonist in sequestered form. Optionally, the opioid agonist composition can cover, e.g., overcoat, the material comprising the antagonist and/or antagonist in sequestered form. A bead, for example, can be prepared by coating a substrate comprising an opioid antagonist and/or an antagonist in sequestered form with a solution comprising an opioid agonist.

The sequestering subunits of the invention are particularly well-suited for use in compositions comprising the sequestering subunit and a therapeutic agent in releasable form. In this regard, the invention also provides a composition comprising any of the sequestering subunits of the invention and a therapeutic agent in releasable form. By “releasable form” is meant to include immediate release, intermediate release, and sustained-release forms. The therapeutic agent can be formulated to provide immediate release of the therapeutic agent. In preferred embodiments, the composition provides sustained-release of the therapeutic agent.

The therapeutic agent applied upon the sequestering subunit may be any medicament. The therapeutic agent of the present inventive compositions can be any medicinal agent used for the treatment of a condition or disease, a pharmaceutically acceptable salt thereof, or an analogue of either of the foregoing. The therapeutic agent can be, for example, an analgesic (e.g., an opioid agonist, aspirin, acetaminophen, non-steroidal anti-inflammatory drugs (“NSAIDS”), N-methyl-D-aspartate (“NMDA”) receptor antagonists, cyclooxygenase-II inhibitors (“COX-II inhibitors”), and glycine receptor antagonists), an antibacterial agent, an anti-viral agent, an anti-microbial agent, anti-infective agent, a chemotherapeutic, an immunosuppressant agent, an antitussive, an expectorant, a decongestant, an antihistamine drugs, a decongestant, antihistamine drugs, and the like. Preferably, the therapeutic agent is one that is addictive (physically and/or psychologically) upon repeated use and typically leads to abuse of the therapeutic agent. In this regard, the therapeutic agent can be any opioid agonist as discussed herein.

The therapeutic agent can be an opioid agonist. By “opioid” is meant to include a drug, hormone, or other chemical or biological substance, natural or synthetic, having a sedative, narcotic, or otherwise similar effect(s) to those containing opium or its natural or synthetic derivatives. By “opioid agonist,” sometimes used herein interchangeably with terms “opioid” and “opioid analgesic,” is meant to include one or more opioid agonists, either alone or in combination, and is further meant to include the base of the opioid, mixed or combined agonist-antagonists, partial agonists, pharmaceutically acceptable salts thereof, stereoisomers thereof, ethers thereof, esters thereof, and combinations thereof.

Opioid agonists include, for example, alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papavereturn, pentazocine, phenadoxone, phenazocine, phenomorphan, phenoperidine, piminodine, piritramide, propheptazine, promedol, properidine, propiram, propoxyphene, sufentanil, tramadol, tilidine, derivatives or complexes thereof, pharmaceutically acceptable salts thereof, and combinations thereof. Preferably, the opioid agonist is selected from the group consisting of hydrocodone, hydromorphone, oxycodone, dihydrocodeine, codeine, dihydromorphine, morphine, buprenorphine, derivatives or complexes thereof, pharmaceutically acceptable salts thereof, and combinations thereof. Most preferably, the opioid agonist is morphine, hydromorphone, oxycodone or hydrocodone. In a preferred embodiment, the opioid agonist comprises oxycodone or hydrocodone and is present in the dosage form in an amount of about 15 to about 45 mg, and the opioid antagonist comprises naltrexone and is present in the dosage form in an amount of about 0.5 to about 5 mg. Equianalgesic calculated doses (mg) of these opioids, in comparison to a 15 mg dose of hydrocodone, are as follows: oxycodone (13.5 mg); codeine (90.0 mg), hydrocodone (15.0 mg), hydromorphone (3.375 mg), levorphanol (1.8 mg), meperidine (15.0 mg), methadone (9.0 mg), and morphine (27.0).

Hydrocodone is a semisynthetic narcotic analgesic and antitussive with multiple nervous system and gastrointestinal actions. Chemically, hydrocodone is 4,5-epoxy-3-methoxy-17-methylmorphinan-6-one, and is also known as dihydrocodeinone. Like other opioids, hydrocodone can be habit-forming and can produce drug dependence of the morphine type. Like other opium derivatives, excess doses of hydrocodone will depress respiration.

Oral hydrocodone is also available in Europe (e.g., Belgium, Germany, Greece, Italy, Luxembourg, Norway and Switzerland) as an antitussive agent. A parenteral formulation is also available in Germany as an antitussive agent. For use as an analgesic, hydrocodone bitartrate is commonly available in the United States only as a fixed combination with non-opiate drugs (e.g., ibuprofen, acetaminophen, aspirin; etc.) for relief of moderate to moderately severe pain.

A common dosage form of hydrocodone is in combination with acetaminophen and is commercially available, for example, as Lortab® in the United States from UCB Pharma, Inc. (Brussels, Belgium), as 2.5/500 mg, 5/500 mg, 7.5/500 mg and 10/500 mg hydrocodone/acetaminophen tablets. Tablets are also available in the ratio of 7.5 mg hydrocodone bitartrate and 650 mg acetaminophen and a 7.5 mg hydrocodone bitartrate and 750 mg acetaminophen. Hydrocodone, in combination with aspirin, is given in an oral dosage form to adults generally in 1-2 tablets every 4-6 hours as needed to alleviate pain. The tablet form is 5 mg hydrocodone bitartrate and 224 mg aspirin with 32 mg caffeine; or 5 mg hydrocodone bitartrate and 500 mg aspirin. Another formulation comprises hydrocodone bitartrate and ibuprofen. Vicoprofen®, commercially available in the U.S. from Knoll Laboratories (Mount Olive, N.J.), is a tablet containing 7.5 mg hydrocodone bitartrate and 200 mg ibuprofen. The invention is contemplated to encompass all such formulations, with the inclusion of the opioid antagonist and/or antagonist in sequestered form as part of a subunit comprising an opioid agonist.

Oxycodone, chemically known as 4,5-epoxy-14-hydroxy-3-methoxy-17-methylmorphinan-6-one, is an opioid agonist whose principal therapeutic action is analgesia. Other therapeutic effects of oxycodone include anxiolysis, euphoria and feelings of relaxation. The precise mechanism of its analgesic action is not known, but specific CNS opioid receptors for endogenous compounds with opioid-like activity have been identified throughout the brain and spinal cord and play a role in the analgesic effects of this drug. Oxycodone is commercially available in the United States, e.g., as Oxycotin® from Purdue Pharma L.P. (Stamford, Conn.), as controlled-release tablets for oral administration containing 10 mg, 20 mg, 40 mg or 80 mg oxycodone hydrochloride, and as OxyIR™, also from Purdue Pharma L.P., as immediate-release capsules containing 5 mg oxycodone hydrochloride. The invention is contemplated to encompass all such formulations, with the inclusion of an opioid antagonist and/or antagonist in sequestered form as part of a subunit comprising an opioid agonist.

Oral hydromorphone is commercially available in the United States, e.g., as Dilaudid® from Abbott Laboratories (Chicago, Ill.). Oral morphine is commercially available in the United States, e.g., as Kadian® from Faulding Laboratories (Piscataway, N.J.).

Exemplary NSAIDS include ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam or isoxicam, and the like. Useful dosages of these drugs are well-known.

Exemplary NMDA receptor medicaments include morphinans, such as dexotromethorphan or dextrophan, ketamine, d-methadone, and pharmaceutically acceptable salts thereof, and encompass drugs that block a major intracellular consequence of NMDA-receptor activation, e.g., a ganglioside, such as (6-aminothexyl)-5-chloro-1-naphthalenesulfonamide. These drugs are stated to inhibit the development of tolerance to and/or dependence on addictive drugs, e.g., narcotic analgesics, such as morphine, codeine; etc., in U.S. Pat. Nos. 5,321,012 and 5,556,838 (both to Mayer et al.), both of which are incorporated herein by reference, and to treat chronic pain in U.S. Pat. No. 5,502,058 (Mayer et al.), incorporated herein by reference. The NMDA agonist can be included alone or in combination with a local anesthetic, such as lidocaine, as described in these patents by Mayer et al.

COX-2 inhibitors have been reported in the art, and many chemical compounds are known to produce inhibition of cyclooxygenase-2. COX-2 inhibitors are described, for example, in U.S. Pat. Nos. 5,616,601; 5,604,260; 5,593,994; 5,550,142; 5,536,752; 5,521,213; 5,475,995; 5,639,780; 5,604,253; 5,552,422; 5,510,368; 5,436,265; 5,409,944 and 5,130,311, all of which are incorporated herein by reference. Certain preferred COX-2 inhibitors include celecoxib (SC-58635), DUP-697, flosulide (CGP-28238), meloxicam, 6-methoxy-2-naphthylacetic acid (6-NMA), MK-966 (also known as Vioxx), nabumetone (prodrug for 6-MNA), nimesulide, NS-398, SC-5766, SC-58215, T-614, or combinations thereof. Dosage levels of COX-2 inhibitor on the order of from about 0.005 mg to about 140 mg per kilogram of body weight per day have been shown to be therapeutically effective in combination with an opioid analgesic. Alternatively, about 0.25 mg to about 7 g per patient per day of a COX-2 inhibitor can be administered in combination with an opioid analgesic.

The treatment of chronic pain via the use of glycine receptor antagonists and the identification of such drugs is described in U.S. Pat. No. 5,514,680 (Weber et al.), which is incorporated herein by reference.

In embodiments in which the opioid agonist comprises hydrocodone, the sustained-release oral dosage forms can include analgesic doses from about 8 mg to about 50 mg of hydrocodone per dosage unit. In sustained-release oral dosage forms where hydromorphone is the therapeutically active opioid, it is included in an amount from about 2 mg to about 64 mg hydromorphone hydrochloride. In another embodiment, the opioid agonist comprises morphine, and the sustained-release oral dosage forms of the invention include from about 2.5 mg to about 800 mg morphine, by weight. In yet another embodiment, the opioid agonist comprises oxycodone and the sustained-release oral dosage forms include from about 2.5 mg to about 800 mg oxycodone. In certain preferred embodiments, the sustained-release oral dosage forms include from about 20 mg to about 30 mg oxycodone. Controlled release oxycodone formulations are known in the art. The following documents describe various controlled-release oxycodone formulations suitable for use in the invention described herein, and processes for their manufacture: U.S. Pat. Nos. 5,266,331; 5,549,912; 5,508,042; and 5,656,295, which are incorporated herein by reference. The opioid agonist can comprise tramadol and the sustained-release oral dosage forms can include from about 25 mg to 800 mg tramadol per dosage unit.

The therapeutic agent in sustained-release form is preferably a particle of therapeutic agent that is combined with a release-retarding or sequestering material. The release-retarding or sequestering material is preferably a material that permits release of the therapeutic agent at a sustained rate in an aqueous medium. The release-retarding or sequestering material can be selectively chosen so as to achieve, in combination with the other stated properties, a desired in vitro release rate.

In a preferred embodiment, the oral dosage form of the invention can be formulated to provide for an increased duration of therapeutic action allowing once-daily dosing. In general, a release-retarding or sequestering material is used to provide the increased duration of therapeutic action. Preferably, the once-daily dosing is provided by the dosage forms and methods described in U.S. Patent Application Pub. No. 2005/0020613 to Boehm, entitled “Sustained-Release Opioid Formulations and Method of Use,” filed on Sep. 22, 2003, and incorporated herein by reference.

Preferred release-retarding or sequestering materials include acrylic polymers, alkylcelluloses, shellac, zein, hydrogenated vegetable oil, hydrogenated castor oil, and combinations thereof. In certain preferred embodiments, the release-retarding or sequestering material is a pharmaceutically acceptable acrylic polymer, including acrylic acid and methacrylic acid copolymers, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, and glycidyl methacrylate copolymers. In certain preferred embodiments, the acrylic polymer comprises one or more ammonio methacrylate copolymers. Ammonio methacrylate copolymers are well-known in the art, and are described in NF21, the 21^{st} edition of the National Formulary, published by the United States Pharmacopeial Convention Inc. (Rockville, Md.), as fully polymerized copolymers of acrylic and methacrylic acid esters with a low content of quaternary ammonium groups. In other preferred embodiments, the release-retarding or sequestering material is an alkyl cellulosic material, such as ethylcellulose. Those skilled in the art will appreciate that other cellulosic polymers, including other alkyl cellulosic polymers, can be substituted for part or all of the ethylcellulose.

Release-modifying agents, which affect the release properties of the release-retarding or sequestering material, also can be used. In a preferred embodiment, the release-modifying agent functions as a pore-former. The pore-former can be organic or inorganic, and include materials that can be dissolved, extracted or leached from the coating in the environment of use. The pore-former can comprise one or more hydrophilic polymers, such as hydroxypropylmethylcellulose. In certain preferred embodiments, the release-modifying agent is selected from hydroxypropylmethylcellulose, lactose, metal stearates, and combinations thereof.

The release-retarding or sequestering material can also include an erosion-promoting agent, such as starch and gums; a release-modifying agent useful for making microporous lamina in the environment of use, such as polycarbonates comprised of linear polyesters of carbonic acid in which carbonate groups reoccur in the polymer chain; and/or a semi-permeable polymer.

The release-retarding or sequestering material can also include an exit means comprising at least one passageway, orifice, or the like. The passageway can be formed by such methods as those disclosed in U.S. Pat. Nos. 3,845,770; 3,916,889; 4,063,064; and 4,088,864, which are incorporated herein by reference. The passageway can have any shape, such as round, triangular, square, elliptical, irregular; etc.

In certain embodiments, the therapeutic agent in sustained-release form can include a plurality of substrates comprising the active ingredient, which substrates are coated with a sustained-release coating comprising a release-retarding or sequestering material.

The sustained-release preparations of the invention can be made in conjunction with any multiparticulate system, such as beads, ion-exchange resin beads, spheroids, microspheres, seeds, pellets, granules, and other multiparticulate systems in order to obtain a desired sustained-release of the therapeutic agent. The multiparticulate system can be presented in a capsule or in any other suitable unit dosage form.

In certain preferred embodiments, more than one multiparticulate system can be used, each exhibiting different characteristics, such as pH dependence of release, time for release in various media (e.g., acid, base, simulated intestinal fluid), release in vivo, size and composition.

To obtain a sustained-release of the therapeutic agent in a manner sufficient to provide a therapeutic effect for the sustained durations, the therapeutic agent can be coated with an amount of release-retarding or sequestering material sufficient to obtain a weight gain level from about 2 to about 30%, although the coat can be greater or lesser depending upon the physical properties of the particular therapeutic agent utilized and the desired release rate, among other things. Moreover, there can be more than one release-retarding or sequestering material used in the coat, as well as various other pharmaceutical excipients.

Solvents typically used for the release-retarding or sequestering material include pharmaceutically acceptable solvents, such as water, methanol, ethanol, methylene chloride and combinations thereof.

In certain embodiments of the invention, the release-retarding or sequestering material is in the form of a coating comprising an aqueous dispersion of a hydrophobic polymer. The inclusion of an effective amount of a plasticizer in the aqueous dispersion of hydrophobic polymer will further improve the physical properties of the film. For example, because ethylcellulose has a relatively high glass transition temperature and does not form flexible films under normal coating conditions, it is necessary to plasticize the ethylcellulose before using the same as a coating material. Generally, the amount of plasticizer included in a coating solution is based on the concentration of the film-former, e.g., most often from about 1 to about 50 percent by weight of the film-former. Concentrations of the plasticizer, however, can be determined by routine experimentation.

Examples of plasticizers for ethylcellulose and other celluloses include dibutyl sebacate, diethyl phthalate, triethyl citrate, tributyl citrate, and triacetin, although it is possible that other plasticizers (such as acetylated monoglycerides, phthalate esters, castor oil; etc.) can be used. A plasticizer that is not leached into the aqueous phase such as DBS is preferred.

Examples of plasticizers for the acrylic polymers include citric acid esters, such as triethyl citrate NF21, tributyl citrate, dibutyl phthalate (DBP), acetyltri-N-butyl citrate (ATBC), and possibly 1,2-propylene glycol, polyethylene glycols, propylene glycol, diethyl phthalate, castor oil, and triacetin, although it is possible that other plasticizers (such as acetylated monoglycerides, phthalate esters, castor oil; etc.) can be used.

The sustained-release profile of drug release in the formulations of the invention (either in vivo or in vitro) can be altered, for example, by using more than one release-retarding or sequestering material, varying the thickness of the release-retarding or sequestering material, changing the particular release-retarding or sequestering material used, altering the relative amounts of release-retarding or sequestering material, altering the manner in which the plasticizer is added (e.g., when the sustained-release coating is derived from an aqueous dispersion of hydrophobic polymer), by varying the amount of plasticizer relative to retardant material, by the inclusion of additional ingredients or excipients, by altering the method of manufacture; etc.

In certain other embodiments, the oral dosage form can utilize a multiparticulate sustained-release matrix. In certain embodiments, the sustained-release matrix comprises a hydrophilic and/or hydrophobic polymer, such as gums, cellulose ethers, acrylic resins and protein-derived materials. Of these polymers, the cellulose ethers, specifically hydroxyalkylcelluloses and carboxyalkylcelluloses, are preferred. The oral dosage form can contain between about 1% and about 80% (by weight) of at least one hydrophilic or hydrophobic polymer.

The hydrophobic material is preferably selected from the group consisting of alkylcellulose, acrylic and methacrylic acid polymers and copolymers, shellac, zein, hydrogenated castor oil, hydrogenated vegetable oil, or mixtures thereof. Preferably, the hydrophobic material is a pharmaceutically acceptable acrylic polymer, including acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylicacid), poly(methacrylic acid), methacrylic acid alkylamine copolymer, poly(methyl methacrylate), poly(methacrylic acid) (anhydride), polymethacrylate, polyacrylamide, poly(methacrylic acid anhydride), and glycidyl methacrylate copolymers. In other embodiments, the hydrophobic material can also include hydrooxyalkylcelluloses such as hydroxypropylmethylcellulose and mixtures of the foregoing.

Preferred hydrophobic materials are water-insoluble with more or less pronounced hydrophobic trends. Preferably, the hydrophobic material has a melting point from about 30° C. to about 200° C., more preferably from about 45° C. to about 90° C. The hydrophobic material can include neutral or synthetic waxes, fatty alcohols (such as lauryl, myristyl, stearyl, cetyl or preferably cetostearyl alcohol), fatty acids, including fatty acid esters, fatty acid glycerides (mono-, di-, and tri-glycerides), hydrogenated fats, hydrocarbons, normal waxes, stearic acid, stearyl alcohol and hydrophobic and hydrophilic materials having hydrocarbon backbones. Suitable waxes include beeswax, glycowax, castor wax, carnauba wax and wax-like substances, e.g., material normally solid at room temperature and having a melting point of from about 30° C. to about 100° C.

Preferably, a combination of two or more hydrophobic materials are included in the matrix formulations. If an additional hydrophobic material is included, it is preferably a natural or synthetic wax, a fatty acid, a fatty alcohol, or mixtures thereof. Examples include beeswax, carnauba wax, stearic acid and stearyl alcohol.

In other embodiments, the sustained-release matrix comprises digestible, long-chain (e.g., C₈-C₅₀, preferably C₁₂-C₄₀), substituted or unsubstituted hydrocarbons, such as fatty acids, fatty alcohols, glyceryl esters of fatty acids, mineral and vegetable oils and waxes. Hydrocarbons having a melting point of between about 25° C. and about 90° C. are preferred. Of these long-chain hydrocarbon materials, fatty (aliphatic) alcohols are preferred. The oral dosage form can contain up to about 60% (by weight) of at least one digestible, long-chain hydrocarbon. Further, the sustained-release matrix can contain up to 60% (by weight) of at least one polyalkylene glycol.

In a preferred embodiment, the matrix comprises at least one water-soluble hydroxyalkyl cellulose, at least one C₁₂-C₃₆, preferably C₁₄-C₂₂, aliphatic alcohol and, optionally, at least one polyalkylene glycol. The at least one hydroxyalkyl cellulose is preferably a hydroxy (C₁-C₆) alkyl cellulose, such as hydroxypropylcellulose, hydroxypropylmethylcellulose and, preferably, hydroxyethyl cellulose. The amount of the at least one hydroxyalkyl cellulose in the oral dosage form will be determined, amongst other things, by the precise rate of opioid release required. The amount of the at least one aliphatic alcohol in the present oral dosage form will be determined by the precise rate of opioid release required. However, it will also depend on whether the at least one polyalkylene glycol is absent from the oral dosage form.

In certain embodiments, a spheronizing agent, together with the active ingredient, can be spheronized to form spheroids. Microcrystalline cellulose and hydrous lactose impalpable are examples of such agents. Additionally (or alternatively), the spheroids can contain a water-insoluble polymer, preferably an acrylic polymer, an acrylic copolymer, such as a methacrylic acid-ethyl acrylate copolymer, or ethyl cellulose. In such embodiments, the sustained-release coating will generally include a water-insoluble material such as (a) a wax, either alone or in admixture with a fatty alcohol, or (b) shellac or zein.

The sustained-release unit can be prepared by any suitable method. For example, a plasticized aqueous dispersion of the release-retarding or sequestering material can be applied onto the subunit comprising the opioid agonist. A sufficient amount of the aqueous dispersion of release-retarding or sequestering material to obtain a predetermined sustained-release of the opioid agonist when the Coated substrate is exposed to aqueous solutions, e.g., gastric fluid, is preferably applied, taking into account the physical characteristics of the opioid agonist, the manner of incorporation of the plasticizer; etc. Optionally, a further overcoat of a film-former, such as Opadry (Colorcon, West Point, Va.), can be applied after coating with the release-retarding or sequestering material.

The subunit can be cured in order to obtain a stabilized release rate of the therapeutic agent. In embodiments employing an acrylic coating, a stabilized product can be preferably obtained by subjecting the subunit to oven curing at a temperature above the glass transition temperature of the plasticized acrylic polymer for the required time period. The optimum temperature and time for the particular formulation can be determined by routine experimentation.

Once prepared, the subunit can be combined with at least one additional subunit and, optionally, other excipients or drugs to provide an oral dosage form. In addition to the above ingredients, a sustained-release matrix also can contain suitable quantities of other materials, e.g., diluents, lubricants, binders, granulating aids, colorants, flavorants and glidants that are conventional in the pharmaceutical art.

Optionally and preferably, the mechanical fragility of any of the sequestering subunits described herein is the same as the mechanical fragility of the therapeutic agent in releasable form. In this regard, tampering with the composition of the invention in a manner to obtain the therapeutic agent will result in the destruction of the sequestering subunit, such that the antagonist is released and mixed in with the therapeutic agent. Consequently, the antagonist cannot be separated from the therapeutic agent, and the therapeutic agent cannot be administered in the absence of the antagonist. Methods of assaying the mechanical fragility of the sequestering subunit and of a therapeutic agent are known in the art.

The composition of the invention can be in any suitable dosage form or formulation, (see, e.g., Pharmaceutics and Pharmacy Practice, J. B. Lippincott Company, Philadelphia, Pa., Banker and Chalmers, eds., pages 238-250 (1982)). Pharmaceutically acceptable salts of the antagonist or agonist agents discussed herein include metal salts, such as sodium salt, potassium salt, cesium salt, and the like; alkaline earth metals, such as calcium salt, magnesium salt, and the like; organic amine salts, such as triethylamine salt, pyridine salt, picoline salt, ethanolamine salt, triethanolamine salt, dicyclohexylamine salt, N,N′-dibenzylethylenediamine salt, and the like; inorganic acid salts, such as hydrochloride, hydrobromide, sulfate, phosphate, and the like; organic acid salts, such as formate, acetate, trifluoroacetate, maleate, tartrate, and the like; sulfonates, such as methanesulfonate, benzenesulfonate, p-toluenesulfonate, and the like; amino acid salts, such as arginate, asparginate, glutamate, and the like. Formulations suitable for oral administration can consist of (a) liquid solutions, such as an effective amount of the inhibitor dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

One of ordinary skill in the art will readily appreciate that the compositions of the invention can be modified in any number of ways, such that the therapeutic efficacy of the composition is increased through the modification. For instance, the therapeutic agent or sequestering subunit could be conjugated either directly or indirectly through a linker to a targeting moiety. The practice of conjugating therapeutic agents or sequestering subunits to targeting moieties is known in the art. See, for instance, Wadwa et al., J. Drug Targeting 3: 111 (1995), and U.S. Pat. No. 5,087,616. The term “targeting moiety” as used herein, refers to any molecule or agent that specifically recognizes and binds to a cell-surface receptor, such that the targeting moiety directs the delivery of the therapeutic agent or sequestering subunit to a population of cells on which the receptor is expressed. Targeting moieties include, but are not limited to, antibodies, or fragments thereof, peptides, hormones, growth factors, cytokines, and any other naturally- or non-naturally-existing ligands, which bind to cell-surface receptors. The term “linker” as used herein, refers to any agent or molecule that bridges the therapeutic agent or sequestering subunit to the targeting moiety. One of ordinary skill in the art recognizes that sites on the therapeutic agent or sequestering subunit, which are not necessary for the function of the agent or sequestering subunit, are ideal sites for attaching a linker and/or a targeting moiety, provided that the linker and/or targeting moiety, once attached to the agent or sequestering subunit, do(es) not interfere with the function of the therapeutic agent or sequestering subunit.

With respect to the present inventive compositions, the composition is preferably an oral dosage form. By “oral dosage form” is meant to include a unit dosage form prescribed or intended for oral administration comprising subunits. Desirably, the composition comprises the sequestering subunit coated with the therapeutic agent in releasable form, thereby forming a composite subunit comprising the sequestering subunit and the therapeutic agent. Accordingly, the invention further provides a capsule suitable for oral administration comprising a plurality of such composite subunits.

Alternatively, the oral dosage form can comprise any of the sequestering subunits of the invention in combination with a therapeutic agent subunit, wherein the therapeutic agent subunit comprises the therapeutic agent in releasable form. In this respect, the invention provides a capsule suitable for oral administration comprising a plurality of sequestering subunits of the invention and a plurality of therapeutic subunits, each of which comprises a therapeutic agent in releasable form.

The invention further provides tablets comprising a sequestering subunit of the invention and a therapeutic agent in releasable form. For instance, the invention provides a tablet suitable for oral administration comprising a first layer comprising any of the sequestering subunits of the invention and a second layer comprising therapeutic agent in releasable form, wherein the first layer is coated with the second layer. The first layer can comprise a plurality of sequestering subunits. Alternatively, the first layer can be or can consist of a single sequestering subunit. The therapeutic agent in releasable form can be in the form of a therapeutic agent subunit and the second layer can comprise a plurality of therapeutic subunits. Alternatively, the second layer can comprise a single substantially homogeneous layer comprising the therapeutic agent in releasable form.

When the blocking agent is a system comprising a first antagonist-impermeable material and a core, the sequestering subunit can be in one of several different forms. For example, the system can further comprise a second antagonist-impermeable material, in which case the sequestering unit comprises an antagonist, a first antagonist-impermeable material, a second antagonist-impermeable material, and a core. In this instance, the core is coated with the first antagonist-impermeable material, which, in turn, is coated with the antagonist, which, in turn, is coated with the second antagonist-impermeable material. The first antagonist-impermeable material and second antagonist-impermeable material substantially prevent release of the antagonist from the sequestering subunit in the gastrointestinal tract for a time period that is greater than 24 hours. In some instances, it is preferable that the first antagonist-impermeable material is the same as the second antagonist-impermeable material. In other instances, the first antagonist-impermeable material is different from the second antagonist-impermeable material. It is within the skill of the ordinary artisan to determine whether or not the first and second antagonist-impermeable materials should be the same or different. Factors that influence the decision as to whether the first and second antagonist-impermeable materials should be the same or different can include whether a layer to be placed over the antagonist-impermeable material requires certain properties to prevent dissolving part or all of the antagonist-impermeable layer when applying the next layer or properties to promote adhesion of a layer to be applied over the antagonist-impermeable layer.

Alternatively, the antagonist can be incorporated into the core, and the core is coated with the first antagonist-impermeable material. In this case, the invention provides a sequestering subunit comprising an antagonist, a core and a first antagonist-impermeable material, wherein the antagonist is incorporated into the core and the core is coated with the first antagonist-impermeable material, and wherein the first antagonist-impermeable material substantially prevents release of the antagonist from the sequestering subunit in the gastrointestinal tract for a time period that is greater than 24 hours. By “incorporate” and words stemming therefrom, as used herein is meant to include any means of incorporation, e.g., homogeneous dispersion of the antagonist throughout the core, a single layer of the antagonist coated on top of a core, or a multi-layer system of the antagonist, which comprises the core.

In another alternative embodiment, the core comprises a water-insoluble material, and the core is coated with the antagonist, which, in turn, is coated with the first antagonist-impermeable material. In this case, the invention further provides a sequestering subunit comprising an antagonist, a first antagonist-impermeable material, and a core, which comprises a water-insoluble material, wherein the core is coated with the antagonist, which, in turn, is coated with the first antagonist-impermeable material, and wherein the first antagonist-impermeable material substantially prevents release of the antagonist from the sequestering subunit in the gastrointestinal tract for a time period that is greater than 24 hours. The term “water-insoluble material” as used herein means any material that is substantially water-insoluble. The term “substantially water-insoluble” does not necessarily refer to complete or 100% water-insolubility. Rather, there are varying degrees of water insolubility of which one of ordinary skill in the art recognizes as having a potential benefit. Preferred water-insoluble materials include, for example, microcrystalline cellulose, a calcium salt, and a wax. Calcium salts include, but are not limited to, a calcium phosphate (e.g., hydroxyapatite, apatite; etc.), calcium carbonate, calcium sulfate, calcium stearate, and the like. Waxes include, for example, carnuba wax, beeswax, petroleum wax, candelilla wax, and the like.

In one embodiment, the sequestering subunit includes an antagonist and a seal coat where the seal coat forms a layer physically separating the antagonist within the sequestering subunit from the agonist which is layered upon the sequestering subunit. In one embodiment, the seal coat comprises one or more of an osmotic pressure regulating agent, a charge-neutralizing additive, a sequestering polymer hydrophobicity-enhancing additive, and a first sequestering polymer (each having been described above). In such embodiments, it is preferred that the osmotic pressure regulating agent, charge-neutralizing additive, and/or sequestering polymer hydrophobicity-enhancing additive, respectively where present, are present in proportion to the first, sequestering polymer such that no more than 10% of the antagonist is released from the intact dosage form. Where an opioid antagonist is used in the sequestering subunit and the intact dosage form includes an opioid agonist, it is preferred that ratio of the osmotic pressure regulating agent, charge-neutralizing additive, and/or sequestering polymer hydrophobicity-enhancing additive, respectively where present, in relation to the first sequestering polymer is such that the physiological effect of the opioid agonist is not diminished when the composition is in its intact dosage form or during the normal course digestion in the patient. Release may be determined as described above using the USP paddle method (optionally using a buffer containing a surfactant such as Triton X-100) or measured from plasma after administration to a patient in the fed or non-fed state. In one embodiment, plasma naltrexone levels are determined; in others, plasma 6-beta naltrexol levels are determined. Standard tests may be utilized to ascertain the antagonist's effect on agonist function (i.e., reduction of pain).

The sequestering subunit of the invention can have a blocking agent that is a tether to which the antagonist is attached. The term “tether” as used herein refers to any means by which the antagonist is tethered or attached to the interior of the sequestering subunit, such that the antagonist is not released, unless the sequestering subunit is tampered with. In this instance, a tether-antagonist complex is formed. The complex is coated with a tether-impermeable material, thereby substantially preventing release of the antagonist from the subunit. The term “tether-impermeable material” as used herein refers to any material that substantially prevents or prevents the tether from permeating through the material. The tether preferably is an ion exchange resin bead.

The invention further provides a tablet suitable for oral administration comprising a single layer comprising a therapeutic agent in releasable form and a plurality of any of the sequestering subunits of the invention dispersed throughout the layer of the therapeutic agent in releasable form. The invention also provides a tablet in which the therapeutic agent in releasable form is in the form of a therapeutic agent subunit and the tablet comprises an at least substantially homogeneous mixture of a plurality of sequestering subunits and a plurality of subunits comprising the therapeutic agent.

In preferred embodiments, oral dosage forms are prepared to include an effective amount of melt-extruded subunits in the form of multiparticles within a capsule. For example, a plurality of the melt-extruded muliparticulates can be placed in a gelatin capsule in an amount sufficient to provide an effective release dose when ingested and contacted by gastric fluid.

In another preferred embodiment, the subunits, e.g., in the form of multiparticulates, can be compressed into an oral tablet using conventional tableting equipment using standard techniques. Techniques and compositions for making tablets (compressed and molded), capsules (hard and soft gelatin) and pills are also described in Remington's Pharmaceutical Sciences, (Aurther Osol., editor), 1553-1593 (1980), which is incorporated herein by reference. Excipients in tablet formulation can include, for example, an inert diluent such as lactose, granulating and disintegrating agents, such as cornstarch, binding agents, such as starch, and lubricating agents, such as magnesium stearate. In yet another preferred embodiment, the subunits are added during the extrusion process and the extrudate can be shaped into tablets as set forth in U.S. Pat. No. 4,957,681 (Klimesch et al.), which is incorporated herein by reference.

Optionally, the sustained-release, melt-extruded, multiparticulate systems or tablets can be coated, or the gelatin capsule can be further coated, with a sustained-release coating, such as the sustained-release coatings described herein. Such coatings are particularly useful when the subunit comprises an opioid agonist in releasable form, but not in sustained-release form. The coatings preferably include a sufficient amount of a hydrophobic material to obtain a weight gain level form about 2 to about 30 percent, although the overcoat can be greater, depending upon the physical properties of the particular opioid analgesic utilized and the desired release rate, among other things.

The melt-extruded dosage forms can further include combinations of melt-extruded multiparticulates containing one or more of the therapeutically active agents before being encapsulated. Furthermore, the dosage forms can also include an amount of an immediate release therapeutic agent for prompt therapeutic effect. The immediate release therapeutic agent can be incorporated or coated on the surface of the subunits after preparation of the dosage forms (e.g., controlled-release coating or matrix-based). The dosage forms can also contain a combination of controlled-release beads and matrix multiparticulates to achieve a desired effect.

The sustained-release formulations preferably slowly release the therapeutic agent, e.g., when ingested and exposed to gastric fluids, and then to intestinal fluids. The sustained-release profile of the melt-extruded formulations can be altered, for example, by varying the amount of retardant, e.g., hydrophobic material, by varying the amount of plasticizer relative to hydrophobic material, by the inclusion of additional ingredients or excipients, by altering the method of manufacture; etc.

In other embodiments, the melt-extruded material is prepared without the inclusion of the subunits, which are added thereafter to the extrudate. Such formulations can have the subunits and other drugs blended together with the extruded matrix material, and then the mixture is tableted in order to provide a slow release of the therapeutic agent or other drugs. Such formulations can be particularly advantageous, for example, when the therapeutically active agent included in the formulation is sensitive to temperatures needed for softening the hydrophobic material and/or the retardant material.

In certain embodiments, the release of the antagonist of the sequestering subunit or composition is expressed in terms of a ratio of the release achieved after tampering, e.g., by crushing or chewing, relative to the amount released from the intact formulation. The ratio is, therefore, expressed as Crushed:Whole, and it is desired that this ratio have a numerical range of at least about 4:1 or greater (e.g., crushed release within 1 hour/intact release in 24 hours). In certain embodiments, the ratio of the therapeutic agent and the antagonist, present in the sequestering subunit, is about 1:1, about 50:1, about 75:1, about 100:1, about 150:1, or about 200:1, for example, by weight, preferably about 1:1 to about 20:1 by weight or 15:1 to about 30:1 by weight. The weight ratio of the therapeutic agent to antagonist refers to the weight of the active ingredients. Thus, for example, the weight of the therapeutic agent excludes the weight of the coating, matrix, or other component that renders the antagonist sequestered, or other possible excipients associated with the antagonist particles. In certain preferred embodiments, the ratio is about 1:1 to about 10:1 by weight. Because in certain embodiments the antagonist is in a sequestered from, the amount of such antagonist within the dosage form can be varied more widely than the therapeutic agent/antagonist combination dosage forms, where both are available for release upon administration, as the formulation does not depend on differential metabolism or hepatic clearance for proper functioning. For safety reasons, the amount of the antagonist present in a substantially non-releasable form is selected as not to be harmful to humans, even if fully released under conditions of tampering.

Thus, in certain embodiments, a pharmaceutical composition comprising an antagonist in direct contact with a seal coat, an agonist in direct contact with the seal coat and a sequestering polymer but not the antagonist, wherein the antagonist and agonist are present within a single multilayer pharmaceutical unit, is provided. In others, pharmaceutical compositions comprising a pharmaceutical dosing unit consisting essentially of a multiple layer bead comprising an antagonist and an agonist that are not in direct contact with one another are provided. In yet others, pharmaceutical composition comprising a plurality of pharmaceutically active units wherein each unit comprises an antagonist, an agonist, a seal coat, and a sequestering polymer wherein the antagonist and the agonist are not in direct contact with one another. In still others, pharmaceutical compositions comprising a pharmaceutically inert support material such as a sugar sphere, an antagonist in direct contact with the support material, a seal coat in direct contact with the antagonist and an agonist, and a sequestering polymer in direct contact with the agonist are provided. In preferred embodiments, multiple layer pharmaceutical compositions comprising an agonist and an antagonist within distinct layers of the composition, wherein at least 90-95% of the antagonist is sequestered for at least 24 hours following administration to a human being are provided. In a particularly preferred embodiment, a pharmaceutical composition comprising naltrexone within a sequestering subunit and morphine in contact with the subunit but not the naltrexone, wherein administration of the composition to a human being results in the release of substantially all of the morphine from the composition but less than 5-10% of the naltrexone from the composition within 24 hours of administration, is provided. Also provided are methods for preparing pharmaceutical compositions by, for example, adhering an antagonist to a pharmaceutically inert support material, coating the antagonist with a seal coat that includes a sequestering polymer, coating the seal coat with an agonist, and coating the agonist with a release-retarding or sequestering material. In another embodiment, a method for measuring the amount of antagonist or derivative thereof in a biological sample, the antagonist or derivative having been released from a pharmaceutical composition in vivo, the method comprising the USP paddle method at 37° C., 100 rpm, but further comprising incubation in a buffer containing a surfactant such as Triton X-100, for example.

A particularly preferred embodiment comprises a multiple layer pharmaceutical is described in the Examples is multi-layer naltrexone/morphine dosing unit in an abuse-resistant dosage form. Naltrexone is contained in a sequestering subunit comprising a seal coat comprising Eudragit® RS and the optimization agents SLS, talc and chloride ions that together prevent release of naltrexone upon hydration. Overlayed onto the sequestering subunit is a layer comprising morphine that is released upon hydration in pH 7.5 buffer; the naltrexone, however, remains within the sequestering subunit under these conditions. It is preferred that if the unit is modified or substantially disrupted by, for example, crushing the unit, the sequestering subunit is crushed as well causing the release of both morphine and naltrexone therefrom.

Thus, the compositions are particularly well-suited for use in preventing abuse of a therapeutic agent. In this regard, the invention also provides a method of preventing abuse of a therapeutic agent by a human being. The method comprises incorporating the therapeutic agent into any of the compositions of the invention. Upon administration of the composition of the invention to the person, the antagonist is substantially prevented from being released in the gastrointestinal tract for a time period that is greater than 24 hours. However, if a person tampers with the compositions, the sequestering subunit, which is mechanically fragile, will break and thereby allow the antagonist to be released. Since the mechanical fragility of the sequestering subunit is the same as the therapeutic agent in releasable form, the antagonist will be mixed with the therapeutic agent, such that separation between the two components is virtually impossible.

A better understanding of the present invention and of its many advantages will be had from the following examples, given by way of illustration. All references cited herein are incorporated by reference in their entirety into this application.

### EXAMPLES

### Example 1

### Optimization Study #4, Morphine Sulfate and Naltrexone HCl 60 mg/4.8 mg (20-780-1N)

TABLE 1

| PI-1495 | | PI-1496 | |
|---|---|---|---|
| mg/unit | Percent | mg/unit | Percent |

Sealed-coated sugar spheres

| | | | | |
|---|---|---|---|---|
| Sugar spheres (#25-30 mesh) | 37.2 | 11.7 | 37.1 | 11.9 |
| Ethylcellulose N50 | 6.2 | 1.9 | 6.2 | 2.0 |
| Mag Stearate | 2.5 | 0.8 | 2.5 | 0.8 |
| DBS | 0.6 | 0.2 | 0.6 | 0.2 |
| Talc | 15.5 | 4.9 | 15.5 | 5.0 |
| Subtotal | 62.0 | 19.4 | 61.9 | 19.9 |

Naltrexone cores

| | | | | |
|---|---|---|---|---|
| Sealed sugar spheres | (62.0) | (19.4) | (61.9) | (19.9) |
| Naltrexone HCl | 4.8 | 1.50 | 4.8 | 1.54 |
| HPC (Klucel LF) | 0.9 | 0.3 | 0.9 | 0.3 |
| Ascorbic acid | 0.5 | 0.2 | 0.5 | 0.2 |
| Talc | 2.27 | 0.7 | 2.24 | 0.7 |
| Subtotal | 70.5 | 22.1 | 70.3 | 22.6 |

Naltrexone pellets

| | | | | |
|---|---|---|---|---|
| Naltrexone cores | (70.5) | (22.1) | (70.3) | (22.6) |
| Eudragit RS PO | 53.3 | 16.7 | 53.3 | 17.1 |
| SLS | 1.8 | 0.6 | 1.8 | 0.6 |
| DBS | 5.36 | 1.7 | 5.36 | 1.7 |
| Talc | 52.1 | 16.3 | 52.1 | 16.8 |
| Subtotal | 183.0 | 57.4 | 182.9 | 58.8 |

Naltrexone-morphine cores

| | | | | |
|---|---|---|---|---|
| Naltrexone pellets | (183.0) | (57.4) | (182.9) | (58.8) |
| Morphine sulfate | 59.9 | 18.8 | 59.7 | 19.2 |
| Sodium chloride | 11.2 | 3.5 | | |
| HPC (Klucel LF) | 7.3 | 2.3 | 4.76 | 1.5 |
| HPMC, 3 cps | | | 7.6 | 2.4 |
| Subtotal | 261.4 | 82.0 | 255.0 | 82.0 |

Naltrexone-morphine pellets

| | | | | |
|---|---|---|---|---|
| Naltrexone-morphine cores | (261.4) | (82.0) | (255.0) | (82.0) |
| Ethylcellulose N50 | 19.81 | 6.2 | 19.31 | 6.2 |
| PEG 6000 | 9.16 | 2.9 | 8.9 | 2.9 |
| Eudragit L100-55 | 4.3 | 1.3 | 4.2 | 1.4 |
| DEP | 4.12 | 1.3 | 4 | 1.3 |
| Talc | 20.13 | 6.3 | 19.62 | 6.3 |
| Total | 319.0 | 100.0 | 311.0 | 100.0 |

### A. Method of Preparation—

1. Dissolve Ethylcellulose and dibutyl sebacate into ethanol, then disperse talc and magnesium stearate into the solution.
2. Spray the dispersion from 1 onto sugar spheres in a Wurster to form seal-coated sugar spheres (50 μm seal coat).
3. Dissolve Klucel LF and ascorbic acid into 20:80 mixture of water and ethanol. Disperse naltrexone HCl and talc into the solution.
4. Spray the naltrexone dispersion from 3 onto seal-coated sugar spheres from 2 in a Wurster to form naltrexone cores.
5. Dissolve Eudragit RS, sodium lauryl sulfate and dibutyl debacate into ethanol. Disperse talc into the solution.
6. Spray the dispersion from 5 onto naltrexone cores from 4 in a Wurster to form naltrexone pellets.
7. The Naltrexone pellets are dried at 50° C. for 48 hours.
8. Resulting pellets have a Eudragit RS coat thickness of 150 μm for both PI-1495 PI-1496.
9. (Only for P1-1495) Dissolve sodium chloride and hydroxypropylcellulose (HPC; Klucel LF) into water.
10. Dissolve hypromellose into 10:90, mixture of water and ethanol. Disperse morphine sulfate into the solution.
11. (Only for PI-1495) Spray the solution from 9 followed by the dispersion from 10 onto naltrexone pellets in 7 in a rotor to form naltrexone-morphine cores.
12. (Only for PI-1496) Spray the dispersion from 10 onto naltrexone pellets in 7 in a rotor to form naltrexone-morphine cores.
13. Dissolve ethylcellulose, PEG 6000, Eudragit L100-55 and diethyl phthalate into ethanol. Disperse talc into the solution.
14. Spray the dispersion from 12 onto naltrexone-morphine cores in 11 or 12 to form naltrexone-morphine pellets.
15. The pellets are filled into capsules.

### B. In-Vitro Drug Release—

1. Method—USP paddle method at 37° C. and 100 rpm
   1 hour in 0.1N HCl, then 72 hours in 0.05M pH 7.5 phosphate buffer
Results—Percent of NT released at 73 hours for PI-1495=0%
   Percent of NT released at 73 hours for PI-1496=0%
2. Method—USP paddle method at 37° C. and 100 rpm
   72 hrs in 0.2% Triton X-100/0.2% sodium acetate/0.002N HCl, pH 5.5
Results—Percent of NT released at 73 hours for PI-1495=0%
   Percent of NT released at 73 hours for PI-1496=0%

### C. In-Vivo Study

This is a single-dose, open-label, two period study in which two groups of eight subjects received one dose of either PI-1495 or PI-1496. Each subject received an assigned treatment sequence based on a randomization schedule under fasting and non-fasting conditions. Blood samples were drawn prior to dose administration and at 0.5 to 168 hours post-dose. Limits of quantitation are 4.00 pg/mL for naltrexone and 0.250 pg/mL for 6-beta-naltrexol.

### 2. Summary of Pharmacokinetic Parameters

TABLE 2
Naltrexone levels

| | PI-1495 | | PI-1496 | |
|---|---|---|---|---|
| | Fast | Fed | Fast | Fed |
| Tmax (hr) | 54.00 (N = 2) | 14.34 (N = 3) | 55.20 (N = 5) | 41.60 (N = 5) |
| Cmax (pg/mL) | 8.53 | 6.32 (N = 7) | 24.23 (N = 7) | 45.67 (N = 7) |
| AUCₗₐₛₜ (pg * h/mL) | 100.8 | 75.9 (N = 7) | 500.6 (N = 7) | 1265 (N = 7) |
| AUC∞ (pg * h/mL) | — | — | 2105.3 (N = 2) | 3737 (N = 2) |
| T½ (hr) | — | — | 44.56 (N = 2) | 33.17 (N = 2) |

Relative Bioavailability to an oral solution (Dose-adjusted)

| | | | | |
|---|---|---|---|---|
| Cmax Ratio (Test/Solution) | 0.29% | 0.21% | 0.82% | 1.55% |
| AUCₗₐₛₜ Ratio (Test/Solution) | 1.13% | 0.85% | 5.61% | 14.17% |
| AUC∞ Ratio (Test/Solution) | — | — | 22.0% | 39.1% |

N = 8, unless specified otherwise

TABLE 3
6-beta naltrexol levels

| | PI-1495 | | PI-1496 | |
|---|---|---|---|---|
| | Fast | Fed | Fast | Fed |
| Tmax (hr) | 69.00 | 41.44 (N = 7) | 70.51 | 67.63 |
| Cmax (pg/mL) | 116.3 | 151.7 (N = 7) | 303.3 | 656.7 |
| AUCₗₐₛₜ (pg * h/mL) | 5043 | 7332 (N-7) | 14653 | 27503 |
| AUC∞ (pg * h/mL) | 5607 | 8449 (N = 6) | 14930 | 27827 |
| T½ (hr) | 20.97 | 16.69 (N = 7) | 16.29 | 22.59 |

Relative Bioavailability to an oral solution (Dose-adjusted)

| | | | | |
|---|---|---|---|---|
| Cmax Ratio | 0.47% | 0.62% | 1.23% | 2.67% |
| (Test/Solution) | | | | |
| AUCₗₐₛₜ Ratio | 2.45% | 3.45% | 7.12% | 13.36% |
| (Test/Solution) | | | | |
| AUC∞ Ratio | 2.64% | 3.97% | 7.02% | 13.08% |
| (Test/Solution) | | | | |

N = 8, unless specified otherwise

### 3. Conclusion

a. Kadian NT pellets with naltrexone pellet coat thickness of 150 μm had comparable naltrexone release as NT pellets with 90 μm coat thickness. This comparable NT release may also be attributed from the presence of 50 μm seal coat on the sugar spheres used in Kadian NT pellets.
b. Significant NT sequestering was observed, both at fasting (>97%) and fed states (>96%).
c. Kadian NT pellets containing sodium chloride immediately above the naltrexone pellet coat (PI-1495) had half the release of naltrexone compared to Kadian NT pellet without sodium chloride (PI-1496), consistent with in vitro results.
d. There is again food effect observed. Lag time was significantly reduced.

### Example 2

### Optimization Study #5, Morphine Sulfate and Naltrexone HCl 60 mg/2.4 mg (20-903-AU)

TABLE 4

PI-1510

| | Mg/unit | Percent |
|---|---|---|
| Sealed sugar spheres | | |
| Sugar spheres (#25-30 mesh) | 39.9 | 12.2 |
| Ethylcellulose N50 | 6.5 | 2.0 |
| Mag Stearate | 2.6 | 0.8 |
| DBS | 0.7 | 0.2 |
| Talc | 16.7 | 5.1 |
| Subtotal | 66.4 | 20.3 |
| Naltrexone cores | | |
| Sealed sugar spheres | (66.4) | (20.3) |
| Naltrexone HCl | 2.4 | 0.73 |
| HPC (Klucel LF) | 0.5 | 0.1 |
| Ascorbic acid | 0.2 | 0.1 |
| Talc | 1.1 | 0.4 |
| Subtotal | 70.6 | 21.6 |
| Naltrexone pellets | | |
| Naltrexone cores | (70.6) | (21.6) |
| Eudragit RS PO | 53.0 | 16.2 |
| SLS | 1.8 | 0.6 |
| DBS | 5.3 | 1.6 |
| Talc | 53.0 | 16.2 |
| Subtotal | 183.7 | 56.2 |
| Naltrexone-morphine cores | | |
| Naltrexone pellets | (183.7) | (56.2) |
| Morphine sulfate | 60.1 | 18.4 |
| Sodium chloride | 12.5 | 3.8 |
| HPC (Klucel LF) | 6.2 | 1.9 |
| Subtotal | 262.4 | 80.2 |
| Naltrexone-morphine pellets | | |
| Naltrexone-morphine cores | (262.4) | (80.2) |
| Ethylcellulose N50 | 22.9 | 7.0 |
| PEG 6000 | 10.6 | 3.2 |
| Eudragit L100-55 | 5.0 | 1.5 |
| DEP | 4.7 | 1.5 |
| Talc | 21.5 | 6.6 |
| Total | 327.1 | 100.0 |

B. Method of preparation for PI-1510—
1. Dissolve Ethylcellulose and dibutyl sebacate into ethanol, then disperse talc and magnesium stearate into the solution. Percent solid in the dispersion is 20%.
2. Spray the dispersion from 1 onto sugar spheres in a Wurster to form seal-coated sugar spheres (50 μm seal coat).
3. Dissolve Klucel LF and ascorbic acid into 20:80 mixture of water and ethanol. Disperse naltrexone HCl and talc into the solution. Percent solid in the dispersion is 21%.
4. Spray the naltrexone dispersion from 3 onto seal-coated sugar spheres from 2 in a Wurster to form naltrexone cores.
5. Dissolve Eudragit RS, sodium lauryl sulfate and dibutyl sebacate into ethanol. Disperse talc into the solution. Percent solid in the dispersion is 19.7%.
6. Spray the dispersion from 5 onto naltrexone cores from 4 in a Wurster to form naltrexone pellets.
7. The Naltrexone pellets are dried at 50° C. for 48 hours.
8. Resulting pellets have a Eudragit RS coat thickness of 150 μm.
9. Dissolve sodium chloride and Hydroxypropyl Cellulose (HPC; Klucel LF) (0.4% of the 1.9%) into water. Percent solid in the solution is 5.9%.
10. Dissolve the remaining 1.5% of the HPC into ethanol. Disperse morphine sulfate into the solution. Percent solid in the dispersion is 24.9%.
11. Spray the solution from 9 followed by the dispersion from 10 onto naltrexone pellets in 7 in a rotor to form naltrexone-morphine cores.
12. Dissolve ethylcellulose, PEG 6000, Eudragit L100-55 and diethyl phthalate into ethanol. Disperse talc into the solution. Percent solid in the dispersion is 14.3%.
13. Spray the dispersion from 12 onto naltrexone-morphine cores in 11 or 12 to form naltrexone-morphine pellets.
14. The pellets are filled into capsules.

### Example 3

### Kadian NT Formulation #6 (AL-01)

TABLE 5

| | | Final |
|---|---|---|
| | | formulation |
| | 15% TPCW | AL-01 |
| Seal-coated Sugar Spheres | | |
| Sugar Spheres (#25-30 mesh) | 11.99 | 11.94 |
| Ethylcellulose NF 50 cps | 2.00 | 1.99 |
| Magnesium Stearate NF | 0.80 | 0.80 |
| Dibutyl Sebacate NF | 0.20 | 0.20 |
| Talc USP (Suzorite 1656) | 5.00 | 4.98 |
| Naltrexone HCl Core | | |
| Seal-coated Sugar Spheres | | (19.90) |
| Naltrexone Hydrochloride USP | 0.73 | 0.72 |
| Hydroxypropyl Cellulose NF | 0.14 | 0.14 |
| Ascorbic Acid USP | 0.07 | 0.07 |
| Talc USP (Suzorite 1656) | 0.34 | 0.34 |
| Naltrexone HCl Intermediate Pellet | | |
| Naltrexone HCl Core | | (21.17) |
| Ammonio Methacrylate Copolymer Type | 6.26 | 6.23 |
| B NF | | |
| Sodium Lauryl Sulfate NF | 0.22 | 0.22 |
| Dibutyl Sebacate NF | 0.63 | 0.62 |
| Talc USP (Suzorite 1656) | 6.08 | 6.05 |
| Naltrexone HCl Finished Pellet | | |
| Naltrexone HCl Intermediate Pellet | | (34.29) |
| Ammonio Methacrylate Copolymer Type | 9.89 | 9.85 |
| B NF | | |
| Sodium Lauryl Sulfate NF | 0.34 | 0.34 |
| Dibutyl Sebacate NF | 0.99 | 0.98 |
| Talc USP (Suzorite 1656) | 9.71 | 9.67 |
| NaCl Overcoated Naltrexone HCl Pellet | | |
| Naltrexone HCl Finished Pellet | | (55.13) |
| Sodium Chloride USP | 3.75 | 3.73 |
| Hydroxypropyl Cellulose NF | 0.42 | 0.41 |
| MS Cores with Sequestered Naltrexone HCl | | |
| NaCl Overcoated Naltrexone HCl Pellet | | (59.28) |
| Morphine Sulfate USP | 18.11 | 18.03 |
| Hydroxypropyl Cellulose NF | 1.42 | 1.42 |
| MS Extended-release with Sequestered | | |
| Naltrexone HCl Pellet | | |
| MS Cores with Sequestered Naltrexone HCl | | (78.73) |
| Component (a): ethylcellulose NF (50 cps) | 7.40 | 7.36 |
| Component (c): polyethylene glycol NF | 3.42 | 3.40 |
| (6000) | | |
| Component (b): methacrylic acid copolymer | 1.60 | 1.60 |
| NF | | |
| (Type C, Powder) | | |
| Diethyl Phthalate NF (plasticizer) | 1.53 | 1.53 |
| Talc USP (Suzorite 1656) (filler) | 6.98 | 7.38 |
| Total | 100.0 | 100.0 |

In certain embodiments, components (a), (b) and/or (c) may be included as described below:
(a) preferably a matrix polymer insoluble at pH of about 1 to about 7.5; preferably ethylcellulose; preferably at least 35% by weight of a+b+c;
(b) preferably an enteric polymer insoluble at pH of about 1 to about 4 but soluble at pH of about 6 to about 7.5; preferably methacrylic acid-ethyl acrylate copolymer (methacrylic acid copolymer type C) preferably about 1 to about 30% of a+b+c; and,
(c) compound soluble at a pH from about 1 to about 4; preferably polyethylene glycol with a molecular weight from about 1700 to about 20,000; preferably from about 1% to about 60% by weight of a+b+c.

### C. Method of Preparation for Final Formulation of ALO-01—

1. Dissolve Ethylcellulose and Dibutyl Sebacate into Alcohol SDA3A, then disperse Talc and Magnesium Stearate into the solution. Percent solid of the dispersion is 20%.
2. Spray the dispersion from 1 onto Sugar Spheres in a Wurster to form Seal-coated Sugar Spheres (approx. 50 μm seal coat).
3. Dissolve Hydroxypropyl Cellulose and Ascorbic Acid into 20:80 mixture of Water and Alcohol SDA3A. Disperse Naltrexone HCl and Talc into the solution. Percent solid of the dispersion is 20.4%.
4. Spray the Naltrexone HCl dispersion from 3 onto Seal-coated Sugar Spheres from 2 in a Wurster to form Naltrexone HCl cores.
5. Dissolve Ammonio Methacrylate Copolymer, Sodium Lauryl Sulfate and Dibutyl Sebacate into 22:78 mixture of Water and Alcohol SDA3A. Disperse Talc into the solution. Percent solid of the dispersion is 20%.
6. Spray the dispersion from 5 onto Naltrexone HCl cores from 4 in a Wurster to form Naltrexone HCl Intermediate Pellets.
7. The Naltrexone HCl Intermediate Pellets are dried in an oven at 50° C. for 24 hours.
8. Dissolve Ammonio Methacrylate Copolymer, Sodium Lauryl Sulfate and Dibutyl Sebacate into 22:78 mixture of Water and Alcohol SDA3A. Disperse Talc into the solution. Percent solid of the dispersion is 20%.
9. Spray the dispersion from 8 onto Naltrexone HCl Intermediate Pellets from 7 in a Wurster to form Naltrexone HCl Finished Pellets.
10. The Naltrexone HCl Finished Pellets are dried in an oven at 50° C. for 24 hours.
11. Resulting pellets have a pellet coat thickness of approximately 150 μm.
12. Dissolve Sodium Chloride (NaCl) and Hydroxypropyl Cellulose into Water. Percent solid in the solution is 6%.
13. Spray the Sodium Chloride solution from 12 onto Naltrexone HCl Finished Pellets from 10 in a Wurster to form Sodium Chloride (NaCl) Overcoated Naltrexone HCl Pellets.
14. Dissolve Hydroxypropyl Cellulose into Alcohol SDA3A. Disperse Morphine Sulfate into the solution. Percent solid in the dispersion is 24.4%.
15. Spray the Morphine Sulfate dispersion from 14 onto NaCl Overcoated Naltrexone HCl Pellets in 13 in a rotor to form Morphine Sulfate Cores with Sequestered Naltrexone HCl.
16. Dissolve Ethylcellulose, Polyethylene Glycol, Methacrylic Acid Copolymer and Diethyl Phthalate into Alcohol SDA3A. Disperse Talc into the solution. Percent solid in the dispersion is 14.3%.
17. Spray the Dispersion from 16 onto Morphine Sulfate Cores with Sequestered Naltrexone HCl in 15 to form Morphine Sulfate Extended-release with Sequestered Naltrexone HCl Pellets.
18. The pellets are filled into capsules.

### Example 4

### Methods for Treating Pain

Kadian NT (60 mg morphine sulfate, 2.4 mg naltrexone HCl) was administered to humans and compared to the previously described product Kadian. Each Kadian sustained release capsule contains either 20, 30, 50, 60, or 100 mg of Morphine Sulfate USP and the following inactive ingredients common to all strengths: hydroxypropyl methylcellulose, ethylcellulose, methacrylic acid copolymer, polyethylene glycol, diethyl phthalate, talc, corn starch, and sucrose. In these studies, the effects of Kadian were compared to those of Kadian NT.

Patients already being treated with Kadian were subjected to a “washout” period of approximately 14 days during which Kadian was not administered. Immediately following this washout period, the trial was begun. Patients were either administered Kadian or Kadian NT at day 0. After a period of up to 28 days treatment with Kadian®, patients were then “crossed-over” to Kadian NT or continued taking Kadian®. The amount of Kadian NT was individually adjusted such that each patient was receiving approximately the same amount of morphine they had previously been receiving while taking Kadian. This cross-over was then repeated after 14 days. Various physiological responses were measured at different timepoints, as discussed below. These responses included morphine blood levels, naltrexone blood levels, 6-O-natrexol blood levels and pain scores.

Mean morphine concentrations were measured and determined to be approxiMately the same for Kadian® and Kadian NT. This observation confirms that the new formulation effectively releases morphine into the blood of patients. This is shown in Table 6 below:

TABLE 6

| | | | | | |
|---|---|---|---|---|---|
| | | | | | AUC |
| Cmax | Cmin | Cavg | Tmax | Fluctuation | (TAU) |
| (pg/mL) | (pg/mL) | (pg/mL) | (hr) | (%) | (hr*pg/mL) |

Kadian

| | | | | | | |
|---|---|---|---|---|---|---|
| N | 68 | 68 | 68 | 68 | 68 | 68 |
| Mean | 12,443 | 6,650 | 9,317 | 4.90 | 66.3 | 111,806 |
| SD | 7,680 | 4,544 | 6,019 | 3.36 | 28.8 | 72,223 |
| Min | 2,630 | 1,000 | 1,758 | 0.00 | 21.4 | 21,100 |
| Median | 9,870 | 5,285 | 7,426 | 5.00 | 63.5 | 89,110 |
| Max | 35,600 | 21,600 | 28,908 | 12.0 | 213 | 346,900 |
| CV % | 61.7 | 68.3 | 64.6 | 68.5 | 43.4 | 64.6 |

Kadian NT

| | | | | | | |
|---|---|---|---|---|---|---|
| N | 68 | 68 | 68 | 68 | 68 | 68 |
| Mean | 13,997 | 6,869 | 10,120 | 4.29 | 71.49 | 121,438 |
| SD | 10,949 | 5,377 | 7,316 | 3.05 | 38.59 | 87,794 |
| Min | 2,420 | 0.00 | 1,815 | 0.00 | 21.04 | 21,775 |
| Median | 10,200 | 5,805 | 7,496 | 4.00 | 65.89 | 89,948 |
| Max | 57,600 | 29,000 | 35,046 | 12.0 | 265 | 420,550 |
| CV % | 78.2 | 78.3 | 72.3 | 71.0 | 54.0 | 72.3 |

It is important that the Kadian NT formulation not release significant amounts of antagonist (i.e., naltrexone or derivatives thereof) into the bloodstream such that the activity of morphine is diminished. Only 14 of 69 patients had quantifiable (>4.0 pg/mL) naltrexone concentrations. The range of quantifiable concentrations was 4.4-25.5 pg/mL. However, the release of some naltrexone into the bloodstream did not significantly affect the pain scores (Table 7).

TABLE 7

| | Naltrexone | |
|---|---|---|
| | Conc | |
| Subject | (pg/mL) | Pain Score* |
| 49411 | 25.5 | 2 |
| 49408 | 16.8 | 3 |
| 59510 | 15.9 | 2 |
| 29218 | 13.5 | 0 |
| 39308 | 7.74 | 0 |
| 39306 | 8.98 | 1 |
| 49422 | 8.12 | 4 |
| 79709 | 7.15 | 2 |
| 89817 | 6.82 | 3 |
| 59509 | 6.29 | 2 |
| 49409 | 6.58 | 2 |
| 49431 | 4.81 | 1 |
| 49430 | 4.58 | 1 |
| 59530 | 4.4 | 3 |

*A pain score of 0-3 is considered “mild” and 4-7 is considered “moderate”.

When provided in an immediate formulation, naltrexone (parent) is rapidly absorbed and converted to the 6-β-naltrexol metabolite. 6-β-naltrexol is a weaker opioid antagonist than naltrexone, having only 2 to 4% the antagonist potency. Most patients had quantifiable levels (>0.25 pg/mL) of 6-β-naltrexol. The incidental presence of 6-β-naltrexol in the plasma had no effect on pain scores.

It was also important to confirm that Kadian NT did not result in a significantly different type, number or severity of common adverse events. This was confirmed, as shown in Table 8:

TABLE 8

| | |
|---|---|
| Open-label | Double-blind |

| | Kadian | Kadian | Kadian NT |
|---|---|---|---|
| Event | (N = 111) | (N = 71) | (N = 71) |
| Any event | 83.8% | 45.1% | 46.5% |
| Constipation | 46.8% | 12.7% | 15.5% |
| Nausea | 40.5% | 8.5% | 9.9% |
| Somnolence | 28.8% | 8.5% | 9.9% |
| Vomiting | 24.3% | 4.2% | 8.5% |
| Dizziness | 20.7% | 7.0% | 1.4% |
| Headache | 16.2% | 8.5% | 4.2% |

In addition, it was important to note whether Kadian NT functioned similarly to Kadian with respect to adverse events typically associated with withdrawal symptoms. This was confirmed as shown in Table 9:

TABLE 9

| | |
|---|---|
| Open-label | Double-blind |

| | Kadian | Kadian | Kadian NT |
|---|---|---|---|
| Event | (N = 111) | (N = 71) | (N = 71) |
| Tremor | 3.6% | 0.0% | 0.0% |
| Anxiety | 2.7% | 2.8% | 1.4% |
| Irritability | 1.8% | 0.0% | 0.0% |
| Restlessness | 0.9% | 0.0% | 0.0% |
| Muscle Twitch | 0.9% | 0.0% | 0.0% |
| Cold Sweat | 0.9% | 0.0% | 1.4% |
| Piloerection | 0.0% | 0.0% | 0.0% |
| Rhinitis | 0.0% | 0.0% | 0.0% |
| Tachycardia | 0.0% | 0.0% | 0.0% |

Other measurements, including In-Clinic Pain, WOMAC Pain, WOMAC Stiffness, WOMAC Daily Activities, and BPI Pain were also made. It was determined that the differences in these measurements in those taking Kadian and those taking Kadian NT was not significant, as shown in Tables 10-13.

TABLE 10
In-Clinic Pain (ITT Population, Completers)

| | Mean | | Treatment | 95% CI for |
|---|---|---|---|---|
| Day | Kadian | Kadian NT | P-value | Difference |
| Baseline | 2.13 | | | |
| Change Day 7 | N = 68 | N = 69 | 0.9773 | −0.32, 0.33 |
| | +0.18 | +0.16 | | |
| Change Day 14 | N = 69 | N = 69 | 0.2176 | −0.13, 0.56 |
| | +0.28 | +0.06 | | |

TABLE 11
WOMAC Pain (ITT Population, Completers)

| | Mean | | Treatment | 95% CI for |
|---|---|---|---|---|
| Day | Kadian | Kadian NT | P-value | Difference |
| Baseline | 98.1 | | | |
| Change Day 14 | N = 69 | N = 69 | 0.0928 | −2.0, 26.0 |
| | +18.1 | +5.9 | | |

TABLE 12
WOMAC Stiffness (ITT Population, Completers)

| | Mean | | Treatment | 95% CI for |
|---|---|---|---|---|
| Day | Kadian | Kadian NT | P-value | Difference |
| Baseline | 51.1 | | | |
| Change Day | N = 69 | N = 69 | 0.0200 | 1.7, 18.5 |
| 14 | +12.3 | +2.1 | | |

TABLE 13
WOMAC Daily Activities (ITT Population, Completers)

| | Mean | | Treatment | 95% CI for |
|---|---|---|---|---|
| Day | Kadian | Kadian NT | P-value | Difference |
| Baseline | 396.6 | | | |
| Change Day | N = 69 | N = 69 | 0.1206 | −11.0, 93.6 |
| 14 | +70.7 | +28.9 | | |

In conclusion, plasma morphine levels for Kadian and Kadian NT are bioequivalent. It was observed that 55 of 69 (80%) patients had no measurable levels of naltrexone. Of the 14 patients with measurable levels of naltrexone, there was no negative effect on pain scores. Seven of these 14 patients had a measurable level at only one time point. Most patients had some level of 6-β-naltrexol, however there was no negative effect on pain scores. In addition, there was no difference in pain scores in individuals taking Kadian or Kadian NT.

### Example 5

### Kadian NT: Resistance to Tampering

To demonstrate that Kadian NT (60 mg morphine sulfate, 2.4 mg naltrexone HCl (PI-1510)) was indeed resistant to tampering by crushing, the formulation was administered to humans either whole or after being crushed. Morphine concentrations over time were ascertained to compare morphine release from intact and crushed Kadian NT. Release of naltrexone was also determined by measuring plasma naltrexone or 6-β-naltrexol levels. Plasma naltrexone and 6-β-naltrexol levels were also compared to the levels observed after administration of an equivalent dose of naltrexone as a solution. The details of this study are provided below.

The study was a single-dose, open-label, randomized, three-period, three-treatment crossover study in which 24 healthy adults received three separate single-dose administrations of crushed Kadian NT (60 mg morphine, 2.4 mg naltrexone; Treatment A) intact Kadian NT (60 mg morphine, 2.4 mg naltrexone; Treatment B), or an oral solution of Natlrexone-HCl (2.4 mg; Treatment C), following an overnight fast. Dosing days were separated by a washout period of at least 14 days. During each study period, three ml blood samples were obtained within 60 minutes prior to each dose administration and following each dose at selected time points through 72 hours post-dose for morphine analysis (Treatments A and B). Six ml blood samples were obtained within 60 minutes prior to each dose administration and following each dose at selected time points through 168 hours post-dose for naltrexone and 6-β-naltrexol analysis (Treatments A, B and C). A total of 84 pharmacokinetic (PK) blood samples were collected from each subject for analysis of naltrexone and 6-β-naltrexol; 28 samples in each study period (Treatments A, B and C). A total of 38 pharmacokinetic (PK) blood samples were collected from each subject for analysis of morphine; 19 samples in each of Treatments A and B. In addition, blood was drawn and urine collected for clinical laboratory testing at screening and study exit. In each study period, subjects were admitted to the study unit in the evening prior to the scheduled dose. Subjects were confined to the research center during each study period until completion of the 36 hour blood collection and other study procedures. Subjects returned to the study center for outpatient PK blood samples at 48, 60, 72, 84, 96, 108, 120, 132, 144, 156 and 158 hours. Twenty-three of the 24 subjects enrolled completed the study.

Blood samples (1×3 ml) were collected in vacutainer tubes containing K₂-EDTA as a preservative at time 0 (pre-dose), and at 2, 4, 6, 6.5, 7, 7.5, 8, 8.5, 9, 9.5, 10, 12, 18, 24, 30, 36, 48 and 72 hours post-dose for PK analysis of morphine (19 samples in each of Treatments A and B). Blood samples (1×3 ml) were also collected in vacutainer tubes containing K₂-EDTA as a preservative at time 0 (pre-dose), and at 0.5, 1, 1.5, 2, 2.5, 3, 3.5, 4, 5, 6, 8, 10, 12, 16, 24, 36, 48, 60, 72, 84, 96, 108, 120, 132, 144, 156 and 168 hours post-dose for PK analysis of naltrexone and 6-β-naltrexol (28 samples in each of Treatments A, B and C).

Plasma samples were analyzed for morphine, naltrexone and 6-β-naltrexol using a validated LC-MS-MS procedure. The methods were validated for a range of 0.200 to 60.0 ng/ml for morphine based on the analysis of 0.250 ml EDTA human plasma; for a range of 4.00 to 500 pg/ml for naltrexone based on the analysis of 0.500 ml EDTA human plasma; for a range of 10.0 to 4000 ng/ml 0.200 to 60.0 ng/ml for naltrexone based on the analysis of 0.500 ml EDTA human plasma; and for a range of 0.250 to 10.0 pg/ml for 6-β-naltrexol based on the analysis of 1.00 ml EDTA human plasma. Data was stored in the Watson LIMS System (Thermo Electron Corp. Version 6.4.0.02 and 7.2).

Data from 23 subjects were included in the PK and statistical analysis. The concentration-time date were transferred from Watson LO<S directly WinNonlin Enterprise Edition (Wersion 4.0), Pharsight Corp.) using the Custom Query Builder option for analysis. Data were analyzed by noncompartmental methods in WinNonlin. Concentration-time data that were below the limit of quantification (BLQ) were treated as zero (0.00 ng/ml or 0.00 pg/ml) in the data summarization and descriptive statistics. In the PK analysis, BLQ concentrations were treated as zero from time-zero up to the time at which the first quantifiable concentration was observed; embedded and/or terminal BLQ concentrations were treated as “missing”. Full precision concentration data (not rounded to three significant figures) and actual sample times were used for all PK and statistical analyses.

The following PK parameters were calculated: peak concentration in plasma (Cₘₐₓ), time to peak concentration (Tₘₐₓ), elimination rate constant (λ_{z}), terminal half-life (T_{1/2}), area under the concentration-time curve from time-zero to the time of the last quantifiable concentration (AUCₗₐₛₜ), and area under the plasma concentration time curve from time-zero extrapolated to infinity (AUC_{inf}). Analysis of variance (ANOVA) and the Schuimann's two one-sided t-test procedures at the 5% significance level were applied to the log-transformed PK exposure parameters Cmax, AUCₗₐₛₜ and AUC_{inf}. The 90% confidence interval for the ratio of the geometric means (Test/Reference) was calculated. Bioequivalence was declared if the lower and upper confidence intervals of the log-transformed parameters were within 80% to 125%. Mean concentration-time data, PK and statistical analysis are shown below.

TABLE 14
Morphine Concentration: Time Data After Administration of
Crushed Kadian NT (Treatment A) or
Intact Kadian NT (Treatment B)

| | Treatment A: | | | | Treatment B: | | | |
|---|---|---|---|---|---|---|---|---|
| | Kadian NT - Crushed | | | | Kadian NT - Whole, Intact | | | |
| Time | | Mean | SD | CV | | Mean | SD | CV |
| (hr) | n | (ng/mL) | (ng/mL) | (%) | n | (ng/mL) | (ng/mL) | (%) |
| 0.00 | 23 | 0.00 | 0.00 | NC | 23 | 0.00 | 0.00 | NC |
| 2.00 | 23 | 26.1 | 9.13 | 34.99 | 23 | 1.75 | 0.950 | 54.38 |
| 4.00 | 23 | 13.0 | 4.86 | 37.35 | 23 | 4.65 | 1.87 | 40.09 |
| 6.00 | 23 | 6.98 | 2.77 | 39.64 | 23 | 7.56 | 3.14 | 41.46 |
| 6.50 | 23 | 6.10 | 2.68 | 43.97 | 23 | 7.66 | 3.46 | 45.25 |
| 7.00 | 23 | 5.52 | 2.32 | 42.05 | 23 | 7.51 | 3.27 | 43.56 |
| 7.50 | 23 | 4.94 | 2.09 | 42.34 | 23 | 7.39 | 3.49 | 47.24 |
| 8.00 | 23 | 4.45 | 1.87 | 41.93 | 23 | 7.33 | 3.69 | 50.36 |
| 8.50 | 23 | 3.98 | 1.71 | 42.93 | 23 | 6.80 | 3.14 | 46.19 |
| 9.00 | 23 | 3.63 | 1.67 | 45.92 | 23 | 6.55 | 2.94 | 44.91 |
| 9.50 | 23 | 3.28 | 1.61 | 48.97 | 23 | 6.32 | 2.82 | 44.56 |
| 10.00 | 23 | 2.96 | 1.47 | 49.75 | 23 | 6.14 | 2.70 | 44.03 |
| 12.00 | 23 | 2.55 | 1.49 | 58.54 | 23 | 6.22 | 2.70 | 43.41 |
| 18.00 | 23 | 1.59 | 0.832 | 52.31 | 23 | 3.60 | 1.54 | 42.85 |
| 24.00 | 23 | 1.86 | 0.800 | 43.09 | 23 | 2.68 | 1.08 | 40.14 |
| 30.00 | 23 | 1.82 | 0.614 | 33.83 | 23 | 2.85 | 1.45 | 51.04 |
| 36.00 | 23 | 1.26 | 0.602 | 47.94 | 23 | 2.07 | 0.756 | 36.42 |
| 48.00 | 21 | 0.820 | 0.516 | 63.00 | 23 | 1.26 | 0.509 | 40.51 |
| 72.00 | 23 | 0.138 | 0.191 | 139.20 | 23 | 0.590 | 0.503 | 85.23 |

Note:
Plasma samples analyzed using a bioanalytical method with a validated range 0.200 to 60.0 ng/mL; concentrations reported in ng/mL to 3 significant figures; concentrations below limit of quantification set to zero (0.00 ng/mL) in the data summarization
NC = Not calculated

TABLE 15
PK Parameters of Morphine After Administration of
Crushed Kadian NT (Treatment A) or
Intact Kadian NT (Treatment B)

| | Treatment A: | | | | Treatment B: | | | |
|---|---|---|---|---|---|---|---|---|
| | Kadian NT - Crushed | | | | Kadian NT - Whole, Intact | | | |
| Parameter | n | Mean | SD | CV % | n | Mean | SD | CV % |
| Tₘₐₓ (hr) | 23 | 2.01 | 0.02 | 1.08 | 23 | 7.76 | 1.84 | 23.68 |
| Cₘₐₓ (ng/mL) | 23 | 26.1 | 9.13 | 34.99 | 23 | 8.37 | 3.55 | 42.36 |
| AUCₗₐₛₜ | 23 | 170.8 | 56.52 | 33.10 | 23 | 181.9 | 57.13 | 31.40 |
| (hr*ng/mL) | | | | | | | | |
| AUC_{inf} | 23 | 184.4 | 54.04 | 29.30 | 23 | 215.2 | 80.76 | 37.53 |
| (hr*ng/mL) | | | | | | | | |
| AUC_{Extrap} (%) | 23 | 8.07 | 7.10 | 88.02 | 23 | 12.61 | 13.92 | 110.35 |
| λ_{z} (hr⁻¹) | 23 | 0.0506 | 0.0221 | 43.56 | 23 | 0.0407 | 0.0197 | 48.28 |
| T_{1/2} (hr) | 23 | 16.75 | 8.56 | 51.11 | 23 | 23.96 | 18.34 | 76.54 |
| Tₗₐₛₜ (hr) | 23 | 56.35 | 13.26 | 23.53 | 23 | 69.92 | 6.92 | 9.89 |
| Cₗₐₛₜ (ng/mL) | 23 | 0.544 | 0.273 | 50.22 | 23 | 0.663 | 0.478 | 72.04 |
| CL/F (L/hr) | 23 | 351.0 | 95.48 | 27.20 | 23 | 316.0 | 111.0 | 35.11 |
| Vz/F (L) | 23 | 8641 | 5360 | 62.03 | 23 | 9885 | 5505 | 55.69 |

Note:
Full precision data used in pharmacokinetic analysis

TABLE 16
Statistical Analysis of the Log-Transformed Systemic Exposure Parameters
of Morphine After Administration of Crushed Kadian NT (Treatment A) or
Intact Kadian NT (Treatment B)

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 24.3842 | 7.7622 | 314.14 | 288.93 | 341.54 | 0.9953 | 16.52 |
| ln(AUCₗₐₛₜ₎ | 162.9555 | 174.5450 | 93.36 | 87.49 | 99.63 | 0.9998 | 12.81 |
| ln(AUC_{inf}) | 177.6866 | 202.8975 | 87.57 | 78.04 | 98.28 | 0.9389 | 22.92 |

^{a}Geometric Mean for Treatment A - Kadian NT Crushed (Test) and Treatment B - Kadian NT Whole, Intact (Ref) based on Least Squares Mean of log-transformed parameter values
^{b}Ratio(%) = Geometric Mean (Test)/Geometric Mean (Ref)
^{c}90% Confidence Interval

TABLE 17
Naltrexone Concentration: Time Data After Administration of Crushed Kadian NT
(Treatment A), Intact Kadian NT (Treatment B), or
Naltrexone HCl solution (Treatment C)

| | Treatment A: | | | | Treatment B: | | | | Treatment C: | | | |
|---|---|---|---|---|---|---|---|---|---|---|---|---|
| | Kadian NT - Crushed | | | | Kadian NT - Whole, Intact | | | | Naltrexone HCl Solution | | | |
| Time | | Mean | SD | CV | | Mean | SD | CV | | Mean | SD | CV |
| (hr) | n | (pg/mL) | (pg/mL) | (%) | n | (pg/mL) | (pg/mL) | (%) | n | (pg/mL) | (pg/mL) | (%) |
| 0.00 | 23 | 0.00 | 0.00 | NC | 23 | 0.00 | 0.00 | NC | 23 | 0.00 | 0.00 | NC |
| 0.50 | 23 | 559 | 351 | 62.75 | 23 | 0.00 | 0.00 | NC | 23 | 455 | 377 | 82.71 |
| 1.00 | 23 | 599 | 408 | 68.10 | 23 | 0.00 | 0.00 | NC | 23 | 629 | 439 | 69.68 |
| 1.50 | 23 | 499 | 354 | 71.03 | 23 | 0.00 | 0.00 | NC | 23 | 565 | 351 | 62.21 |
| 2.00 | 23 | 403 | 289 | 71.75 | 23 | 0.00 | 0.00 | NC | 23 | 465 | 269 | 57.89 |
| 2.50 | 23 | 313 | 210 | 67.18 | 23 | 0.00 | 0.00 | NC | 23 | 361 | 203 | 56.37 |
| 3.00 | 23 | 249 | 160 | 64.31 | 23 | 0.00 | 0.00 | NC | 23 | 286 | 156 | 54.44 |
| 3.50 | 23 | 207 | 134 | 64.87 | 23 | 0.00 | 0.00 | NC | 23 | 231 | 117 | 50.48 |
| 4.00 | 23 | 164 | 93.9 | 57.33 | 23 | 0.00 | 0.00 | NC | 23 | 182 | 82.1 | 44.99 |
| 5.00 | 23 | 112 | 64.6 | 57.82 | 23 | 0.00 | 0.00 | NC | 23 | 133 | 65.8 | 49.46 |
| 6.00 | 23 | 78.2 | 42.9 | 54.82 | 23 | 0.00 | 0.00 | NC | 23 | 95.7 | 47.6 | 49.77 |
| 8.00 | 23 | 41.6 | 23.1 | 55.62 | 23 | 0.00 | 0.00 | NC | 23 | 51.8 | 23.5 | 45.41 |
| 10.00 | 23 | 20.3 | 8.07 | 39.79 | 23 | 0.00 | 0.00 | NC | 23 | 28.7 | 13.0 | 45.29 |
| 12.00 | 23 | 18.1 | 13.2 | 72.90 | 23 | 0.00 | 0.00 | NC | 23 | 20.5 | 11.0 | 53.81 |
| 16.00 | 23 | 9.27 | 8.95 | 96.58 | 23 | 0.00 | 0.00 | NC | 23 | 9.96 | 7.42 | 74.52 |
| 24.00 | 23 | 5.36 | 7.11 | 132.67 | 23 | 0.00 | 0.00 | NC | 23 | 3.16 | 4.71 | 149.37 |
| 36.00 | 23 | 2.75 | 5.46 | 198.45 | 23 | 0.00 | 0.00 | NC | 23 | 0.607 | 2.03 | 333.81 |
| 48.00 | 23 | 0.741 | 2.47 | 333.62 | 23 | 0.00 | 0.00 | NC | 23 | 0.00 | 0.00 | NC |
| 60.00 | 23 | 0.372 | 1.23 | 331.50 | 23 | 0.00 | 0.00 | NC | 23 | 0.00 | 0.00 | NC |
| 72.00 | 23 | 0.00 | 0.00 | NC | 23 | 0.239 | 1.15 | 479.58 | 22 | 0.00 | 0.00 | NC |
| 84.00 | 23 | 0.00 | 0.00 | NC | 23 | 0.00 | 0.00 | NC | 23 | 0.00 | 0.00 | NC |
| 96.00 | 23 | 0.00 | 0.00 | NC | 23 | 0.00 | 0.00 | NC | 23 | 0.00 | 0.00 | NC |
| 108.00 | 23 | 0.00 | 0.00 | NC | 23 | 0.00 | 0.00 | NC | 23 | 0.00 | 0.00 | NC |
| 120.00 | 23 | 0.00 | 0.00 | NC | 23 | 0.00 | 0.00 | NC | 22 | 0.00 | 0.00 | NC |
| 132.00 | 23 | 0.00 | 0.00 | NC | 23 | 0.00 | 0.00 | NC | 23 | 0.00 | 0.00 | NC |
| 144.00 | 23 | 0.00 | 0.00 | NC | 22 | 0.00 | 0.00 | NC | 23 | 0.00 | 0.00 | NC |
| 156.00 | 23 | 0.00 | 0.00 | NC | 22 | 0.00 | 0.00 | NC | 23 | 0.00 | 0.00 | NC |
| 168.00 | 23 | 0.00 | 0.00 | NC | 22 | 0.00 | 0.00 | NC | 23 | 0.00 | 0.00 | NC |

Note:
Plasma samples analyzed using a bioanalytical method with a validated range 4.00 to 500 pg/mL; concentrations reported in ng/mL to 3 significant figures; concentrations below limit of quantification set to zero (0.00 pg/mL) in the data summarization
NC = Not calculated

TABLE 18
PK Parameters of Naltrexone After Administration of Crushed Kadian NT (Treatment A),
Intact Kadian NT (Treatment B), or Naltrexone HCl solution (Treatment C)

| | Treatment A: | | | | Treatment B: | | | | Treatment C: | | | |
|---|---|---|---|---|---|---|---|---|---|---|---|---|
| | Kadian NT - Crushed | | | | Kadian NT - Whole, Intact | | | | Naltrexone HCl Solution | | | |
| Parameter | n | Mean | SD | CV (%) | n | Mean | SD | CV (%) | n | Mean | SD | CV (%) |
| Tₘₐₓ (hr) | 23 | 0.96 | 0.43 | 44.56 | 1 | 72.00 | NC | NC | 23 | 1.13 | 0.43 | 38.07 |
| Cₘₐₓ | 23 | 685 | 430 | 62.81 | 23 | 0.239 | 1.15 | 479.58 | 23 | 689 | 429 | 62.27 |
| (pg/mL) | | | | | | | | | | | | |
| AUC₀₋ₜ | 23 | 2079 | 1272 | 61.19 | 23 | 1.436 | 6.885 | 479.58 | 23 | 2198 | 1266 | 57.60 |
| (hr*pg/mL) | | | | | | | | | | | | |
| AUC_{inf} | 23 | 2145 | 1315 | 61.29 | 0 | NC | NC | NC | 23 | 2241 | 1276 | 56.92 |
| (hr*pg/mL) | | | | | | | | | | | | |
| AUC_{Extrap} | 23 | 3.15 | 2.06 | 65.49 | 0 | NC | NC | NC | 23 | 2.27 | 1.63 | 71.69 |
| (%) | | | | | | | | | | | | |
| λ_{z} (hr⁻¹) | 23 | 0.1541 | 0.1091 | 70.77 | 0 | NC | NC | NC | 23 | 0.2013 | 0.0801 | 39.79 |
| T_{1/2} (hr) | 23 | 7.45 | 5.32 | 71.37 | 0 | NC | NC | NC | 23 | 4.04 | 1.72 | 42.64 |
| Tₗₐₛₜ (hr) | 23 | 27.15 | 14.26 | 52.54 | 1 | 72.00 | NC | NC | 23 | 20.00 | 6.38 | 31.89 |
| Cₗₐₛₜ (pg/mL) | 23 | 6.22 | 2.54 | 40.89 | 1 | 5.50 | NC | NC | 23 | 7.31 | 2.31 | 31.57 |
| CL/F (L/hr) | — | — | — | — | — | — | — | — | 23 | 1439 | 631.7 | 43.91 |
| Vz/F (L) | — | — | — | — | — | — | — | — | 23 | 13230 | 11150 | 84.33 |

Note:
Full precision data used in pharmacokinetic analysis

TABLE 19
Statistical Analysis of the Log-Transformed Systemic Exposure
Parameters of Naltrexone After Administration of Crushed Kadian
NT (Treatment A) and Naltrexone HCl solution (Treatment C)

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 571.2954 | 579.8535 | 98.52 | 83.79 | 115.85 | 0.7390 | 32.61 |
| ln(AUCₗₐₛₜ) | 1798.1676 | 1949.0311 | 92.26 | 83.34 | 102.14 | 0.9736 | 20.16 |
| ln(AUC_{inf}) | 1857.1264 | 1994.4908 | 93.11 | 84.43 | 102.69 | 0.9804 | 19.39 |

^{a}Geometric Mean for Tteatment A - Kadian NT Crushed (Test) and Naltrexone HCl solution (Ref) based on Least Squares Mean of log-transformed parameter values
^{b}Ratio(%) = Geometric Mean (Test)/Geometric Mean (Ref)
^{c}90% Confidence Interval

TABLE 20
6-β-Naltrexol Concentration: Time Data After Administration of Crushed
Kadian NT (Treatment A), Intact Kadian NT (Treatment B), or
Naltrexone HCl solution (Treatment C)

| | Treatment A: | | | | Treatment B: | | | | Treatment C: | | | |
|---|---|---|---|---|---|---|---|---|---|---|---|---|
| | Kadian NT - Crushed | | | | Kadian NT - Whole, Intact | | | | Naltrexone HCl Solution | | | |
| Time | | Mean | SD | CV | | Mean | SD | CV | | Mean | SD | CV |
| (hr) | n | (pg/mL) | (pg/mL) | (%) | n | (pg/mL) | (pg/mL) | (%) | n | (pg/mL) | (pg/mL) | (%) |
| 0.00 | 23 | 0.231 | 0.501 | 216.82 | 23 | 0.128 | 0.332 | 258.90 | 23 | 0.00 | 0.00 | NC |
| 0.50 | 23 | 3020 | 1450 | 48.01 | 23 | 0.262 | 0.432 | 164.79 | 23 | 2440 | 1360 | 55.63 |
| 1.00 | 23 | 3120 | 994 | 31.88 | 23 | 0.821 | 1.82 | 222.36 | 23 | 3330 | 1320 | 39.77 |
| 1.50 | 23 | 3010 | 1110 | 36.80 | 23 | 1.64 | 3.98 | 243.48 | 23 | 3570 | 1360 | 38.12 |
| 2.00 | 23 | 2720 | 914 | 33.56 | 23 | 1.99 | 4.48 | 225.22 | 23 | 3250 | 1120 | 34.55 |
| 2.50 | 23 | 2450 | 833 | 33.97 | 23 | 2.27 | 5.13 | 225.94 | 23 | 2860 | 902 | 31.60 |
| 3.00 | 23 | 2270 | 813 | 35.87 | 23 | 1.99 | 4.51 | 227.10 | 23 | 2600 | 859 | 33.01 |
| 3.50 | 23 | 2070 | 764 | 36.86 | 23 | 1.91 | 4.41 | 230.62 | 23 | 2400 | 799 | 33.23 |
| 4.00 | 23 | 1880 | 617 | 32.77 | 23 | 1.73 | 3.98 | 229.82 | 23 | 2170 | 686 | 31.63 |
| 5.00 | 23 | 1680 | 625 | 37.28 | 23 | 1.61 | 3.73 | 232.08 | 23 | 1980 | 685 | 34.60 |
| 6.00 | 23 | 1470 | 524 | 35.65 | 23 | 1.33 | 3.08 | 231.06 | 23 | 1770 | 604 | 34.01 |
| 8.00 | 23 | 1150 | 448 | 39.08 | 23 | 1.05 | 2.42 | 229.39 | 23 | 1410 | 482 | 34.27 |
| 10.00 | 23 | 922 | 381 | 41.29 | 23 | 0.855 | 1.96 | 228.66 | 23 | 1160 | 354 | 30.43 |
| 12.00 | 23 | 800 | 331 | 41.32 | 23 | 0.736 | 1.61 | 218.58 | 23 | 1040 | 323 | 30.91 |
| 16.00 | 23 | 626 | 254 | 40.63 | 23 | 0.559 | 1.19 | 213.59 | 23 | 301 | 250 | 31.25 |
| 24.00 | 23 | 476 | 155 | 32.62 | 23 | 0.524 | 0.979 | 186.85 | 23 | 562 | 161 | 28.65 |
| 36.00 | 23 | 332 | 106 | 31.82 | 23 | 0.674 | 1.39 | 206.83 | 23 | 290 | 98.4 | 33.88 |
| 48.00 | 23 | 202 | 71.7 | 35.44 | 23 | 1.25 | 3.30 | 264.82 | 23 | 154 | 59.9 | 38.97 |
| 60.00 | 23 | 121 | 57.3 | 47.46 | 23 | 2.96 | 10.2 | 346.26 | 23 | 82.0 | 40.8 | 49.75 |
| 72.00 | 23 | 75.0 | 40.1 | 53.47 | 23 | 4.53 | 8.76 | 193.18 | 22 | 47.5 | 25.1 | 52.88 |
| 84.00 | 23 | 40.3 | 23.3 | 57.91 | 23 | 3.38 | 6.53 | 193.00 | 23 | 27.0 | 15.7 | 58.06 |
| 96.00 | 23 | 24.5 | 15.1 | 61.69 | 23 | 1.89 | 3.58 | 189.63 | 23 | 16.6 | 9.63 | 58.11 |
| 108.00 | 23 | 15.0 | 9.25 | 61.83 | 23 | 0.975 | 1.95 | 200.24 | 23 | 10.6 | 6.26 | 59.23 |
| 120.00 | 23 | 10.1 | 5.86 | 58.02 | 23 | 0.523 | 1.04 | 197.97 | 22 | 7.56 | 4.56 | 60.34 |
| 132.00 | 23 | 6.81 | 3.51 | 51.56 | 23 | 0.341 | 0.634 | 185.78 | 23 | 5.41 | 2.73 | 50.58 |
| 144.00 | 23 | 5.04 | 2.47 | 49.08 | 22 | 0.168 | 0.417 | 247.82 | 23 | 4.65 | 2.03 | 43.71 |
| 156.00 | 23 | 3.55 | 1.79 | 50.47 | 22 | 0.177 | 0.340 | 191.96 | 23 | 3.37 | 1.67 | 49.52 |
| 168.00 | 23 | 2.88 | 1.58 | 54.84 | 22 | 0.089 | 0.251 | 283.02 | 23 | 2.46 | 1.72 | 69.91 |

Note:
Plasma samples analyzed using a bioanalytical method with a validated range 10.0 to 4000 or 0.250 to 10.0 pg/mL; concentrations reported in ng/mL to 3 significant figures; concentrations below limit of quantification set to zero (0.00 pg/mL) in the data summarization
NC = Not calculated

TABLE 21
PK Parameters of 6-β-naltrexol After Administration of Crushed Kadian
NT (Treatment A), Intact Kadian NT (Treatment B), or Naltrexone HCl
solution (Treatment C)

| | Treatment A: | | | | Treatment B: | | | | Treatment C: | | | |
|---|---|---|---|---|---|---|---|---|---|---|---|---|
| | Kadian NT - crushed | | | | Kadian NT - Whole, Intact | | | | Naltrexone HCl Solution | | | |
| Parameter | n | Mean | SD | CV (%) | n | Mean | SD | CV (%) | n | Mean | SD | CV (%) |
| Tₘₐₓ (hr) | 23 | 1.00 | 0.50 | 50.28 | 14 | 44.36 | 34.89 | 78.64 | 23 | 1.31 | 0.53 | 39.95 |
| Cₘₐₓ | 23 | 3740 | 1320 | 35.43 | 23 | 7.61 | 11.5 | 150.50 | 23 | 3920 | 1350 | 34.39 |
| (ng/mL) | | | | | | | | | | | | |
| AUC₀₋ₜ | 23 | 39740 | 12110 | 30.48 | 23 | 273.2 | 477.3 | 174.74 | 23 | 43050 | 12760 | 29.64 |
| (hr*pg/mL) | | | | | | | | | | | | |
| AUC_{inf} | 23 | 39830 | 12130 | 30.47 | 12 | 531.5 | 567.9 | 106.85 | 23 | 43170 | 12800 | 29.65 |
| (hr*pg/mL) | | | | | | | | | | | | |
| AUC_{Extrap} | 23 | 0.20 | 0.10 | 49.91 | 13 | 4.36 | 4.07 | 93.37 | 23 | 0.27 | 0.23 | 85.37 |
| (%) | | | | | | | | | | | | |
| λ_{z} (hr⁻¹) | 23 | 0.0371 | 0.0049 | 13.16 | 12 | 0.0415 | 0.0125 | 30.03 | 23 | 0.0294 | 0.0088 | 30.00 |
| T_{1/2} (hr) | 23 | 19.03 | 2.92 | 15.35 | 12 | 19.69 | 12.16 | 61.77 | 23 | 26.32 | 10.32 | 39.22 |
| Tₗₐₛₜ (hr) | 23 | 168.00 | 0.00 | 0.00 | 14 | 126.06 | 40.64 | 32.24 | 23 | 166.44 | 4.13 | 2.48 |
| Cₗₐₛₜ | 23 | 2.88 | 1.53 | 54.84 | 14 | 0.453 | 0.199 | 44.04 | 23 | 2.78 | 1.46 | 52.54 |
| (pg/mL) | | | | | | | | | | | | |

Note:
Full precision data used in pharmacokinetic analysis

TABLE 22
Statistical Analysis of the Log-Transformed Systemic Exposure
Parameters of 6-β-naltrexol After Administration of Crushed Kadian
NT (Treatment A), Intact Kadian NT (Treatment B), or Naltrexone
HCl solution (Treatment C)

| Dependent | Geometric Mean^{a} | | Ratio (%)^{b} | 90% CI^{c} | | | ANOVA |
|---|---|---|---|---|---|---|---|
| Variable | Test | Ref | (Test/Ref) | Lower | Upper | Power | CV % |
| ln(Cₘₐₓ) | 3500.9867 | 3696.3140 | 94.72 | 86.30 | 103.95 | 0.9872 | 18.42 |
| ln(AUCₗₐₛₜ₎ | 38132.6717 | 41339.2194 | 92.24 | 85.52 | 99.50 | 0.9984 | 14.94 |
| ln(AUC_{inf}) | 38211.3223 | 41451.0000 | 92.18 | 85.45 | 99.45 | 0.9984 | 14.98 |

^{a}Geometric Mean for Treatment A - Kadian NT Crushed (Test) and Naltrexone HCl solution (Ref) based on Least Squares Mean of log-transformed parameter values
^{b}Ratio(%) = Geometric Mean (Test)/Geometric Mean (Ref)
^{c}90% Confidence Interval

The data presented above demonstrates that morphine is released more rapidly from the crushed formulation than from the intact pellet. The data also clearly demonstrates that administration of crushed Kadian NT results in similar plasma levels of naltrexone and 6-naltrexol as is observed following oral administration of naltrexone HCl. Thus, tampering with Kadian NT by crushing has been demonstrated to result in the concomitant release of both morphine and its antagonist naltrexone.

### Example 6

### Comparison of Morphine Levels from Morphine Immediate Release Preparations, Whole Kadian NT, Crushed Kadian NT and Placebo

In this study, ALO-01 (see Example 3), an extended release (ER) morphine formulation with an abuse deterrent naltrexone core, was orally administered whole or after tampering with the formulation by crushing and compared to a morphine sulphate immediate release (MSIR) product. For crushed study drug administration, ALO-01 and matching placebo capsules were emptied to release the inner pellets. The pellets were manually crushed for over 2 minutes using a mortar and pestle; the mortar was then rinsed with apple juice to remove all crushed ALO-01. Along with whole and crushed ALO-01, MSIR, and placebo were orally administered in a randomized, double-blind, triple-dummy, 4-way crossover manner to evaluate the effects of tampering with the abuse deterrent formulation of morphine and naltrexone on subjective drug measures, including Drug Liking, and on the pharmacokinetics of morphine, naltrexone, and the naltrexone metabolite (6-β-naltrexol) in healthy volunteers with a history of non-therapeutic recreational opioid use. This was a single center study.

This study consisted of three periods: a screening/qualifying period, a double-blind treatment period, and a post-treatment follow-up period. The screening/qualifying period lasted up to 56 days and consisted of a screening session and a 3-night inpatient double-blind qualifying session. The treatment period consisted of four 2-night inpatient treatment sessions for which subjects were randomly selected for one of the four dosings described below. Each double-blind treatment session consisted of a single dose of eac study drug administered on Dosting Day (day 1) with assessments performed pre-dosing and for 24 hours post-dosing. Subjects remained at the study center from the day prior to dosing until completion of the 24 hour post-dosing procedures in each period. The washout period between dosing was 14 to 21 days. The post-treatment follow-up period consisted of safety assessments between 3 to 14 days after the last dose treatment visit. The follow-up session occurred following wash-out or at early withdrawal. Sixty-four subjects were planned to participate in the qualifying session, with the intent to identify approximately 38 qualified subjects. Approximately 32 of these qualified subjects were to be enrolled in the treatment period, with the intent to complete 24 subjects. The total duration of the study including the screening/qualifying period, treatment period, and follow-up period was approximately 19 weeks. No interim analysis was planned or performed for this study.

The treatment period study drugs included Kadian NT (otherwise known as ALO-01), consisting of a 60 mg morphine sulfate (ER) pellet and a naltrexone core inner pellet (Alpharma Pharmaceuticals LLC, Piscataway, N.J., U.S.A), and MSIR solution (Statex Oral Drops, 50 mg/mL, Pharmascience Inc., Montreal, Canada). Matching placebo capsules (matched to ALO-01) were administered throughout the treatment period (placebo capsules, Alpharma Pharmaceuticals LLC, Piscataway, N.J., U.S.A). The morphine sulfate was prepared in a solution of sugar-free apple juice (room temperature). The crushed placebo and crushed ALO-01 were dissolved in a separate aliquot of sugar-free apple juice (room temperature).

During the qualifying session, all eligible subjects randomly received single doses of MSIR 120 mg containing beverage and placebo beverage, administered once over 2 days. The morphine beverage was prepared by diluting 2.4 mL of Statex® Oral Drops 50 mg/mL in 148 mL of room temperature sugar-free apple juice shortly before administration. The placebo beverage was comprised of 150 mL of sugar-free apple juice. During each treatment session, all eligible subjects received two whole capsules (with active drug or placebo) and two beverages (with active drug and/or placebo) orally. All eligible subjects received each of the four following treatments, one per treatment session:
Treatment A: 2×Placebo capsules (whole)+ALO-01 2×60 mg capsules (crushed) in apple juice (Beverage 1)+apple juice (MSIR Placebo) (Beverage 2)
Treatment B: 2×60 mg ALO-01 (whole)+2×Placebo capsules (crushed) in apple juice (Beverage 1)+apple juice (MSIR Placebo) (Beverage 2)
Treatment C: 2×Placebo capsules (whole)+2×Placebo capsules (crushed) in apple juice (Beverage 1)+120 mg Morphine Sulfate IR in apple juice (Beverage 2)
Treatment D: 2×Placebo capsules (whole)+2×Placebo capsules (crushed) in apple juice (Beverage 1)+apple juice (MSIR Placebo) (Beverage 2)

For crushed drug administration, ALO-01 or placebo capsules were opened to release the inner pellets. The pellets were completely crushed manually using a mortar and pestle over 2 minutes and were then dissolved in 150 mL of sugar-free apple juice at room temperature, the mortar then was rinsed with apple juice to remove all crushed ALO-01. Placebo capsules were administered whole and/or crushed, in order to maintain blinding and to mask for texture (crushed capsule administration).

MSIR 120 mL oral solution was prepared by diluting 2.4 mL of Statex Oral Drops (50 mg/mL) in 148 mL of room temperature sugar-free apple juice shortly before administration. Subjects were instructed to swallow the whole capsules with Beverage 2, 150 mL apple juice treatment containing either MSIR or MSIR Placebo. Subjects were then instructed to ingest Beverage 1, containing either crushed ALO-01 or Placebo. Following administration of Beverage 1, an additional 50 mL of apple juice was provided to rinse any residual capsule fragments. Subjects were instructed to swirl the apple juice and immediately ingest the remaining apple juice. Clinic staff checked the cup to ensure that all study drug had been administered. An additional 50 mL of apple juice could be used for rinsing, if needed; however, the total amount of apple juice consumed at each treatment should not exceed 400 mL or an amount equivalent to approximately 12 to 14 fluid ounces.

This study is considered a within-subject, 4 period crossover design. Each subject belonged to 1 of 4 dosing sequences. Analysis of each primary and secondary endpoint was done using a linear mixed effect Analysis of Covariance (ANCOVA) model. The model included treatment, period, and sequence as the fixed effects and subjects nested within sequence as a random effect. For pharmacodynamic measures that have pre-dose values, the model included the pre-dose baseline value as a covariate. The linear mixed effect model was based on the per protocol population. A 5% Type I error rate with a p-value less than 0.05 was considered as statistically significance for all individual hypothesis tests. All statistical tests were performed using two-tailed significance criteria. For each of the main effects, the null hypothesis was “there was no main effect,” and the alternative hypothesis was “there was a main effect.” For each of the contrasts the null hypothesis was “there was no effect difference between the tested pair,” and the alternative hypothesis was “there was effect difference between the tested pair.” Data for all analysis were included as far as possible. No subjects discontinued during the study. No imputations were performed. Benjamin and Hochberg procedure was used to control for Type I error arising from multiple treatment comparisons for all primary endpoints.

### A. Summary of Efficacy Data

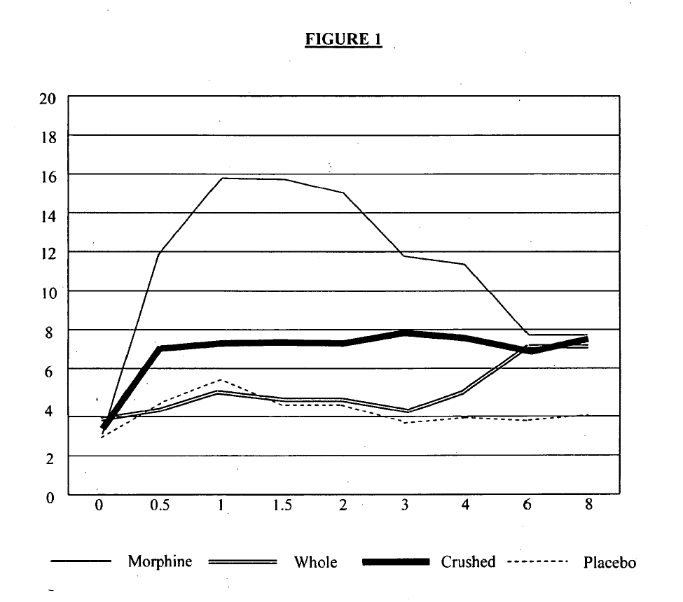

A study of 32 opioid-abusing, non-dependent subjects was performed to compare the release profile of whole Kadian NT and crushed Kadian NT to immediate release preparation of morphine sulfate (“MSIR”). Placebo was also tested. FIG. 1 demonstrates the data for the Cole/ARCI Stimulation Euphoria index after up to eight hours following administration of IR Morphine, crushed or whole Kadian NT or placebo. The most significant differences were observed between Morphine IR and placebo (p<0.001), crushed Kadian NT (p<0.001; “AL-01 crushed”), and whole Kadian NT (p<0.001; “AL-01 whole”) 1.5 hours after administration. Differences were observed between placebo and crushed Kadian NT (“Crushed AL-01”; p=0.089) and whole Kadian NT (“Whole AL-01”; p=0.755) at the 1.5 hour and other timepoints. Results from this study are also shown in Table 23. Immediate release morphine showed statistically significant measures versus whole Kadian NT, crushed Kadian NT and placebo. These measures include “VAS Drug Liking”, “VAS Overall Drug Liking”, “Cole ARCI Stimulation (Euphoria)”, “Subjective Drug Value”, “Cole ARCI-Abuse Potential”, “ARCI MBG”, “VAS Good Effects”, and “VAS Feeling High”.

TABLE 23

| | | Positive measures | | | | | | | |
|---|---|---|---|---|---|---|---|---|---|
| | | | VAS | | | Cole | | | |
| | | VAS | Overall | Cole ARCI | Subjective | ARCI - | | VAS | VAS |
| | | Drug | Drug | Stimulation | Drug | Abuse | ARCI | Good | Feeling |
| Analysis | Treatment | Liking | Liking | Euphoria | Value | Potential | MBG | Effects | High |
| E_{MAX} | Treatment | | | | | | | | |
| | effect | | | | | | | | |
| | Morphine IR - | <.001 | <.001 | <.001 | <.001 | <.001 | <.001 | <.001 | <.001 |
| | Placebo | | | | | | | | |
| | Morphine IR - | <.001 | <.001 | <.001 | <.001 | 0.002 | <.001 | <.001 | <.001 |
| | ALO-01 | | | | | | | | |
| | crushed | | | | | | | | |
| | Morphine IR - | <.001 | <.001 | <.001 | <.001 | <.001 | <.001 | <.001 | <.001 |
| | ALO-01 whole | | | | | | | | |
| AUE_{0-2 h}^{a} | Treatment | | | | | | | | |
| | effect | | | | | | | | |
| | Morphine IR - | <.001 | <.001 | <.001 | <.001 | <.001 | <.001 | <.001 | <.001 |
| | Placebo | | | | | | | | |
| | Morphine IR - | <.001 | <.001 | <.001 | <.001 | 0.001 | <.001 | <.001 | <.001 |
| | ALO-01 | | | | | | | | |
| | crushed | | | | | | | | |
| | Morphine IR - | <.001 | <.001 | <.001 | <.001 | <.001 | <.001 | <.001 | <.001 |
| | ALO-01 whole | | | | | | | | |
| 1.5 h | Treatment | | | | | | | | |
| | effect | | | | | | | | |
| | Morphine IR- | <.001 | | <.001 | | <.001 | <.001 | <.001 | <.001 |
| | Placebo | | | | | | | | |
| | Morphine IR - | <.001 | | <.001 | | <.001 | <.001 | <.001 | <.001 |
| | ALO-01 | | | | | | | | |
| | crushed | | | | | | | | |
| | Morphine IR - | <.001 | | <.001 | | <.001 | <.001 | <.001 | <.001 |
| | ALO-01 whole | | | | | | | | |

### B. Efficacy Data

The safety population was defined as all randomized subjects who receive any study drug; these subjects were used for the analysis and presentation of the safety data. All 32 (100.0%) randomized subjects received all doses of study drugs and were included in the safety population.

The per protocol population (i.e., evaluable population) was defined as all subjects in the safety population who completed the study and had no major protocol violations that would exclude the subjects from analysis. This population was used for the analysis and presentation of the summary and statistical inference for pharmacokinetic and pharmacodynamic parameters. All 32 (100.0%) subjects in the safety population are included in the per protocol population.

The safety and per protocol populations (i.e., all randomized subjects) were comprised of 26 (81.3%) male subjects and 6 (18.8%) female subjects. The majority of subjects were identified as white (22 (68.8%) of 32 subjects), followed by multiracial/other (4 (12.5%) of 32 subjects), black or African American (3 (9.4%) of 32 subjects), Hispanic/Latino (2 (6.3%) of 32 subjects), and Asian (1 (3.1%) of 32 subjects). Since the same subjects comprise both the safety and per protocol populations, demographic characteristics of age, weight, height, and BMI are identical between the populations. Overall, the average age and BMI (mean (SD)) of subjects in the study was 35.0 (7.59) years and 26.42 (2.751) kg/m², respectively. The average BMI was similar between male and female subjects, while the average age of female subjects was slightly older than that of male subjects (i.e., 37.3 (6.89) years vs. 34.5 (7.77) years). Ranges in BMI and age were similar for both genders.

The nomenclature to describe the treatment groups has been abbreviated as outlined in Table 24:

TABLE 24

| Treatment administered | Abbreviated name |
|---|---|
| ALO-01 (120 mg) whole | ALO-01 whole |
| ALO-01 (120 mg) crushed | ALO-01 crushed |
| Morphine sulfate IR (120 mg) | MSIR |
| Placebo | Placebo |

The objective of this study was to determine the relative pharmacodynamic effects and safety of crushed and whole ALO-01 compared to MSIR and Placebo and of crushed ALO-01 to whole ALO-01. Therefore, the pharmacodynamic results have been organized primarily by pharmacologic effects, with emphasis on the positive effects. However, to fully characterize the drug effect, negative and other (i.e., neither positive nor negative) drug effects were also examined. The primary endpoints examined in this study include some of the positive measures and measure of physiologic effect (pupillometry), while the secondary endpoints include the remaining positive measures, as well as the negative and other measures. Subjective measures of positive response (i.e., liking or enjoyment of the study drugs' acute effects) are the measures that bear most directly on questions of drug induced euphoria. The subjective measures of negative effects (i.e., disliking or dysphoria) were assessed as they could counteract positive subjective effects. Additionally, the subjective measures of other drug effects, including stimulation and sedation (i.e., effects that may be perceived as either positive or negative, depending on the context) and ability to distinguish any drug effects were also examined. Table 25 provides classification of the collected endpoints into positive, negative, and other measures. For some pharmacodynamic assessments, baseline measures were collected and significant baseline effect was found; however, the treatment effect was evaluated after the baseline covariate adjustment was made in the analysis of covariance (ANCOVA) model. Table 25 showing the classification of outcome measures is provided below:

TABLE 25

| | |
|---|---|
| Positive measures | VAS-Drug Liking* |
| | Cole/ARCI-Stimulation-Euphoria* |
| | Cole/ARCI-Abuse Potential* |
| | Subjective Drug Value* |
| | ARCI-MBG* |
| | VAS-Good Effects |
| | VAS-High |
| Negative measures | VAS-Bad Effects |
| | VAS-Feel Sick |
| | VAS-Nausea |
| | ARCI-LSD |
| | Cole/ARCI-Unpleasantness-Dysphoria |
| | Cole/ARCI-Unpleasantness-Physical |
| Other measures | |
| Other drug effects: | VAS-Any Drug Effects |
| | VAS-Dizziness |
| | Pupillometry* |
| Stimulant effects: | ARCI-BG |
| | ARCI-Amphetamine |
| | Cole/ARCI-Stimulation-Motor |
| Sedation effects: | VAS-Sleepy |
| | ARCI-PCAG |
| | Cole/ARCI-Sedation-Motor |
| | Cole/ARCI-Sedation-Mental |

*Primary measures

Each pharmacodynamic test cycle lasted approximately 15 minutes and included (1) a series of rating scales and questionnaires, in which subjects rated their current perceptions of their subjective state and of the drug's effects, and (2) one objective measure of pharmacological effect, namely pupillometry. Note that for the VAS for Overall Liking and SDV assessments carried out at 12 and 24 hours post-dose, the subjects were instructed to base their responses on the cumulative or overall assessment of the drug's effects from dosing on Day 1. Measures (except pupillometry) were administered and data were captured electronically using proprietary computerized software (Scheduled Measurement System [SMS], DecisionLine Clinical Research Corporation).

The “VAS for Drug Liking” assessment was chosen as one of the primary measures in the study because the degree of subject liking is one of the most sensitive indicators of abuse liability (Balster & Bigelow, 2003; Griffiths et al. 2003). VAS for Drug Liking assessed the subject's liking of the drug at the moment the question was asked, while Overall Drug Liking VAS assessed the subject's global experience of the drug. In both cases, the VAS is bipolar (e.g., strong disliking to strong liking). These scales were not administered pre-dose as they refer specifically to the effect of drug taken. The other VASs assess positive, negative, and other subjective effects to assess the subjective pharmacologic response to the study drugs.

Each VAS consisted of a horizontal line with a statement presented above the bar (e.g., “I can feel a drug effect”, etc.). The ends of the line for all scales were marked with descriptive anchors (e.g., “not at all” and “extremely” for some unipolar scales). Participants were instructed to click and drag the computer mouse to the appropriate position along the line, according to how they felt at that moment (with respect to the statement presented above the line). Each scale was scored as an integer from 0 to 100, representing the position on the line. Each VAS was presented one at a time. Note that scales that refer specifically to drug (i.e., Good Effects, Bad Effects, and Any Effects) were not administered pre-dose.

The Subjective Drug Value (SDV) involves a series of independent, theoretical forced choices between the drug administered and different monetary values, as described below. The subjects did not receive either the drug or the money described in the choices. Subjects were asked to choose between receiving another dose of the same drug to take home or an envelope containing a specified amount of money. Depending on the answer to each question, the monetary value in the next question is either higher or lower. At the end of 6 questions, the procedure estimated the crossover point at which the subject was indifferent between choosing drug (as would be done for all smaller values) and choosing money (as would be done for all larger values). The crossover point is the proxy index of reinforcing efficacy that was used as an outcome measure for estimating abuse potential. This test was adapted from a similar procedure utilized by Griffiths and colleagues (Girffiths, et al, 1993; Griffiths, et al. 1996).

The Addiction Research Center Inventory (ARCI) short form (Martin et al., 1971) consists of 77 questions extracted from the much larger (550 question) ARCI. The short form contains the following 5 subscales that are important to the evaluation of abuse potential: Morphine-Benzedrine Group scale (the MBG or “euphoria” scale); Amphetamine (A) scale; Benzedrine Group scale (the BG or “stimulant” scale); Lysergic Acid Diethylamide scale (the LSD or “dysphoria” scale); and Pentobarbital-Chlorpromazine-Alcohol Group scale (the PCAG or “sedation” scale).

Cole and colleagues (Cole et al., 1982) later developed a different subset of the original ARCI (Cole/ARCI) using a new factor analysis of responses to some of the 550 questions. This newer form includes 7 scales: Sedation-Motor, Sedation-Mental, Unpleasantness-Physical, Unpleasantness-Mental, Stimulation-Motor, Stimulation-Euphoria, and Abuse Potential. The combined 5 scale ARCI (short form) and the 7 Cole/ARCI scales together consist of 77 questions and 12 scales. The questions were presented to the subject on a computer screen as multiple choice, using a large font. Subjects selected their responses by pointing to them with the cursor controlled by a mouse to select one of the four responses: “False”, “More false than true”, “More true than false”, or “True”.

### Pupillometry

Pupillometry is a measure of miosis, a physiologic measure of opiate effect. Pupillary diameter was evaluated during the qualifying session, as well as the treatment period. Measurement of pupillary diameter at pre-dose and following administration of the study treatment allowed evaluation of general physiologic opiate activity (Knaggs et al., 2004). To measure the pupil diameter, the NeurOptics Pupillometer (model: 59001-IFU, NeurOptics, Inc, Irvine, USA) was used; it is a handheld optical scanner which captures and analyzes a series of digital images to obtain a measurement of the diameter of a human pupil. The system acquires images using a self-contained infrared illumination source and a digital camera. Data from a total of 41 frames captured over approximately 3 seconds was used in the calculation and the final display shows the weighted average and standard deviation of the pupil size. Measures were collected under mesopic lighting conditions. Descriptive statistics for pupil diameter (mm) raw scores at scheduled time points and summary parameters (per protocol population) were generated. Analyses of covariance for the mean PCₘᵢₙ, PAOC₍₀₋₂ₕ₎, PAOC₍₀₋₈ₕ₎, PAOC₍₀₋₂₄ₕ₎, and HR1.5 (pupil diameter at 1.5 hours post-dose) (per protocol population) were also made.

The proportion of subjects (per protocol population) who had a 10 to 100% change in pupil diameter in post-dose maximum change from baseline compared to MSIR 120 mg are listed in Tables 26. Generally, the majority of subjects (percentage [number of subjects/total number of subjects]) had at least a 20% minimum reduction in Eₘₐₓ following ALO-01 whole administration (56.3% [ 18/32]) and at least a 10% minimum reduction following ALO-01 crushed administration (65.6% [ 21/32]) relative to MSIR. The highest reductions were seen as a 100% reduction in the ALO-01 whole group (3.1% [ 1/32]) and a 70-79% reduction in the ALO-01 crushed group (6.3% [ 2/32]).

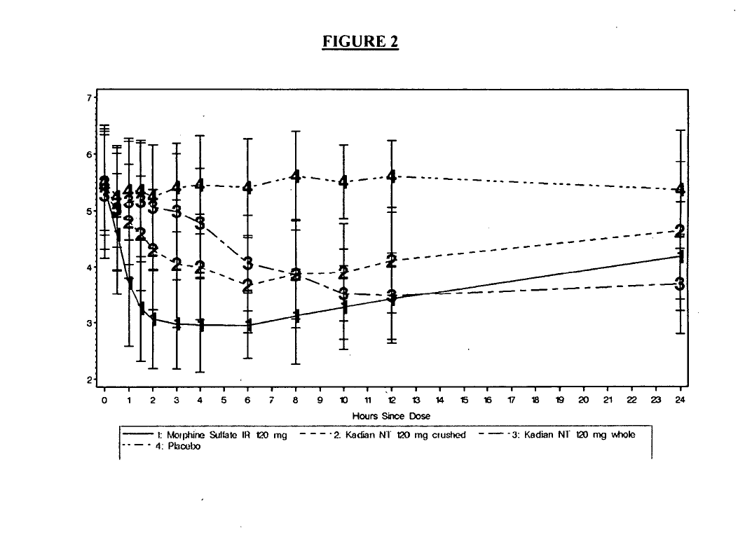

Summary parameters of pupil diameter for the per protocol population are listed below in Table 27. The greatest reduction in pupil diameter, including parameters of HR1.5 and PT25, was observed in the MSIR group, followed by ALO-01 crushed, ALO-01 whole, and Placebo (FIG. 2 and Table 27). This order was observed for the PAOC values, which were the lowest in the Placebo group and increased in the ALO-01 whole, ALO-01 crushed, and MSIR groups, respectively. The exception to this was observed for PAOC₍₀₋₂₄ₕ₎, which had slightly higher value (mean [SD]) in the ALO-01 whole group (32.38 [21.43]) compared to the ALO-01 crushed group (30.69 [17.89]) (Table 27). The PCₘᵢₙ (mean [SD]) ranged from 2.70 (0.64) in the Placebo group to 4.71 (0.64) in the MSIR group. The PTₘᵢₙ (hours) median was the lowest in the MSIR (3.13) and ALO-01 crushed (6.10) groups and highest in the ALO-01 whole group (12.07) (FIG. 2).

TABLE 26
Pupil Diameter (mm) proportion of subjects (per protocol population) who
had a 10-100% reduction in post-dose maximum change from baseline
compared to Morphine Sulfate IR 120 mg

| | |
|---|---|
| ALO-01 120 mg crushed | ALO-01 120 mg |
| (N = 32) | whole (N = 32) |

Maximum change of Pupil Diameter

| | | |
|---|---|---|
| At least 10% reduction | 21 (65.6%) | 20 (62.5%) |
| At least 20% reduction | 14 (43.8%) | 18 (56.3%) |
| At least 30% reduction | 12 (37.5%) | 13 (40.6%) |
| At least 40% reduction | 9 (28.1%) | 10 (31.3%) |
| At least 50% reduction | 6 (18.8%) | 7 (21.9%) |
| At least 60% reduction | 4 (12.5%) | 3 (9.4%) |
| At least 70% reduction | 2 (6.3%) | 3 (9.4%) |
| At least 80% reduction | 0 (0.0%) | 2 (6.3%) |
| At least 90% reduction | 0 (0.0%) | 1 (3.1%) |
| At least 100% reduction | 0 (0.0%) | 1 (3.1%) |

Note:
Percentage is calculated based on the number of subjects in the Per Protocol Population as the denominator

TABLE 27
Pupil Diameter (mm) descriptive statistics of summary parameters for the per protocol
population

| | | | |
|---|---|---|---|
| | ALO-01 120 mg | ALO-01 120 mg | Morphine Sulfate IR |
| Placebo | whole | crushed | 120 mg |

PCₘᵢₙ

| | | | | |
|---|---|---|---|---|
| N | 32 | 32 | 32 | 32 |
| Mean | 4.71 (0.92) | 3.20 (0.81) | 3.43 (0.81) | 2.70 (0.64) |
| (SD) | | | | |
| Median | 4.85 | 3.00 | 3.30 | 2.60 |
| Range | 2.7-6.0 | 2.1-6.0 | 2.2-5.8 | 1.7-5.0 |

PTₘᵢₙ

| | | | | |
|---|---|---|---|---|
| N | 32 | 32 | 32 | 32 |
| Mean | 8.64 (9.08) | 13.54 (6.63) | 7.75 (5.86) | 4.11 (2.67) |
| (SD) | | | | |
| Median | 6.07 | 12.07 | 6.10 | 3.13 |
| Range | 0.57-24.10 | 2.10-24.15 | 2.10-24.08 | 1.12-12.07 |

PAOC_{(0-2 h)}

| | | | | |
|---|---|---|---|---|
| N | 32 | 32 | 32 | 32 |
| Mean | 0.35 (0.84) | 0.30 (0.85) | 1.38 (1.03) | 2.98 (1.72) |
| (SD) | | | | |
| Median | 0.29 | 0.39 | 1.35 | 2.71 |
| Range | −1.61-1.83 | −1.00-1.90 | −0.59-3.72 | −0.04-8.42 |

PAOC_{(0-8 h)}

| | | | | |
|---|---|---|---|---|
| N | 32 | 32 | 32 | 32 |
| Mean | 0.69 (3.80) | 5.29 (5.36) | 10.99 (5.88) | 17.51 (7.99) |
| (SD) | | | | |
| Median | 0.78 | 6.61 | 10.61 | 18.17 |
| Range | −9.35-7.79 | −5.32-13.00 | −2.38-23.73 | 3.27-37.10 |

PAOC_{(0-24 h)}

| | | | | |
|---|---|---|---|---|
| N | 32 | 32 | 32 | 32 |
| Mean | 0.44 (11.99) | 32.38 (21.43) | 30.69 (17.89) | 45.38 (21.70) |
| (SD) | | | | |
| Median | 2.01 | 38.89 | 31.96 | 42.55 |
| Range | −32.02-20.70 | −11.74-65.37 | −2.77-65.92 | 7.55-99.77 |

PT25

| | | | | |
|---|---|---|---|---|
| N | 5 | 25 | 27 | 31 |
| Mean | 10.81 (12.13) | 6.75 (4.71) | 2.983 (2.51) | 1.31 (0.57) |
| (SD) | | | | |
| Median | 2.12 | 6.08 | 2.10 | 1.13 |
| Range | 1.67-24.10 | 0.62-24.10 | 0.58-12.07 | 0.58-3.08 |

HR1.5

| | | | | |
|---|---|---|---|---|
| N | 32 | 32 | 32 | 32 |
| Mean | 5.36 (0.84) | 5.17 (1.08) | 4.59 (1.02) | 3.25 (0.94) |
| (SD) | | | | |
| Median | 5.55 | 5.35 | 4.70 | 3.00 |
| Range | 3.7-6.6 | 2.7-7.2 | 2.4-6.5 | 2.2-5.6 |

Note:
Pre-dose time set to 0.0 hr for Pupillometry Area Over the Curve (PAOC) calculation

The analyses of covariance revealed a significant treatment effect for the mean PCₘᵢₙ, PAOC₍₀₋₂ₕ₎, PAOC₍₀₋₈ₕ₎, PAOC₍₀₋₂₄ₕ₎, and HR1.5 (all P<0.001). Statistically significant changes in pupil diameter PCₘᵢₙ were observed for all treatment group comparisons (adjusted P<0.001), except for the ALO-01 whole vs. ALO-01 crushed groups which were not significantly different (adjusted P=0.262). For PAOC₍₀₋₂ₕ₎, PAOC₍₀₋₈ₕ₎, PAOC₍₀₋₂₄ₕ₎, and HR1.5 statistically significant changes were observed for all treatment group comparisons (adjusted P<0.001), with the exception of the ALO-01 whole vs. Placebo comparison for PAOC₍₀₋₂ₕ₎ (adjusted P=0.667) and HR1.5 (adjusted P=0.798), as well as the PAOC₍₀₋₂₄ₕ₎ for ALO-01 whole vs. ALO-01 crushed groups (adjusted P=0.077).

### VAS Scales

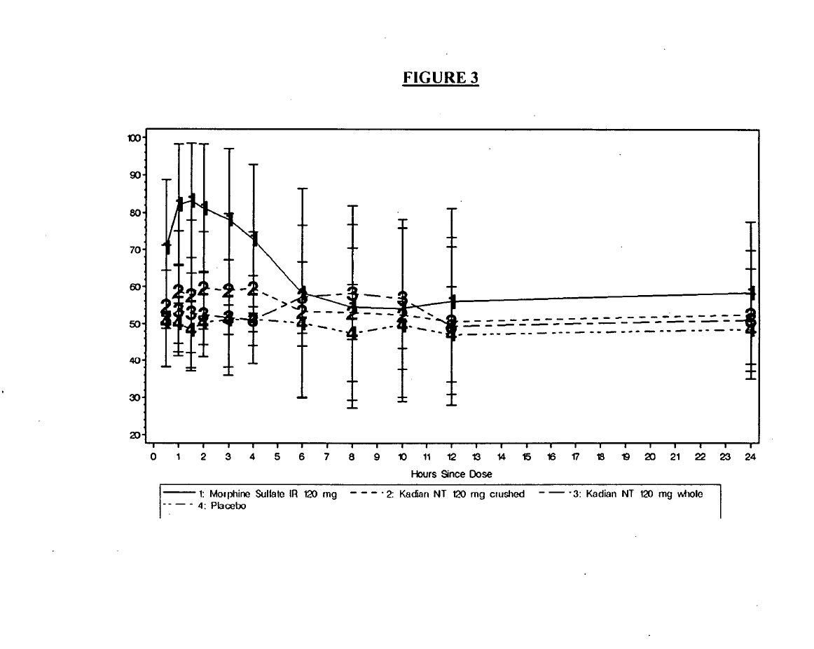

Visual analog scales (VAS) are used to directly ask the subjects how they perceive the study drug or their own subjective state. VAS for Drug Liking is assessed by the response on a scale of 0 to 100 to the item “Overall, my liking for this drug is”, where 0 is anchored by “Strong disliking”, 50 is anchored by “Neutral”, and 100 is anchored by “Strong liking”. Descriptive statistics for Drug Liking raw scores and summary parameters (per protocol population) were generated. Analysis of variance was completed for Drug Liking Eₘₐₓ, AUE₍₀₋₂ₕ₎, AUE₍₀₋₈ₕ₎, AUE₍₀₋₂₄ₕ₎, and at 1.5 hours post-dose (HR1.5). Drug Liking mean (SD) raw scores plotted over time for the per protocol population are illustrated in FIG. 3.

The proportion of subjects (per protocol population) who had a 10-100% reduction in post-dose Drug Liking Eₘₐₓ compared to MSIR 120 mg are listed in Table 28 below. Generally, the majority of subjects (presented as percentage [number of subjects/total number of subjects]) had at least a 20% minimum reduction in Eₘₐₓ following ALO-01 whole administration (65.1% [ 21/32]) and at least a 30% minimum reduction following ALO-01 crushed administration (53.1% [ 17/32]) relative to MSIR. The highest percent reductions observed were in the 40-49% range, occurring at an incidence of 15.6% ( 5/32 subjects) following ALO-01 whole administration and in 25.0% ( 8/32) of subjects following ALO-01 crushed administration.

Summary parameters of Drug Liking for the per protocol population are listed below in Table 29. Drug Liking scores showed a standard dose-response curve for each treatment group for up to and including 24 hours post-dose (FIG. 3). The Eₘₐₓ ranged from a mean (SD) of 52.2 (4.51) in the Placebo group to 89.5 (12.63) in the MSIR group. The Eₘₐₓ (mean [SD]) was similar for both ALO-01 whole (67.6 [13.12]) and ALO-01 crushed (68.1 [17.51]). Generally, Drug Liking Eₘₐₓ, AUE₍₀₋₂ₕ₎, AUE₍₀₋₈ₕ₎, and AUE₍₀₋₂₄ₕ₎ at 1.5 h post-dose increased from the lowest to highest across Placebo, ALO-01 whole, ALO-01 crushed, and MSIR treatments, respectively. For active treatments, TEₘₐₓ (hours) (mean [SD]) was lowest in the MSIR group (3.22 [4.90]) and highest in the ALO-01 whole group (6.61 [4.15]).

The analysis of variance revealed a significant treatment effect for Drug Liking Eₘₐₓ, AUE₍₀₋₂ₕ₎, AUE₍₀₋₈ₕ₎, AUE₍₀₋₂₄ₕ₎, and at 1.5 hours post-dose (HR1.5) (all P<0.001). Drug Liking Eₘₐₓ was statistically significant for all treatment combinations (adjusted P<0.001), except for ALO-01 whole vs. ALO-01 crushed (adjusted P=0.875). AUE₍₀₋₂ₕ₎, AUE₍₀₋₈ₕ₎, AUE₍₀₋₂₄ₕ₎, and HR1.5 for Drug liking were statistically significant for the treatment comparisons of MSIR vs. Placebo, ALO-01 whole, and ALO-01 crushed (adjusted P≦0.015) and for ALO-01 crushed vs. Placebo (AUE₍₀₋₂ₕ₎, AUE₍₀₋₈ₕ₎, and HR1.5 adjusted P≦0.029) but not for the treatment comparisons of ALO-01 whole vs. Placebo (adjusted P≧0.176), ALO-01 crushed vs. Placebo (AUE₍₀₋₂₄ₕ₎ adjusted P=0.136), and ALO-01 whole vs. ALO-01 crushed (adjusted P>0.074).

TABLE 28
Proportion of subjects (per protocol population) who had a 10-100%
reduction in post-dose Drug Liking Eₘₐₓ compared to Morphine
Sulfate IR 120 mg

| | ALO-01 120 mg crushed | ALO-01 120 mg |
|---|---|---|
| | (N = 32) | whole (N = 32) |
| Eₘₐₓ of Drug Liking | | |
| At least 10% reduction | 23 (71.9%) | 26 (81.3%) |
| At least 20% reduction | 21 (65.6%) | 21 (65.6%) |
| At least 30% reduction | 17 (53.1%) | 12 (37.5%) |
| At least 40% reduction | 8 (25.0%) | 5 (15.6%) |
| At least 50% reduction | 0 (0.0%) | 0 (0.0%) |
| At least 60% reduction | 0 (0.0%) | 0 (0.0%) |
| At least 70% reduction | 0 (0.0%) | 0 (0.0%) |
| At least 80% reduction | 0 (0.0%) | 0 (0.0%) |
| At least 90% reduction | 0 (0.0%) | 0 (0.0%) |
| At least 100% reduction | 0 (0.0%) | 0 (0.0%) |

Note:
Percentage is calculated based on the number of subjects in the Per Protocol Population as the denominator

TABLE 29
VAS-Drug Liking descriptive statistics of summary parameters for the per protocol
population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | |
|---|---|---|---|---|
| Mean | 52.2 (4.51) | 67.6 (13.12) | 68.1 (17.51) | 89.5 (12.63) |
| (SD) | | | | |
| Median | 51.0 | 66.0 | 62.0 | 92.5 |
| Range | 50-75 | 51-100 | 50-100 | 57-100 |

TEₘₐₓ

| | | | | |
|---|---|---|---|---|
| Mean | 2.19 (1.90) | 6.61 (4.15) | 3.47 (4.75) | 3.22 (4.90) |
| (SD) | | | | |
| Median | 1.500 | 8.000 | 2.000 | 1.492 |
| Range | 0.48-8.00 | 0.50-12.00 | 0.48-23.98 | 0.48-24.00 |

AUE_{(0-2 h)}

| | | | | |
|---|---|---|---|---|
| Mean | 74.54 (6.58) | 79.09 (14.54) | 86.73 (23.35) | 120.68 (20.87) |
| (SD) | | | | |
| Median | 75.38 | 75.75 | 77.38 | 121.68 |
| Range | 50.75-93.12 | 54.42-145.75 | 39.04-146.25 | 75.25-150.00 |

AUE_{(0-8 h)}

| | | | | |
|---|---|---|---|---|
| Mean | 375.45 (33.69) | 405.85 (62.39) | 424.29 (128.57) | 519.67 (140.64) |
| (SD) | | | | |
| Median | 376.75 | 392.92 | 397.50 | 523.11 |
| Range | 278.01-514.29 | 260.07-598.76 | 171.41-745.25 | 219.15-747.50 |

AUE_{(0-24 h)}

| | | | | |
|---|---|---|---|---|
| Mean | 1143.67 (180.82) | 1229.05 (277.89) | 1251.03 (411.70) | 1425.04 (431.24) |
| (SD) | | | | |
| Median | 1176.16 | 1213.87 | 1200.38 | 1358.73 |
| Range | 324.01-1563.29 | 326.07-1799.76 | 180.25-2272.81 | 533.73-2347.50 |

HR1.5

| | | | | |
|---|---|---|---|---|
| Mean | 48.4 (10.51) | 52.9 (10.78) | 57.6 (20.43) | 83.2 (15.38) |
| (SD) | | | | |
| Median | 50.0 | 50.0 | 50.5 | 87.5 |
| Range | 0-66 | 27-100 | 11-100 | 50-100 |

Note:
AUE calculation starts at 0.5 hr (no pre-dose value)

### Overall Drug Liking

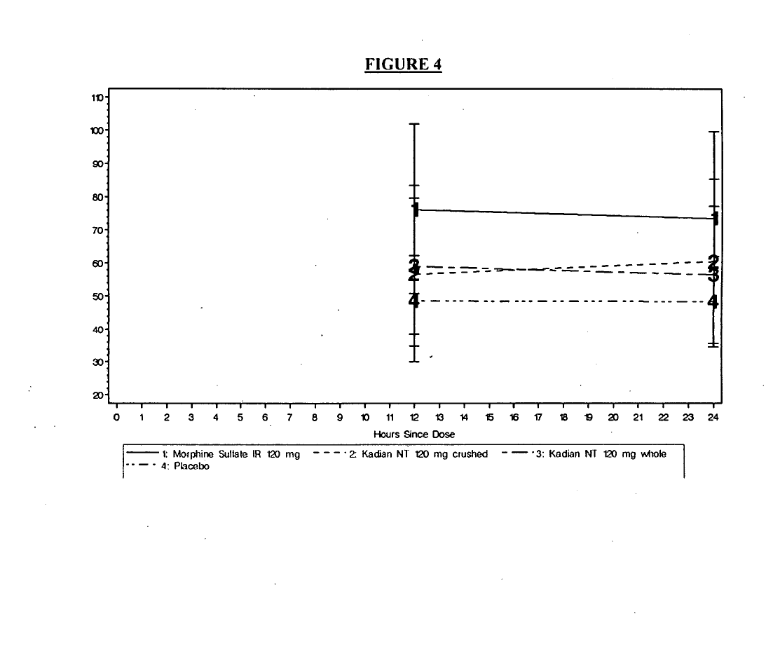

Descriptive statistics for Overall Drug Liking raw scores and summary parameters (per protocol population) were generated. Analysis of variance for Overall Drug Liking Eₘₐₓ and mean (per protocol population) was also performed (Table 30). Overall Drug Liking mean (SD) raw scores plotted at 12 and 24 hours post-dose (per protocol population) are illustrated in FIG. 4.

The proportion of subjects (per protocol population) who had a 10-100% reduction in post-dose Overall Drug Liking Eₘₐₓ compared to MSIR 120 mg are listed below in Table 31. Generally, the majority of subjects (percentage [number of subjects/total number of subjects]) had at least a 20% minimum reduction in Eₘₐₓ following both ALO-01 whole (56.3% [ 18/32]) and ALO-01 crushed (53.1% [ 17/32]) administration relative to MSIR. The highest percent reductions observed were in the 100% range, occurring at an incidence of 3.1% ( 1/32 subjects) following ALO-01 whole administration and in 6.3% ( 2/32) of subjects following ALO-01 crushed administration.

Summary parameters of Overall Drug Liking for the per protocol population are listed below in Table. The mean (SD) ranged from 48.48 (13.69) in the Placebo group to 75.02 (25.19) in the MSIR group, whereas the Eₘₐₓ mean (SD) ranged from 48.7 (13.79) in the Placebo group to 78.0 (25.00) in the MSIR group. Overall Drug Liking Mean (SD) and Eₘₐₓ generally increased from lowest to highest in the following group order: Placebo, ALO-01 whole, ALO-01 crushed, and MSIR. ALO-01 whole and ALO-01 crushed showed similar Eₘₐₓ and mean values.

The analysis of variance revealed a significant treatment effect for both Overall Drug Liking Mean and Eₘₐₓ (P<0.001) (Tables 14.2.2.10.3 and 14.2.2.10.4). Overall Drug Liking mean was significantly different for all treatment comparisons (adjusted P≦0.034) except for ALO-01 whole vs. Placebo (adjusted P=0.051) and ALO-01 whole vs. ALO-01 crushed (adjusted P=0.869). Overall Drug Liking Eₘₐₓ was significantly different between all comparisons of treatment groups (adjusted P≦0.011), except for the ALO-01 whole vs. ALO-01 crushed treatments (adjusted P=0.868).

TABLE 30
Overall Drug Liking descriptive statistics of summary
parameters for the per protocol population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | | | | | |
|---|---|---|---|---|---|---|---|---|
| Mean (SD) | 48.7 | (13.79) | 60.9 | (20.34) | 61.8 | (25.36) | 78.0 | (25.00) |

| | | | | |
|---|---|---|---|---|
| Median | 50.0 | 62.0 | 62.0 | 82.5 |
| Range | 0-77 | 0-100 | 0-100 | 6-100 |

Mean

| | | | | | | | | |
|---|---|---|---|---|---|---|---|---|
| Mean (SD) | 48.48 | (13.69) | 57.80 | (20.11) | 58.63 | (24.98) | 75.02 | (25.19) |

| | | | | |
|---|---|---|---|---|
| Median | 50.00 | 60.50 | 59.50 | 77.75 |
| Range | 0.00-76.50 | 0.00-100.00 | 0.00-100.00 | 3.00-100.00 |

TABLE 31
Proportion of subjects (per protocol population) who
had a 10-100% reduction in post-dose Overall Drug Liking
Eₘₐₓ compared to Morphine Sulfate IR 120 mg

| | |
|---|---|
| ALO-01 120 mg | ALO-01 120 mg |
| crushed (N = 32) | whole (N = 32) |

Eₘₐₓ of Overall Liking

| | | | | |
|---|---|---|---|---|
| At least 10% reduction | 19 | (59.4%) | 23 | (71.9%) |
| At least 20% reduction | 17 | (53.1%) | 18 | (56.3%) |
| At least 30% reduction | 13 | (40.6%) | 13 | (40.6%) |
| At least 40% reduction | 9 | (28.1%) | 5 | (15.6%) |
| At least 50% reduction | 4 | (12.5%) | 3 | (9.4%) |
| At least 60% reduction | 4 | (12.5%) | 1 | (3.1%) |
| At least 70% reduction | 3 | (9.4%) | 1 | (3.1%) |
| At least 80% reduction | 3 | (9.4%) | 1 | (3.1%) |
| At least 90% reduction | 2 | (6.3%) | 1 | (3.1%) |
| At least 100% reduction | 2 | (6.3%) | 1 | (3.1%) |

Note:
Percentage is calculated based on the number of subjects in the Per Protocol Population as the denominator.

### Subjective Drug Value Scale (SDV)

The Subjective Drug Value (SDV) scale involves a series of independent, theoretical forced choices between the drug administered and different monetary values. At the end of six questions, the procedure has estimated the crossover point at which the subject is indifferent between choosing drug (as would be done for all smaller values) and choosing money (as would be done for all larger values). The range of possible values is between $0.25 and $50.00.

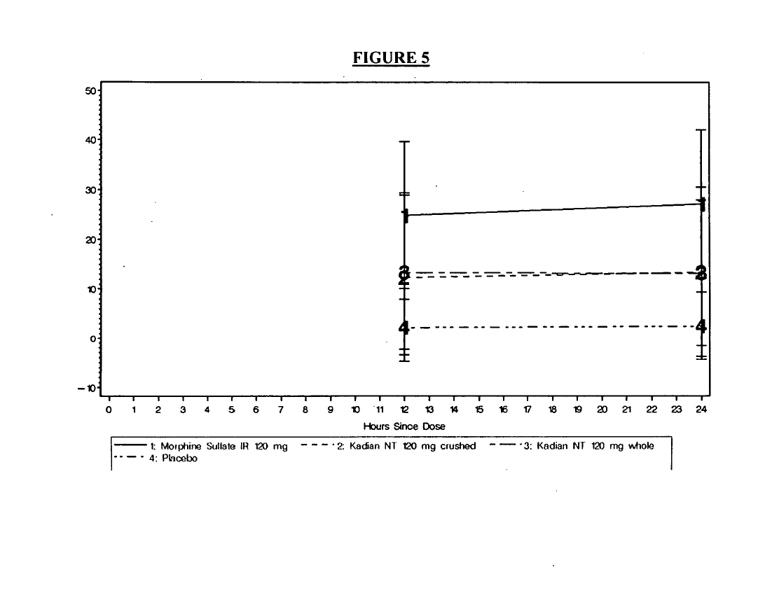

Descriptive statistics for SDV raw scores and summary parameters (per protocol population) were generated. Analysis of variance for SDV Eₘₐₓ and mean (per protocol population) was also performed. SDV mean (SD) raw scores plotted at 12 and 24 hours post-dose (per protocol population) are illustrated in FIG. 5. SDV Eₘₐₓ and mean for each treatment group (per protocol population) were also calculated.

The proportion of subjects (per protocol population) who had a 10-100% reduction in post-dose SDV Eₘₐₓ compared to MSIR 120 mg are listed below in Table 32. Half of the subjects (50% ( 16/32) had at least a 50% minimum reduction in Eₘₐₓ following either ALO-01 whole or ALO-01 crushed administration relative to MSIR. The highest percent reductions observed were in the 90-99% range, occurring at an incidence of 25.0% ( 8/32 subjects) following ALO-01 whole administration and in 37.5% ( 12/32) of subjects following ALO-01 crushed administration.

Summary parameters of SDV for the per protocol population are listed below in Table 33. The SDV ($) mean (SD) ranged from 2.40 (6.18) in the Placebo group to 26.02 (13.72) in the MSIR group; whereas, the Eₘₐₓ mean (SD) ranged from 14.22 (15.46) in the Placebo group to 28.85 (14.55) in the MSIR group. ALO-01 whole SDV was slightly higher for both mean SDV (13.31. [15.06]) and Eₘₐₓ (14.22 [15.46]) compared to ALO-01 crushed mean SDV (12.92 [16.93]) and Eₘₐₓ (13.72 [16.98]).

The analysis of variance revealed a significant treatment effect for both SDV Mean and Eₘₐₓ (P<0.001). SDV mean and Eₘₐₓ were significantly different for all treatment comparisons (adjusted P<0.001) except for ALO-01 whole vs. ALO-01 crushed (adjusted P≧0.876).

TABLE 32
Proportion of subjects (per protocol population) who
had a 10-100% reduction in post-dose Subjective Drug Value
Eₘₐₓ compared to Morphine Sulfate IR 120 mg

| | |
|---|---|
| ALO-01 120 mg | ALO-01 120 mg |
| crushed (N = 32) | whole (N = 32) |

Eₘₐₓ of Subjective Drug Value

| | | | | |
|---|---|---|---|---|
| At least 10% reduction | 23 | (71.9%) | 23 | (71.9%) |
| At least 20% reduction | 22 | (68.8%) | 21 | (65.6%) |
| At least 30% reduction | 20 | (62.5%) | 20 | (62.5%) |
| At least 40% reduction | 18 | (56.3%) | 19 | (59.4%) |
| At least 50% reduction | 17 | (53.1%) | 18 | (56.3%) |
| At least 60% reduction | 16 | (50.0%) | 16 | (50.0%) |
| At least 70% reduction | 15 | (46.9%) | 14 | (43.8%) |
| At least 80% reduction | 15 | (46.9%) | 10 | (31.3%) |
| At least 90% reduction | 12 | (37.5%) | 8 | (25.0%) |
| At least 100% reduction | 0 | (0.0%) | 0 | (0.0%) |

Note:
Percentage is calculated based on the number of subjects in the Per Protocol Population as the denominator

TABLE 33
Subjective Drug Value descriptive statistics of summary
parameters for the per protocol population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | |
|---|---|---|---|---|
| Mean (SD) | 2.73 (7.08) | 14.22 (15.46) | 13.72 (16.98) | 28.85 (14.55) |
| Median | 0.25 | 8.25 | 4.75 | 29.25 |
| Range | 0.25-26.75 | 0.25-48.00 | 0.25-48.00 | 0.25-48.00 |

Mean

| | | | | |
|---|---|---|---|---|
| Mean (SD) | 2.40 (6.18) | 13.31 (15.06) | 12.92 (16.93) | 26.02 (13.72) |
| Median | 0.25 | 8.19 | 3.81 | 25.94 |
| Range | 0.25-25.75 | 0.25-48.00 | 0.25-48.00 | 0.25-48.00 |

### Addiction Research Center Inventory (ARCI) Scales

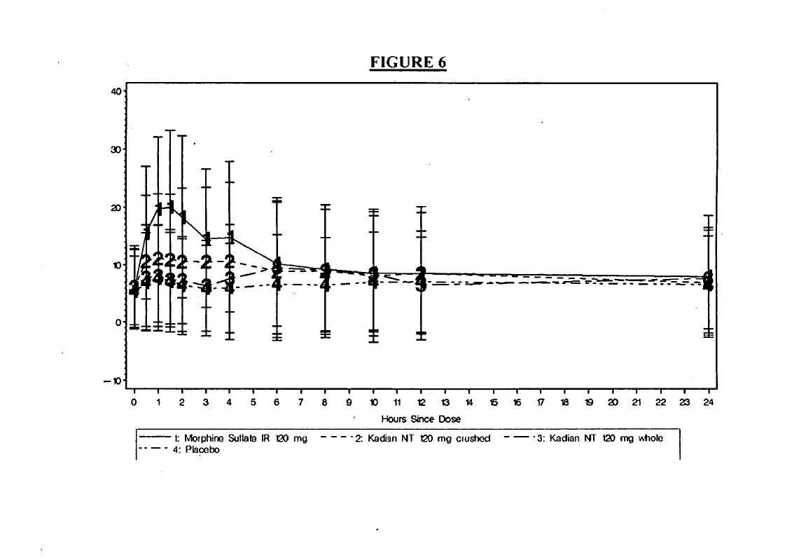

The Addiction Research Center Inventory (ARCI) scales are presented as a multiple-choice questionnaire. The responses “False” through “True” are scored as 0 through 3. The ARCI-Morphine Benzedrine Group (MBG) scale reflects feelings of euphoria and well-being. The ARCI-MBG scale is comprised of 17 questions. Scores for this scale can range from 0 to 51. Descriptive statistics for ARCI-MBG raw scores and summary parameters (per protocol population) were generated. Analysis of variance for ARCI-MBG Eₘₐₓ, AUE₍₀₋₂ₕ₎, AUE₍₀₋₈ₕ₎, AUE₍₀₋₂₄ₕ₎, and at 1.5 hours post-dose (HR1.5) was also performed. ARCI-MBG mean (SD) raw scores plotted over time for the per protocol population are illustrated in FIG. 6. ARCI-MBG Eₘₐₓ, AUE₍₀₋₂ₕ₎, AUE₍₀₋₈ₕ₎, AUE₍₀₋₂₄ₕ₎, HR1.5, and TEₘₐₓ were calculated for each treatment group.

The proportion of subjects (per protocol population) who had a 10-100% reduction in post-dose ARCI-MBG Eₘₐₓ compared to MSIR 120 mg are listed below in Table 34. Generally, the majority of subjects (percentage [number of subjects/total number of subjects]) had at least a 40% minimum reduction in Eₘₐₓ following ALO-01 whole administration (53.1% [ 17/32]) and at least a 30% minimum reduction following ALO-01 crushed administration (53.1% [ 17/32]) relative to MSIR. The highest percent reductions observed were in the 100% range, occurring at an incidence of 6.3% ( 2/32 subjects) following both ALO-01 whole and ALO-01 crushed administration.

Summary parameters of the ARCI-MBG for the per protocol population are listed below in Table 35. The ARCI-MBG Eₘₐₓ ranged from a mean (SD) of 9.4 (9.76) in the Placebo group to 23.0 (12.79) in the MSIR group. Generally, Eₘₐₓ, AUE₍₀₋₂ₕ₎, A₍₀₋₈ₕ₎, AUE₍₀₋₂₄ₕ₎, and mean at 1.5 hours post-dose increased from the lowest to highest across Placebo, ALO-01 whole, ALO-01 crushed, and MSIR treatments, respectively. For active treatments, TEₘₐₓ (hours) (mean [SD]) was lowest in the MSIR group (2.11 [4.21]) and highest in the ALO-01 whole group (5.51 [6.78]). The analysis of covariance revealed a significant treatment effects for ARCI-MBG Eₘₐₓ, AUE₍₀₋₂ₕ₎, AUE₍₀₋₈ₕ₎, AUE₍₀₋₂₄ₕ₎ and at 1.5 hours post-dose (HR1.5) (P≦0.001) (Tables 14.2.2.20.3 through 14.2.2.20.7). Eₘₐₓ, AUE₍₀₋₂ₕ₎, AUE₍₀₋₈ₕ₎, AUE₍₀₋₂₄ₕ₎ and at 1.5 hours post-dose (HR1.5) were statistically significant for the following treatment contrasts: MSIR vs. Placebo (adjusted P<0.001), MSIR vs. ALO-01 whole (adjusted P≦0.048), and MSIR vs. ALO-01 whole (adjusted P≦0.002). Statistically significant changes were also seen for the ALO-01 crushed vs. Placebo for both the Eₘₐₓ (adjusted P=0.002) and AUE₍₀₋₈ₕ₎ (adjusted P=0.047).

TABLE 34
Proportion of subjects (per protocol population)
who had a 10-100% reduction in post-dose ARCI-MBG
Eₘₐₓ compared to Morphine Sulfate IR 120 mg

| | |
|---|---|
| ALO-01 120 mg | ALO-01 120 mg |
| crushed (N = 32) | whole (N = 32) |

Eₘₐₓ of ARCI-Morphine Benzedrine Group (MBG)

| | | | | |
|---|---|---|---|---|
| At least 10% reduction | 19 | (59.4%) | 23 | (71.9%) |
| At least 20% reduction | 19 | (59.4%) | 21 | (65.6%) |
| At least 30% reduction | 17 | (53.1%) | 17 | (53.1%) |
| At least 40% reduction | 14 | (43.8%) | 17 | (53.1%) |
| At least 50% reduction | 13 | (40.6%) | 13 | (40.6%) |
| At least 60% reduction | 10 | (31.3%) | 12 | (37.5%) |
| At least 70% reduction | 8 | (25.0%) | 12 | (37.5%) |
| At least 80% reduction | 4 | (12.5%) | 8 | (25.0%) |
| At least 90% reduction | 3 | (9.4%) | 3 | (9.4%) |
| At least 100% reduction | 2 | (6.3%) | 2 | (6.3%) |

Note:
Percentage is calculated based on the number of subjects in the Per Protocol Population as the denominator.

TABLE 35
ARCI-Morphine Benzedrine Group descriptive statistics of summary
parameters for the per protocol population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | | | | | |
|---|---|---|---|---|---|---|---|---|
| Mean (SD) | 9.4 | (9.76) | 13.4 | (12.48) | 15.7 | (13.46) | 23.0 | (12.79) |

| | | | | |
|---|---|---|---|---|
| Median | 5.0 | 7.5 | 13.5 | 24.5 |
| Range | 0-34 | 0-48 | 0-46 | 0-45 |

TEₘₐₓ

| | | | | | | | | |
|---|---|---|---|---|---|---|---|---|
| Mean (SD) | 3.42 | (5.03) | 5.51 | (6.78) | 4.87 | (7.70) | 2.11 | (4.21) |

| | | | | |
|---|---|---|---|---|
| Median | 1.50 | 3.00 | 1.49 | 1.49 |
| Range | 0.48-23.98 | 0.48-24.00 | 0.48-24.02 | 0.48-24.00 |

AUE_{(0-2 h)}

| | | | | | | | | |
|---|---|---|---|---|---|---|---|---|
| Mean (SD) | 13.88 | (16.37) | 14.98 | (15.96) | 20.53 | (20.16) | 33.64 | (21.46) |

| | | | | |
|---|---|---|---|---|
| Median | 6.00 | 5.90 | 12.25 | 36.88 |
| Range | 0.00-54.02 | 0.00-46.75 | 0.00-75.99 | 0.00-70.85 |

AUE_{(0-8 h)}

| | | | | | | | | |
|---|---|---|---|---|---|---|---|---|
| Mean (SD) | 51.86 | (61.30) | 64.53 | (68.72) | 79.02 | (91.57) | 109.11 | (82.76) |

| | | | | |
|---|---|---|---|---|
| Median | 26.00 | 38.38 | 34.56 | 116.84 |
| Range | 0.00-214.99 | 0.00-222.50 | 0.00-309.99 | 0.00-276.53 |

AUE_{(0-24 h)}

| | | | | | | | | |
|---|---|---|---|---|---|---|---|---|
| Mean (SD) | 161.66 | (195.97) | 182.12 | (195.14) | 205.57 | (245.35) | 242.95 | (229.92) |

| | | | | |
|---|---|---|---|---|
| Median | 77.48 | 90.49 | 102.00 | 179.97 |
| Range | 0.00-639.99 | 0.00-581.25 | 0.00-794.94 | 0.00-748.26 |

HR1.5

| | | | | | | | | |
|---|---|---|---|---|---|---|---|---|
| Mean (SD) | 7.2 | (8.94) | 7.4 | (8.28) | 10.9 | (11.32) | 20.1 | (13.17) |

| | | | | |
|---|---|---|---|---|
| Median | 3.0 | 3.0 | 6.5 | 20.5 |
| Range | 0-29 | 0-30 | 0-45 | 0-41 |

Note:
Pre-dose time set to 0.0 hr for AUE calculation

### Cole/ARCI Abuse Potential Scale

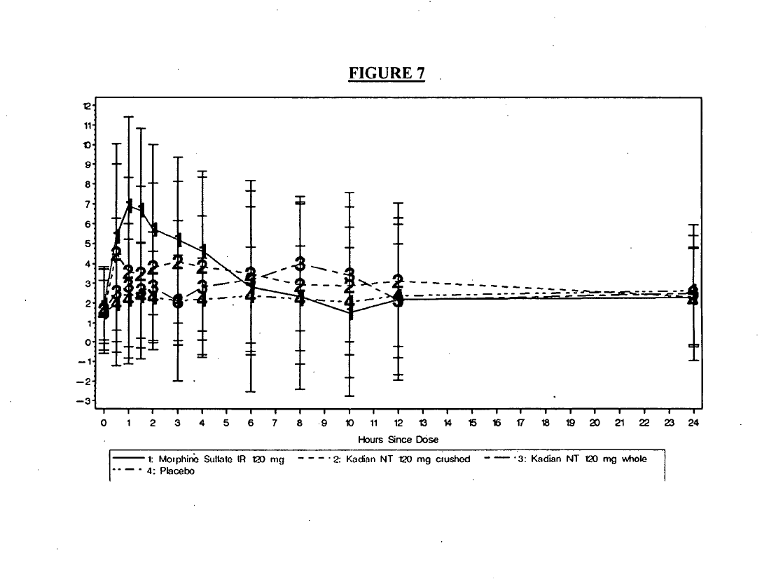

The items contributing to the Cole/ARCI-Abuse Potential scale are a mixture of positive and negative effects. Interpretation of this scale reflects a net balance among such effects. This scale includes 12 questions and scores for this scale can range from −18 to 18. Descriptive statistics for Cole/ARCI-Abuse Potential raw scores and summary parameters (per protocol population) were generated. Analysis of variance for Cole/ARCI-Abuse Potential Eₘₐₓ, AUE₍₀₋₂ₕ₎, AUE₍₀₋₈ₕ₎, AUE₍₀₋₂₄ₕ₎, and at 1.5 hours post-dose (HR1.5) was also performed. Cole/ARCI-Abuse Potential mean (SD) raw scores plotted over time for the per protocol population are illustrated in FIG. 7. Cole/ARCI-Abuse Potential Eₘₐₓ, AUE₍₀₋₂ₕ₎, ALTE₍₀₋₈ₕ₎, AUE₍₀₋₂₄ₕ₎, HR1.5 and TEₘₐₓ for each treatment group were calculated. The proportion of subjects who experienced 10% to 100% reduction in post-dose Cole/ARCI-Potential Eₘₐₓ compared to MSIR is presented below in Table 36.

Summary parameters of the Cole/ARCI-Abuse Potential for the per protocol population are listed below in Table 37. The Cole/ARCI-Abuse Potential Eₘₐₓ ranged from a mean (SD) of 3.4 (2.94) in the Placebo group to 8.7 (4.03) in the MSIR group. Generally, Eₘₐₓ, AUE₍₀₋₂ₕ₎, AUE₍₀₋₈ₕ₎, AUE₍₀₋₂₄ₕ₎, and at 1.5 hours post-dose increased from the lowest to highest across Placebo, ALO-01 whole, ALO-01 crushed, and MSIR treatments, respectively. For active treatments, TEₘₐₓ (hours) (mean [SD]) was lowest in the MSIR group (2.15 [2.28]) and highest in the ALO-01 whole group (6.17 [6.72]). The analysis of covariance revealed a significant treatment effect for Cole/ARCI-Abuse Potential E., AUE₍₀₋₂ₕ₎, AUE₀₋₈₀, and at 1.5 hours post-dose (HR1.5) (P<0.001) but not for AUE₍₀₋₂₄ₕ₎ (P=0.249). Eₘₐₓ was statistically significant for all treatment group contrasts (adjusted P≦0.002) except for the ALO-01 whole vs. ALO-01 crushed treatments (adjusted P=0.562). Cole/ARCI-Abuse Potential AUE₍₀₋₂ₕ₎ and AUE₍₀₋₈ₕ₎ were significantly different for ALO-01 crushed vs. Placebo (adjusted P≦0.019), MSIR vs. Placebo (adjusted P<0.001) and MSIR vs. ALO-01 whole (adjusted P≦0.011). Mean Cole/ARCI-Abuse Potential scores at 1.5 hours post-dose (HR1.5) were statistically significant (adjusted P<0.001) for the following treatment contrasts: MSIR vs. Placebo, MSIR vs. ALO-01 whole, and MSIR vs. ALO-01 whole.

Generally, the majority of subjects (percentage [number of subjects/total number of subjects]) had at least a 20% minimum reduction in Eₘₐₓ following both ALO-01 whole (59.4% [ 19/32]) and ALO-01 crushed (53.1% [ 17/32]) administration relative to MSIR. The highest percent reductions observed were in the 100% range, occurring at an incidence of 3.1% ( 1/32) following ALO-01 whole administration and at an incidence of 12.5% ( 4/32) following ALO-01 crushed administration.

TABLE 36
Proportion of subjects (per protocol population) who
had a 10-100% reduction in post-dose Cole/ARCI-Abuse Potential
Eₘₐₓ compared to Morphine Sulfate IR 120 mg

| | |
|---|---|
| ALO-01 120 mg | ALO-01 120 mg |
| crushed (N = 32) | whole (N = 32) |

Eₘₐₓ of Cole/ARCI-Abuse Potential

| | | | | |
|---|---|---|---|---|
| At least 10% reduction | 20 | (62.5%) | 20 | (62.5%) |
| At least 20% reduction | 17 | (53.1%) | 19 | (59.4%) |
| At least 30% reduction | 15 | (46.9%) | 15 | (46.9%) |
| At least 40% reduction | 13 | (40.6%) | 15 | (46.9%) |
| At least 50% reduction | 13 | (40.6%) | 12 | (37.5%) |
| At least 60% reduction | 8 | (25.0%) | 7 | (21.9%) |
| At least 70% reduction | 6 | (18.8%) | 6 | (18.8%) |
| At least 80% reduction | 4 | (12.5%) | 3 | (9.4%) |
| At least 90% reduction | 4 | (12.5%) | 1 | (3.1%) |
| At least 100% reduction | 4 | (12.5%) | 1 | (3.1%) |

Note:
Percentage is calculated based on the number of subjects in the Per Protocol Population as the denominator.

TABLE 37
Cole/ARCI-Abuse Potential descriptive statistics of summary
parameters for the per protocol population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | | | | | |
|---|---|---|---|---|---|---|---|---|
| Mean (SD) | 3.4 | (2.94) | 5.9 | (3.66) | 6.3 | (4.65) | 8.7 | (4.03) |

| | | | | |
|---|---|---|---|---|
| Median | 3.0 | 5.0 | 6.0 | 10.0 |
| Range | 0-11 | 0-14 | 0-18 | 0-16 |

TEₘₐₓ

| | | | | | | | | |
|---|---|---|---|---|---|---|---|---|
| Mean (SD) | 5.50 | (7.78) | 6.17 | (6.72) | 3.045 | (4.52) | 2.15 | (2.28) |

| | | | | |
|---|---|---|---|---|
| Median | 1.75 | 5.00 | 1.75 | 1.49 |
| Range | 0.48-24.00 | 0.48-24.00 | 0.48-24.00 | 0.48-10.00 |

AUE_{(0-2 h)}

| | | | | | | | | |
|---|---|---|---|---|---|---|---|---|
| Mean (SD) | 4.20 | (4.71) | 5.12 | (5.25) | 7.15 | (7.62) | 11.38 | (7.09) |

| | | | | |
|---|---|---|---|---|
| Median | 4.00 | 4.00 | 5.88 | 11.45 |
| Range | −1.75-16.23 | −2.76-20.50 | −7.71-29.03 | −5.50-25.99 |

AUE_{(0-8 h)}

| | | | | | | | | |
|---|---|---|---|---|---|---|---|---|
| Mean (SD) | 17.72 | (17.87) | 23.22 | (22.39) | 28.85 | (30.39) | 34.33 | (27.98) |

| | | | | |
|---|---|---|---|---|
| Median | 16.00 | 17.00 | 23.10 | 29.49 |
| Range | −4.26-63.02 | −13.26-79.50 | −19.00-112.03 | −19.00-97.99 |

AUE_{(0-24 h)}

| | | | | | | | | |
|---|---|---|---|---|---|---|---|---|
| Mean (SD) | 56.43 | (59.55) | 64.18 | (65.93) | 73.29 | (75.13) | 68.73 | (71.15) |

| | | | | |
|---|---|---|---|---|
| Median | 48.75 | 51.47 | 72.98 | 72.61 |
| Range | −1.00-217.02 | −26.28-230.50 | −54.25-271.03 | −69.49-226.87 |

HR1.5

| | | | | | | | | |
|---|---|---|---|---|---|---|---|---|
| Mean (SD) | 2.3 | (2.67) | 2.6 | (2.43) | 3.5 | (4.38) | 6.7 | (4.15) |

| | | | | |
|---|---|---|---|---|
| Median | 2.0 | 2.0 | 3.0 | 6.0 |
| Range | 0-10 | −1-11 | −6-15 | −3-13 |

Note:
Pre-dose time set to 0.0 hr for AUE calculation

As with the ARCI, the Cole-ARCI is a multiple-choice questionnaire. The responses “False” through “True” are scored as 0 through 3. The Cole/ARCI-Stimulation Euphoria is comprised of 16 questions, all weighted as positive in scoring. Thus, scores can range from 0 to 45.

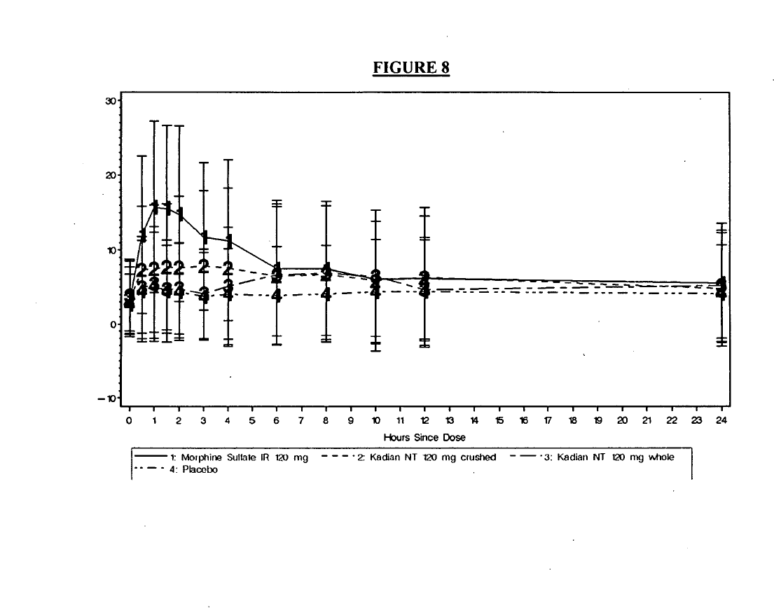

Descriptive statistics for Cole/ARCI-Stimulation Euphoria raw scores and summary parameters (per protocol population) were generated. Analysis of variance for Cole/ARCI-Stimulation Euphoria Eₘₐₓ, AUE₍₀₋₂ₕ₎, AUE₍₀₋₈ₕ₎, AUE₍₀₋₂₄ₕ₎, and at 1.5 hours post-dose (HR1.5) was also completed. Cole/ARCI-Stimulation Euphoria mean (SD) raw scores plotted over time for the per protocol population are illustrated below in FIG. 8.

The proportion of subjects (per protocol population) who had a 10-100% reduction in post-dose Cole/ARCI-Stimulation and Euphoria Eₘₐₓ compared to MSIR 120 mg are listed below in Table 38. Generally, the majority of subjects (percentage [number of subjects/total number of subjects]) had at least a 40% minimum reduction in Eₘₐₓ following ALO-01 whole administration (53.1% [ 17/32]) and at least a 400% minimum reduction following ALO-01 crushed administration (50.0% [ 16/32]) relative to MSIR. The highest percent reductions observed were in the 100% range, occurring at an incidence of 15.6.1% ( 5/32) following ALO-01 whole administration and at an incidence of 12.5% ( 4/32) following ALO-01 crushed administration. Summary parameters of the Cole/ARCI-Stimulation Euphoria for the per protocol population are listed below in Table 39. The Cole/ARCI-Stimulation Euphoria Eₘₐₓ ranged from a mean (SD) of 6.9 (8.24) in the Placebo group to 18.4 (11.64) in the MSIR group. The Eₘₐₓ (mean [SD]) was similar for both ALO-01 whole (10.8 [11.18]) and ALO-01 crushed (11.9 [11.34]). Generally, Eₘₐₓ, AUE₍₀₋₂ₕ₎, AUE₍₀₋₈ₕ₎, AUE₍₀₋₂₄ₕ₎, and mean at 1.5 hours post-dose increased from the lowest to highest across Placebo, ALO-01 whole, ALO-01 crushed, and MSIR treatments, respectively. For active treatments, TEₘₐₓ (hours) (mean [SD]) was lowest in the MSIR group (2.14 [4.15]) and highest in the ALO-01 whole group (5.08 [6.16]).

The analysis of covariance revealed a significant treatment effects for Cole/ARCI-Stimulation Euphoria AUE₍₀₋₂ₕ₎, AUE₍₀₋₈ₕ₎, AUE₍₀₋₂₄ₕ₎, and mean at 1.5 hours post-dose (HR1.5) (P<0.001), and for Eₘₐₓ treatment effects only (P<0.0.001). All parameters showed statistically significant differences for the MSIR vs. Placebo and MSIR vs. ALO-01 whole treatment contrasts (adjusted P≦0.002). In addition, significant differences were found for the ALO-01 crushed vs. Placebo treatments (Eₘₐₓ and AUE₍₀₋₈ₕ₎ [adjusted p<0.047]), MSIR vs. ALO-01 crushed treatments (Eₘₐₓ, AUE₍₀₋₂ₕ₎, AUE₍₀₋₈ₕ₎, and HR1.5 [adjusted P≦0.001], and ALO-01 whole vs. ALO-01 crushed treatment for HR 1.5 (adjusted P=0.042).

TABLE 38
Proportion of subjects (per protocol population) who had
a 10-100% reduction in post-dose Cole/ARCI-Stimulation and Euphoria
Eₘₐₓ compared to Morphine Sulfate IR 120 mg

| | |
|---|---|
| ALO-01 120 mg | ALO-01 120 mg |
| crushed (N = 32) | whole (N = 32) |

Eₘₐₓ of Cole/ARCI-Stimulation Euphoria

| | | | | |
|---|---|---|---|---|
| At least 10% reduction | 24 | (75.0%) | 22 | (68.8%) |
| At least 20% reduction | 21 | (65.6%) | 19 | (59.4%) |
| At least 30% reduction | 16 | (50.0%) | 19 | (59.4%) |
| At least 40% reduction | 16 | (50.0%) | 17 | (53.1%) |
| At least 50% reduction | 14 | (43.8%) | 16 | (50.0%) |
| At least 60% reduction | 10 | (31.3%) | 15 | (46.9%) |
| At least 70% reduction | 9 | (28.1%) | 13 | (40.6%) |
| At least 80% reduction | 7 | (21.9%) | 11 | (34.4%) |
| At least 90% reduction | 4 | (12.5%) | 6 | (18.8%) |
| At least 100% reduction | 4 | (12.5%) | 5 | (15.6%) |

Note:
Percentage is calculated based on the number of subjects in the Per Protocol Population as the denominator.

TABLE 39
Cole/ARCI-Stimulation and Euphoria descriptive statistics of
summary parameters for the per protocol population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | | | | | |
|---|---|---|---|---|---|---|---|---|
| Mean (SD) | 6.9 | (8.24) | 10.8 | (11.18) | 11.9 | (11.34) | 18.4 | (11.64) |

| | | | | |
|---|---|---|---|---|
| Median | 3.0 | 5.5 | 6.5 | 16.0 |
| Range | 0-29 | 0-39 | 0-40 | 0-38 |

TEₘₐₓ

| | | | | | | | | |
|---|---|---|---|---|---|---|---|---|
| Mean (SD) | 3.93 | (6.08) | 5.08 | (6.16) | 4.30 | (6.97) | 2.14 | (4.15) |

| | | | | |
|---|---|---|---|---|
| Median | 1.00 | 1.75 | 1.49 | 1.00 |
| Range | 0.48-24.00 | 0.48-24.00 | 0.48-24.00 | 0.48-24.00 |

AUE_{(0-2 h)}

| | | | | | | | | |
|---|---|---|---|---|---|---|---|---|
| Mean (SD) | 8.77 | (12.52) | 9.62 | (12.43) | 13.99 | (14.88) | 26.17 | (18.82) |

| | | | | |
|---|---|---|---|---|
| Median | 4.13 | 3.61 | 7.50 | 23.15 |
| Range | 0.00-48.28 | 0.00-37.25 | 0.00-48.73 | 0.00-62.00 |

AUE_{(0-8 h)}

| | | | | | | | | |
|---|---|---|---|---|---|---|---|---|
| Mean (SD) | 32.46 | (47.17) | 43.99 | (53.49 | 56.75 | (69.54) | 84.71 | (68.91) |

| | | | | |
|---|---|---|---|---|
| Median | 15.49 | 18.61 | 21.00 | 75.75 |
| Range | 0.00-189.23 | 0.00-171.00 | 0.00-229.75 | 0.00-216.50 |

AUE_{(0-24 h)}

| | | | | | | | | |
|---|---|---|---|---|---|---|---|---|
| Mean (SD) | 100.39 | (153.86) | 127.26 | (154.41) | 147.83 | (197.73) | 180.36 | (179.64) |

| | | | | |
|---|---|---|---|---|
| Median | 34.74 | 58.13 | 64.95 | 122.65 |
| Range | 0.00-539.23 | 0.00-438.78 | 0.00-607.18 | 0.00-603.04 |

HR1.5

| | | | | | | | | |
|---|---|---|---|---|---|---|---|---|
| Mean (SD) | 4.4 | (6.96) | 4.7 | (5.94) | 7.7 | (8.52) | 15.6 | (11.05) |

| | | | | |
|---|---|---|---|---|
| Median | 2.0 | 2.0 | 4.5 | 13.0 |
| Range | 0-29 | 0-18 | 0-28 | 0-37 |

Note:
Pre-dose time set to 0.0 hr for AUE calculation

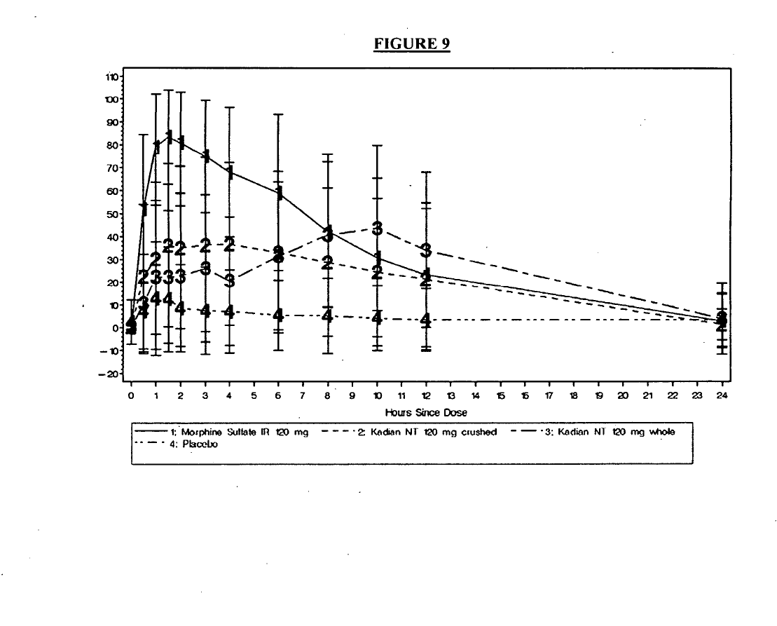

The drug-induced good effects were assessed using VAS: “I am feeling high” scored as 0 for “definitely not” and 100 for “definitely so”. Descriptive statistics for VAS-High raw scores and summary parameters (per protocol population) were generated. Analysis of covariance for High Eₘₐₓ, AUE₍₀₋₂ₕ₎, AUE₍₀₋₈ₕ₎, AUE₍₀₋₂₄ₕ₎, and at 1.5 hours post-dose (HR 1.5) was also made. High mean (SD) raw scores plotted over time for the per protocol population are illustrated in FIG. 9. High Eₘₐₓ, AUE₍₀₋₂ₕ₎, AUE₍₀₋₈ₕ₎, AUE₍₀₋₂₄ₕ₎, HR1.5 and TEₘₐₓ for each treatment group were calculated.

The proportion of subjects (per protocol population) who had a 10-100% reduction in post-dose High Eₘₐₓ compared to MSIR 120 mg are listed in Table 40. Generally, the majority of subjects (percentage [number of subjects/total number of subjects]) had at least a 20% minimum reduction in Eₘₐₓ following ALO-01 whole administration (53.1% [ 17/32]) and at least a 30% minimum reduction following ALO-01 crushed administration (53.1% [ 17/32]) relative to MSIR. The highest percent reductions observed were in the 100% range, occurring at an incidence of 9.4% ( 3/32 subjects) following ALO-01 whole administration and at an incidence of 15.6% ( 5/32) following ALO-01 crushed administration.

Summary parameters of VAS-High for the per protocol population are listed below in Table 41. High scores showed a standard dose-response curve for each treatment group for up to and including 24 hours post-dose. The Eₘₐₓ ranged from a mean (SD) of 15.2 (25.36) in the Placebo group to 90.4 (11.60) in the MSIR group. The Eₘₐₓ (mean [SD]) was higher for ALO-01 whole (60.6 [30.43]) compared to ALO-01 crushed (55.0 [34.59]). For all parameters, the lowest values were seen in the Placebo treatment and the highest in the MSIR treatment, with the exception of TEₘₐₓ which was highest in the ALO-01 whole treatment (6.41 [4.05]). Generally, High Eₘₐₓ, TEₘₐₓ, and AUE₍₀₋₂₄ₕ₎ were higher in the ALO-01 whole treatment compared to ALO-01 crushed treatment. The reverse was seen for High AUE₍₀₋₂ₕ₎, AUE₍₀₋₈ₕ₎, and HR1.5.

The analysis of covariance revealed a significant treatment effect for High Eₘₐₓ, AUE₍₀₋₂ₕ₎, AUE₍₀₋₈ₕ₎, AUE₍₀₋₂₄ₕ₎, and at 1.5 hours post-dose (HR 1.5) (P<0.001). Eₘₐₓ was found to be significantly different between all treatment contrasts (P<0.001) except for ALO-01 whole vs. ALO-01 crushed (P=0.335). The AUE₍₀₋₂ₕ₎ was found to be significantly different for all treatment contrasts (P≦0.015); whereas, the AUE₍₀₋₈ₕ₎, and AUE₍₀₋₂₄ₕ₎ were significantly different for all treatment contrasts (P≦0.011) except for ALO-01 whole vs. ALO-01 crushed (P≧0.106). At 1.5 hours post-dose, mean High was found to be significantly different for all treatment contrasts (P≦0.021) except for ALO-01 whole vs. Placebo (P=0.065).

TABLE 40
Proportion of subjects (per protocol population)
who had a 10-100% reduction in post-dose VAS-High
Eₘₐₓ compared to Morphine Sulfate IR 120 mg

| | |
|---|---|
| ALO-01 120 mg | ALO-01 120 mg |
| crushed (N = 32) | whole (N = 32) |

Eₘₐₓ of High

| | | | | |
|---|---|---|---|---|
| At least 10% reduction | 24 | (75.0%) | 26 | (81.3%) |
| At least 20% reduction | 21 | (65.6%) | 17 | (53.1%) |
| At least 30% reduction | 17 | (53.1%) | 12 | (37.5%) |
| At least 40% reduction | 12 | (37.5%) | 9 | (28.1%) |
| At least 50% reduction | 9 | (28.1%) | 6 | (18.8%) |
| At least 60% reduction | 9 | (28.1%) | 6 | (18.8%) |
| At least 70% reduction | 9 | (28.1%) | 6 | (18.8%) |
| At least 80% reduction | 8 | (25.0%) | 6 | (18.8%) |
| At least 90% reduction | 7 | (21.9%) | 4 | (12.5%) |
| At least 100% reduction | 5 | (15.6%) | 3 | (9.4%) |

Note:
Percentage is calculated based on the number of subjects in the Per Protocol Population as the denominator.

TABLE 41
VAS-High descriptive statistics of summary parameters
for the per protocol population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | | | | | |
|---|---|---|---|---|---|---|---|---|
| Mean (SD) | 15.2 | (25.36) | 60.6 | (30.43) | 55.0 | (34.59) | 90.4 | (11.60) |

| | | | | |
|---|---|---|---|---|
| Median | 1.0 | 68.5 | 64.0 | 97.0 |
| Range | 0-74 | 0-100 | 0-100 | 61-100 |

TEₘₐₓ

| | | | | | | | | |
|---|---|---|---|---|---|---|---|---|
| Mean (SD) | 1.48 | (2.41) | 6.41 | (4.05) | 3.03 | (2.70) | 1.69 | (1.27) |

| | | | | |
|---|---|---|---|---|
| Median | 0.50 | 8.00 | 2.00 | 1.49 |
| Range | 0.48-12.00 | 0.50-12.00 | 0.48-10.00 | 0.50-6.00 |

AUE_{(0-2 h)}

| | | | | | | | | |
|---|---|---|---|---|---|---|---|---|
| Mean (SD) | 19.20 | (36.06) | 33.32 | (44.28) | 53.35 | (53.54) | 127.69 | (34.02) |

| | | | | |
|---|---|---|---|---|
| Median | 0.25 | 6.33 | 49.82 | 130.77 |
| Range | 0.00-119.24 | 0.00-134.75 | 0.00-169.00 | 0.00-175.00 |

AUE_{(0-8 h)}

| | | | | | | | | |
|---|---|---|---|---|---|---|---|---|
| Mean (SD) | 58.18 | (128.25) | 205.48 | (177.32) | 257.49 | (229.64) | 506.20 | (180.99) |

| | | | | |
|---|---|---|---|---|
| Median | 0.50 | 173.74 | 197.38 | 498.13 |
| Range | 0.00-511.13 | 0.00-700.78 | 0.00-752.49 | 136.34-775.00 |

AUE_{(0-24 h)}

| | | | | | | | | |
|---|---|---|---|---|---|---|---|---|
| Mean (SD) | 117.76 | (320.42) | 597.90 | (480.50) | 494.70 | (520.16) | 792.79 | (451.41) |

| | | | | |
|---|---|---|---|---|
| Median | 0.62 | 533.10 | 276.71 | 712.22 |
| Range | 0.00-1352.13 | 0.00-1720.01 | 0.00-1598.05 | 136.34-1662.50 |

HR1.5

| | | | | | | | | |
|---|---|---|---|---|---|---|---|---|
| Mean (SD) | 12.6 | (23.10) | 22.3 | (29.00) | 36.2 | (35.78) | 83.4 | (20.68) |

| | | | | |
|---|---|---|---|---|
| Median | 0.0 | 2.5 | 34.0 | 87.5 |
| Range | 0-74 | 0-79 | 0-100 | 0-100 |

Note:
Pre-dose time set to 0.0 hr for AUE calculation

### VAS-Good Effects

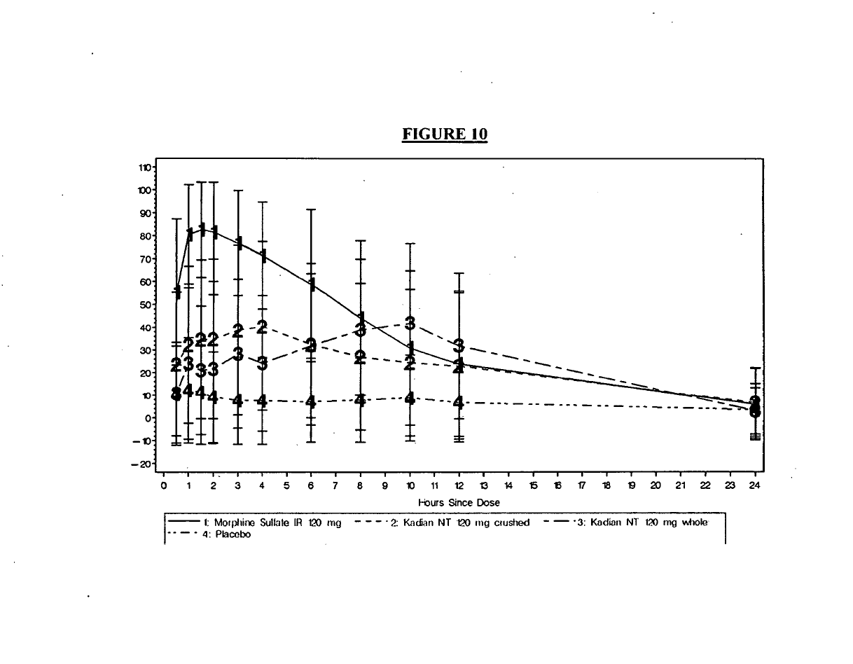

The drug induced good effects were assessed using the VAS: “I can feel good drug effects” scored as 0 for “definitely not” and 100 for “definitely so”. Descriptive statistics for VAS-Good Effects raw scores and summary parameters (per protocol population) were generated. Analysis of variance for Good Effects Eₘₐₓ, AUE₍₀₋₂ₕ₎, AUE₍₀₋₈ₕ₎, AUE₍₀₋₂₄ₕ₎, and at 1.5 hours post-dose (HR1.5) was also performed. Good Effects mean (SD) raw scores plotted over time for the per protocol population are illustrated in FIG. 10. Good Effects Eₘₐₓ, AUE₍₀₋₂ₕ₎, AUE₍₀₋₈ₕ₎, AUE₍₀₋₂₄ₕ₎, HR1.5, and TEₘₐₓ for each treatment group were calculated.

The proportion of Subjects (per protocol population) who had a 10-100% reduction in post-dose Good Effects Eₘₐₓ compared to MSIR 120 mg are listed below in Table 42. Generally, the majority of subjects (percentage [number of subjects/total number of subjects]) had at least a 20% minimum reduction in Eₘₐₓ following ALO-01 whole administration (56.3% [ 18/32]) and following ALO-01 crushed administration (65.6% [ 21/32]) relative to MSIR. The highest percent reductions observed were in the 100% range, occurring at an incidence of 12.5% ( 4/32 subjects) following both ALO-01 whole and ALO-01 crushed administration.

Summary parameters of VAS-Good Effects for the per protocol population are listed below in Table 43. Good Effects scores showed a standard dose-response curve for each treatment group, for up to and including 24 hours post-dose. The Eₘₐₓ ranged from a mean (SD) of 13.7 (24.35) in the Placebo group to 89.7 (11.40) in the MSIR group. The Eₘₐₓ (mean [SD]) was higher for ALO-01 whole (59.4 [31.77]) compared to ALO-01 crushed (52.1 [35.86]). For all parameters, the lowest values were seen in the Placebo treatment and the highest in the MSIR treatment, with the exception of TEₘₐₓ which was highest in the ALO-01 whole treatment (5.55 [4.20]). Generally, Good Effects Eₘₐₓ, TEₘₐₓ, and AUE₍₀₋₂₄ₕ₎ were higher in the ALO-01 whole treatment compared to ALO-01 crushed treatment. The reverse was seen for Good Effects AUE₍₀₋₂ₕ₎, and AUE₍₀₋₈ₕ₎, and HR1.5.

The analysis of variance revealed a significant treatment effect for Good Effects Eₘₐₓ, AUE₍₀₋₂ₕ₎, AUE₍₀₋₈ₕ₎, AUE₍₀₋₂₄ₕ₎, and at 1.5 hours post-dose (HR1.5) (P<0.001). Eₘₐₓ was found to be significantly different between all treatment contrasts (P<0.001) except for ALO-01 whole vs. ALO-01 crushed (P=0.216). The AUE₍₀₋₂ₕ₎ was found to be significantly different for all treatment contrasts (P≦0.025), except for ALO-01 whole vs. Placebo (P=0.070). Both the AUE₍₀₋₈ₕ₎ and AUE₍₀₋₂₄ₕ₎ were significantly different for all treatment contrasts (P≦0.003), except for ALO-01 whole vs. ALO-01 crushed (P≧0.148). At 1.5 hours post-dose, mean Good Effects were found to be significantly different for all treatment contrasts (P≦0.022), except for ALO-01 whole vs. Placebo (P=0.095).

TABLE 42
Proportion of subjects (per protocol population) who
had a 10-100% reduction in post-dose VAS-Good Effects
Eₘₐₓ compared to Morphine Sulfate IR 120 mg

| | |
|---|---|
| ALO-01 120 mg | ALO-01 120 mg |
| crushed (N = 32) | whole (N = 32) |

Eₘₐₓ of Good effects

| | | | | |
|---|---|---|---|---|
| At least 10% reduction | 24 | (75.0%) | 25 | (78.1%) |
| At least 20% reduction | 21 | (65.6%) | 18 | (56.3%) |
| At least 30% reduction | 16 | (50.0%) | 12 | (37.5%) |
| At least 40% reduction | 13 | (40.6%) | 9 | (28.1%) |
| At least 50% reduction | 12 | (37.5%) | 8 | (25.0%) |
| At least 60% reduction | 11 | (34.4%) | 7 | (21.9%) |
| At least 70% reduction | 10 | (31.3%) | 7 | (21.9%) |
| At least 80% reduction | 10 | (31.3%) | 6 | (18.8%) |
| At least 90% reduction | 8 | (25.0%) | 5 | (15.6%) |
| At least 100% reduction | 4 | (12.5%) | 4 | (12.5%) |

Note:
Percentage is calculated based on the number of subjects in the Per Protocol Population as the denominator

TABLE 43
VAS-Good Effects descriptive statistics of summary
parameters for the per protocol population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | | | | | |
|---|---|---|---|---|---|---|---|---|
| Mean (SD) | 13.7 | (24.35) | 59.4 | (31.77) | 52.1 | (35.86) | 89.7 | (11.40) |

| | | | | |
|---|---|---|---|---|
| Median | 1.0 | 66.5 | 62.5 | 93.0 |
| Range | 0-79 | 0-100 | 0-100 | 61-100 |

TEₘₐₓ

| | | | | | | | | |
|---|---|---|---|---|---|---|---|---|
| Mean (SD) | 1.83 | (2.77) | 5.55 | (4.20) | 3.01 | (3.10) | 1.42 | (0.81) |

| | | | | |
|---|---|---|---|---|
| Median | 0.50 | 6.00 | 1.50 | 1.00 |
| Range | 0.48-10.00 | 0.48-12.00 | 0.48-12.00 | 0.50-4.00 |

AUE_{(0-2 h)}

| | | | | | | | | |
|---|---|---|---|---|---|---|---|---|
| Mean (SD) | 16.63 | (32.25) | 30.56 | (39.80) | 47.93 | (47.43) | 116.00 | (28.75) |

| | | | | |
|---|---|---|---|---|
| Median | 0.00 | 3.13 | 43.63 | 122.08 |
| Range | 0.00-103.00 | 0.00-124.25 | 0.00-146.50 | 0.00-150.00 |

AUE_{(0-8 h)}

| | | | | | | | | |
|---|---|---|---|---|---|---|---|---|
| Mean (SD) | 62.18 | (138.54) | 208.19 | (178.24) | 256.42 | (229.85) | 502.38 | (166.07) |

| | | | | |
|---|---|---|---|---|
| Median | 0.50 | 186.43 | 214.49 | 492.30 |
| Range | 0.00-503.46 | 0.00-682.08 | 0.00-739.75 | 131.00-745.00 |

AUE_{(0-24 h)}

| | | | | | | | | |
|---|---|---|---|---|---|---|---|---|
| Mean (SD) | 156.60 | (362.97) | 572.00 | (462.54) | 532.97 | (579.35) | 812.12 | (443.58) |

| | | | | |
|---|---|---|---|---|
| Median | 0.75 | 468.28 | 231.86 | 739.69 |
| Range | 0.00-1324.46 | 0.00-1465.08 | 0.00-1790.75 | 131.00-1745.00 |

HR1.5

| | | | | | | | | |
|---|---|---|---|---|---|---|---|---|
| Mean (SD) | 11.2 | (22.76) | 21.0 | (28.27) | 34.5 | (34.85) | 82.6 | (20.74) |

| | | | | |
|---|---|---|---|---|
| Median | 0.0 | 0.5 | 28.0 | 87.5 |
| Range | 0-79 | 0-79 | 0-100 | 0-100 |

Note:
AUE calculation starts at 0.5 hr (no pre-dose value)

### Measures of Negative Effect

The measures of negative response evaluate undesirable drug effects that can potentially diminish abuse potential of the drug. These measures include: VAS for Bad Effect, Feel Sick, and Nausea, ARCI-LSD, Cole/ARCI-Unpleasantness-Dysphoria and Cole/ARCI-Unpleasantness-Physical.

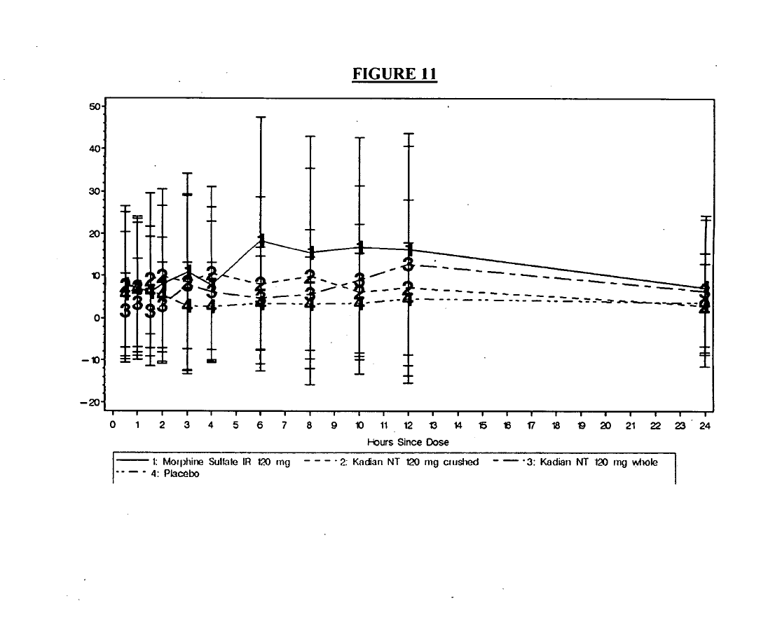

The drug-induced bad effects were assessed using VAS: “I can feel bad drug effects” scored as 0 for “definitely not” and 100 for “definitely so”. Descriptive statistics for VAS-Bad Effects raw scores and summary parameters (per protocol population) were generated. Analysis of variance for Bad Effects Eₘₐₓ, AUE₍₀₋₂ₕ₎, AUE₍₀₋₈ₕ₎, AUE₍₀₋₂₄ₕ₎, and at 1.5 hours post-dose (HR1.5) was also made. Bad Effects mean (SD) raw scores plotted over time for the per protocol population are illustrated in FIG. 11. Bad Effects Eₘₐₓ, AUE₍₀₋₂ₕ₎, AUE₍₀₋₈ₕ₎, AUE₍₀₋₂₄ₕ₎, HR 1.5, and TEₘₐₓ for each treatment group were calculated.

The proportion of subjects who experienced 10% to 100% reduction in post-dose ratings of Bad Effects Eₘₐₓ compared to MSIR is presented below in Table 44. Generally, 43.8% ( 14/32) of subjects and 50% ( 16/32) of subjects had at least a 20% and 30% minimum reduction in Eₘₐₓ following ALO-01 crushed and ALO-01 whole administration, respectively. The highest percent reductions observed were in the 100% range, occurring at an incidence of 12.5% ( 4/32 subjects) following both ALO-01 whole and ALO-01 crushed administration.

Summary parameters of VAS-Bad Effects for the per protocol population are listed below in Table 45. The Eₘₐₓ ranged from a mean (SD) of 8.0 (17.52) in the Placebo group to 35.7 (34.63) in the MSIR group. The Eₘₐₓ for Bad Effects (mean [SD]) was higher for ALO-01 whole (23.1 [31.49]) compared to ALO-01 crushed (20.9 [31.63]). Generally, for most parameters the lowest values were seen in the Placebo treatment, and the highest in the MSIR treatment, with the exception of AUE₍₀₋₂ₕ₎ which was lowest in the ALO-01 whole treatment (3.68 [10.18]) and highest in the ALO-01 crushed treatment (12.57 [25.18]) and with HR1.5 which was lowest in the ALO-01 whole treatment (1.5 [5.40]) and highest in the ALO-01 crushed treatment (9.1 [20.49]). Generally, Bad Effects Eₘₐₓ, TEₘₐₓ, and AUE₍₀₋₂₄ₕ₎ were higher in the ALO-01 whole treatment compared to ALO-01 crushed treatment. The reverse was seen for Bad Effects AUE₍₀₋₂ₕ₎, and AUE₍₀₋₈ₕ₎, and HR1.5.

The analysis of variance revealed a significant treatment effect for Bad Effects Eₘₐₓ, AUE₍₀₋₈ₕ₎, and AUE₍₀₋₂₄ₕ₎ (P≦0.006). Eₘₐₓ was found to be significantly different between all treatment contrasts (P≦0.041) except for ALO-01 whole vs. ALO-01 crushed (P=0.714). The AUE₍₀₋₈ₕ₎ was significantly different for ALO-01 crushed vs. Placebo (P=0.041), MSIR vs. Placebo (P=0.002) and MSIR vs. ALO-01 whole (P=0.006). The AUE₍₀₋₂₄ₕ₎ was significantly different for the MSIR vs. Placebo (P<0.001) and MSIR vs. ALO-01 crushed treatments (P=0.016), exclusively.

TABLE 44
Proportion of subjects (per protocol population) who
had a 10-100% reduction in post-dose VAS-Bad Effects
Eₘₐₓ compared to Morphine Sulfate IR 120 mg

| | |
|---|---|
| ALO-01 120 mg | ALO-01 120 mg |
| crushed (N = 32) | whole (N = 32) |

Eₘₐₓ of Bad effects

| | | | | |
|---|---|---|---|---|
| At least 10% reduction | 14 | (43.8%) | 16 | (50.0%) |
| At least 20% reduction | 14 | (43.8%) | 16 | (50.0%) |
| At least 30% reduction | 13 | (40.6%) | 16 | (50.0%) |
| At least 40% reduction | 12 | (37.5%) | 13 | (40.6%) |
| At least 50% reduction | 11 | (34.4%) | 12 | (37.5%) |
| At least 60% reduction | 10 | (31.3%) | 11 | (34.4%) |
| At least 70% reduction | 10 | (31.3%) | 10 | (31.3%) |
| At least 80% reduction | 8 | (25.0%) | 9 | (28.1%) |
| At least 90% reduction | 7 | (21.9%) | 7 | (21.9%) |
| At least 100% reduction | 4 | (12.5%) | 4 | (12.5%) |

Note:
Percentage is calculated based on the number of subjects in the Per Protocol Population as the denominator

TABLE 45
VAS-Bad Effects descriptive statistics of summary parameters
for the per protocol population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | |
|---|---|---|---|---|
| N | 32 | 32 | 32 | 32 |
| Mean | 8.0 (17.52) | 23.1 (31.49) | 20.9 (31.63) | 35.7 (34.63) |
| (SD) | | | | |
| Median | 0.0 | 5.5 | 2.0 | 36.5 |
| Range | 0-51 | 0-100 | 0-100 | 0-100 |

TEₘₐₓ

| | | | | |
|---|---|---|---|---|
| N | 32 | 32 | 32 | 32 |
| Mean | 1.12 (1.85) | 4.73 (6.39) | 2.62 (3.18) | 5.50 (6.46) |
| (SD) | | | | |
| Median | 0.50 | 1.74 | 0.98 | 3.00 |
| Range | 0.48-10.00 | 0.48-24.00 | 0.48-10.00 | 0.48-24.00 |

AUE_{(0-2 h)}

| | | | | |
|---|---|---|---|---|
| N | 32 | 32 | 32 | 32 |
| Mean | 9.49 (22.38) | 3.68 (10.18) | 12.57 (25.18) | 10.65 (19.38) |
| (SD) | | | | |
| Median | 0.00 | 0.00 | 0.38 | 0.00 |
| Range | 0.00-76.00 | 0.00-48.50 | 0.00-99.87 | 0.00-75.50 |

AUE_{(0-8 h)}

| | | | | |
|---|---|---|---|---|
| N | 32 | 32 | 32 | 32 |
| Mean | 29.48 (81.73) | 37.48 (79.71) | 67.908 (128.0152) | 89.39 (138.51) |
| (SD) | | | | |
| Median | 0.00 | 0.13 | 1.13 | 9.15 |
| Range | 0.00-379.50 | 0.00-306.42 | 0.00-469.00 | 0.00-505.63 |

AUE_{(0-24 h)}

| | | | | |
|---|---|---|---|---|
| N | 32 | 32 | 32 | 32 |
| Mean | 93.42 (265.77) | 188.97 (342.15) | 158.30 (346.82) | 296.02 (381.68) |
| (SD) | | | | |
| Median | 0.00 | 9.13 | 2.13 | 64.25 |
| Range | 0.00-1181.50 | 0.00-1241.42 | 0.00-1717.00 | 0.00-1228.68 |

HR1.5

| | | | | |
|---|---|---|---|---|
| N | 32 | 32 | 32 | 32 |
| Mean | 6.3 (15.42) | 1.5 (5.40) | 9.1 (20.49) | 6.1 (13.27) |
| (SD) | | | | |
| Median | 0.0 | 0.0 | 0.0 | 0.0 |
| Range | 0-51 | 0-29 | 0-94 | 0-51 |

Note:
AUE calculation starts at 0.5 hr (no pre-dose value)

### VAS-Feel Sick

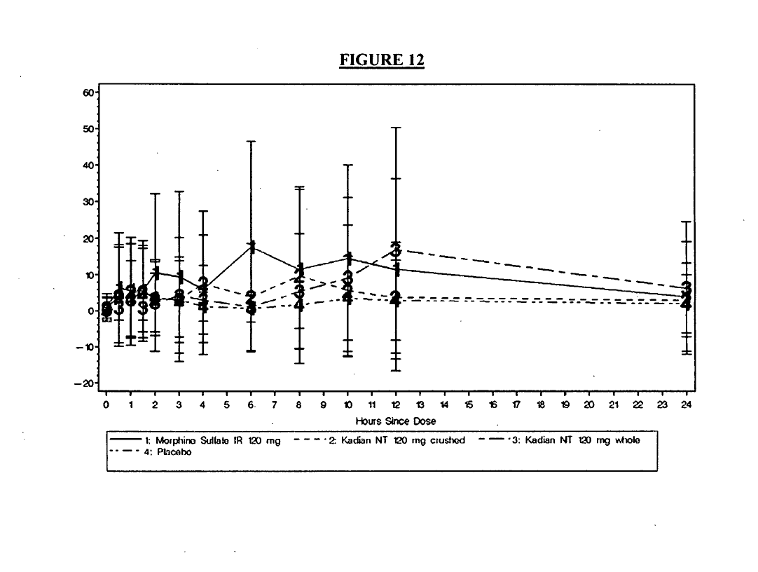

The drug effect associated with feeling sick was assessed using VAS: “I am feeling sick” scored as 0 for “definitely not” and 100 for “definitely so”. Descriptive statistics for VAS-Feel Sick raw scores and summary parameters (per protocol population) were generated. Analysis of covariance for Feel Sick Eₘₐₓ, AUE₍₀₋₂ₕ₎, AUE₍₀₋₈ₕ₎, AUE₍₀₋₂₄ₕ₎, and at 1.5 hours post-dose (HR1.5) was also made. Feel Sick mean (SD) raw scores plotted over time for the per protocol population are illustrated below in FIG. 12. Feel Sick Eₘₐₓ, AUE₍₀₋₂ₕ₎, AUE₍₀₋₈ₕ₎, AUE₍₀₋₂₄ₕ₎, HR1.5, and TEₘₐₓ for each treatment group were calculated.

The proportion of subjects who experienced 10% to 100% reduction in post-dose ratings of Feel Sick Eₘₐₓ compared to MSIR is presented below in Table 46. Generally, 43.8% ( 14/32) of subjects and 37.5% ( 12/32) of subjects had at least a 20% minimum reduction in Eₘₐₓ following ALO-01 crushed and ALO-01 whole administration, respectively. The highest percent reductions observed were in the 100% range, occurring at an incidence of 12.5% ( 4/32) following ALO-01 whole and at an incidence of 18.8% ( 6/32) following ALO-01 crushed administration.

Summary parameters of VAS-Feel Sick for the per protocol population are listed below in Table 47. The Eₘₐₓ ranged from a mean (SD) of 7.8 (17.45) in the Placebo group to 28.3 (33.64) in the MSIR group. The Eₘₐₓ for Feel Sick (mean [SD]) was higher for ALO-01 whole (24.7 [35.37]) compared to ALO-01 crushed (17.0 [28.54]). Generally, for most parameters the lowest values were seen in the Placebo treatment and the highest in the MSIR treatment, with the exception of TEₘₐₓ (4.69 [5.89]) which was highest in the ALO-01 whole treatment and AUE₍₀₋₂ₕ₎ (2.87 [8.66]) and HR1.5 (0.8 [3.23]) which were lowest in the ALO-01 whole treatment. Generally, Feel Sick Eₘₐₓ, TEₘₐₓ, and AUE₍₀₋₂₄ₕ₎ were higher in the ALO-01 whole treatment compared to ALO-01 crushed treatment. The reverse was seen for Feel Sick AUE₍₀₋₂ₕ₎, and AUE₍₀₋₈ₕ₎ and HR 1.5.

The analysis of covariance revealed a significant treatment effect for Feel Sick Eₘₐₓ, AUE₍₀₋₈ₕ), and AUE₍₀₋₂₄ₕ₎ (P≦0.014). Eₘₐₓ was found to be significantly different for the ALO-01 whole vs. Placebo (P=0.004) and MSIR vs. Placebo (P<0.001) treatment contrasts. The AUE₍₀₋₈ₕ₎ was significantly different for MSIR vs. Placebo (P<0.001), MSIR vs. ALO-01 crushed (P=0.013), and MSIR vs. ALO-01 whole (P<0.001). The AUE₍₀₋₂₄ₕ₎ was significantly different for the ALO-01 whole vs. Placebo (P=0.013), MSIR vs. Placebo (P=0.005), and MSIR vs. ALO-01 crushed treatments (P=0.048), exclusively.

TABLE 46
VAS-Feel Sick descriptive statistics of summary parameters
for the per protocol population (N = 32)

| | | | |
|---|---|---|---|
| | ALO-01 120 mg | ALO-01 120 mg | Morphine Sulfate IR |
| Placebo | whole | crushed | 120 mg |

Eₘₐₓ

| | | | | |
|---|---|---|---|---|
| Mean | 7.8 (17.45) | 24.7 (35.37) | 17.0 (28.54) | 28.3 (33.64) |
| (SD) | | | | |
| Median | 0.0 | 1.0 | 1.0 | 16.0 |
| Range | 0-63 | 0-100 | 0-97 | 0-93 |

TEₘₐₓ

| | | | | |
|---|---|---|---|---|
| Mean | 1.75 (4.18) | 4.69 (5.89) | 2.94 (3.33) | 4.28 (5.40) |
| (SD) | | | | |
| Median | 0.50 | 1.75 | 1.24 | 1.01 |
| Range | 0.48-24.00 | 0.48-24.00 | 0.50-12.00 | 0.48-24.00 |

AUE_{(0-2 h)}

| | | | | |
|---|---|---|---|---|
| Mean | 7.57 (19.30) | 2.87 (8.66) | 8.35 (18.45) | 11.71 (21.25) |
| (SD) | | | | |
| Median | 0.00 | 0.00 | 0.00 | 0.25 |
| Range | 0.00-74.50 | 0.00-41.75 | 0.00-63.00 | 0.00-91.50 |

AUE_{(0-8 h)}

| | | | | |
|---|---|---|---|---|
| Mean | 17.29 (47.42) | 20.31 (48.97) | 42.38 (85.85) | 82.36 (129.45) |
| (SD) | | | | |
| Median | 0.00 | 0.00 | 1.00 | 14.84 |
| Range | 0.00-182.35 | 0.00-238.75 | 0.00-398.00 | 0.00-416.00 |

AUE_{(0-24 h)}

| | | | | |
|---|---|---|---|---|
| Mean | 58.64 (185.11) | 200.14 (359.99) | 107.92 (294.46) | 227.56 (402.00) |
| (SD) | | | | |
| Median | 0.00 | 1.38 | 1.50 | 18.63 |
| Range | 0.00-808.35 | 0.00-1376.98 | 0.00-1578.00 | 0.00-1471.41 |

HR1.5

| | | | | |
|---|---|---|---|---|
| Mean | 5.5 (13.90) | 0.8 (3.23) | 5.3 (12.85) | 5.7 (11.59) |
| (SD) | | | | |
| Median | 0.0 | 0.0 | 0.0 | 0.0 |
| Range | 0-51 | 0-18 | 0-51 | 0-50 |

Note:
Pre-dose time set to 0.0 hr for AUE calculation

TABLE 47
Proportion of subjects (per protocol population) who had a 10-100%
reduction in post-dose VAS-Feel Sick Eₘₐₓ compared to
Morphine Sulfate IR 120 mg

| | |
|---|---|
| ALO-01 | |
| 120 mg crushed | ALO-01 120 mg whole |
| (N = 32) | (N = 32) |

Eₘₐₓ of Feel sick

| | | |
|---|---|---|
| At least 10% reduction | 14 (43.8%) | 12 (37.5%) |
| At least 20% reduction | 14 (43.8%) | 12 (37.5%) |
| At least 30% reduction | 13 (40.6%) | 11 (34.4%) |
| At least 40% reduction | 13 (40.6%) | 11 (34.4%) |
| At least 50% reduction | 13 (40.6%) | 11 (34.4%) |
| At least 60% reduction | 13 (40.6%) | 11 (34.4%) |
| At least 70% reduction | 12 (37.5%) | 11 (34.4%) |
| At least 80% reduction | 12 (37.5%) | 9 (28.1%) |
| At least 90% reduction | 11 (34.4%) | 8 (25.0%) |
| At least 100% reduction | 6 (18.8%) | 4 (12.5%) |

Note:
Percentage is calculated based on the number of subjects in the Per Protocol Population as the denominator

### VAS-Nausea

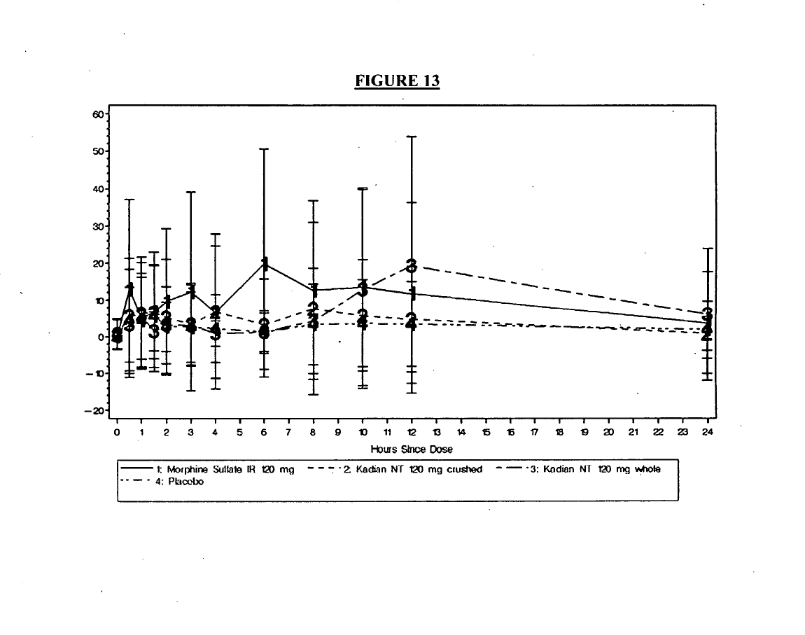

The drug-induced nausea was assessed using VAS: “I am feeling nausea” scored as 0 for “definitely not” and 100 for “definitely so”. Descriptive statistics for VAS-Nausea raw scores and summary parameters (per protocol population) were generated. Analysis of covariance for Nausea Eₘₐₓ, AUE₍₀₋₂ₕ₎, AUE₍₀₋₈ₕ₎, AUE₍₀₋₂₄ₕ₎, and at 1.5 hours post-dose (HR1.5) was also made. Nausea mean (SD) raw scores plotted over time for the per protocol population are illustrated below in FIG. 13. Nausea Eₘₐₓ, AUE₍₀₋₂ₕ₎, AUE₍₀₋₈ₕ₎, AUE₍₀₋₂₄ₕ₎, HR1.5 and TEₘₐₓ for each treatment group were calculated.

The proportion of subjects who experienced 10% to 100% reduction in post-dose ratings of feeling Nausea Eₘₐₓ compared to MSIR is presented below in Table 48. Generally, the majority of subjects had at least a 30% reduction following ALO-01 crushed administration [56.3% ( 18/32)] and ALO-01 whole administration [50.0% ( 16/32)] compared to MSIR. The highest percent reductions observed were in the 100% range, occurring at an incidence of 25.0% ( 8/32) following both ALO-01 whole and ALO-01 crushed administration.

Summary parameters of VAS-Nausea for the per protocol population are listed below in Table 49. The Eₘₐₓ ranged from a mean (SD) of 8.5 (17.64) in the Placebo group to 40.0 (37.31) in the MSIR group. The Eₘₐₓ for Nausea (mean [SD]) was higher for ALO-01 whole (27.8 [35.18]) compared to ALO-01 crushed (19.1 [30.51]). Generally, for most parameters the lowest values were seen in the Placebo treatment and the highest in the MSIR treatment, with the exception of TEₘₐₓ (4.89 [6.62]) which was highest in the ALO-01 whole treatment and AUE₍₀₋₂ₕ₎ (6.08 [12.80]) and HR1.5 (1.5 [5.29]) which were lowest in the ALO-01 whole treatment. Generally, Nausea Eₘₐₓ, TEₘₐₓ, and AUE₍₀₋₂₄ₕ₎ were higher in the ALO-01 whole treatment compared to ALO-01 crushed treatment. The reverse was seen for Nausea AUE₍₀₋₂ₕ₎, and AUE₍₀₋₈ₕ₎ and HR1.5.

The analysis of covariance revealed a significant treatment effect for Nausea Eₘₐₓ, AUE₍₀₋₂ₕ₎, AUE₍₀₋₈ₕ₎, and AUE₍₀₋₂₄ₕ₎ (10.022) and significant baseline effects for AUE₍₀₋₂ₕ₎, AUE₍₀₋₂₄ₕ₎, and at 1.5 hours post-dosing (P≦0.031). Eₘₐₓ was found to be significantly different for the ALO-01 whole vs. Placebo (P=0.003), MSIR vs. Placebo (P<0.001), and for MSIR vs. ALO-01 crushed (P=0.001) treatment contrasts. The AUE₍₀₋₂ₕ₎ was found to be significantly different for the MSIR vs. Placebo (P=0.015) and MSIR vs. ALO-01 whole treatment contrast (P=0.004). The AUE₍₀₋₈ₕ₎ was significantly different for all treatment contrasts against MSIR (P<0.001). The AUE₍₀₋₂₄ₕ₎ was significantly different for all treatment contrasts (P≦0.018), with the exception of ALO-01 crushed vs. Placebo (P=0.558) and MSIR vs. ALO-01 whole (P=0.717).

TABLE 48
VAS-Nausea descriptive statistics of summary parameters for
the per protocol population (N = 32)

| | | | |
|---|---|---|---|
| | ALO-01 120 mg | ALO-01 120 mg | Morphine Sulfate IR |
| Placebo | whole | crushed | 120 mg |

Eₘₐₓ

| | | | | |
|---|---|---|---|---|
| Mean | 8.5 (17.64) | 27.8 (35.18) | 19.1 (30.51) | 40.0 (37.31) |
| (SD) | | | | |
| Median | 0.0 | 4.5 | 1.0 | 30.0 |
| Range | 0-51 | 0-100 | 0-100 | 0-100 |

TEₘₐₓ

| | | | | |
|---|---|---|---|---|
| Mean | 1.17 (1.80) | 4.89 (6.62) | 2.92 (3.76) | 3.97 (4.13) |
| (SD) | | | | |
| Median | 0.50 | 1.00 | 0.50 | 2.00 |
| Range | 0.48-10.00 | 0.48-24.02 | 0.48-12.00 | 0.48-12.00 |

AUE_{(0-2 h)}

| | | | | |
|---|---|---|---|---|
| Mean | 8.19 (19.56) | 6.08 (12.80) | 10.75 (21.30) | 14.89 (21.53) |
| (SD) | | | | |
| Median | 0.00 | 0.00 | 0.00 | 0.50 |
| Range | 0.00-75.19 | 0.00-49.25 | 0.00-70.50 | 0.00-81.00 |

AUE_{(0-8 h)}

| | | | | |
|---|---|---|---|---|
| Mean | 21.47 (59.41) | 18.88 (42.19) | 41.23 (86.81) | 93.99 (126.27) |
| (SD) | | | | |
| Median | 0.00 | 2.00 | 1.13 | 43.66 |
| Range | 0.00-240.83 | 0.00-223.75 | 0.00-409.00 | 0.00-454.23 |

AUE_{(0-24 h)}

| | | | | |
|---|---|---|---|---|
| Mean | 67.69 (207.17) | 219.49 (347.74) | 98.55 (237.97) | 238.35 (382.59) |
| (SD) | | | | |
| Median | 0.00 | 22.25 | 4.25 | 53.70 |
| Range | 0.00-803.47 | 0.00-1189.88 | 0.00-1175.00 | 0.00-1421.86 |

HR1.5

| | | | | |
|---|---|---|---|---|
| Mean | 5.6 (13.97) | 1.5 (5.29) | 6.7 (16.24) | 6.8 (12.59) |
| (SD) | | | | |
| Median | 0.0 | 0.0 | 0.0 | 0.0 |
| Range | 0-51 | 0-23 | 0-63 | 0-50 |

Note:
Pre-dose time set to 0.0 hr for AUE calculation

TABLE 49
Proportion of subjects (per protocol population) who had a 10-100%
reduction in post-dose VAS-Nausea Eₘₐₓ compared to
Morphine Sulfate IR 120 mg

| | |
|---|---|
| ALO-01 | |
| 120 mg crushed | ALO-01 120 mg whole |
| (N = 32) | (N = 32) |

Eₘₐₓ of Nausea

| | | |
|---|---|---|
| At least 10% reduction | 19 (59.4%) | 17 (53.1%) |
| At least 20% reduction | 19 (59.4%) | 16 (50.0%) |
| At least 30% reduction | 18 (56.3%) | 16 (50.0%) |
| At least 40% reduction | 15 (46.9%) | 15 (46.9%) |
| At least 50% reduction | 15 (46.9%) | 14 (43.8%) |
| At least 60% reduction | 15 (46.9%) | 12 (37.5%) |
| At least 70% reduction | 15 (46.9%) | 11 (34.4%) |
| At least 80% reduction | 14 (43.8%) | 11 (34.4%) |
| At least 90% reduction | 11 (34.4%) | 10 (31.3%) |
| At least 100% reduction | 8 (25.0%) | 8 (25.0%) |

Note:
Percentage is calculated based on the number of subjects in the Per Protocol Population as the denominator

### ARCI-LSD Scale

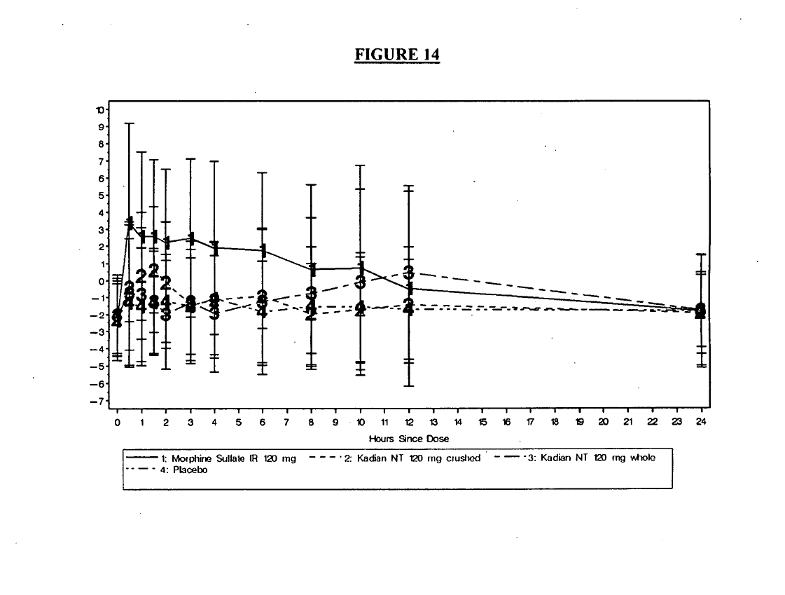

The ARCI-LSD scale may reflect dysphoria and feelings of fear and is comprised of 14 questions, 10 of which are weighted as positive in scoring. Thus, scores for this scale can range from −12 to 30. Descriptive statistics for ARCI-LSD raw scores and summary parameters (per protocol population) were generated. Analysis of covariance for ARCI-LSD Eₘₐₓ, AUE₍₀₋₂ₕ₎, AUE₍₀₋₈ₕ₎, AUE₍₀₋₂₄ₕ₎, and at 1.5 hours post-dose (HR1.5) was also completed. ARCI-LSD mean (SD) raw scores plotted over time for the per protocol population are illustrated in FIG. 14. ARCI-LSD box plots for Eₘₐₓ, TEₘₐₓ, AUE₍₀₋₂ₕ₎, AUE₍₀₋₈ₕ₎, AUE₍₀₋₂₄ₕ₎, and HR1.5 for each treatment group was calculated.

The proportion of subjects who had a 10-100% reduction in Eₘₐₓ after administration of ALO-01 whole or crushed compared to Eₘₐₓ after MSIR administration are listed below in Table 50. Relative to Eₘₐₓ for MSIR, the majority of subjects (percentage [number of subjects/total number of subjects]) had at least a 60% minimum reduction in Eₘₐₓ following ALO-01 whole administration (53.1% [ 17/32]) and following ALO-01 crushed administration (50.0% [ 16/32]). The highest reductions were seen as a 100% reduction in the ALO-01 whole group (37.5% [ 12/32]) and the ALO-01 crushed group (21.9% [ 7/32]).

Summary parameters of ARCI-LSD for the per protocol population are listed below in Table 51. The Eₘₐₓ ranged from a mean (SD) of 0.3 (3.35) in the Placebo group to 7.4 (5.58) in the MSIR group. The Eₘₐₓ mean [SD] for ALO-01 crushed treatment was lower than for ALO-01 whole group (2.9 [4.14] and 3.5 [5.93], respectively). Generally, for most parameters the lowest values were seen in the Placebo treatment and the highest in the MSIR treatment, with the exception of TEₘₐₓ, which was lowest for MSIR and the highest for the ALO-01 whole treatment. AUE₍₀₋₂ₕ₎ and AUE₍₀₋₈ₕ₎ mean ARCI-LSD scores were lower for ALO-01 whole than for ALO-01 crushed, while for AUE₍₀₋₂₄ₕ₎ the reverse pattern was observed. For Placebo and ALO-01 whole mean response [SD] at 1.5 hours post-dose was the same (−1.3 [3.00] and −1.3 [3.12], respectively)

The analysis of covariance revealed a significant treatment effect for ARCI-LSD Eₘₐₓ, AUE₍₀₋₂ₕ₎, AUE₍₀₋₈ₕ₎, AUE₍₀₋₂₄ₕ₎, and at 1.5 hours post-dose (P≦0.003). For Eₘₐₓ, AUE₍₀₋₂ₕ₎, and HR1.5, all treatments contrasts reached statistical significance (P<0.032) except for Eₘₐₓ ALO-01 whole vs. ALO-01 crushed treatment comparison (P=0.574) and AUE₍₀₋₂ₕ₎ and HR1.5 ALO-01 whole vs. Placebo comparison (P=0.664 and P=0.808, respectively). Additionally, the following treatment contrasts were significantly different: AUE₍₀₋₈ₕ₎ for MSIR vs. all treatments contrasts (P<0.001) and AUE₍₀₋₂₄ₕ₎ MSIR vs. Placebo and ALO-01 crushed (P<0.001 and P=0.002, respectively).

TABLE 50
ARCI-LSD descriptive statistics of summary parameters for
the per protocol population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | |
|---|---|---|---|---|
| Mean | 0.3 (3.35) | 3.5 (5.93) | 2.9 (4.14) | 7.4 (5.58) |
| (SD) | | | | |
| Median | 0.0 | 1.5 | 2.0 | 6.0 |
| Range | −4-11 | −4-21 | −4-15 | 0-23 |

TEₘₐₓ

| | | | | |
|---|---|---|---|---|
| Mean | 3.043 (4.7188) | 5.767 (5.6935) | 2.608 (4.2709) | 2.548 (2.9092) |
| (SD) | | | | |
| Median | 1.500 | 6.000 | 1.492 | 1.258 |
| Range | 0.48-24.00 | 0.48-24.00 | 0.48-24.00 | 0.50-10.02 |

AUE_{(0-2 h)}

| | | | | |
|---|---|---|---|---|
| Mean | −2.877 (5.6898) | −2.417 (6.0894) | −0.346 (5.8469) | 4.263 (7.7134) |
| (SD) | | | | |
| Median | −2.350 | −2.488 | 0.000 | 2.796 |
| Range | −13.75-14.77 | −12.50-18.57 | −11.00-12.21 | −14.40-24.00 |

AUE_{(0-8 h)}

| | | | | |
|---|---|---|---|---|
| Mean | −11.803 (22.0695) | −10.861 (23.5669) | −7.363 (21.7359) | 14.858 (26.4456) |
| (SD) | | | | |
| Median | −10.121 | −13.129 | −5.338 | 6.375 |
| Range | −50.25-66.25 | −47.50-45.25 | −48.25-53.83 | −22.00-76.45 |

AUE_{(0-24 h)}

| | | | | |
|---|---|---|---|---|
| Mean | −38.949 (61.8819) | −19.013 (72.1801) | −34.271 (61.2450) | 3.055 (89.2391) |
| (SD) | | | | |
| Median | −33.746 | −16.129 | −29.871 | −11.663 |
| Range | −160.38-134.25 | −163.50-136.67 | −145.00-128.76 | −117.43-248.76 |

HR1.5

| | | | | |
|---|---|---|---|---|
| Mean | −1.3 (3.00) | −1.3 (3.12) | 0.6 (3.68) | 2.6 (4.48) |
| (SD) | | | | |
| Median | −1.0 | −2.0 | 0.0 | 2.0 |
| Range | −7-10 | −6-6 | −6-9 | −7-12 |

Note:
Pre-dose time set to 0.0 hr for AUE calculation

TABLE 51
For ARCI-LSD proportion of subjects (per protocol population)
who had a 10-100% reduction in post-dose Eₘₐₓ compared to
Morphine Sulfate IR 120 mg

| | |
|---|---|
| ALO-01 | |
| 120 mg crushed | ALO-01 120 mg whole |
| (N = 32) | (N = 32) |

Eₘₐₓ of ARCI-LSD

| | | |
|---|---|---|
| At least 10% reduction | 25 (78.1%) | 22 (68.8%) |
| At least 20% reduction | 24 (75.0%) | 22 (68.8%) |
| At least 30% reduction | 23 (71.9%) | 20 (62.5%) |
| At least 40% reduction | 21 (65.6%) | 20 (62.5%) |
| At least 50% reduction | 20 (62.5%) | 19 (59.4%) |
| At least 60% reduction | 16 (50.0%) | 17 (53.1%) |
| At least 70% reduction | 13 (40.6%) | 15 (46.9%) |
| At least 80% reduction | 12 (37.5%) | 12 (37.5%) |
| At least 90% reduction | 9 (28.1%) | 12 (37.5%) |
| At least 100% reduction | 7 (21.9%) | 12 (37.5%) |

Note:
Percentage is calculated based on the number of subjects in the Per Protocol Population as the denominator

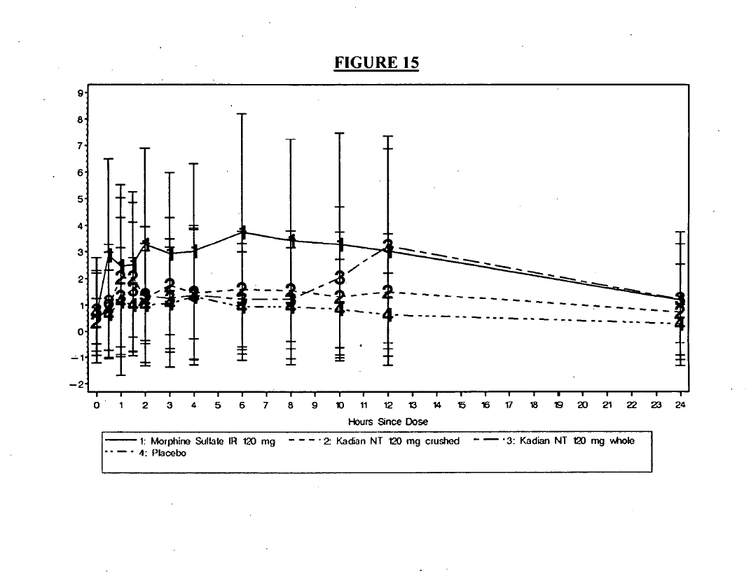

The Cole/ARCI-Unpleasantness-Physical scale is comprised of eight questions, all weighted as positive in scoring. Thus, scores for this scale can range from 0 to 24. Descriptive statistics for Cole/ARCI-Unpleasantness-Physical raw scores and summary parameters (per protocol population) were generated. Analysis of covariance for Cole/ARCI-Unpleasantness-Physical Eₘₐₓ, AUE₍₀₋₂ₕ₎, AUE₍₀₋₈ₕ₎, AUE₍₀₋₂₄ₕ₎, and at 1.5 hours post-dose (HR1.5) was also completed. Cole/ARCI-Unpleasantness-Physical mean (SD) raw scores plotted over time for the per protocol population are illustrated in FIG. 15. Cole/ARCI-Unpleasantness-Physical box plots for Eₘₐₓ, TEₘₐₓ, AUE₍₀₋₂ₕ₎, AUE₍₀₋₈ₕ₎, AUE₍₀₋₂₄ₕ₎, and HR1.5 for each treatment group were calculated.

The proportion of subjects who had a 10-100% reduction in Eₘₐₓ after administration of ALO-01 whole or crushed compared to Eₘₐₓ after MSIR administration are listed below in Table 52. Relative to Eₘₐₓ for MSIR, the majority of subjects had at least a 10% reduction following ALO-01 whole administration [50.0% ( 16/32)] and at least a 30% reduction following ALO-01 crushed administration [62.5% ( 20/32)]. The highest percent reductions observed were in the 100% range, occurring at an incidence of 18.8% ( 6/32) following ALO-01 whole and 9.4% ( 3/32) ALO-01 crushed administration.

Summary parameters of Cole/ARCI-Unpleasantness-Physical for the per protocol population are listed below in Table 53. The Eₘₐₓ ranged from a mean (SD) of 2.3 (2.84) in the Placebo group to 7.0 (5.30) in the MSIR group. The Eₘₐₓ mean [SD] for ALO-01 crushed treatment was lower than for the ALO-01 whole treatment (3.9 [3.50] and 4.7 [4.23], respectively). Generally, for most parameters the lowest values were seen in the Placebo treatment and the highest in the MSIR treatment, with the exception of TEₘₐₓ, which was lowest for Placebo followed by ALO-01 crushed, MSIR and ALO-01 whole. The AUE₍₀₋₂ₕ₎, AUE₍₀₋₈ₕ₎, and HR1.5 was greater for ALO-01 crushed than for ALO-01 whole treatment; however, the pattern was reversed for AUE₍₀₋₂₄ₕ₎.

The analysis of covariance revealed a significant treatment effect for Eₘₐₓ, AUE₍₀₋₂ₕ₎, AUE₍₀₋₈ₕ₎, AUE₍₀₋₂₄ₕ₎, and HR1.5 (P≦0.023). For Eₘₐₓ all treatment contrasts reached statistical significance (P≦0.027) except for ALO-01 whole vs. ALO-01 crushed treatment contrasts (P=0.464). For AUE₍₀₋₂ₕ₎ and AUE₍₀₋₈ₕ₎ all contrasts against MSIR treatment (P≦0.02 and P≦0.001, respectively). For AUE₍₀₋₂₄ₕ₎, all contrasts against MSIR treatment (P≦0.047) and ALO-01 whole vs. Placebo contrast (P=0.003) were statistically significant. At 1.5 hours post-dosing time point, only ALO-01 crushed vs. Placebo (P=0.038) and MSIR vs. Placebo (P=0.003) contrasts were significantly different.

TABLE 52
Cole/ARCI-Unpleasantness-Physical descriptive statistics of
summary parameters for the per protocol population (N = 32)

| | | | |
|---|---|---|---|
| | ALO-01 120 mg | ALO-01 120 mg | Morphine Sulfate IR |
| Placebo | whole | crushed | 120 mg |

Eₘₐₓ

| | | | | |
|---|---|---|---|---|
| Mean | 2.3 (2.84) | 4.7 (4.23) | 3.9 (3.50) | 7.0 (5.30) |
| (SD) | | | | |
| Median | 1.0 | 3.5 | 3.0 | 6.5 |
| Range | 0-12 | 0-16 | 0-12 | 0-19 |

TEₘₐₓ

| | | | | |
|---|---|---|---|---|
| Mean | 1.919 (2.3230) | 7.298 (7.9755) | 3.967 (4.9016) | 5.078 (4.3187) |
| (SD) | | | | |
| Median | 1.000 | 3.500 | 1.500 | 4.000 |
| Range | 0.48-12.00 | 0.50-24.02 | 0.48-24.00 | 0.48-12.00 |

AUE_{(0-2 h)}

| | | | | |
|---|---|---|---|---|
| Mean | 1.823 (2.9066) | 2.560 (4.4355) | 2.918 (3.8119) | 4.931 (5.0539) |
| (SD) | | | | |
| Median | 0.375 | 0.500 | 1.500 | 3.375 |
| Range | 0.00-11.32 | 0.00-20.34 | 0.00-16.75 | 0.00-17.50 |

AUE_{(0-8 h)}

| | | | | |
|---|---|---|---|---|
| Mean | 8.163 (14.8236) | 10.216 (13.4148) | 12.265 (13.1682) | 24.992 (22.4801) |
| (SD) | | | | |
| Median | 1.500 | 2.258 | 8.638 | 20.233 |
| Range | 0.00-67.26 | 0.00-49.25 | 0.00-56.57 | 0.00-72.15 |

AUE_{(0-24 h)}

| | | | | |
|---|---|---|---|---|
| Mean | 16.852 (31.6225) | 45.343 (48.8634) | 31.260 (37.3776) | 63.329 (65.2520) |
| (SD) | | | | |
| Median | 1.763 | 33.500 | 22.196 | 40.850 |
| Range | 0.00-125.99 | 0.00-193.70 | 0.00-154.48 | 0.00-261.23 |

HR1.5

| | | | | |
|---|---|---|---|---|
| Mean | 1.0 (1.78) | 1.6 (2.54) | 2.0 (2.83) | 2.5 (2.74) |
| (SD) | | | | |
| Median | 0.0 | 0.0 | 0.5 | 2.0 |
| Range | 0-6 | 0-9 | 0-11 | 0-9 |

Note:
Pre-dose time set to 0.0 hr for AUE calculation

TABLE 53
For Cole/ARCI-Unpleasantness-Physical, the proportion of subjects
(per protocol population) who had a 10-100% reduction in post-dose
Eₘₐₓ compared to Morphine Sulfate IR 120 mg

| | |
|---|---|
| ALO-01 | |
| 120 mg crushed | ALO-01 120 mg whole |
| (N = 32) | (N = 32) |

Eₘₐₓ of Cole/ARCI-Unpleasantness-Physical

| | | |
|---|---|---|
| At least 10% reduction | 23 (71.9%) | 16 (50.0%) |
| At least 20% reduction | 23 (71.9%) | 15 (46.9%) |
| At least 30% reduction | 20 (62.5%) | 15 (46.9%) |
| At least 40% reduction | 14 (43.8%) | 14 (43.8%) |
| At least 50% reduction | 12 (37.5%) | 12 (37.5%) |
| At least 60% reduction | 11 (34.4%) | 10 (31.3%) |
| At least 70% reduction | 8 (25.0%) | 10 (31.3%) |
| At least 80% reduction | 6 (18.8%) | 9 (28.1%) |
| At least 90% reduction | 3 (9.4%) | 7 (21.9%) |
| At least 100% reduction | 3 (9.4%) | 6 (18.8%) |

Note:
Percentage is calculated based on the number of subjects in the Per Protocol Population as the denominator

### Cole/ARCI-Unpleasantness-Dysphoria Scale

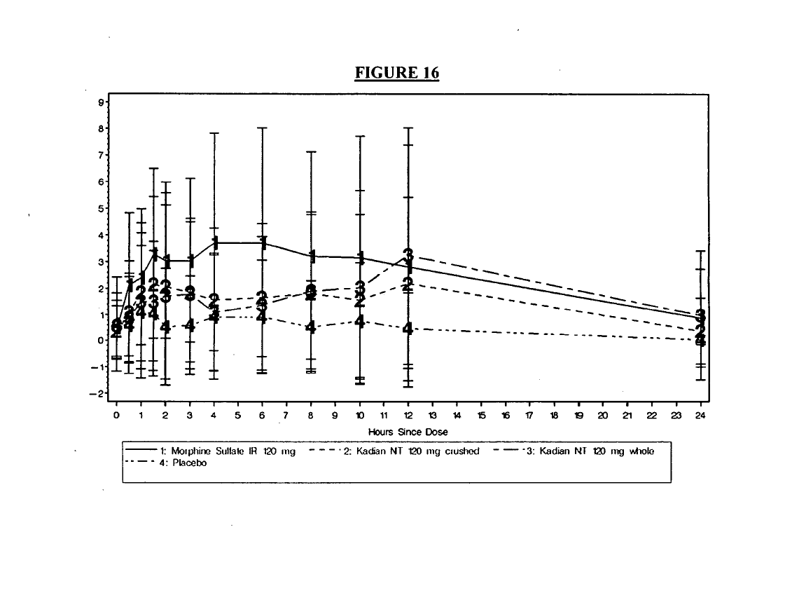

The Cole/ARCI-Unpleasantness-Dysphoria scale is comprised of six questions, all weighted as positive in scoring. Thus, scores for this scale can range from 0 to 18. Descriptive statistics for Cole/ARCI-Unpleasantness-Dysphoria raw scores and summary parameters (per protocol population) were generated. Analysis of covariance for Cole/ARCI-Unpleasantness-Dysphoria Eₘₐₓ, AUE₍₀₋₂ₕ₎, AUE₍₀₋₈ₕ₎, AUE₍₀₋₂₄ₕ₎ and at 1.5 hours post-dose (HR1.5) was also completed. Cole/ARCI-Unpleasantness-Dysphoria mean (SD) raw scores plotted over time for the per protocol population are illustrated in FIG. 16. Cole/ARCI-Unpleasantness-Dysphoria box plots for Eₘₐₓ, TEₘₐₓ, AUE₍₀₋₂ₕ₎, AUE₍₀₋₈ₕ₎, AUE₍₀₋₂₄ₕ₎, and HR1.5 were also calculated.

The proportion of subjects who had a 10-100% reduction in Eᵣₙₐₓ after administration of ALO-01 whole or crushed compared to Eₘₐₓ after MSIR administration are listed below in Table 54. Relative to Eₘₐₓ for MSIR, a 20% Eₘₐₓ reduction was reported by 16 subjects [50.0% ( 16/32)] administered ALO-01 whole and 15 subjects [46.9% ( 15/32)] administered ALO-01 crushed. Furthermore, 5 subjects (15.6%) administered ALO-01 crushed and 8 subjects (25.0%) administered ALO-01 whole reported 100% Eₘₐₓ reduction.

Summary parameters of Cole/ARCI-Unpleasantness-Dysphoria for the per protocol population are listed below in Table 55. The Eₘₐₓ ranged from a mean (SD) of 1.9 (3.21) in the Placebo group to 5.8 (4.90) in the MSIR group. The Eₘₐₓ mean [SD] for ALO-01 crushed treatment was lower than for the ALO-01 whole treatment (4.2 [4.09] and 4.8 [4.98], respectively). Generally, for most parameters the lowest values were seen in the Placebo treatment and the highest in the MSIR treatment, with the exception of TEₘₐₓ, which was lowest for Placebo followed by ALO-01 crushed, MSIR, and ALO-01 whole. The AUE₍₀₋₂ₕ₎, AUE₍₀₋₈ₕ₎, and HR1.5 was greater for ALO-01 crushed than for ALO-01 whole treatment; however, the pattern was reversed for AUE₍₀₋₂₄ₕ₎.

The analysis of covariance revealed a significant treatment effect for Eₘₐₓ, AUE₍₀₋₂ₕ₎, AUE₍₀₋₈ₕ₎, AUE₍₀₋₂₄ₕ₎, and HR1.5 (P<0.001). For Eₘₐₓ all treatment contrasts against Placebo reached statistical significance (P≦0.001). For AUE₍₀₋₂ₕ₎, AUE₍₀₋₈ₕ₎, and HR1.5 all contrasts against MSIR treatment (P≦0.038, P<0.001 and P≦0.046, respectively) and ALO-01 crushed vs. Placebo contrast (P=0.024, P=0.012 and P=0.034, respectively) were significant. For AUE₍₀₋₂₄ₕ₎ all treatment contrasts against Placebo (P≦0.011) and MSIR vs. ALO-01 crushed (P=0.019) reached statistical significance.

TABLE 54
Cole/ARCI-Unpleasantness-Dysphoria descriptive statistics of
summary parameters for the per protocol population (N = 32)

| | | | |
|---|---|---|---|
| | ALO-01 120 mg | ALO-01 120 mg | Morphine Sulfate IR |
| Placebo | whole | crushed | 120 mg |

Eₘₐₓ

| | | | | |
|---|---|---|---|---|
| Mean | 1.9 (3.21) | 4.8 (4.98) | 4.2 (4.09) | 5.8 (4.90) |
| (SD) | | | | |
| Median | 0.0 | 3.0 | 3.0 | 4.0 |
| Range | 0-12 | 0-15 | 0-12 | 0-18 |

TEₘₐₓ

| | | | | |
|---|---|---|---|---|
| Mean | 2.231 (2.9256) | 4.517 (5.7800) | 3.781 (4.4304) | 3.860 (3.5651) |
| (SD) | | | | |
| Median | 0.508 | 1.750 | 1.500 | 2.508 |
| Range | 0.48-12.00 | 0.50-24.00 | 0.48-12.00 | 0.48-12.00 |

AUE_{(0-2 h)}

| | | | | |
|---|---|---|---|---|
| Mean | 1.651 (3.8187) | 2.510 (3.9375) | 3.139 (4.3182) | 4.780 (4.4812) |
| (SD) | | | | |
| Median | 0.000 | 0.375 | 1.000 | 3.658 |
| Range | 0.00-16.29 | 0.00-13.56 | 0.00-16.00 | 0.00-16.18 |

AUE_{(0-8 h)}

| | | | | |
|---|---|---|---|---|
| Mean | 6.211 (14.9203) | 11.449 (15.6176) | 13.487 (17.5329) | 25.567 (24.3814) |
| (SD) | | | | |
| Median | 0.000 | 2.750 | 7.629 | 16.638 |
| Range | 0.00-73.27 | 0.00-47.83 | 0.00-60.59 | 0.00-73.33 |

AUE_{(0-24 h)}

| | | | | |
|---|---|---|---|---|
| Mean | 11.713 (29.2086) | 45.981 (61.9014) | 35.984 (48.2978) | 60.033 (74.2336) |
| (SD) | | | | |
| Median | 0.125 | 20.375 | 10.879 | 26.483 |
| Range | 0.00-125.27 | 0.00-256.04 | 0.00-177.48 | 0.00-249.53 |

HR1.5

| | | | | |
|---|---|---|---|---|
| Mean | 1.0 (2.39) | 1.5 (2.27) | 2.2 (3.28) | 3.3 (3.21) |
| (SD) | | | | |
| Median | 0.0 | 0.0 | 0.0 | 2.0 |
| Range | 0-9 | 0-7 | 0-12 | 0-11 |

Note:
Pre-dose time set to 0.0 hr for AUE calculation

TABLE 55
For Cole/ARCI-Unpleasantness-Dysphoria, the proportion of subjects
(per protocol population) who had a 10-100% reduction in post-dose
Eₘₐₓ compared to Morphine Sulfate IR 120 mg

| | |
|---|---|
| ALO-01 | |
| 120 mg crushed | ALO-01 120 mg whole |
| (N = 32) | (N = 32) |

Eₘₐₓ of Cole/ARCI-Unpleasantness-Dysphoria

| | | |
|---|---|---|
| At least 10% reduction | 15 (46.9%) | 17 (53.1%) |
| At least 20% reduction | 15 (46.9%) | 16 (50.0%) |
| At least 30% reduction | 12 (37.5%) | 15 (46.9%) |
| At least 40% reduction | 12 (37.5%) | 13 (40.6%) |
| At least 50% reduction | 10 (31.3%) | 12 (37.5%) |
| At least 60% reduction | 8 (25.0%) | 11 (34.4%) |
| At least 70% reduction | 7 (21.9%) | 10 (31.3%) |
| At least 80% reduction | 7 (21.9%) | 10 (31.3%) |
| At least 90% reduction | 5 (15.6%) | 8 (25.0%) |
| At least 100% reduction | 5 (15.6%) | 8 (25.0%) |

Note:
Percentage is calculated based on the number of subjects in the Per Protocol Population as the denominator

### VAS-Any Effects

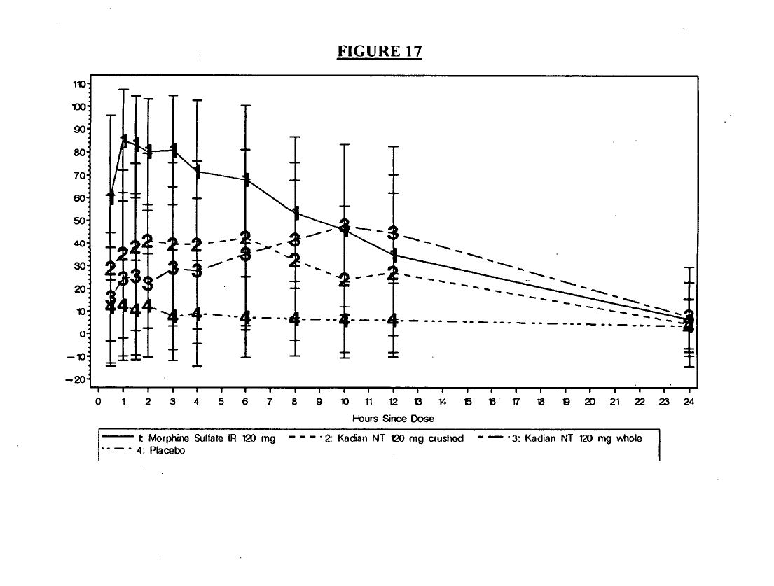

The drug-induced any drug effects were assessed using VAS: “I can feel a drug effect” scored as 0 for “definitely not” and 100 for “definitely so.” Descriptive statistics for VAS-Any Effects raw scores and summary parameters (per protocol population) were generated. Analysis of variance for Any Effects Eₘₐₓ, AUE₍₀₋₂ₕ₎, AUE₍₀₋₈ₕ₎, AUE₍₀₋₂₄ₕ₎, and at 1.5 hours post-dose (HR1.5) was also completed. Any Effects mean (SD) raw scores plotted over time for the per protocol population are illustrated in FIG. 17. Any Effects Eₘₐₓ, AUE₍₀₋₂ₕ₎, AUE₍₀₋₈ₕ₎, AUE₍₀₋₂₄ₕ₎, HR 1.5, and TEₘₐₓ for each treatment group were calculated.

The proportion of subjects who experienced 10% to 100% reduction in post-dose ratings of Any Effects Eₘₐₓ compared to MSIR is presented below in Table 56. Generally, the majority of subjects (percentage [number of subjects/total number of subjects]) had at least a 20% minimum reduction in Eₘₐₓ following both ALO-01 whole (53.1% [ 17/32]) and ALO-01 crushed (56.3% [ 18/32]) administration relative to MSIR. The highest percent reductions observed were in the 100% range, occurring at an incidence of 6.3% ( 2/32 subjects) following both ALO-01 whole and ALO-01 crushed administration.

Summary parameters of VAS-Any Effects for the per protocol population are listed below in Table 57. Any Effects scores showed a standard dose-response curve for each treatment group for up to and including 24 hours post-dose (FIG. 17). The Eₘₐₓ ranged from a mean (SD) of 17.1 (29.55) in the Placebo group to 92.3 (11.93) in the MSIR group. The Eₘₐₓ (mean [SD]) was higher for ALO-01 whole (66.8 [33.02]) compared to ALO-01 crushed (59.1 [36.74]). For all parameters, the lowest values were seen in the Placebo treatment and the highest in the MSIR treatment, with the exception of TEₘₐₓ, which was highest in the ALO-01 whole group (6.05 [4.73]). For Any Effects Eₘₐₓ, TEₘₐₓ, and AUE₍₀₋₂₄ₕ₎, ALO-01 whole had higher values compared to ALO-01 crushed. The reverse was seen for Any Effects AUE₍₀₋₂ₕ₎, AUE₍₀₋₈ₕ₎, and HR1.5.

The analysis of variance revealed a significant treatment effect for Any Effects Eₘₐₓ, AUE₍₀₋₂ₕ₎, AUE₍₀₋₈ₕ₎, AUE₍₀₋₂₄ₕ₎ and at 1.5 hours post-dose (HR1.5) (P<0.001) (Tables 14.2.2.9.3 through 14.2.2.9.7). Statistically significant differences were found for all parameters for the following treatment contrasts: ALO-01 crushed vs. Placebo (P<0.001), MSIR vs. Placebo (P<0.001), MSIR vs. ALO-01 crushed (P<0.001), and MSIR vs. ALO-01 whole (P≦0.008). In addition, statistically significant differences were found for ALO-01 whole vs. Placebo (Eₘₐₓ, AUE₍₀₋₈ₕ₎, AUE₍₀₋₂₄ₕ₎, and HR1.5 [P≦0.023]) and for ALO-01 whole vs. ALO-01 crushed (AUE₍₀₋₂ₕ₎ and HR 15 [P≦0.048]).

TABLE 56
Proportion of subjects (per protocol population) who had a 10-100%
reduction in post-dose VAS-Any Effects Eₘₐₓ compared
to Morphine Sulfate IR 120 mg

| | |
|---|---|
| ALO-01 | |
| 120 mg crushed | ALO-01 120 mg whole |
| (N = 32) | (N = 32) |

Eₘₐₓ of Any effects

| | | |
|---|---|---|
| At least 10% reduction | 22 (68.8%) | 19 (59.4%) |
| At least 20% reduction | 18 (56.3%) | 17 (53.1%) |
| At least 30% reduction | 15 (46.9%) | 10 (31.3%) |
| At least 40% reduction | 12 (37.5%) | 8 (25.0%) |
| At least 50% reduction | 11 (34.4%) | 7 (21.9%) |
| At least 60% reduction | 10 (31.3%) | 6 (18.8%) |
| At least 70% reduction | 9 (28.1%) | 5 (15.6%) |
| At least 80% reduction | 8 (25.0%) | 5 (15.6%) |
| At least 90% reduction | 5 (15.6%) | 5 (15.6%) |
| At least 100% reduction | 2 (6.3%) | 2 (6.3%) |

Note:
Percentage is calculated based on the number of subjects in the Per Protocol Population as the denominator

TABLE 57
VAS-Any Effects descriptive statistics of summary parameters
for the per protocol population (N = 32)

| | | | |
|---|---|---|---|
| | ALO-01 120 mg | ALO-01 120 mg | Morphine Sulfate IR |
| Placebo | whole | crushed | 120 mg |

Eₘₐₓ

| | | | | |
|---|---|---|---|---|
| Mean | 17.1 (29.55) | 66.8 (33.02) | 59.1 (36.74) | 92.3 (11.93) |
| (SD) | | | | |
| Median | 0.0 | 74.0 | 73.5 | 100.0 |
| Range | 0-100 | 0-100 | 0-100 | 59-100 |

TEₘₐₓ

| | | | | |
|---|---|---|---|---|
| Mean | 1.01 (1.47) | 6.05 (4.73) | 3.97 (3.34) | 1.52 (1.748) |
| (SD) | | | | |
| Median | 0.50 | 7.00 | 3.00 | 1.00 |
| Range | 0.48-8.00 | 0.48-12.02 | 0.48-12.00 | 0.48-10.00 |

AUE_{(0-2 h)}

| | | | | |
|---|---|---|---|---|
| Mean | 17.65 (33.58) | 34.40 (43.78) | 54.20 (49.56) | 119.14 (30.69) |
| (SD) | | | | |
| Median | 0.00 | 8.00 | 53.56 | 126.37 |
| Range | 0.00-98.11 | 0.00-139.50 | 0.00-150.00 | 0.00-150.00 |

AUE_{(0-8 h)}

| | | | | |
|---|---|---|---|---|
| Mean | 66.50 (142.59) | 227.77 (189.59) | 290.80 (234.85) | 537.36 (180.46) |
| (SD) | | | | |
| Median | 0.00 | 205.75 | 318.67 | 552.37 |
| Range | 0.00-498.43 | 0.00-724.17 | 0.00-750.00 | 158.93-750.00 |

AUE_{(0-24 h)}

| | | | | |
|---|---|---|---|---|
| Mean | 149.69 (337.56) | 722.73 (543.92) | 587.29 (547.94) | 965.70 (447.24) |
| (SD) | | | | |
| Median | 0.00 | 715.13 | 463.96 | 1003.55 |
| Range | 0.00-1314.43 | 0.00-2324.17 | 0.00-1773.00 | 158.93-1750.00 |

HR1.5

| | | | | |
|---|---|---|---|---|
| Mean | 10.4 (21.85) | 25.4 (34.70) | 38.4 (36.82) | 83.2 (21.34) |
| (SD) | | | | |
| Median | 0.0 | 0.0 | 42.0 | 90.0 |
| Range | 0-66 | 0-100 | 0-100 | 0-100 |

Note:
AUE calculation starts at 0.5 hr (no pre-dose value)

### VAS-Dizziness

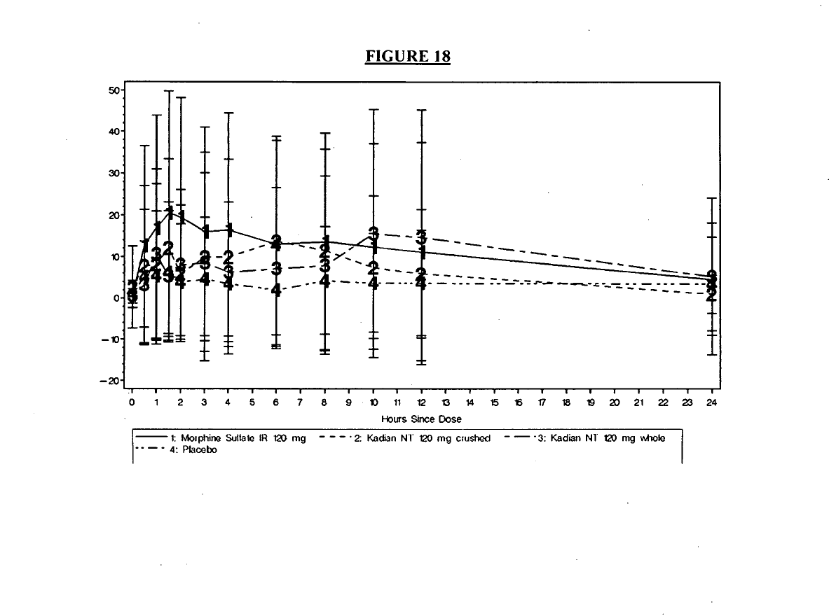

The drug-induced dizziness effects were assessed using VAS: “I am feeling dizzy” scored as 0 for “definitely not” and 100 for “definitely so.” Descriptive statistics for VAS-Dizziness raw scores and summary parameters (per protocol population) were generated. Analysis of covariance for Dizziness Eₘₐₓ, AUE₍₀₋₂ₕ₎, AUE₍₀₋₈ₕ₎, AUE₍₀₋₂₄ₕ₎, and at 1.5 hours post-dose (HR1.5) was also completed. Dizziness mean (SD) raw scores plotted over time for the per protocol population are illustrated in FIG. 18. Dizziness Eₘₐₓ, AUE₍₀₋₂ₕ₎, AUE₍₀₋₈ₕ₎, AUE₍₀₋₂₄ₕ₎, HR1.5, and TEₘₐₓ for each treatment group were calculated.

The proportion of subjects who experienced 10% to 100% reduction in post-dose ratings of Dizziness Eₘₐₓ compared to MSIR is presented below in Table 58. Relative to Eₘₐₓ for MSIR, the majority of subjects had at least a 20% reduction following ALO-01 whole administration [50.0% ( 16/32)] and at least a 40% reduction following ALO-01 crushed administration [50.0% ( 16/32)]. Furthermore, 6 subjects (18.8%) administered ALO-01 whole and 7 subjects (21.9%) administered ALO-01 crushed reported 100% Eₘₐₓ reductions.

Summary parameters of VAS-Dizziness for the per protocol population are listed below in Table 59. The Eₘₐₓ ranged from a mean (SD) of 9.1 (19.80) in the Placebo group to 37.8 (36.63) in the MSIR group. The Eₘₐₓ for dizziness (mean [SD]) was slightly higher for ALO-01 whole (26.9 [33.95]) compared to ALO-01 crushed (23.8 [30.90]). Generally, for all parameters the lowest values were seen in the Placebo treatment, and the highest in the MSIR treatment, with the exception of TEₘₐₓ which was highest in the ALO-01 whole treatment (3.23 [4.14]) and 1.5 hours post-dosing at which point the lowest mean was recorded for ALO-01 whole (5.3 [15.64]. Generally, Dizziness Eₘₐₓ, TEₘₐₓ, and AUE₍₀₋₂₄ₕ₎ were higher in the ALO-01 whole treatment compared to ALO-01 crushed treatment. The reverse was seen for Dizziness AUE₍₀₋₂ₕ₎ and AUE₍₀₋₈ₕ₎. The analysis of covariance revealed a significant treatment effect for VAS-Dizziness Eₘₐₓ, AUE₍₀₋₂ₕ₎, AUE₍₀₋₈ₕ₎, AUE₍₀₋₂₄ₕ₎, and at 1.5 hours post-dose (P≦0.01) and significant baseline effects for AUE₍₀₋₈ₕ₎ (P=0.027). Eₘₐₓ was found to be significantly different for all contrasts (P≦0.043) except for ALO-01 whole vs. ALO-01 crushed (P=0.473). The AUE₍₀₋₂ₕ₎ was found to be significantly different for all comparisons against MSIR (P≦0.005). A comparison of ALO-01 whole vs. ALO-01 crushed was not significant (P=0.581). The AUE₍₀₋₈ₕ₎ was significantly different for all comparisons against MSIR (P≦0.029) and ALO-01 crushed vs. Placebo (P<0.019). The AUE₍₀₋₂₄ₕ₎ was significantly different for the ALO-01 whole vs. Placebo (P<0.012), MSIR vs. Placebo (P<0.002), and MSIR vs. ALO-01 crushed (P<0.046). VAS-Dizziness, at 1.5 hours post-dose, was found to be significantly different for all contrasts against MSIR treatment (P≦0.037).

TABLE 58
Proportion of subjects (per protocol population) who had a 10-100%
reduction in post-dose VAS-Dizziness Eₘₐₓ compared
to Morphine Sulfate IR 120 mg

| | |
|---|---|
| ALO-01 | |
| 120 mg crushed | ALO-01 120 mg whole |
| (N = 32) | (N = 32) |

Eₘₐₓ of Dizziness

| | | |
|---|---|---|
| At least 10% reduction | 18 (56.3%) | 17 (53.1%) |
| At least 20% reduction | 16 (50.0%) | 16 (50.0%) |
| At least 30% reduction | 16 (50.0%) | 15 (46.9%) |
| At least 40% reduction | 16 (50.0%) | 13 (40.6%) |
| At least 50% reduction | 15 (46.9%) | 12 (37.5%) |
| At least 60% reduction | 13 (40.6%) | 11 (34.4%) |
| At least 70% reduction | 12 (37.5%) | 11 (34.4%) |
| At least 80% reduction | 12 (37.5%) | 10 (31.3%) |
| At least 90% reduction | 10 (31.3%) | 9 (28.1%) |
| At least 100% reduction | 7 (21.9%) | 6 (18.8%) |

Note:
Percentage is calculated based on the number of subjects in the Per Protocol Population as the denominator.

TABLE 59
VAS-Dizziness descriptive statistics of summary parameters for
the per protocol population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | |
|---|---|---|---|---|
| Mean | 9.1 (19.80) | 26.9 (33.95) | 23.8 (30.90) | 37.8 (36.63) |
| (SD) | | | | |
| Median | 0.0 | 5.0 | 5.5 | 27.0 |
| Range | 0-69 | 0-100 | 0-97 | 0-100 |

TEₘₐₓ

| | | | | |
|---|---|---|---|---|
| Mean | 1.122 (1.4922) | 3.234 (4.1426) | 2.858 (2.9512) | 2.969 (3.4003) |
| (SD) | | | | |
| Median | 0.500 | 1.000 | 1.492 | 1.500 |
| Range | 0.48-8.00 | 0.48-12.00 | 0.48-10.00 | 0.48-12.00 |

AUE_{(0-2 h)}

| | | | | |
|---|---|---|---|---|
| Mean | 9.581 (26.2288) | 11.732 (23.3844) | 16.069 (32.2359) | 30.027 (42.3038) |
| (SD) | | | | |
| Median | 0.000 | 0.000 | 0.000 | 6.854 |
| Range | 0.00-109.78 | 0.00-90.00 | 0.00-104.75 | 0.00-133.47 |

AUE_{(0-8 h)}

| | | | | |
|---|---|---|---|---|
| Mean | 29.321 (90.5185) | 55.831 (113.8779) | 82.710 (145.0664) | 119.867 (157.6414) |
| (SD) | | | | |
| Median | 0.000 | 1.750 | 8.008 | 41.250 |
| Range | 0.00-465.18 | 0.00-417.00 | 0.00-503.78 | 0.00-535.62 |

AUE_{(0-24 h)}

| | | | | |
|---|---|---|---|---|
| Mean | 86.762 (276.9275) | 228.913 (462.0691) | 156.969 (288.1047) | 263.950 (442.2145) |
| (SD) | | | | |
| Median | 0.000 | 4.500 | 10.008 | 45.454 |
| Range | 0.00-1186.18 | 0.00-1852.34 | 0.00-1120.23 | 0.00-1496.91 |

HR1.5

| | | | | |
|---|---|---|---|---|
| Mean | 6.2 (16.86) | 5.3 (15.64) | 12.0 (21.42) | 20.5 (29.24) |
| (SD) | | | | |
| Median | 0.0 | 0.0 | 0.0 | 0.0 |
| Range | 0-67 | 0-55 | 0-65 | 0-83 |

Note:
Pre-dose time set to 0.0 hr for AUE calculation

### ARCI-Amphetamine Scale

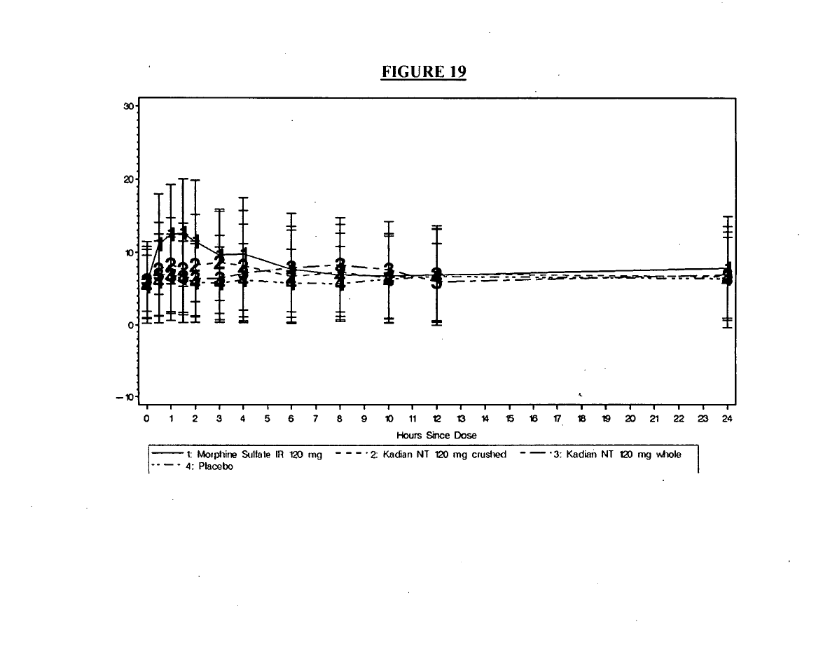

The ARCI-Amphetamine (A) scale is a measure of stimulant, amphetamine-like effects. It is comprised of 11 questions, all weighted as positive in scoring. Thus, scores for this scale can range from 0 to 33. Descriptive statistics for ARCI-A raw scores and summary parameters (per protocol population) were generated. Analysis of covariance for ARCI-A Eₘₐₓ, AUE₍₀₋₂ₕ₎, AUE₍₀₋₈ₕ₎, AUE₍₀₋₂₄ₕ₎, and at 1.5 hours post-dose (HR1.5) was also completed. ARCI-A mean (SD) raw scores plotted over time for the per protocol population are illustrated in FIG. 19. ARCI-A Eₘₐₓ, TEₘₐₓ, AUE₍₀₋₂ₕ₎, AUE₍₀₋₈ₕ₎, AUE₍₀₋₂₄ₕ₎, and at the 1.5 hours post-dosing time point for each treatment group were calculated.

The proportion of subjects from the ALO-01 whole and ALO-01 crushed who had a 10-100% reduction in post-dose ARCI-A scores Eₘₐₓ compared to MSIR are listed below in Table 60. Relative to Eₘₐₓ for MSIR, the majority of subjects had at least a 10% reduction following ALO-01 whole administration [65.6% ( 21/32)] and following ALO-01 crushed administration [59.4% ( 19/32)]. Only one subject (3.1%) administered ALO-01 crushed reported 100% Eₘₐₓ reduction, while at least 80% Eₘₐₓ reduction was the greatest reduction reported by one subject (3.1%) administered the ALO-01 whole treatment.

Summary parameters of ARCI-A for the per protocol population are listed below in Table 61. The Eₘₐₓ ranged from a mean (SD) of 8.5 (6.74) in the Placebo group to 15.3 (8.32) in the MSIR group. The Eₘₐₓ for ARCI-A (mean [SD]) was slightly higher for ALO-01 crushed (12.3 [7.30]) compared to ALO-01 whole (11.5 [7.83]). The same pattern of mean responses (MSIR>ALO-01 crushed>ALO-01 whole>Placebo) was observed for remaining parameters except for TEₘₐₓ. For TEₘₐₓ the following pattern of mean responses was observed: ALO-01 whole>ALO-01 crushed>Placebo>MSIR.

The analysis of covariance revealed a significant treatment effect for ARCI-A Eₘₐₓ, AUE₍₀₋₂ₕ₎, AUE₍₀₋₈ₕ₎, AUE₍₀₋₂₄ₕ₎, and at 1.5 hours post-dose (P≦0.008). Eₘₐₓ was found to be significantly different for all treatment contrasts (P≦0.01) except for ALO-01 whole vs. ALO-01 crushed (P=0.384). The AUE₍₀₋₂ₕ₎, AUE₍₀₋₈ₕ₎ and at 1.5 hours post-dosing time point were found to be significantly different for all treatments contrasts against MSIR (P<0.001 for AUE₍₀₋₂ₕ₎ and HR1.5 and P≦0.016 for AUE₍₀₋₈ₕ₎). The AUE₍₀₋₂₄ₕ₎ was significantly different for the MSIR vs. Placebo (P<0.001) and MSIR vs. ALO-01 whole (P<0.007).

TABLE 60
Proportion of subjects (per protocol population)
who had a 10-100% reduction in post-dose ARCI-A Eₘₐₓ
compared to Morphine Sulfate IR 120 mg

| | |
|---|---|
| ALO-01 120 mg | ALO-01 120 mg |
| crushed (N = 32) | whole (N = 32) |

Eₘₐₓ of ARCI-Amphetamine (A)

| | | | | |
|---|---|---|---|---|
| At least 10% reduction | 19 | (59.4%) | 21 | (65.6%) |
| At least 20% reduction | 15 | (46.9%) | 14 | (43.8%) |
| At least 30% reduction | 12 | (37.5%) | 14 | (43.8%) |
| At least 40% reduction | 9 | (28.1%) | 9 | (28.1%) |
| At least 50% reduction | 8 | (25.0%) | 8 | (25.0%) |
| At least 60% reduction | 3 | (9.4%) | 3 | (9.4%) |
| At least 70% reduction | 1 | (3.1%) | 2 | (6.3%) |
| At least 80% reduction | 1 | (3.1%) | 1 | (3.1%) |
| At least 90% reduction | 1 | (3.1%) | 0 | (0.0%) |
| At least 100% reduction | 1 | (3.1%) | 0 | (0.0%) |

Note:
Percentage is calculated based on the number of subjects in the Per Protocol Population as the denominator

TABLE 61
ARCI-Amphetamine (A) descriptive statistics of summary
parameters for the per protocol population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | | | | | |
|---|---|---|---|---|---|---|---|---|
| Mean (SD) | 8.5 | (6.74) | 11.5 | (7.83) | 12.3 | (7.30) | 15.3 | (8.32) |

| | | | | |
|---|---|---|---|---|
| Median | 6.5 | 9.0 | 10.5 | 14.0 |
| Range | 0-27 | 0-33 | 0-30 | 0-33 |

TEₘₐₓ

| | | | | | | | | |
|---|---|---|---|---|---|---|---|---|
| Mean (SD) | 4.201 | (5.3952) | 6.311 | (7.3924) | 4.546 | (6.9580) | 2.983 | (5.8085) |

| | | | | |
|---|---|---|---|---|
| Median | 1.500 | 3.500 | 1.492 | 1.000 |
| Range | 0.48-23.98 | 0.50-24.00 | 0.48-24.00 | 0.48-24.00 |

AUE_{(0-2 h)}

| | | | | | | | | |
|---|---|---|---|---|---|---|---|---|
| Mean (SD) | 12.186 | (10.8625) | 13.276 | (9.4390) | 15.319 | (11.4480) | 22.464 | (12.5229) |

| | | | | |
|---|---|---|---|---|
| Median | 10.000 | 10.942 | 11.992 | 21.917 |
| Range | 0.00-44.50 | 0.00-32.25 | 0.00-44.00 | 0.00-53.65 |

AUE_{(0-8 h)}

| | | | | | | | | |
|---|---|---|---|---|---|---|---|---|
| Mean (SD) | 47.237 | (37.9554) | 57.654 | (44.5147) | 61.001 | (49.8217) | 74.849 | (44.1791) |

| | | | | |
|---|---|---|---|---|
| Median | 42.129 | 49.333 | 46.246 | 70.725 |
| Range | 0.00-141.73 | 0.75-171.00 | 0.00-206.50 | 0.00-165.62 |

AUE_{(0-24 h)}

| | | | | | | | | |
|---|---|---|---|---|---|---|---|---|
| Mean (SD) | 150.377 | (130.6211) | 164.198 | (128.4260) | 170.147 | (147.8046) | 190.438 | (132.8000) |

| | | | | |
|---|---|---|---|---|
| Median | 121.625 | 149.258 | 124.888 | 162.000 |
| Range | 0.00-480.78 | 0.75-506.80 | 0.00-653.50 | 0.00-538.13 |

HR1.5

| | | | | | | | | |
|---|---|---|---|---|---|---|---|---|
| Mean (SD) | 6.4 | (6.07) | 6.6 | (4.92) | 7.7 | (6.28) | 12.7 | (7.39) |

| | | | | |
|---|---|---|---|---|
| Median | 5.0 | 6.5 | 6.0 | 11.0 |
| Range | 0-23 | 0-17 | 0-23 | 0-31 |

Note:
Pre-dose time set to 0.0 hr for AUE calculation

### ARCI-BG Scale

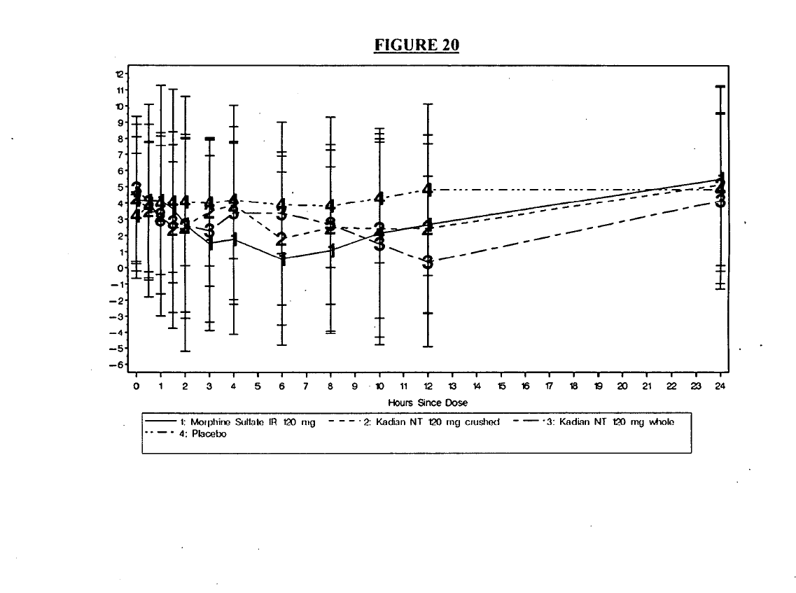

The ARCI-BG scale is a measure of drug stimulant effects. It is comprised of 13 questions, 9 of which are weighted as positive in scoring. Scores for this scale can range from −12 to 27. Descriptive statistics for ARCI-BG raw scores and summary parameters (per protocol population) were generated. Analysis of covariance for ARCI-BG Eₘₐₓ, Eₘᵢₙ, AUE₍₀₋₂ₕ₎, AUE₍₀₋₈ₕ₎, AUE₍₀₋₂₄ₕ₎, and at 1.5 hours post-dose (HR1.5) was also completed. ARCI-BG mean (SD) raw scores plotted over time for the per protocol population are illustrated below in FIG. 20. ARCI-BG Eₘₐₓ, TEₘₐₓ, Eₘᵢₙ, TEₘᵢₙ, AUE₍₀₋₂ₕ₎, AUE₍₀₋₈ₕ₎, AUE₍₀₋₂₄ₕ₎, and at the 1.5 hours post-dosing time point for each treatment group were calculated.

The proportion of subjects (per protocol population) who had a 10-100% reduction in post-dose ARCI-Benzedrine Group (BG) Eₘₐₓ compared to Morphine Sulfate IR 120 mg is shown in Table 62. Relative to Eₘₐₓ for MSIR, 13 subjects [40.6% ( 13/32)] in the ALO-01 whole group and 14 subjects [43.8% ( 14/32)] in the ALO-01 crushed group had at least 10% ARCI-BG Eₘₐₓ reduction. The highest percent reductions observed for the ALO-01 whole group were in the 100% range, occurring at an incidence of 3.1% ( 1/32), while the highest percent reductions observed for the ALO-01 crushed group were in the at least 70% Eₘₐₓ reduction range occurring at an incidence of 3.1% ( 1/32).

Summary parameters of ARCI-BG for the per protocol population are listed below in Table 63. The Eₘₐₓ ranged from a mean (SD) of 6.3 (5.08) in the Placebo group to 9.0 (6.37) in the MSIR group. The Eₘₐₓ for ARCI-BG (mean [SD]) was slightly lower for ALO-01 whole (7.3 [5.44]) compared to ALO-01 crushed (7.8 [6.01]). TEₘₐₓ for Placebo was reached the earliest followed by TEₘₐₓ for ALO-01 whole, MSIR and ALO-01 crushed while TEₘᵢₙ for Placebo was reached the earliest followed by TEₘₐₓ for ALO-01 crushed, MSIR, and ALO-01 whole. Mean AUE₍₀₋₂ₕ₎ and mean response at 1.5 hours post-dose was the lowest for ALO-01 crushed followed by ALO-01 whole, MSIR, and Placebo, while mean AUE₍₀₋₈ₕ₎ was the lowest for MSIR followed by ALO-01 crushed, ALO-01 whole, and Placebo. Mean AUE₍₀₋₂ₕ₎ was the lowest for ALO-01 whole followed by MSIR, ALO-01 crushed, and Placebo.

The analysis of covariance revealed a significant treatment effect for ARCI-BG Eₘₐₓ, Eₘᵢₙ, AUE₍₀₋₈ₕ₎, and AUE₍₀₋₂₄ₕ₎ (P≦0.013). Eₘₐₓ was found to be significantly different for MSIR vs. Placebo and ALO-01 whole treatments contrasts (P<0.001 and P=0.01, respectively). For Eₘᵢₙ, AUE₍₀₋₈ₕ₎ and AUE₍₀₋₂₄ₕ₎ all treatments significantly differed from Placebo (P<0.001, P≦0.036 and P≦0.009, respectively). Additionally, for Eₘᵢₙ MSIR significantly differed from ALO-01 crushed (P=0.021)

TABLE 62
Proportion of subjects (per protocol population) who had a 10-100%
reduction in post-dose ARCI-Benzedrine Group (BG) Eₘₐₓ compared
to Morphine Sulfate IR 120 mg

| | |
|---|---|
| ALO-01 120 mg crushed | ALO-01 120 mg |
| (N = 32) | whole (N = 32) |

Eₘₐₓ of ARCI-Benzedrine Group (BG)

| | | |
|---|---|---|
| At least 10% reduction | 14 (43.8%) | 13 (40.6%) |
| At least 20% reduction | 12 (37.5%) | 11 (34.4%) |
| At least 30% reduction | 9 (28.1%) | 9 (28.1%) |
| At least 40% reduction | 7 (21.9%) | 6 (18.8%) |
| At least 50% reduction | 7 (21.9%) | 6 (18.8%) |
| At least 60% reduction | 2 (6.3%) | 3 (9.4%) |
| At least 70% reduction | 1 (3.1%) | 3 (9.4%) |
| At least 80% reduction | 0 (0.0%) | 2 (6.3%) |
| At least 90% reduction | 0 (0.0%) | 1 (3.1%) |
| At least 100% | 0 (0.0%) | 1 (3.1%) |
| reduction | | |

Note:
Percentage is calculated based on the number of subjects in the Per Protocol Population as the denominator

TABLE 63
ARCI-Benzedrine Group (BG) descriptive statistics of summary
parameters for the per protocol population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | |
|---|---|---|---|---|
| Mean (SD) | 6.3 (5.08) | 7.3 (5.44) | 7.8 (6.01) | 9.0 (6.37) |
| Median | 5.5 | 6.5 | 6.0 | 8.0 |
| Range | 0-24 | 0-25 | 0-27 | 0-26 |

TEₘₐₓ

| | | | | |
|---|---|---|---|---|
| Mean (SD) | 6.061 (7.9762) | 6.482 (8.1417) | 8.344 (10.2662) | 7.391 (9.4699) |
| Median | 1.500 | 3.000 | 2.000 | 2.000 |
| Range | 0.48-24.00 | 0.48-24.00 | 0.48-24.00 | 0.48-24.00 |

Emin

| | | | | |
|---|---|---|---|---|
| Mean (SD) | 1.7 (3.24) | −1.7 (4.77) | −0.8 (4.75) | −2.8 (4.12) |
| Median | 2.0 | −0.5 | 0.0 | −1.0 |
| Range | −7-12 | −12-7 | −9-17 | −11-6 |

Temin

| | | | | |
|---|---|---|---|---|
| Mean (SD) | 2.231 (2.7881) | 6.250 (6.3580) | 4.170 (5.1338) | 5.516 (4.6659) |
| Median | 1.242 | 3.008 | 1.992 | 5.000 |
| Range | 0.48-12.00 | 0.50-24.00 | 0.50-24.00 | 0.50-24.00 |

AUE_{(0-2 h)}

| | | | | |
|---|---|---|---|---|
| Mean (SD) | 7.720 (7.6450) | 6.786 (7.8906) | 6.362 (8.3027) | 7.710 (11.3739) |
| Median | 6.875 | 6.638 | 5.263 | 5.750 |
| Range | −3.75-26.50 | −10.47-24.50 | −8.75-35.25 | −8.94-43.18 |

AUE_{(0-8 h)}

| | | | | |
|---|---|---|---|---|
| Mean (SD) | 31.710 (25.9130) | 25.001 (36.9688) | 23.122 (34.7232) | 15.467 (37.4764) |
| Median | 31.121 | 22.767 | 23.350 | 18.496 |
| Range | −1.50-105.25 | −65.97-114.11 | −23.50-165.75 | −56.50-81.90 |

AUE_{(0-24 h)}

| | | | | |
|---|---|---|---|---|
| Mean (SD) | 107.133 (98.1450) | 58.265 (95.3538) | 78.736 (108.2577) | 72.659 (103.2648) |
| Median | 95.875 | 58.850 | 78.446 | 71.571 |
| Range | −18.18-392.21 | −113.18-251.11 | −68.50-556.75 | −143.58-341.03 |

HR1.5

| | | | | |
|---|---|---|---|---|
| Mean (SD) | 4.1 (4.35) | 2.8 (3.74) | 2.4 (5.17) | 3.7 (7.39) |
| Median | 4.0 | 3.0 | 1.5 | 2.0 |
| Range | −3-14 | −6-10 | −7-18 | −9-24 |

Note:
Pre-dose time set to 0.0 hr for AUE calculation

### Cole/ARCI-Stimulation-Motor

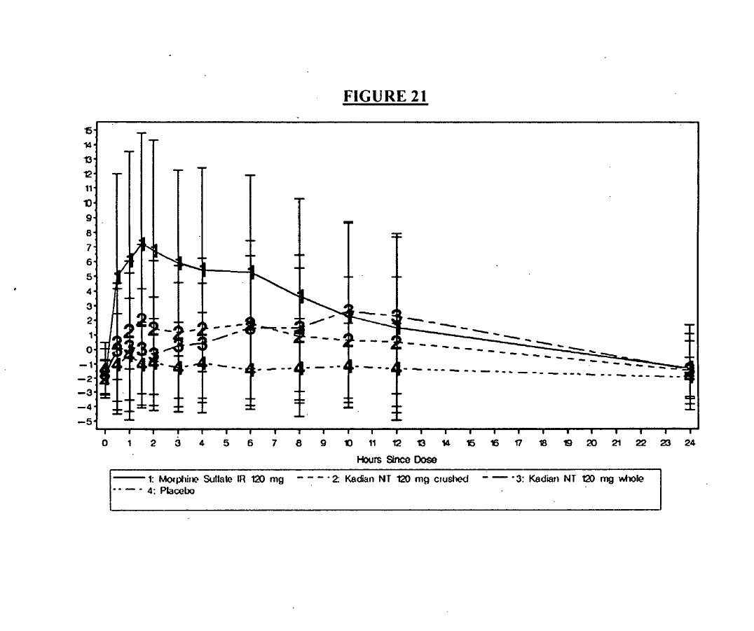

The Cole/ARCI-Stimulation-Motor is comprised of 4 questions, all weighted as positive, and, thus, scoring can range from 0 to 12. Descriptive statistics for Cole/ARCI-Stimulation-Motor raw scores and summary parameters (per protocol population) were generated. Analysis of covariance for Cole/ARCI-Stimulation-Motor Eₘₐₓ, AUE₍₀₋₂ₕ₎, AUE₍₀₋₈ₕ₎, AUE₍₀₋₂₄ₕ₎, and at 1.5 hours post-dose (HR1.5) were also calculated. Cole/ARCI-Stimulation-Motor mean (SD) raw scores plotted over time for the per protocol population are illustrated in FIG. 21. Cole/ARCI-Stimulation-Motor box plots for Eₘₐₓ, TEₘₐₓ, AUE₍₀₋₂ₕ₎) AUE₍₀₋₈ₕ₎, AUE₍₀₋₂₄ₕ₎, and HR 1.5 for each treatment group were calculated.

The proportion of subjects who had a 10-100% reduction in Eₘₐₓ after adminitration of ALO-01 whole or crushed compared to Eₘₐₓ after MSIR administration are listed below in Table 64. Relative to Eₘₐₓ for MSIR treatment, the majority of subjects (percentage [number of subjects/total number of subjects]) had at least a 20% minimum reduction in Eₘₐₓ following ALO-01 whole administration (62.5% [ 20/32]) and at least a 30% reduction in Eₘₐₓ following ALO-01 crushed administration (56.3% [ 18/32]). The highest percent reductions observed were in the 100% range, occurring at an incidence of 12.5% ( 4/32) following both ALO-01 whole and ALO-01 crushed administration.

Summary parameters of Cole/ARCI-Stimulation-Motor for the per protocol population are listed below in Table 65. The Eₘₐₓ ranged from a mean (SD) of 2.3 (2.41) in the Placebo group to 5.5 (2.66) in the MSIR group. The Eₘₐₓ mean [SD] for ALO-01 whole and ALO-01 crushed treatments was the same (3.7 [3.01] and 3.7 [2.55], respectively). Generally, for most parameters the lowest values were seen in the Placebo treatment and the highest in the MSIR treatment, with the exception of TEₘₐₓ which was lowest for MSIR followed by Placebo, ALO-01 crushed, and ALO-01 whole treatment. The AUE₍₀₋₂ₕ₎, AUE₍₀₋₈ₕ₎, and HR1.5 was greater for ALO-01 crushed than ALO-01 whole; however, the pattern was reversed for AUE₍₀₋₂₄ₕ₎.

The analysis of covariance revealed significant treatment effect and baseline effect for Eₘₐₓ, AUE₍₀₋₂ₕ₎, AUE₍₀₋₈ₕ₎, AUE₍₀₋₂₄ₕ₎ and HR1.5 (P<0.001). For Eₘₐₓ all treatment contrasts reached statistical significance (P≦0.006) except for ALO-01 whole vs. ALO-01 crushed treatment contrast (P=0.522). For AUE₍₀₋₂ₕ₎, AUE₍₀₋₈ₕ₎, AUE₍₀₋₂₄ₕ₎, and HR1.5 all treatment contrast against MSIR were significant (P≦0.005). Additionally, for AUE₍₀₋₂ₕ₎ and AUE₍₀₋₈ₕ₎ ALO-01 crushed vs. Placebo treatment contrasts were statistically significant (P≦0.048).

TABLE 64
For Cole/ARCI-Stimulation-Motor proportion of subjects (per protocol
population) who had a 10-100% reduction in post-dose Eₘₐₓ compared
to Morphine Sulfate IR 120 mg

| | |
|---|---|
| ALO-01 120 mg crushed | ALO-01 120 mg |
| (N = 32) | whole (N = 32) |

Eₘₐₓ of Cole/ARCI Stimulation-Motor

| | | |
|---|---|---|
| At least 10% reduction | 21 (65.6%) | 20 (62.5%) |
| At least 20% reduction | 21 (65.6%) | 20 (62.5%) |
| At least 30% reduction | 18 (56.3%) | 15 (46.9%) |
| At least 40% reduction | 13 (40.6%) | 11 (34.4%) |
| At least 50% reduction | 12 (37.5%) | 11 (34.4%) |
| At least 60% reduction | 8 (25.0%) | 7 (21.9%) |
| At least 70% reduction | 5 (15.6%) | 7 (21.9%) |
| At least 80% reduction | 4 (12.5%) | 7 (21.9%) |
| At least 90% reduction | 4 (12.5%) | 4 (12.5%) |
| At least 100% reduction | 4 (12.5%) | 4 (12.5%) |

Note:
Percentage is calculated based on the number of subjects in the Per Protocol Population as the denominator.

TABLE 65
Cole/ARCI-Stimulation-Motor descriptive statistics of summary parameters for the per
protocol population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | |
|---|---|---|---|---|
| Mean (SD) | 2.3 (2.41) | 3.7 (3.01) | 3.7 (2.55) | 5.5 (2.66) |
| Median | 1.5 | 3.0 | 3.0 | 6.0 |
| Range | 0-8 | 0-9 | 0-9 | 0-10 |

TEₘₐₓ

| | | | | |
|---|---|---|---|---|
| Mean (SD) | 2.546 (4.8769) | 3.657 (4.0087) | 3.437 (5.7954) | 2.468 (4.4327) |
| Median | 0.508 | 1.500 | 1.033 | 1.000 |
| Range | 0.48-24.00 | 0.48-12.00 | 0.48-24.00 | 0.48-24.00 |

AUE_{(0-2 h)}

| | | | | |
|---|---|---|---|---|
| Mean (SD) | 2.951 (3.6660) | 3.536 (3.7468) | 4.110 (3.9878) | 7.224 (4.3269) |
| Median | 1.879 | 2.250 | 2.625 | 7.850 |
| Range | 0.00-13.53 | 0.00-12.25 | 0.00-11.50 | 0.00-15.80 |

AUE_{(0-8 h)}

| | | | | |
|---|---|---|---|---|
| Mean (SD) | 11.186 (14.7983) | 14.366 (14.5785) | 16.062 (17.1294) | 24.911 (15.6194) |
| Median | 3.254 | 11.521 | 7.967 | 23.625 |
| Range | 0.00-54.51 | 0.00-50.25 | 0.00-52.05 | 0.00-54.63 |

AUE_{(0-24 h)}

| | | | | |
|---|---|---|---|---|
| Mean (SD) | 32.373 (44.1415) | 42.709 (44.1670) | 39.841 (47.7220) | 55.059 (48.6139) |
| Median | 5.871 | 24.646 | 18.875 | 38.729 |
| Range | 0.00-128.51 | 0.00-140.26 | 0.00-148.95 | 0.00-160.38 |

HR1.5

| | | | | |
|---|---|---|---|---|
| Mean (SD) | 1.5 (1.92) | 1.9 (2.20) | 2.1 (2.24) | 4.3 (2.53) |
| Median | 1.0 | 1.0 | 1.5 | 5.0 |
| Range | 0-8 | 0-8 | 0-7 | 0-8 |

Note:
Pre-dose time set to 0.0 hr for AUE calculation

### VAS-Sleepy

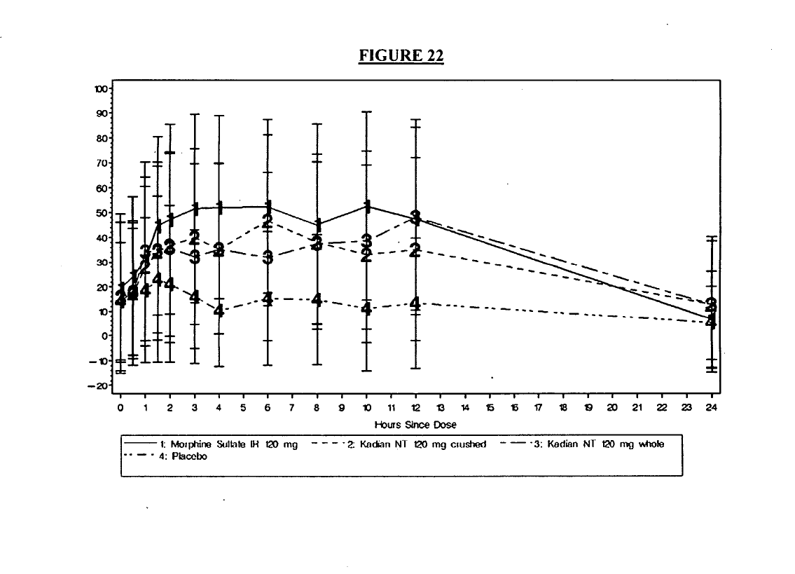

The drug-induced sleepy effects were assessed using VAS: “I am feeling sleepy” scored as 0 for “definitely not” and 100 for “definitely so.” Descriptive statistics for VAS-Sleepy raw scores were generated. Analysis of covariance for Sleepy Eₘₐₓ, AUE₍₀₋₂ₕ₎, AUE₍₀₋₈ₕ₎, AUE₍₀₋₂₄ₕ₎, and at 1.5 hours post-dose (HR1.5) were completed. Sleepy mean (SD) raw scores plotted over time for the per protocol population are illustrated in below in FIG. 22. Sleepy Eₘₐₓ, AUE₍₀₋₂ₕ₎, AUE₍₀₋₈ₕ₎, AUE₀₋₂₄ₕ₎, HR1.5, and TEₘₐₓ were calculated for each treatment group.

The proportion of subjects who experienced 10% to 100% reduction in post-dose ratings of feeling Sleepy Eₘₐₓ compared to MSIR is presented below in Table 66. Relative to Eₘₐₓ for the MSIR treatment, 13 out of 32 subjects (40.6%) experienced at least 10% reduction in Eₘₐₓ following ALO-01 whole and ALO-01 crushed treatments. The highest reductions were seen as a 100% reduction in the ALO-01 whole group (12.5% [ 4/32]) and in the ALO-01 crushed group (6.3% [ 2/32]).

Summary parameters of VAS-Sleepy for the per protocol population are listed below in Table 67. The Eₘₐₓ ranged from a mean (SD) of 38.4 (36.19) in the Placebo group to 79.3 (24.97) in the MSIR group. The Eₘₐₓ for sleepy (mean [SD]) was similar for both ALO-01 whole (67.1 [37.16]) compared to ALO-01 crushed (68.1 [33.32]). Generally, for all parameters the lowest values were seen in the Placebo treatment, and the highest in the MSIR treatment, with the exception of TEₘₐₓ, which was highest in the ALO-01 crushed treatment (6.65 [6.57]). Generally, Sleepy Eₘₐₓ) TEₘₐₓ, and AUE₍₀₋₈ₕ₎ were higher in the ALO-01 crushed treatment compared to ALO-01 whole treatment; the reverse was seen for Sleepy AUE₍₀₋₂ₕ₎ and AUE₍₀₋₂₄ₕ₎ Whereas, HR1.5 means were similar for both ALO-01 whole (34.8 [33.81]) and ALO-01 crushed (34.3 [36.0]).

The analysis of covariance revealed a significant treatment effect for VAS-Sleepy Eₘₐₓ, AUE₍₀₋₂ₕ₎, AUE₍₀₋₈ₕ₎, AUE₍₀₋₂₄ₕ₎, and at 1.5 hours post-dose (P≦0.024). Eₘₐₓ was found to be significantly different for the ALO-01 crushed vs. Placebo (P<0.001), ALO-01 whole vs. Placebo (P<0.001), MSIR vs. Placebo (P<0.001), and for MSIR vs. ALO-01 whole (P=0.01) treatment contrasts. The AUE₍₀₋₂ₕ₎ was found to be almost significantly different for the ALO-01 whole vs. Placebo (P=0.05) and significantly different for MSIR vs. Placebo treatment contrast (P=0.004). The AUE₍₀₋₈ₕ₎ was significantly different for ALO-01 crushed vs. Placebo (P<0.001), ALO-01 whole vs. Placebo (P<0.001), MSIR vs. Placebo (P<0.001), and for MSIR vs. ALO-01 whole (P=0.007) treatment contrasts. The AUE₍₀₋₂₄ₕ₎ was significantly different for the ALO-01 crushed vs. Placebo (P<0.001), ALO-01 whole vs. Placebo (P<0.001), and MSIR vs. Placebo (P<0.001). The VAS-Sleepy score, at 1.5 hours post-dose, was found to be significantly different for MSIR vs. Placebo (P=0.002) treatment contrasts.

TABLE 66
Proportion of subjects (per protocol population) who had a 10-100%
reduction in post-dose VAS-Sleepy Eₘₐₓ compared to Morphine
Sulfate IR 120 mg

| | |
|---|---|
| ALO-01 120 mg crushed | ALO-01 120 mg |
| (N = 32) | whole (N = 32) |

Eₘₐₓ of Sleepy

| | | |
|---|---|---|
| At least 10% reduction | 13 (40.6%) | 13 (40.6%) |
| At least 20% reduction | 12 (37.5%) | 9 (28.1%) |
| At least 30% reduction | 8 (25.0%) | 8 (25.0%) |
| At least 40% reduction | 7 (21.9%) | 6 (18.8%) |
| At least 50% reduction | 7 (21.9%) | 6 (18.8%) |
| At least 60% reduction | 6 (18.8%) | 6 (18.8%) |
| At least 70% reduction | 6 (18.8%) | 6 (18.8%) |
| At least 80% reduction | 5 (15.6%) | 6 (18.8%) |
| At least 90% reduction | 3 (9.4%) | 5 (15.6%) |
| At least 100% reduction | 2 (6.3%) | 4 (12.5%) |

Note:
Percentage is calculated based on the number of subjects in the Per Protocol Population as the denominator

TABLE 67
VAS-Sleepy descriptive statistics of summary parameters for the per protocol
population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | |
|---|---|---|---|---|
| Mean (SD) | 38.4 (36.19) | 67.1 (37.16) | 68.1 (33.32) | 79.3 (24.97) |
| Median | 36.0 | 77.0 | 74.5 | 87.0 |
| Range | 0-100 | 0-100 | 0-100 | 3-100 |

TEₘₐₓ

| | | | | |
|---|---|---|---|---|
| Mean (SD) | 3.01 (3.78) | 6.02 (4.86) | 6.65 (6.57) | 4.61 (3.36) |
| Median | 1.25 | 6.00 | 4.00 | 4.00 |
| Range | 0.48-12.00 | 0.48-12.02 | 0.50-24.00 | 0.50-12.00 |

AUE_{(0-2 h)}

| | | | | |
|---|---|---|---|---|
| Mean (SD) | 38.63 (51.79) | 56.17 (52.01) | 53.09 (54.41) | 66.53 (57.07) |
| Median | 9.00 | 45.38 | 36.60 | 68.63 |
| Range | 0.00-172.30 | 0.00-161.33 | 0.00-160.75 | 0.00-191.50 |

AUE_{(0-8 h)}

| | | | | |
|---|---|---|---|---|
| Mean (SD) | 125.74 (173.70) | 260.47 (226.30) | 295.99 (199.93) | 369.89 (216.78) |
| Median | 59.71 | 253.86 | 295.65 | 347.47 |
| Range | 0.00-573.87 | 0.00-673.00 | 0.00-673.84 | 6.23-736.25 |

AUE_{(0-24 h)}

| | | | | |
|---|---|---|---|---|
| Mean (SD) | 288.38 (450.15) | 789.04 (599.95) | 723.10 (594.09) | 893.92 (522.92) |
| Median | 64.88 | 795.88 | 563.76 | 1010.56 |
| Range | 0.00-1556.13 | 0.00-1748.08 | 0.00-1929.54 | 6.23-2015.68 |

HR1.5

| | | | | |
|---|---|---|---|---|
| Mean (SD) | 23.0 (33.74) | 34.8 (33.81) | 34.3 (36.09) | 44.6 (35.97) |
| Median | 0.0 | 36.5 | 19.5 | 54.0 |
| Range | 0-100 | 0-100 | 0-100 | 0-100 |

Note:
Pre-dose time set to 0.0 hr for AUE calculation

### ARCI-PCAG Scale

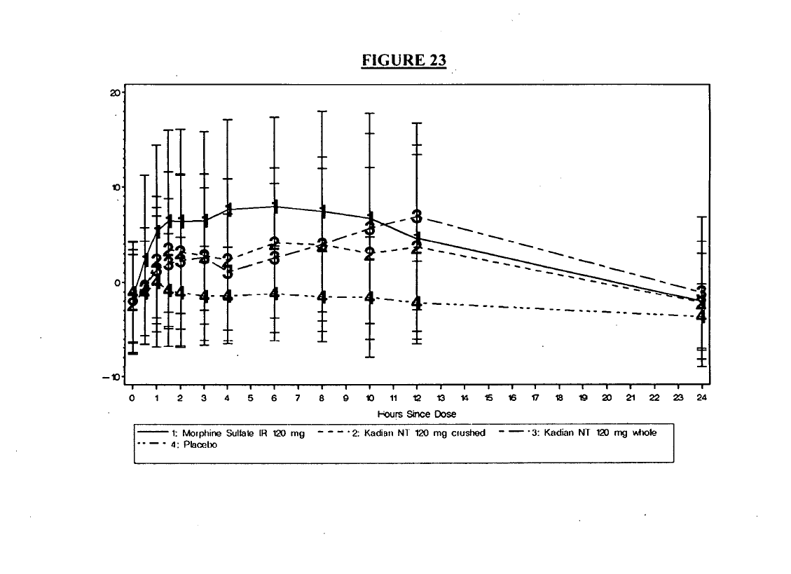

The ARCI-PCAG scale reflects sedation and intoxication. This scale is comprised of 15 questions, with 11 weighted as positive in scoring. Scores for this scale can range from −12 to 33. Descriptive statistics for ARCI-PCAG raw scores and summary parameters (per protocol population) were generated. Analysis of covariance for ARCI-PCAG Eₘₐₓ, AUE₍₀₋₂ₕ₎, AUE₍₀₋₈ₕ₎, AUE₍₀₋₂₄ₕ₎, and at 1.5 hours post-dose (HR1.5) were also completed. ARCI-PCAG mean (SD) raw scores plotted over time for the per protocol population are illustrated below in FIG. 23. ARCI-PCAG box plots for Eₘₐₓ, TEₘₐₓ, AUE₍₀₋₂ₕ₎, AUE₍₀₋₈ₕ₎, AUE₍₀₋₂₄ₕ₎ and at the 1.5 hours post-dosing time point were determined for each treatment group.

The proportion of subjects from the ALO-01 whole and ALO-01 crushed who had a 10-100% reduction in post-dose ARCI-PCAG scores Eₘₐₓ compared to MSIR are listed in below in Table 68. Relative to Eₘₐₓ for the MSIR treatment, the majority of subjects from the ALO-01 whole treatment experienced at least 10% reduction in ARCI-PCAG Eₘₐₓ (56.3% [ 18/32]). At least 10% reduction in ARCI-PCAG Eₘₐₓ was also reported by 15 of 32 subjects (40.6%) from the ALO-01 crushed treatment. The highest reductions were seen as a 100% reduction in both the ALO-01 whole group and in the ALO-01 crushed group (12.5% [ 4/32] and 3.1% [ 1/32], respectively).

Summary parameters of ARCI-PCAG for the per protocol population are listed below in Table 69. The Eₘₐₓ ranged from a mean (SD) of 2.3 (7.16) in the Placebo group to 13.6 (9.73) in the MSIR group. The Eₘₐₓ mean [SD] for ALO-01 crushed and ALO-01 whole groups was similar (10.3 [8.70] and 10.6 [9.69], respectively). Generally, for most parameters the lowest values were seen in the Placebo treatment and the highest in the MSIR treatment, with the exception of TEₘₐₓ which was the highest value was for the ALO-01 whole treatment followed by ALO-01 crushed, MSIR, and Placebo. AUE₍₀₋₂ₕ₎, AUE₍₀₋₈ₕ₎, and HR1.5 mean ARCI-PCAG score were greater for ALO-01 crushed than for ALO-01 whole, while for AUE₍₀₋₂₄ₕ₎ the reversed pattern was observed.

The analysis of covariance revealed a significant treatment effect for ARCI-PCAG Eₘₐₓ, AUE₍₀₋₂ₕ₎, AUE₍₀₋₈ₕ₎, AUE₍₀₋₂₄ₕ₎, and at 1.5 hours post-dose (P<0.001). For Eₘₐₓ, AUE₍₀₋₈ₕ₎, and AUE₍₀₋₂₄ₕ₎, all treatments contrasts against Placebo reached statistical significance (P≦0.002). Additionally, for AUE₍₀₋₈ₕ₎ MSIR was significantly different than ALO-01 whole (P<0.001) and ALO-01 crushed (P=0.001). AUE₍₀₋₂ₕ₎ was found to be significantly different between ALO-01 crushed vs. Placebo (P=0.036) and MSIR vs. all treatments (P≦0.027), while HR1.5 was found to be significantly different between ALO-01 crushed vs. Placebo (P=0.012) and MSIR vs. Placebo (P<0.001) and ALO-01 whole (P=0.009).

TABLE 68
Proportion of subjects (per protocol population) who had a 10-100%
reduction in post-dose ARCI-PCAG Eₘₐₓ compared to Morphine
Sulfate IR 120 mg

| | |
|---|---|
| ALO-01 120 mg crushed | ALO-01 120 mg |
| (N = 32) | whole (N = 32) |

Eₘₐₓ of ARCI-Pent. Chlorpromazine Alcohol (PCAG)

| | | |
|---|---|---|
| At least 10% reduction | 15 (46.9%) | 18 (56.3%) |
| At least 20% reduction | 15 (46.9%) | 14 (43.8%) |
| At least 30% reduction | 13 (40.6%) | 10 (31.3%) |
| At least 40% reduction | 9 (28.1%) | 9 (28.1%) |
| At least 50% reduction | 6 (18.8%) | 9 (28.1%) |
| At least 60% reduction | 5 (15.6%) | 8 (25.0%) |
| At least 70% reduction | 4 (12.5%) | 7 (21.9%) |
| At least 80% reduction | 1 (3.1%) | 6 (18.8%) |
| At least 90% reduction | 1 (3.1%) | 4 (12.5%) |
| At least 100% | 1 (3.1%) | 4 (12.5%) |
| reduction | | |

Note:
Percentage is calculated based on the number of subjects in the Per Protocol Population as the denominator

TABLE 69
ARCI-PCAG descriptive statistics of summary parameters for the per protocol
population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | |
|---|---|---|---|---|
| Mean (SD) | 2.3 (7.16) | 10.6 (9.69) | 10.3 (8.70) | 13.6 (9.73) |
| Median | 0.0 | 9.0 | 9.5 | 13.5 |
| Range | −6-27 | −6-33 | −6-33 | −5-33 |

TEₘₐₓ

| | | | | |
|---|---|---|---|---|
| Mean (SD) | 2.481 (3.0685) | 6.235 (4.3934) | 6.091 (6.0130) | 4.874 (3.5186) |
| Median | 1.000 | 6.000 | 3.992 | 4.000 |
| Range | 0.48-12.00 | 0.50-12.00 | 0.50-24.00 | 0.50-12.00 |

AUE_{(0-2 h)}

| | | | | |
|---|---|---|---|---|
| Mean (SD) | −1.523 (10.7766) | 1.581 (11.6119) | 2.749 (10.9392) | 8.269 (14.8590) |
| Median | −0.371 | 0.129 | 0.625 | 9.821 |
| Range | −18.00-34.78 | −13.73-37.09 | −15.50-28.25 | −17.92-43.63 |

AUE_{(0-8 h)}

| | | | | |
|---|---|---|---|---|
| Mean (SD) | −9.608 (36.5745) | 16.109 (49.2863) | 22.856 (48.0594) | 52.798 (63.4020) |
| Median | −11.750 | 6.721 | 11.475 | 46.875 |
| Range | −54.00-105.96 | −66.73-150.09 | −69.50-149.75 | −48.88-220.00 |

AUE_{(0-24 h)}

| | | | | |
|---|---|---|---|---|
| Mean (SD) | −51.158 (95.7282) | 73.689 (156.9838) | 46.169 (135.6071) | 94.562 (177.2918) |
| Median | −64.975 | 33.138 | 16.458 | 51.746 |
| Range | −169.23-253.23 | −202.59-654.63 | −213.50-478.75 | −167.00-675.08 |

HR1.5

| | | | | |
|---|---|---|---|---|
| Mean (SD) | −0.8 (5.97) | 2.0 (6.81) | 3.5 (8.10) | 6.4 (9.58) |
| Median | −0.5 | 0.5 | 0.5 | 7.0 |
| Range | −9-19 | −7-24 | −9-23 | −12-26 |

Note:
Pre-dose time set to 0.0 hr for AUE calculation

### Cole/ARCI-Sedation-Mental

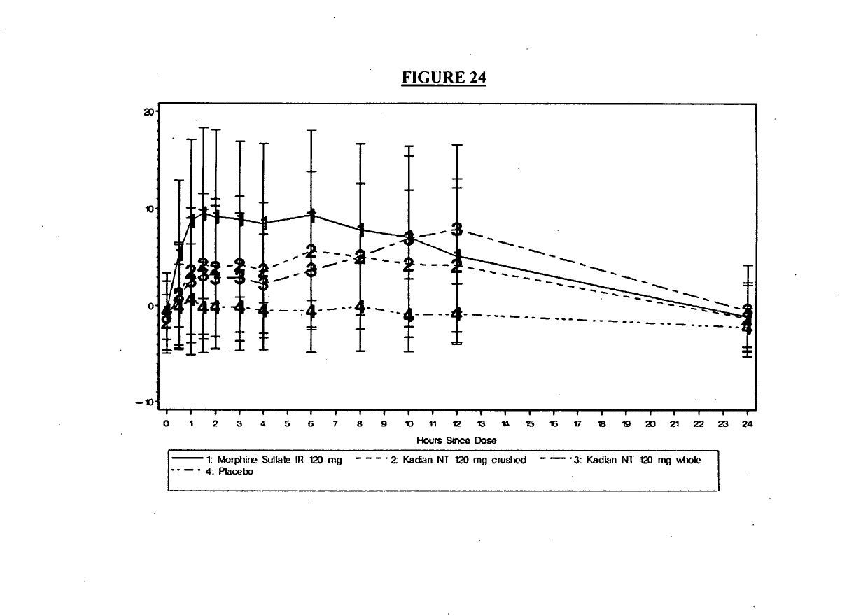

The Cole/ARCI-Sedation-Mental scale is comprised of 11 questions, 9 of which are weighted as positive in scoring. Scores for this scale can range from −6 to 27. Descriptive statistics for Cole/ARCI-Sedation-Mental raw scores and summary parameters (per protocol population) were generated. Analysis of covariance for Cole/ARCI-Sedation-Mental Eₘₐₓ AUE₍₀₋₂ₕ₎, AUE₍₀₋₈ₕ₎, AUE₍₀₋₂₄ₕ₎, and at 1.5 hours post-dose (HR1.5) were completed. Cole/ARCI-Sedation-Mental mean (SD) raw scores plotted over time for the per protocol population are illustrated below in FIG. 24. Cole/ARCI-Sedation-Mental box plots for Eₘₐₓ, TEₘₐₓ, AUE₍₀₋₂ₕ₎, AUE₍₀₋₈ₕ₎, AUE₍₀₋₂₄ₕ₎, and HR1.5 were calculated for each treatment group.

The proportion of subjects who had a 10-100% reduction in Eₘₐₓ after administration of ALO-01 whole or crushed compared to Eₘₐₓ after MSIR administration are listed below in Table 70. Relative to Eₘₐₓ for the MSIR treatment, the majority of subjects from the ALO-01 whole treatment (50.0% [ 16/32]) experienced at least 20% reduction in Cole/ARCI-Sedation-Mental Eₘₐₓ, while the majority of subjects from the ALO-01 crushed treatment (50.0% [ 16/32]) experienced at least 30% reduction in Cole/ARCI-Sedation-Mental Eₘₐₓ. The highest reductions were seen as a 100% reduction in the ALO-01 whole group (12.5% [ 4/32] and in the ALO-01 crushed group (6.2% [ 2/32], respectively).

Summary parameters of Cole/ARCI-Sedation-Mental for the per protocol population are listed below in Table 71. The Eₘₐₓ ranged from a mean (SD) of 3.1 (5.84) in the Placebo group to 14.3 (8.17) in the MSIR group. The Eₘₐₓ mean [SD] for ALO-01 whole and ALO-01 crushed treatments were similar (10.9 [8.54] and 10.7 [7.61], respectively). Generally, for most parameters the lowest values were seen in the Placebo treatment and the highest in the MSIR treatment, with the exception of TE, which was lowest for Placebo followed by MSIR, ALO-01 whole, and ALO-01 crushed treatment. The AUE₍₀₋₂ₕ₎, AUE₍₀₋₈ₕ₎, and HR1.5 were lower for ALO-01 whole than ALO-01 crushed treatment; however, the pattern was reversed for AUE₍₀₋₂₄ₕ₎.

The analysis of covariance revealed significant treatment effects for Eₘₐₓ, AUE₍₀₋₂ₕ₎, AUE₍₀₋₈ₕ₎, AUE₍₀₋₂₄ₕ₎, and HR1.5 (P<0.001). For Eₘₐₓ, AUE₍₀₋₂ₕ₎, AUE₍₀₋₈ₕ₎, AUE₍₀₋₂₄ₕ₎, and HR1.5 all treatment contrasts reached statistical significance (P≦0.040) except for ALO-01 whole vs. ALO-01 crushed treatment contrasts (P≧0.242), for AUE₍₀₋₂ₕ₎ ALO-01 whole vs. Placebo treatment contrast (P=0.071), and for AUE₍₀₋₂₄ₕ₎ MSIR vs.AL0-01 whole (P=0.356).

TABLE 70
For Cole/ARCI-Sedation-Mental, the proportion of subjects (per
protocol population )who had a 10-100% reduction in post-dose Eₘₐₓ
compared to Morphine Sulfate IR 120 mg

| | |
|---|---|
| ALO-01 120 mg crushed | ALO-01 120 mg |
| (N = 32) | whole (N = 32) |

Eₘₐₓ of Cole/ARCI-Sedation-Mental

| | | |
|---|---|---|
| At least 10% reduction | 21 (65.6%) | 18 (56.3%) |
| At least 20% reduction | 17 (53.1%) | 16 (50.0%) |
| At least 30% reduction | 16 (50.0%) | 11 (34.4%) |
| At least 40% reduction | 14 (43.8%) | 10 (31.3%) |
| At least 50% reduction | 9 (28.1%) | 7 (21.9%) |
| At least 60% reduction | 4 (12.5%) | 6 (18.8%) |
| At least 70% reduction | 3 (9.4%) | 6 (18.8%) |
| At least 80% reduction | 2 (6.3%) | 6 (18.8%) |
| At least 90% reduction | 2 (6.3%) | 4 (12.5%) |
| At least 100% | 2 (6.3%) | 4 (12.5%) |
| reduction | | |

Note:
Percentage is calculated based on the number of subjects in the Per Protocol Population as the denominator

TABLE 71
Cole/ARCI-Sedation-Mental descriptive statistics of summary parameters for the per
protocol population (N = 32)

| | | | |
|---|---|---|---|
| | ALO-01 120 mg | ALO-01 120 mg | Morphine Sulfate |
| Placebo | whole | crushed | IR 120 mg |

Eₘₐₓ

| | | | | |
|---|---|---|---|---|
| Mean (SD) | 3.1 (5.84) | 10.9 (8.54) | 10.7 (7.61) | 14.3 (8.17) |
| Median | 0.5 | 12.0 | 8.0 | 15.0 |
| Range | −4-18 | −4-27 | 0-27 | −1-27 |

TEₘₐₓ

| | | | | |
|---|---|---|---|---|
| Mean (SD) | 2.636 (3.0685) | 5.860 (4.2800) | 6.357 (5.8374) | 3.921 (3.2980) |
| Median | 1.000 | 6.000 | 5.992 | 2.000 |
| Range | 0.48-12.00 | 0.48-12.02 | 0.50-24.00 | 0.50-12.00 |

AUE_{(0-2 h)}

| | | | | |
|---|---|---|---|---|
| Mean (SD) | 0.027 (7.9769) | 3.894 (10.4195) | 5.123 (10.0581) | 14.003 (13.0374) |
| Median | −0.129 | 1.888 | 2.871 | 14.683 |
| Range | −9.50-24.99 | −9.24-41.50 | −7.75-29.25 | −8.69-38.70 |

AUE_{(0-8 h)}

| | | | | |
|---|---|---|---|---|
| Mean (SD) | −1.934 (27.8233) | 24.362 (37.9097) | 33.477 (46.2672) | 66.933 (55.4737) |
| Median | −0.221 | 16.625 | 15.996 | 63.971 |
| Range | −38.50-112.99 | −42.24-122.78 | −42.00-139.93 | −21.18-190.50 |

AUE_{(0-24 h)}

| | | | | |
|---|---|---|---|---|
| Mean (SD) | −22.768 (63.7635) | 96.522 (110.7878) | 69.321 (115.3565) | 119.120 (135.8828) |
| Median | −28.375 | 77.625 | 27.763 | 88.575 |
| Range | −111.23-172.99 | −122.16-344.78 | −114.00-319.77 | −99.25-425.54 |

HR1.5

| | | | | |
|---|---|---|---|---|
| Mean (SD) | −0.2 (4.64) | 3.3 (6.65) | 4.3 (7.24) | 9.5 (8.74) |
| Median | −0.5 | 0.5 | 2.0 | 10.0 |
| Range | −5-16 | −4-25 | −5-23 | −5-26 |

Note:
Pre-dose time set to 0.0 hr for AUE calculation

### Sedation-Motor Scale

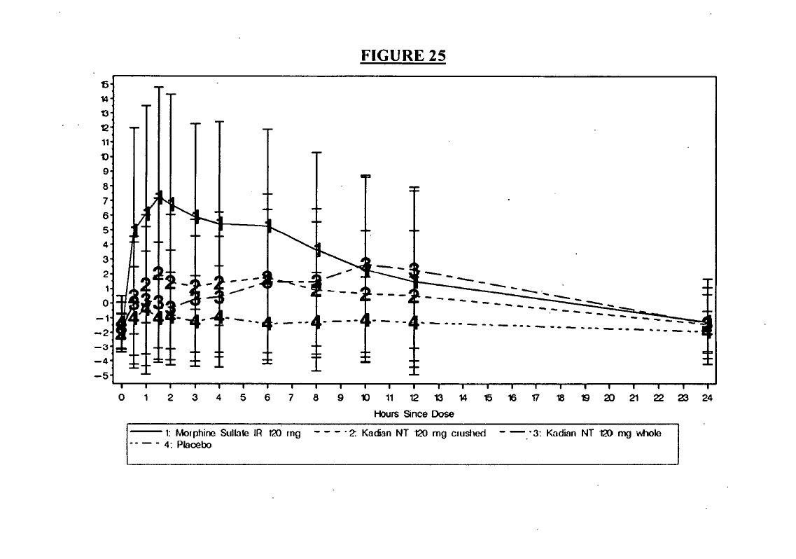

The Sedation-Motor scale is comprised of 10 questions, 9 of which are weighted as positive in scoring. Scores for this scale can range from −3 to 27. Descriptive statistics for Cole/ARCI Sedation-Motor raw scores and summary parameters were generated. Analysis of covariance for Cole/ARCI Sedation-Mental Eₘₐₓ, AUE₍₀₋₂ₕ₎, AUE₍₀₋₈ₕ₎, AUE₍₀₋₂₄ₕ₎, and at 1.5 hours post-dose (HR1.5) were determined. Cole/ARCI Sedation-Motor mean (SD) (raw scores) plotted over time for the per protocol population are illustrated in FIG. 25. Cole/ARCI Sedation-Motor box plots for E., TEₘₐₓ, AUE₍₀₋₂ₕ₎, AUE₍₀₋₈ₕ₎, AUE₍₀₋₂₄ₕ₎, and HR1.5 were calculated for each treatment group.

The proportion of subjects who had a 10-100% reduction in Eₘₐₓ after administration of ALO-01 whole or ALO-01 crushed compared to Eₘₐₓ after MSIR administration are listed below in Table 72. Relative to Eₘₐₓ for the MSIR treatment, the majority of subjects from the ALO-01 whole and ALO-01 crushed treatments (53.1% [ 17/32] and 50.0% [ 16/32]) experienced at least 50% reduction in Cole/ARCI Sedation-Motor Eₘₐₓ. The highest reductions were seen as a 100% reduction in the ALO-01 whole group and in the ALO-01 crushed group (28.1% [ 9/32] and 21.9% [ 7/32] subjects, respectively).

Summary parameters of Cole/ARCI Sedation-Motor for the per protocol population are listed below in Table 73. The Eₘₐₓ ranged from a mean (SD) of 0.7 (3.83) in the Placebo group to 10.0 (7.64) in the MSIR group. The Eₘₐₓ mean [SD] for ALO-01 whole and ALO-01 crushed treatments was the same (5.0 [6.29] and 5.0 [5.54], respectively). Generally, for most parameters the lowest values were seen in the Placebo treatment, and the highest in the MSIR treatment, with the exception of TEₘₐₓ, which was lowest for MSIR followed by Placebo, ALO-01 crushed, and ALO-01 whole treatment. The AUE₍₀₋₂ₕ₎, AUE₍₀₋₈ₕ₎, and HR1.5 were lower for ALO-01 whole than ALO-01 crushed treatment; however, the pattern was reversed for AUE₍₀₋₂₄ₕ₎.

The analysis of covariance revealed significant treatment effects for Eₘₐₓ, AUE₍₀₋₂ₕ₎) AUE₍₀₋₈ₕ₎, AUE₍₀₋₂₄ₕ₎, and HR1.5 (P<0.001). For Eₘₐₓ, AUE₍₀₋₂ₕ₎, AUE₍₀₋₈ₕ₎, AUE₍₀₋₂₄ₕ₎, and HR1.5 all treatment contrasts reached statistical significance (P≦0.018) except for ALO-01 whole vs. ALO-01 crushed treatment contrasts (P≧0.0.51), for AUE₍₀₋₂ₕ₎ ALO-01 whole vs. Placebo treatment contrast (P=0.322), and for 1.5 hours post dose ALO-01 whole vs. Placebo (P=0.279).

TABLE 72
For Cole/ARCI-Sedation-Motor, the proportion of subjects (per protocol
population) who had a 10-100% reduction in post-dose Eₘₐₓ compared to
Morphine Sulfate IR 120 mg

| | |
|---|---|
| ALO-01 120 mg crushed | ALO-01 120 mg |
| (N = 32) | whole (N = 32) |

Emax of Cole/ARCI Sedation-Motor

| | | |
|---|---|---|
| At least 10% reduction | 23 (71.9%) | 24 (75.0%) |
| At least 20% reduction | 23 (71.9%) | 21 (65.6%) |
| At least 30% reduction | 22 (68.8%) | 19 (59.4%) |
| At least 40% reduction | 17 (53.1%) | 18 (56.3%) |
| At least 50% reduction | 16 (50.0%) | 17 (53.1%) |
| At least 60% reduction | 13 (40.6%) | 13 (40.6%) |
| At least 70% reduction | 11 (34.4%) | 12 (37.5%) |
| At least 80% reduction | 9 (28.1%) | 11 (34.4%) |
| At least 90% reduction | 7 (21.9%) | 11 (34.4%) |
| At least 100% reduction | 7 (21.9%) | 9 (28.1%) |

Note:
Percentage is calculated based on the number of subjects in the Per Protocol Population as the denominator.

TABLE 73
Cole/ARCI-Sedation-Motor descriptive statistics of summary parameters for the per
protocol population (N = 32)

| | | | |
|---|---|---|---|
| | ALO- | ALO- | Morphine Sulfate |
| Placebo | 01 120 mg whole | 01 120 mg crushed | IR 120 mg |

Eₘₐₓ

| | | | | |
|---|---|---|---|---|
| Mean (SD) | 0.7 (3.83) | 5.0 (6.29) | 5.0 (5.54) | 10.0 (7.64) |
| Median | 0.0 | 2.5 | 3.0 | 9.0 |
| Range | −3-14 | −3-16 | −3-17 | −1-23 |

T_{Emax}

| | | | | |
|---|---|---|---|---|
| Mean (SD) | 2.388 (4.5379) | 6.250 (5.2701) | 4.297 (4.8831) | 2.219 (2.2297) |
| Median | 1.000 | 6.000 | 2.000 | 1.500 |
| Range | 0.48-24.00 | 0.48-24.00 | 0.50-24.00 | 0.48-10.00 |

AUE_{(0-2 h)}

| | | | | |
|---|---|---|---|---|
| Mean (SD) | −1.793 (5.6648) | −0.420 (7.4870) | 1.722 (7.5759) | 10.307 (11.9729) |
| Median | −3.500 | −2.625 | −1.500 | 8.663 |
| Range | −6.00-23.25 | −6.00-22.00 | −6.00-21.89 | −6.00-36.76 |

AUE_{(0-8 h)}

| | | | | |
|---|---|---|---|---|
| Mean (SD) | −9.005 (22.5945) | 4.701 (28.5359) | 10.117 (33.0805) | 41.970 (45.8979) |
| Median | −15.750 | −5.496 | 0.513 | 36.663 |
| Range | −24.00-91.70 | −24.00-75.00 | −23.95-117.65 | −14.50-141.06 |

AUE_{(0-24 h)}

| | | | | |
|---|---|---|---|---|
| Mean (SD) | −33.400 (58.8111) | 19.203 (81.8467) | 7.147 (80.6984) | 53.003 (112.7733) |
| Median | −48.367 | −4.625 | −14.771 | 17.433 |
| Range | −72.00-205.70 | −72.00-203.43 | −72.00-273.50 | −61.49-336.39 |

HR1.5

| | | | | |
|---|---|---|---|---|
| Mean (SD) | −1.0 (2.89) | 0.0 (4.13) | 2.0 (5.13) | 7.2 (7.58) |
| Median | −2.0 | −1.0 | 0.0 | 7.0 |
| Range | −3-13 | −3-13 | −3-15 | −3-22 |

Note:
Pre-dose time set to 0.0 hr for AUE calculation

### Summary of Pharmacodynamic Studies

The objective of this study was to determine the relative pharmacodynamic effects of crushed and whole ALO-01 (120 mg) compared to Morphine Sulfate IR (120 mg) and Placebo and of crushed ALO-01 to whole ALO-01. Therefore, the pharmacodynamic results have been organized primarily by pharmacologic effects, with the emphasis on the positive effects (as assessed by VAS-Liking, VAS-High, VAS-Good Effects, Subjective Drug Value, ARCI-Morphine Benzedrine Group, Cole\ARCI-Stimulation-Euphoria, and Cole\ARCI-Abuse Potential). Administration of MSIR resulted in a characteristic and expected increase for the positive effects scales. The mean positive effects for the MSIR treatment peaked sharply at approximately 1.5 hours post-dose and were significantly elevated in comparison to the placebo induced positive effect, thus, confirming the validity of this study. Administration of ALO-01 whole and crushed resulted in lower level of response and flatter profile on measures of the positive effects than administration of MSIR. That is, the release of naltrexone in the crushing process resulted in Eₘₐₓ lower than Eₘₐₓ for MSIR; however, the TEₘₐₓ for both treatments was similar. Such a response pattern is indicative of ALO-01 whole and crushed having a lower abuse potential than MSIR. Generally, the distinct response patterns were confirmed by the significant treatment effects and treatment contrasts between MSIR vs. ALO-01 whole and crushed on all measures and all variables (maximum effect [Eₘₐₓ], area under the response curve 0-2 h post-dose [AUE₍₀₋₂ₕ₎], 0-8 h post-dose [AUE₍₀₋₈ₕ₎], 0-24 h post-dose [AUE₍₀₋₂₄ₕ₎], and at the 1.5 hours post-dose time point [HR1.5]). Overall, treatment differences between ALO-01 crushed vs. whole were not significant suggesting similar abuse potential. A summary of the Eₘₐₓ treatment effects and contrasts for each measure is displayed in Table 74.

Examination of the negative drug effect measures (as assessed by VAS-Bad Effects, VAS-Feel Sick, VAS-Nausea, ARCI-LSD, Cole/ARCI-Unpleasantness Physical and Cole/ARCI-Unpleasantness-Dysphoria) indicated that administration of MSIR was associated with a strong negative response that peaked at approximately 6.0 hours post-dose. Administration of ALO-01 whole and crushed induced similar levels of negative response; the response levels were lower than those seen after administration of MSIR but higher than after administration of Placebo.

The patterns of responses on the measures of other drug effects were similar to the positive and negative measures. Examination of pupillometry, a measure of opiate physiologic effect, demonstrated characteristic morphine induced miosis following administration of MSIR. Administration of ALO-01 whole and crushed resulted in less pupillary constriction, presumably because of the slow morphine release due to the extended release formulation (ALO-01 whole condition) and the release of naltrexone (ALO-01 crushed condition). No significant differences between the ALO-01 whole and crushed treatments were observed.

TABLE 74
A summary of the Eₘₐₓ treatment effects and contrasts for measure of positive effects
and pupillometry

| | | VAS- | Cole | | Cole AR | | | | |
|---|---|---|---|---|---|---|---|---|---|
| | VAS- | Overall | ARCI- | Subjective | CI- | | VAS- | VAS- | Pupil |
| Treatment | Drug | Drug | Stimulation | Drug | Abuse | ARCI- | Good | Feeling | Diameter |
| effect | Liking | Liking | Euphoria | Value | Potential | MBG | Effects | High | (PCmin) |
| ALO-01 | <.001 | 0.006 | 0.007 | <.001 | <.001 | 0.002 | <.001 | <.001 | <.001 |
| crushed | | | | | | | | | |
| vs. Placebo | | | | | | | | | |
| ALO-01 | <.001 | 0.011 | 0.056 | <.001 | <.001 | 0.068 | <.001 | <.001 | <.001 |
| whole | | | | | | | | | |
| vs. Placebo | | | | | | | | | |
| MSIR | <.001 | <.001 | <.001 | <.001 | <.001 | <.001 | <.001 | <.001 | <.001 |
| vs. Placebo | | | | | | | | | |
| MSIR | <.001 | <.001 | <.001 | <.001 | 0.002 | <.001 | <.001 | <.001 | <.001 |
| vs. ALO-01 | | | | | | | | | |
| crushed | | | | | | | | | |
| MSIR | <.001 | <.001 | <.001 | <.001 | <.001 | <.001 | <.001 | <.001 | <.001 |
| vs. ALO-01 | | | | | | | | | |
| whole | | | | | | | | | |
| ALO-01 | 0.875 | 0.868 | 0.458 | 0.875 | 0.562 | 0.215 | 0.216 | 0.335 | 0.262 |
| crushed | | | | | | | | | |
| vs. ALO-01 | | | | | | | | | |
| whole | | | | | | | | | |

### Pharmacokinetic Studies

Throughout the study the levels of morphine, naltrexone and 6-β-naltrexol were measured. The analyses of the pharmacokinetic results were based on summary statistics and analysis of variance. Pharmacokinetic parameters for morphine, naltrexone, and 6-β-naltrexol, including Cₘₐₓ, Tₘₐₓ, area under the curve from 0-8 hours post-dose (AUC₍₀₋₈ₕ₎), area under the curve to the last measurement (AUCₗₐₛₜ), area under the curve to infinity (AUCᵢₙₜ), half life (t_{1/2}), elimination rate (kₑ), clearance (for morphine and naltrexone only), and volume of distribution (for morphine and naltrexone only) were determined.

Serial blood samples (10 mL each) for determination of plasma concentration of morphine, naltrexone and 6-β-naltrexol were taken in each treatment session approximately 1 hour pre-dose and at approximately 0.5, 1, 1.5, 2, 3, 4, 6, 8, 10, 12, and 24 hours post-dose. The blood samples were obtained with an intravenous catheter or by direct venipuncture. The total volume of blood drawn from each subject during this study for pharmacokinetic analysis was approximately 480 mL. The blood samples were drawn in K2 EDTA tubes. The time and date of collection for each sample were recorded. Blood samples were placed on ice prior to being centrifuged. The samples were centrifuged under the following approximated conditions: 3000 rpm for 15 minutes at 4° C. and placed on ice. The resulting plasma samples ware harvested and transferred into appropriately labeled polypropylene screw-cap tubes and placed in a storage freezer at approximately −20° C. or colder, within 60 minutes of blood draw.

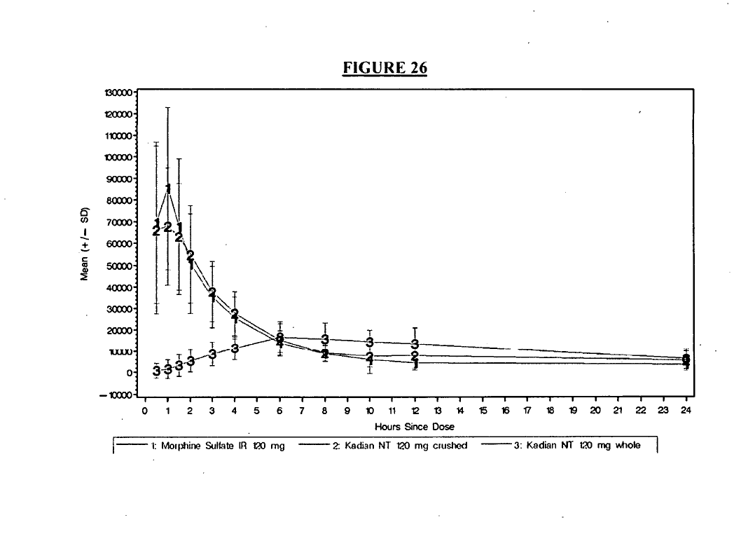

Morphine mean plasma concentrations over time were calculated and are shown in FIG. 26. The time course data demonstrates that for MSIR and ALO-01 crushed, morphine concentration increased sharply within the first hour post-dosing followed by a gradual decline over the next 5 hours, while administration of ALO-01 whole resulted in slow and stable release of morphine. A summary of the estimated parameters for morphine plasma concentrations are displayed in Table 75. There was a significant treatment effect for Cₘₐₓ, AUC₍₀₋₈ₕ₎, AUCₗₐₛₜ and AUC_{inf} (P≦0.037). Cₘₐₓ (pg/mL) ranged from mean (SD) of 92515.6 (38051.35) for MSIR to 19256.3 (7682.99) for ALO-01 whole. Mean Cₘₐₓ, for ALO-01 crushed was 80587.5 (38804.53). All examined treatment contrasts (ALO-01 crushed vs. MSIR, ALO-01 whole vs. MSIR and ALO-01 crushed vs. ALO-01 whole) were significantly different (P<0.037). Mean AUC₍₀₋₈ₕ₎ was the highest for ALO-01 crushed followed by ALO-01 whole, while mean AUCₗₐₛₜ for the MSIR treatment was the highest followed by ALO-01 crushed and ALO-01 whole. For both MSIR and ALO-01 crushed treatments, contrasts against ALO-01 whole were significant (P<0.001). Mean AUC_{inf} for ALO-01 crushed was the highest followed by the mean for ALO-01 whole and mean for MSIR; however, only the contrast between ALO-01 crushed and MSIR was statistically significant (P=0.011). Median Tₘₐₓ (hours) was similar between ALO-01 crushed and MSIR (1.109 and 1.150, respectively) and lower than for ALO-01 whole (8.125).

Examination of morphine bioavailability for ALO-01 whole and ALO-01 crushed indicated that for all parameters (Cₘₐₓ, AUC₍₀₋₈ₕ₎, AUCₗₐₛₜ, and AUC_{inf}) morphine bioavailability for ALO-01 crushed was greater than for ALO-01 whole; however, the differences were consistently diminishing from AUC₍₀₋₈ₕ₎ to AUC_{inf}.

TABLE 75
Pharmacokinetics of Morphine for the per protocol population

| | | | |
|---|---|---|---|
| Parameter/ | ALO-01 120 mg | ALO-01 120 mg | Morphine Sulfate IR |
| Statistics | Whole | crushed | 120 mg |

Cₘₐₓ (pg/mL)

| | | | |
|---|---|---|---|
| N | 32 | 32 | 32 |
| Mean (SD) | 19256.3 (7682.99) | 80587.5 (38804.53) | 92515.6 (38051.35) |
| Range | 8000 to 46600 | 25500 to 212000 | 30900 to 184000 |
| Median | 18150.0 | 75850.0 | 88350.0 |
| Geo Mean | 17946.9 | 72556.8 | 84367.3 |
| Geo. CV | 39.3 | 49.8 | 48.0 |

Tₘₐₓ (h)

| | | | |
|---|---|---|---|
| N | 32 | 32 | 32 |
| Range | 4.07 to 12.23 | 0.58 to 2.18 | 0.62 to 2.07 |
| Median | 8.125 | 1.109 | 1.150 |
| Lower Quartile | 6.133 | 0.642 | 1.109 |
| Upper Quartile | 10.117 | 1.775 | 1.183 |

AUC_{(0-8 h)} (pg * h/mL)

| | | | |
|---|---|---|---|
| N | 32 | 32 | 32 |
| Mean (SD) | 80657.656 (42240.1222) | 259742.355 (90766.2669 | 262621.229 (92799.3352) |
| Range | 33440.93 to 261755.83 | 106374.56 to 486495.92 | 87858.77 to 502634.26 |
| Median | 70817.248 | 252638.154 | 253625.381 |
| Geo Mean | 72996.815 | 244583.086 | 246792.485 |
| Geo. CV | 45.9 | 37.0 | 38.0 |

AUClast (pg * h/mL)

| | | | |
|---|---|---|---|
| N | 32 | 32 | 32 |
| Mean (SD) | 251305.547 (121746.208) | 369205.307 (160735.649) | 334907.013 (109536.865) |
| Range | 124688.59 to 854649.12 | 157173.84 to 1020905.3 | 115815.86 to 596688.12 |
| Median | 238367.990 | 360352.715 | 342584.090 |
| Geo Mean | 235182.403 | 342499.577 | 316521.933 |
| Geo. CV | 34.6 | 40.1 | 36.6 |

AUC_{inf} (pg * h/mL)

| | | | |
|---|---|---|---|
| N | 26 | 31 | 30 |
| Mean (SD) | 427229.599 (327435.759 | 480740.612 (330135.446 | 362597.043 (119507.483 |
| Range | 184004.78 to 1782909.6 | 174851.83 to 1957378.2 | 135317.83 to 626522.61 |
| Median | 331119.981 | 397883.791 | 367024.253 |
| Geo Mean | 366419.855 | 420018.947 | 342447.335 |
| Geo. CV | 52.9 | 51.4 | 36.7 |

T_{1/2} (h)

| | | | |
|---|---|---|---|
| N | 26 | 31 | 30 |
| Mean (SD) | 17.658 (22.8910) | 11.695 (10.7395) | 5.872 (1.7219) |
| Range | 4.08 to 118.25 | 5.09 to 48.90 | 3.97 to 10.17 |
| Median | 10.997 | 6.944 | 5.358 |
| Geo Mean | 12.244 | 9.104 | 5.666 |
| Geo. CV | 87.5 | 71.1 | 26.7 |

Elimination Rate (kₑ) (l/h)

| | | | |
|---|---|---|---|
| N | 26 | 31 | 30 |
| Mean (SD) | 0.069994 (0.0412343) | 0.088477 (0.0385201) | 0.126178 (0.0297311) |
| Range | 0.00586 to 0.16974 | 0.01417 to 0.13630 | 0.06817 to 0.17455 |
| Median | 0.063030 | 0.099820 | 0.129385 |
| Geo Mean | 0.056608 | 0.076135 | 0.122344 |
| Geo. CV | 87.5 | 71.1 | 26.7 |

Clearance (L/h)

| | | | |
|---|---|---|---|
| N | 26 | 31 | 30 |
| Mean (SD) | 271.0042 (101.81168) | 237.7622 (103.80910) | 319.0701 (127.49074) |
| Range | 50.676 to 491.020 | 46.159 to 516.723 | 163.442 to 756.737 |
| Median | 272.8655 | 227.0760 | 279.1145 |
| Geo Mean | 246.5752 | 215.1093 | 299.0241 |
| Geo. CV | 52.9 | 51.4 | 36.7 |

Volume of Distribution (L)

| | | | |
|---|---|---|---|
| N | 26 | 31 | 30 |
| Mean (SD) | 4687.0434 (1762.90624) | 3301.8665 (2298.32248) | 2666.9694 (1199.09909) |
| Range | 2057.855 to 8645.214 | 1506.145 to 11963.810 | 1065.353 to 6501.261 |
| Median | 4388.1530 | 2241.6110 | 2444.5665 |
| Geo Mean | 4355.7654 | 2825.3713 | 2444.1278 |
| Geo. CV | 41.8 | 55.7 | 43.9 |

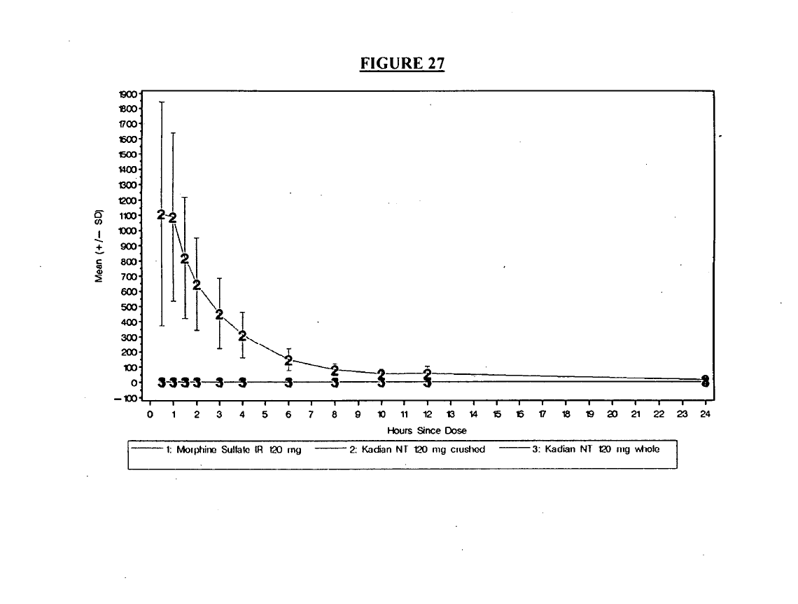

Summary statistics and estimated parameters of naltrexone concentration for the per protocol population were determined for each treatment group. A summary of the estimated parameters for naltrexone plasma concentrations are displayed in FIG. 27 and Table 76. Naltrexone was present in subjects from the ALO-01 crushed treatment, but only trace amounts of the substance were detected in 5 of 32 subjects from the ALO-01 whole treatment. Specifically, only 1 concentration just above the limit of quantification level was reported for each of the 5 subjects; thus, for ALO-01 whole pharmacokinetic parameters for naltrexone were not computed. For ALO-01 crushed, the naltrexone Cₘₐₓ, AUC₍₀₋₈ₕ₎, AUC_{inf}, elimination rate, clearance, and volume of distribution are within expected levels.

TABLE 76
Pharmacokinetics of Naltrexone for the per protocol population

| | |
|---|---|
| Parameter/ | |
| Statistics | ALO-01 120 mg crushed |

Cₘₐₓ (pg/mL)

| | |
|---|---|
| N | 32 |
| Mean (SD) | 1265.344 (706.3226) |
| Range | 316.00 to 3320.00 |
| Median | 1135.000 |
| Geo Mean | 1073.226 |
| Geo. CV | 67.2 |

Tₘₐₓ (h)

| | |
|---|---|
| N | 32 |
| Range | 0.58 to 1.17 |
| Median | 1.083 |
| Lower Quartile | 0.642 |
| Upper Quartile | 1.109 |

AUC_{(0-8 h)} (pg * h/mL)

| | |
|---|---|
| N | 32 |
| Mean (SD) | 3943.793 (1927.8448) |
| Range | 1488.80 to 10573.66 |
| Median | 3867.204 |
| Geo Mean | 3527.652 |
| Geo. CV | 51.7 |

AUCₗₐₛₜ (pg * h/mL)

| | |
|---|---|
| N | 32 |
| Mean (SD) | 3942.581 (1927.8376) |
| Range | 1487.60 to 10572.26 |
| Median | 3866.037 |
| Geo Mean | 3526.287 |
| Geo. CV | 51.7 |

AUC_{inf} (pg * h/mL)

| | |
|---|---|
| N | 32 |
| Mean (SD) | 4074.944 (1996.4402) |
| Range | 1564.67 to 11034.07 |
| Median | 3995.740 |
| Geo Mean | 3649.091 |
| Geo. CV | 51.2 |

t_{1/2} (h)

| | |
|---|---|
| N | 32 |
| Mean (SD) | 4.946 (1.8580) |
| Range | 2.16 to 10.41 |
| Median | 4.246 |
| Geo Mean | 4.639 |
| Geo. CV | 37.5 |

Elimination Rate (kₑ) (1/h)

| | |
|---|---|
| N | 32 |
| Mean (SD) | 0.159207 (0.0592789) |
| Range | 0.06661 to 0.32145 |
| Median | 0.163300 |
| Geo Mean | 0.149417 |
| Geo. CV | 37.5 |

Clearance (L/h)

| | |
|---|---|
| N | 32 |
| Mean (SD) | 1331.6917 (661.58377) |
| Range | 393.327 to 2773.750 |
| Median | 1086.8475 |
| Geo Mean | 1189.3373 |
| Geo. CV | 51.2 |

Volume of Distribution (L)

| | |
|---|---|
| N | 32 |
| Mean (SD) | 9965.0378 (7416.62034) |
| Range | 2276.209 to 34591.448 |
| Median | 7594.4395 |
| Geo Mean | 7959.8421 |
| Geo. CV | 75.2 |

Summary statistics and estimated parameters of 6-β-naltrexonol concentration for the per protocol population were determined for each treatment group. A summary of the estimated parameters for 6-β-naltrexonol plasma concentrations is displayed in Table 77. Naltrexone was present in subjects from the ALO-01 crushed treatment, but only trace amounts of the substance was detected in 14 subjects from the ALO-01 whole treatment. For 8 of the subjects at least 3 6-β-naltrexonol concentration values were obtained. For ALO-01 crushed, the naltrexone Cₘₐₓ, AUC₍₀₋₈ₕ₎, AUC_{inf}, elimination rate, clearance, and volume of distribution are within expected levels.

TABLE 77
Pharmacokinetics of 6-β-Naltrexol (pg/mL) for the per protocol population

| | | |
|---|---|---|
| Parameter/ | | |
| Statistics | ALO-01 120 mg whole | ALO-01 120 mg crushed |

Cₘₐₓ (pg/mL)

| | | |
|---|---|---|
| N | 14 | 32 |
| Mean (SD) | 12.1379 (14.67564) | 6958.4375 (2380.62219) |
| Range | 0.320 to 45.500 | 3200.000 to 11100.000 |
| Median | 8.1400 | 6645.0000 |
| Geo Mean | 3.9964 | 6540.8678 |
| Geo. CV | 552.6 | 38.0 |

Tₘₐₓ (h)

| | | |
|---|---|---|
| N | 14 | 32 |
| Range | 0.58 to 24.17 | 0.60 to 2.13 |
| Median | 2.667 | 1.100 |
| Lower Quartile | 2.083 | 0.900 |
| Upper Quartile | 24.100 | 1.150 |

AUC_{(0-8 h)} (pg * h/mL)

| | | |
|---|---|---|
| N | 14 | 32 |
| Mean (SD) | 82.301 (94.7646) | 50958.899 (14195.0200) |
| Range | 0.22 to 276.03 | 25638.11 to 77044.44 |
| Median | 36.587 | 51942.161 |
| Geo Mean | 22.750 | 48955.359 |
| Geo. CV | 1331.9 | 30.0 |

AUCₗₐₛₜ (pg * h/mL)

| | | |
|---|---|---|
| N | 14 | 32 |
| Mean (SD) | 80.634 (93.6902) | 50958.823 (14195.0253) |
| Range | 0.14 to 271.48 | 25638.02 to 77044.44 |
| Median | 34.835 | 51942.084 |
| Geo Mean | 20.012 | 48955.279 |
| Geo. CV | 1899.7 | 30.0 |

AUC_{inf} (pg * h/mL)

| | | |
|---|---|---|
| N | 7 | 32 |
| Mean (SD) | 136.847 (103.4176) | 73630.891 (19191.6446) |
| Range | 11.32 to 293.27 | 38238.44 to 116698.95 |
| Median | 133.400 | 73170.109 |
| Geo Mean | 93.024 | 71144.227 |
| Geo. CV | 157.9 | 27.6 |

t_{1/2} (h)

| | | |
|---|---|---|
| N | 7 | 32 |
| Mean (SD) | 49.818 (51.4851) | 16.447 (8.0876) |
| Range | 5.98 to 142.30 | 8.35 to 52.30 |
| Median | 26.942 | 13.893 |
| Geo Mean | 29.088 | 15.208 |
| Geo. CV | 173.1 | 38.7 |

Elimination Rate (kₑ) (1/h)

| | | |
|---|---|---|
| N | 7 | 32 |
| Mean (SD) | 0.040021 (0.0400307) | 0.048386 (0.0157226) |
| Range | 0.00487 to 0.11584 | 0.01325 to 0.08300 |
| Median | 0.025730 | 0.049890 |
| Geo Mean | 0.023828 | 0.045576 |
| Geo. CV | 173.2 | 38.7 |

As shown above, administration of ALO-01 crushed resulted in similar morphine pharmacokinetics as administration of MSIR and different than administration of ALO-01 whole. Specifically, for the ALO-01 crushed and the MSIR treatments AUC₍₀₋₈ₕ₎ and AUC_{inf} were statistically different from the ALO-01 whole treatment but not statistically different from each other. Although Cₘₐₓ for all the treatments were significantly different from each other, in comparison to MSIR (Cₘₐₓ) relative bioavailability of ALO-01 crushed was 94.3, while relative bioavailability of ALO-01 whole was 23.4. Median Tₘₐₓ was approximately 1 hour for ALO-01 crushed and MSIR and 8 hours for ALO-01 whole. Examination of naltrexone and 6-β-naltrexol pharmacokinetic profile revealed that only trace amounts of the substance was detected after administration of the ALO-01 whole treatment, and the pattern of results observed for the ALO-01 crushed treatment were within expected levels.

### Efficacy Conclusions (Pharmacodynamics and Pharmacokinetics)

The primary objective of this study was to determine the relative pharmacodynamic effects and safety of crushed and whole ALO-01 compared to Morphine Sulfate IR and to Placebo and of crushed ALO-01 to whole ALO-01. Pharmaceokinitecs was also studied.

To examine the pharmacodynamic effects, the results have been organized primarily by pharmacologic effects, with the emphasis on the positive effects (as assessed by VAS-Liking, VAS-High, VAS-Good Effects, Subjective Drug Value, ARCI-Morphine Benzedrine Group, Cole\ARCI-Stimulation-Euphoria, and Cole\ARCI-Abuse Potential). Administration of MSIR resulted in a characteristic and expected increase for the positive effects scales: the responses were significantly elevated in comparison to the Placebo induced positive effect, thus, confirming the validity of this study. Administration of ALO-01 whole and ALO-01 crushed resulted in lower level of response and flatter profile on measures of the positive effects than administration of MSIR. Such a response pattern is indicative of ALO-01 whole and crushed having a lower abuse potential than MSIR. The distinct response patterns were confirmed by the significant treatment effects and treatment comparisons between MSIR vs. ALO-01 whole and crushed on all measures and all variables (except for Cole/ARCI-Abuse Potential for AUE₍₀₋₂₄ₕ₎). Generally, treatment differences between ALO-01 crushed vs. whole were not significant, suggesting similar abuse potential. However, administration of ALO-01 induced positive subjective effects that were more similar to the Placebo induced effects than administration of ALO-01 crushed.

Overall, evaluation of the negative and other drug effects confirmed that the response patterns for ALO-01 whole and ALO-01 crushed were similar and less extreme than responses for the MSIR treatment. Examination of pupillometry, a measure of opiate physiologic effect, demonstrated characteristic morphine induced miosis following administration of MSIR. Administration of ALO-01 whole and crushed resulted in less pupillary constriction, presumably because of the slow morphine release due to the extended release formulation (ALO-01 whole condition) and the release of opiate agonist (ALO-01 crushed condition).

The most common side effects observed during this study were consistent with the expected profile of MSIR side effects and included euphoric mood, pruritus, somnolence, vomiting, and nausea. The most adverse events (AEs) were observed following MSIR treatment, Subjects administered ALO-01 crushed reported lower incidences and frequencies of AEs than subjects administered ALO-01 whole. All AEs experienced were mild to moderate in severity, and no subjects discontinued from the study because of an AE.

The secondary objective of this study was to compare pharmacokinetic measures including relative bioavailability of plasma morphine, naltrexone, and 6-β-naltrexol from crushed and whole ALO-01 compared to MSIR and from crushed ALO-01 to whole ALO-01. Administration of ALO-01 crushed resulted in comparable morphine pharmacokinetics as administration of MSIR and different than administration of ALO-01 whole. For instance, for the ALO-01 crushed and the MSIR treatments, AUC₍₀₋₈ₕ₎ and AUC_{inf} were statistically different (higher) from the ALO-01 whole treatment but not statistically different from each other. In comparison to MSIR, the Cₘₐₓ relative bioavailability of ALO-01 crushed was 94.3, while relative bioavailability of ALO-01 whole was 23.4. Median Tₘₐₓ was approximately 1 hour for ALO-01 crushed and MSIR and 8 hours for ALO-01 whole; Similar patterns were observed for AUC₍₀₋₈ₕ₎ and AUCₗₐₛₜ. Examination of the 6-β-naltrexol pharmacokinetic profile revealed that only trace amounts of the substance was detected after administration of the ALO-01 whole treatment, and the pattern of results observed for the ALO-01 crushed treatment was within expected levels. These pharmacokinetic results confirmed that tampering with ALO-01 destroyed the controlled release formulation and released sequestered morphine and naltrexone.

In conclusion, although the same amount of morphine sulfate (120 mg) was administered in the MSIR, ALO-01 whole, and ALO-01 crushed treatments, the naltrexone released after crushing ALO-01 significantly abated the morphine induced subjective effects. ALO-01 whole and crushed induced similar level of subjective effects on positive, as well as negative and other measures of drug effects, however, these subjective effects were lower than MSIR induced subjective effects and blunting of the subjective effects reflects decreased abuse potential in comparison to MSIR. ALO-01 after tampering (crushing) has comparable abuse potential as ALO-01 intact, since the dose of naltrexone included in the ALO-01 formulation is sufficient to abate the euphoria induced by the released morphine. Thus, crushing the ALO-01 formulation did not increase ALO-01 abuse potential.

While the present invention has been described in terms of the preferred embodiments, it is understood that variations and modifications will occur to those skilled in the art. Therefore, it is intended that the appended claims cover all such equivalent variations that come within the scope of the invention as claimed.

What is claimed is:

1. A multi-layer pharmaceutical composition comprising an antagonist in a first layer and an agonist in a second layer upon said first layer such that the antagonist is substantially sequestered when administered to a human being in an intact form, such that physical disruption of the dosage form decreases the euphoric effect of the agonist when administered to a person as compared to an immediate release agonist composition.

2. The composition of claim 1 wherein the euphoric effect is measured by Eₘₐₓ from a test selected from the group consisting of VAS-Drug Liking, VAS-Overall Drug Liking, Cole/ARCI-Stimulation Euphoria, Subjective Drug Value, Cole/ARCI Abuse Potential, ARCI-MBG, VAS-Good Effects, VAS-Feeling High, and pupillometry.

3. The composition of claim 1 wherein the Eₘₐₓ of at least one of the tests is reduced by a percentage selected from the group consisting of about 10%, about 20%, about 30%, about 40%, about 50%, about 60%, about 70%, about 80%, about 90% and about 100%.

4. The composition of claim 2 or claim 3 wherein the agonist is morphine.

5. The composition of claim 4 wherein the antagonist is naltrexone.

6. A multi-layer pharmaceutical composition comprising an antagonist in a first layer and an agonist in a second layer upon said first layer such that the antagonist is substantially sequestered when administered to a human being in an intact form, such that physical disruption of the dosage form alters the pharmacokinetic parameters of the dosage form as compared to the intact dosage form.

7. The composition of claim 6 wherein the pharmacokinetic parameter is selected from the group consisting of Cₘₐₓ, Tₘₐₓ, λ_{z}, T_{1/2}, AUC₀₋₈ₕ, AUCₗₐₛₜ, AUC_{inf}, elimination rate, clearance and volume of distribution (L).

8. The composition of claim 7 wherein the difference is calculated based on the mean or median of the pharmacokinetic parameter.

9. The composition of claim 8 wherein the difference is statistically significant.

10. The composition of claim 8 wherein the median Cₘₐₓ of the intact dosage form is less than one-half the median Cₘₐₓ of the intact dosage form.

11. The composition of claim 8 wherein the median Tₘₐₓ of the substantially disrupted dosage form is approximately one-seventh that of the intact dosage form.

12. The composition of claim 8 wherein the median AUC₍₀₋₈ₕ₎ of the intact dosage form is approximately one-third that of the intact dosage form.

13. The composition of claim 8 wherein the median T_{1/2} of the intact dosage form is greater than that of the intact dosage form.

14. The composition of claim 8 wherein the pharmacokinetic parameter is the mean or median of a measurement selected from the group consisting of Cₘₐₓ, Tₘₐₓ, AUC₍₀₋₈ₕ₎, and T_{1/2}.

15. The pharmaceutical composition of claim 7 wherein the Tₘₐₓ of the antagonist released from the disrupted composition following administration to a subject is approximately equivalent to the Tₘₐₓ of an equivalent amount of antagonist orally administered to the subject.

16. The pharmaceutical composition of claim 7 wherein the Tₘₐₓ of the antagonist released from the disrupted composition following administration to a subject is within approximately 30% of the Tₘₐₓ of an equivalent amount of antagonist orally administered to the subject.

17. The pharmaceutical composition of claim 7 wherein the Tₘₐₓ of the antagonist released from the disrupted composition following administration to a subject is within approximately 20% of the Tₘₐₓ of an equivalent amount of antagonist orally administered to the subject.

18. The pharmaceutical composition of claim 7 wherein the Tₘₐₓ of the antagonist released from the disrupted composition following administration to a subject is within approximately 10% of the Tₘₐₓ of an equivalent amount of antagonist orally administered to the subject.

19. The pharmaceutical composition of claim 6 wherein the Cₘₐₓ of the antagonist released from the disrupted composition following administration to a subject is approximately equivalent to the Cₘₐₓ of an equivalent amount of antagonist orally administered to the subject.

20. The pharmaceutical composition of claim 6 wherein the Cₘₐₓ of the antagonist released from the disrupted composition following administration to a subject is within approximately 30% of the Cₘₐₓ of an equivalent amount of antagonist orally administered to the subject.

21. The pharmaceutical composition of claim 6 wherein the Cₘₐₓ of the antagonist released from the disrupted composition following administration to a subject is within approximately 20% of the Cₘₐₓ of an equivalent amount of antagonist orally administered to the subject.

22. The pharmaceutical composition of claim 6 wherein the Cₘₐₓ of the antagonist released from the disrupted composition following administration to a subject is within approximately 10% of the Cₘₐₓ of an equivalent amount of antagonist orally administered to the subject.

23. The composition of claim 6 wherein the agonist is morphine.

24. The composition of claim 23 wherein the antagonist is naltrexone.
